SEQUENCE LISTING (26 sequences)

SEQ ID NO: 1 - DNA, 86592 bp, Homo sapiens
  misc_feature 38388..40388: 5′regulatory region
attattggaacaggccacacttgcgagggaagtccctgcctcagaaagattcagaaaagctagacagtcactggaagaacaattacaaccgcaagacggtcaaacactaaacaccgctatgcctcagaaccgtacagataatggccaaatagatggggctctgggcatttctgagagcacctgcctggtggcaccccatcctaatggaccatgccctccagtctccaagtggctcttcagagctcacatccgaacacctcctatgctacaggttcttctagccccaggttcccaaccaccccaaggccacagaggccagccccaactccatcttctacatgtgtcacaggaaactttctcatagtgctatttattatgtactgcgggggtgggggccatgtcataaaagaaatgtcctcccttttttattcatctccttctaacaagcatcaaagtctcagtcgctagcatgtgacttacagaagctctcatgggaacaagacaagaccatactgttaccgtgacactcacggcctccctgactggtttctgctgttgattctgcctcaaatgctcctcaaatgcaccttgctgctccgcctccaccctagagctcgcctgactgcccacttgcccgttaagagtcggcttaggcttcactcctgccagaaaggtcctgccaggtgctctcaacagtcaccccctcctgtggtctcacaaaaccccagcacctctcggtcactctctccctcctatctggttgtgactgtcttccatgctcacttagaagctctctgaggccaagaactgtgtgtactgttgcttctttgtttacctgggcctagcccattgcctcatacacaggagaatgcaaataaatcatatgcttaatgaatgagtcgatgaatgaatgatgaataaagggaatctaatctagttttaacaaatccaggttttgcaatgatctcacaggcattcatttatcttgtgatgtcaggggagtgactccaccctcatttcacacgcatcttggggtcaatgctctaacttacttggcctccagttagtgggaaattacaagctacacttcaagcctctgactaggacctgccatgaagtacttgggaatcagtggagtatcactgtggggtgaggtgtctgaggcgaggcccaccaatctccatacttctccccgggcccctctgcctgagagggtctccctgcttcccttggcagactctggtttggccttctgggttcggcgttgttgtcacctccttcaggaagcatttctggctaaggtgccccactctatagcagctggtgtaaaacctctctaagcaaacagcataactttctgtcctctcaattgactctgagttctgagagcacagcctggagctggcacggtgcctggcacagagagctgaaatggcacaccctagtgttcccagtggctcgactccccaggctctccatcaggacgcagccctctcccacctctgatggatatgggaccatggaatgctttgtccagcagcaactcttgcctccctcacagaagggaacacctagcccatcagactcacctttccttactggaaaagtccactcccagcaagatattctcctcggtgtcctggcgcccgctgctgtacaccaccaccatgtaccggacccggtccgcccaggcgctctccaggcgcactgcctggaacagggcagacatgctctcactaacctgcctttggaggtggtgcctccctcccatctccaatgcaagatcaacactttcagtgttctacctttccctctgggagttaaaaatgaagagaaaattcttggctgggcatggtggttcaggcctgtaatcccagcactttgggaggccaaggtaggcagatcacttgaggtcaggagttcgagaccagcctggccaacatggcaaaaccccatctcttactaaaaatacaaaaattagctgggcatggtggcgtgcgcctgtaatcccagctactcaggggactgaggcacgagaatctcttgaacccgggaagcggaggttgcagtgagctgagatcatgccaccacatgccagccagagcgacagagtgagattctgtctcaaacaacaacaacacaacaaaacacaaagcggaagttcttgacagcaggaaccaggcctcgtttctctctgtagcaccagggacgccgcctggctcagaggaatcacccaaaatgcaagaaatcagtgaacacatgaaatccaaagaaagttcgtatttagcttatttaactgccgtggagacctgtttcatccctcctcccgcccctctggggaactgaggagtcaacctggctttggctttagtgcacaatttgagaatttgttgtaacctaaaagcttttccccttatcattcacgaatggttccccaccaggtttcacaattaaaaattaaaacttgctggctgggcacggtggcttacacctataactccaggactttgggaggcagaggcaggagaatcatttgaggccaggagttcaagaccagcctgggcaaaatagcaagaccccatatccacaaaatttttttaaaaataaggcagggtggtacacacttgtagtcccagctatccaggaggctgaggtgggaagattgcttgaacccaggagtttgaggctgtagtgagctaagatcatgccactgcactccagcctgggcaacagagcaagaccctcatctcacaaaaattaaaaaaaaattttttaacttgacattctcactgcttcttaccagcttgattctgtcttcgcaacgcagaaggttgatcatcacctgaagatgttgaggcagatcacctgttggaccaataaagaaagctttaaaaggtctcttacctactctctaggaaaaaaaacctctgaaaggctgactttgagggcttggaaaaagattgagaagttaaaatttgtctacctacaccacaggagaatcaccacaaaaacttcaagtctgaatttctcttacaccactctgaatactgtgcgacgtggatgggtgacatggagcttactgtcatgttgttaaaagttgctcttatttcctgaaatacatacagtataggtttccaaatacaaaatgtgaaaaatacaggcaagcctagagaaaaatgttatttcattcaagccaatgttactcggcaggttggggtgcctagaaacgacagctgtggctggaagtaaggcatttgctaagagttaatcattagagaaaaaggacagagcatcacgtttcctcttcaaacaacttcttcttctatacagagtctcgcactgtcacccaggctggagtgcagtggtacgatctcagctcactgcaacctccgcctcctggattcaacagattcttctgcttcagcctcctaagtagctgggattacaggtgcccatcaccagacccggctaatttttgtatttatagtagagatggagtttcaccatgttggccaggctggtctcgaactcctgacctcaagtgatctgcacgcctcagcctcccaaagtgctgggattacaggcataagctaccccacccaggccccacttcaaacttctgcattttccactggaggcagacattatttccataaccgggggggcggggggaaatgtttaagtgactctacagatagcagctgtatgctggttgcccagagaaataatttgaatagaaaccaatctgtcattttctcttttcttgctaaaaattatgtactcttttttcttcactatgtaaaacaggcagtaaccagggacggcttctgaacttctctgagctgccccagggttcaggaggtgttcctggagtgcagtgaggaaagtctcttactggccatgagtctcgcgcgaagcagagaccctgtcagaagaagcgcacactttcacggaggggaaagttgtaagggaggtgcataattagtaagtagcaggtgtgactccaaggttgctttttttctctagcttacacatttttctttatatctgcaaggatttctttctgaagaaagggtcatctgtagagatgctaatatcagcctggtgtggtggctcacacctgtaatcccagtgctttgggaggccgaggcaggagactcacttgaggccaggcattcaagaccagtctgggcaacatggcaagaccccatctctacagaaaagtaaaaaattagctgggctttctggttcacatctgtagtcccagctacttgggaggccaaggcaggaggatcgctggagcccaggagtttgagatcaccctgggcaacacgataagaccctgtctctacaaaggaaaaaaaattactctatacatcacaattacaaccccaaaaggatcaataatgcttacacactcaaatgctccaaaaggaaacattgtgtttgttccttttgcaaaagcatctttttcattttaagggagaaggacagatgatgtccaaattgcacttcctgtctcagagaggaattgggtcattagaaatttgtgcctctagccaggagggtagatctcatgttaagcgttctttctttttctttttttttcaatagagacagggttttgccatgttgcccaggctggtctcgaactcctggactcaaatgatcctctcacctcagcctcccaaagtgctgggattataggcatgagccaccaaacccaggccaattaagcattctttccacaataagtaaaatttaaaaaagaaaagaaccatgcccctcttatctgtcctctccagttatacaattccacagtgtataacaccctgtgttgaccctgcttcctatgatgagcgatttggagataagggttcacattaaagaaagccatagacctccccagccccttcctccacccgtcatgtcaccaatgcaacacaacgacaacgaccatgagctggttcttcacctgcctgggccctcccaccatctacccgagtcacagaactgcattggggaaagcaaaaacaaacccctgtctgataaatgcctaaatgaaagggacattttccacacagataaacttctttcagtgggattgtttgctgagatatggaactgctgacagacagaaatccaaaccccagtctgacatccacacacaaaaaaatcagagaatataagccctagaaagggtctcaaattgactggactggctgaaacaaactgaactacttttccaaggacagaattaaccctcaattgtactcagctctgcacagtggttactggggggcctctggtacattcaggagacttgatggtaattctagggaaaaaaaggaactaacgtaagtctagtctgcgtctgtcccaaggtcatttacagaccaactgtggacagctggcggcccctctgccttccgacctcatcgtccactccagacctcagggcacaagagtcagccagctggtggcttgcatcctacccttctagtctttggattagaggaaggaggtatctgacacttagtgagcagagcttgagcatttgctttgtcatatgtgttacaattaaaacatgaacaacagctacatttctaagagggcagaataattagcaaattcaagaacgaagaatctggctgagtatggcacctcaaacctataatcccaatgctttgagaggctgaggtggggagatggcttgaggccaggagttggagaccagcctgagcaacatagtgagtgagacctcatctacacacacacacacacacacacacacaactagctgggtgtggtgacacgcatctatggtcccagctactcaggaagctaaggctggacaatcacttgagcccaggaggtcgaggctgcagtgagctatgatcaggccactgcacgccagcctgggcaagagaatgagatccgtctctaaaaaaacttttcatataattaaaaaaaaaaaaagaatgaagggtctgtttatagctgtattgtactagaagtcatcgtaataacaatgatagttacccatatatatatacagcacctactacaggtaggtatgttacacgcataactctaaatttccatattgtctgaggcaccagtatttgatgcccattgtaaagactaggaaactgaggcttagaagtcgacctgttacggcttagtaagttggagaacyaggatcagaagacaggtctgcctggcttcaaaacaaatactatttccacaaaccacactgcctccttgtacaggacagttattttctttgcttaaaacagacctaaatattatcaacatcagtatgtgaaaatactgactgagccttggtgtttgctataaattgcatggtgtagaattctaacctgagcactcagatctaaaatgaagctgaatgacttgaggttaaacaaacaaaatgttcacaagaaaactggccacaatagctggttggtttcacctgctgctgttctgaaaggtaaaggccttctcagctcacagacattcaattatgcactgcctctccaagaaatgccctgagatgctgtccacctacgacaaagatccacttacatgcaagcactttttcctctttctttctttttgagatagggtccttttcttttgtcacccaggctggagtgcagtggcgcaatcgtggctcactgagcaacacagtgagcaacatagtgagacctcatttacatacacacccacaaaaaactagctggctgtggtgacacatcagcctcgacctcctgggctcaagcaatcctcccacctcagcccccaaccttgctgggattacaggcatgcgccaccacgcccagctaatttttgtattttttgcagagatagggtttcactgtgttgttcgggttagtctggaactcctgggttcaagcgagatctgcccaccttggcctcccaaatcctggaattacaggcaagagccaccgtgcctggccataagtgtgttttgttgttattgtttttaagaaacagagtctctctctgtcacccaggctggagtgcagtggcgtgatcctagcttgctgcagcctcaaactcctgggctcaagcgatcctcccaactcagcctcccaaagcactgggattacaggtgtgagataccatgcagggccacgcaagcatttcttgaattcctctttctaactgccttcagctctgagtcaagtctcctaagaaaaccagtcttactacttagtaggcacttcttatttaaactcagtttgatcctcaccctattacttctgtctacttcctaaaaacaaactattacagaatcaagacttcctactacagtgtctatctcagagttggagccaaaggcccttcaagaaattctccaaatgagtgtttttcaaatgcttggagaaatccatcccaagattaggtatacagcactccagatggttattttcaagtggacgacatctggctataattcattttggtgcatttgttaaaaagtcaggctgtaacttacagcctgcaattaactgataaactacagagaggaaatctttgcatcccagcaggatgctgctgaccttactcctgacgcagacagacatgacataaaaggttggaaaatgtgcgtggtctgctcaagagagagcatctgagcctctgcctgcactggtcactgcaaacctgcgtccactatgtctaaggccttcaaactcagcaacatcaccaacaatggaagtttcctctgctgtccagaaaagaagctccaatgtaagagtatcaacttagagccctcacctgcatgcttgtgggggtgctgaagactccgctggccttgagggctgcttccctgttgtaagaagagggctgcgcctttcaccatgaaaaagctctcacttaagctgggaaggataagaccagagcacagttagaccggaattcagacaggaaaatggacaaagaattactgcaggggaaaaagctttagcgtggacaaatggcatgtaaaatgcaaataggatgaaactgcttttataataattccacgtagtacttttctcaaaccttgcttttgctaaaagcttgctgctggagaattttcgtgacaaaataatgcttctgtgacaacacccaaagttctacataggctctccagggcccctttctgcagaatactggacagggatctcactgtcatataacattttcttctttcttttttttttgagacgcagtttcactctgtcatccaggctggagtacagtggtgtgatctcaactcactgcaacctctgcctcctgggttcaagcgattctcatgtctcagcctccccagaagctgagattacaggcatgtgccaccatggccagctaattattgtattattagtagagacatggttttaccatgttggccaggctggtctcaaactcctgacctccagcaatctgcctgcttaggcctcctggagtgctgtgattacaggcgtgaccacgcccagccataacattttctaagaaaagagaacaactccctgattaggagagggcagtctactttgtgaattctcatgctcttgctgttgatctctgcttctaactctctggcttttaacaactccattgtttcttggtgacttcccttgatggaatacaaggatgaaattacactttcactagttgtttgcattttaagaaaagtggggaggggccgggtggctcaagcctgtaatcccagcattttgggaggccaaggcagtggatcacttgaggtcaggagttcgagaccagtctggccaacatggtgaaaccctgtctctactaaaaatgcaaaaattagccaggcgtggtggcacatgcctgtaatcccagctactcagaaggctgaggcacaagaatcgcttacttgagccccagggacggaggttggagtgagccaagatcgcaccacgcaccactgcactccagcctgggcgacagagcaagactgtgtctcaaaaaaaaaaaaagaaagaaagaaagaaagaaaaaagtgggtggatactgacttgtgatttaacttagtcaaggttgtcctgtccactattcttgaggaaaacctcaagttggcccaatgaatttctcagcagaatgaatctttggcctttgttattttagctagcaataacatttataactacctataactttaaaaattacaattaaaaaatgtttatttgggaggctggggtggaaggatcatttgagcccaggagttcgagaccagcctgggcaacgttgtgagaccccgtcgtacatcaaaagtttttatttttaatttcactttcatgacttggctatcaagtctggcttttgcaaaaattaagacataagaaaagaatgcttcagctatgaattactatcaattgttcaaaaataccatcaactctcaaaattatgcataaaatacaccaaaattattaacaacggctttgcgggaggtgggggaggaggaggaatagattatcttctagtattttccaaatgttctatattaaacatatattaacctttaaaacatctacttttgtttgattctcaaaataatataaaacactactatataatttaaaaagaacattctaatcttaataatttcataaaaggaggtcacagttcaaattgtaggcaactataaaaatttcgctcttgaacaaccaatgaacatatacatgatttgaaggaaaaatccctaagaaaaagcagtcttctaattaaagagaaccttgaaattaagtaaatcaattcctgacagaaagacgaagatgttttctgtaatacaagaaagcaagatcacctttgccccagacatctaatgttagtagttaaacgttcgaattctggaataaaaaactcagcaaagtctaaagtatgactctgggtgccaagaaaatgccacaggaactagcatttccaatcagcagctcctgagatcaggaagactgttatgttctatgatataaagtccacaataaaatctgttagtttttctggttaaatgctcatgctaaaaatagtgactgctcaaatattaagtaagaagacttagttttgccttcttgttcagtcctctgaattccaggcaattggttttcgatatcttgtgacaccaatacttgacatctaacagcattttgtccactactgcagatgcactgccgagtcatcctttccaccctctcacaggcatatatttgtgctgcaaggttcaagtgttgaggagctcaggattataaataacgaaagaaacgagaagcagcctttctttgctgtctcaccctcactcataggaagtaaaaagctctttagcatccatctggccgatctcatttcacaggctgcagaatcacctaaccctttccacctgcaaagcttgtcactctctccttccttagaatctcacagctgagtatgttttcagaactgttcttagacacagatcatttactatttattctcatcaaaatctgaaacagctatgcgagaggttccaaactcatgaaacctaaaacaaccatcagttcatcgaagcagctgggaaaatcttttcgagacaacatcaactgcttttgttcatgagattaaaaaaaaaaaattcatactgaccagaaacccaagcacgctggaaacagccaaccattaacgatgacctttgccttggaaaccatgagcaaaaattccccttggtttcccttatatttcctttggaaaaaaaaaggaacaatgcaacagactaggctggtttcactctgtgatcacttacaaggccagctgttcctcctccatgttcctacactgataagaatcagggactcctgctctacgcatgaagtcaggatggcattgattggggccctggaacactctgcctctgttcccccacgacaatcaagtaacaggcatttactgtaaaaagcaagactggaagctgcagggaagcccaagtagcagcgcattatcccgaagctgtgagatcaccctgcgtcctgcaaatacagtcaggagatacagccagaggaaaccgcacgacatgactctccgggtggggggtggggtgggaggccgcagagcatggtcagtcacaggatttatgaaaacaagatgcagaaagtctctgtgacccggcttcctggcttctcttctgagctcactctgggcccagagcctcatgcgccctctgcgtggctgacctgaatactgtatctgacgactgcagcttctgatgcccagaggcacaggctcccgattcatcagaccctcaaagtgtcccactggggaagtccatgaagaaatccacattggtgatggcacgctcactttaccaggtgtctggggccaggaagcccaaacccacaagccatccatcccagccacccagaagtcactcttctcacaaaagatctgagtgtcctaaaaggagtgactaaagttacaaaaggtcagacgcagacagacaaaacggaaatgtcttcctccaccgctgtaagaaaaatcttgatgagggataaaaaaaaaaaaagccgctgccctctctacccgccaactggaatgtttttatctccaccacacagatctgttctcggacactgattactgccattcgggaagcttcataagattaaagtttctccaaagcattgaagacagacaaaaaacctcaatcaatgctcctcaaaaaaccccaggcccccaaaatataaacagccagtgtcatccagaaaccaagccatggcaggaaaccagtaatcagggtggtcatacgtactaatttgagctggaaacctctggacagcagaagcagtgggttggctgaaggaagatgcagaagtcggtaaaataaaagaggttcgtggctgcagtgctcacatctctaacgctccctacaactgccctccgagctctggccatctgctccctatggagatcaggaaaagccaggaggctgccgagtgcttccacgagggctggggagccaactcctcctcagagtcctacccgaaaagcaaatggctcttgtggaactcttgtcttcctctgatattttggctgaaaaaggcccttgtcccagcacatcctgatgaaagagggccattcagcaaaacagctgaggttcctctaatcactgcactcctacgggcttttctgtaggccggagaaacaagcaccggggtgtgcattcgacattgtgagggcaaacaactggccccaaggaaccaaccccaagcaacaagacccccttccgattcaaatcaacattctgaaggatgactctttctttcaaatcagcatccatttacccaacggtgacggtgacgtgggcagctgccgcagttagttattctgcgtactcaaagcacggttacatcctgaaaattcttcagtcatgctaacagctatctgaggggacacgccaggtagaggggaccacatgcacacctatgaggagctctgggatacgcacggtgcccaaggcaggtcaggctgcaaaggtcctaaaggttggaggtgtgatcccaaaccctccaggcacaagccagccaagagctgtgtttttagcgtttctttcagtgagagaaataagttcaggatgtgaataaccatgacgcaggagagaatggaataagtaccctaagaaaggggctcggctagggtttacaagagggaggagggagcatttaactggtgacttctggaacaattcctgaaggaagcagcactgagtaggggcttctcttccctcggctcacaagtgaccaagcgatcctccctacggattaagtgaaacacacattaccatgattctggttttgcaggtgaggaaaccccagcttgcctaggagcacatatctctacaagatggggctggactcacatctatctgccccacgcccacctgcttaacccctgttaagcagctgttctactcatccagaatgaaaatcagagccattatgctgcggtcacatccgctcatgcctgcccaggtgcctaatggcaaagccactaaggcactgagaagtcagaatgtggatcacatcttccgtccttcttcccagtgtgtgaatgcatcatgcgtgggaaagagagagaaggaaccattcaagcaaacagaactccaggaagacgagactgtgccggggttcttccatctgcccaagtagaaatcagaagggcaggggacccacagccttatcctacccaccactgccgtcatagttgggggacaggacacatcctttggcccttctgcactgcatagaggctaaggagttctgtaaaccacacagccacgctgaccaagaagtcgctttcaaggtaagtttctcatcaacaggactattatttactgaggatctcccatgtggccaaggctgtaggaggtacttagctacgccacgtcattgaactctggcagttctgcagggtaaggtattttctccatatgacaaacgaaggaagcccgtcaacaattccaaaatagaatcaccagggatagcatggacaacgcccatggtgactgccgcgctttaaggtttaagaaaagtaaaaactgggggtgatgactcattcctgtaatcccagcactttgggaggctgagatgggtggatcatttgaggtcaggagttcgagactagcatggtcaacatggcaaaaccctgtctctactaaaaatacaaaaattagccaggtgtggtggtgcatgcctgtaatcccagctactcaggaggctgaggcagagaatcacttgaacccaggaggcggaggttgcagtgagccaagatcgcaccactgtactccagcctgggcgacaaagtgagacactgtcttgggcaggggcggtggggaacaaaagtaaaaacaatggtctgggaattcatatttctgggttccaatttacattctaccatatatactctgattaacccctagaattaacccctagaattccttacagggttctgttcattcatccaaacaggcaaacatttgcagagcatggagcacagggtaagccaagccagcccaagctctgataagggcaaagacagccatcctctttaaggaatgggtatatgtgctggtgatctgggtgtctgccctgctgcatagaaacagcatttcttgaagaacaaaaatagtaggtatagaaacatcacagtatggaatatccaaacacccctgaattccaactctggtcatacattgaaacaacctatcaaactcctaaaacacattcatgcccaggtccagcctcagcagagtctaattcggaaggtctgtgatgagtcctgggcatctacttttttaaaaagttccagggagctgggcatggtagctcatgcctgtaatcacagcactttgggaggtcaaagtgggagaatcagttgacccctggagttcaagattaacctgggcaacgtaacaagatcccatctctacaaaaaaataaaaataaaattagctaggcttggtggtgtgtgcctgtagtcccagctgctcaggaggctgaggtgggagaatcacttgagcctggtgaggtcaaggctacagtgagctgtgaccacaccactgcactccaacctgggagacagatcttgtctccagaaagttccagggggtgcttctgatgcacagccaagttttaaaaacctcagaatcaaataacatcatggccaggcatggtggctcacgcctgtaatcctggcactttgggaggccaaggtgggtggatcacttgaggtcaggagttcaagaccagcctggaaaacatggtgaaacccagtctttactaaaaatacaaaaattagctgagcgtggtgacgcacacttgtagtcccagcttcttgggaagctgaggcacgagaatcacttgtaccctggaggtcgaggctgcactgagtggagattgtgatcctggagtccccactgcactccagcctggatgagagtgagactgtctcaaaaacaaacacacaaacaaacaacatcagaagacacagagaaaacagtcttctccatgggcttcataaagatacctctcacataggtacacgtcgatgttttctgctggtaaaaggtaacaccaacaaaaaggcatggtgctctcagaaggtgggtgatgtgattaggtgcaataaagggaggtcatgctagggtcaaaaacaaaataatactctctttggaagcagtaaaacagatgctagtcttctactacacactttcagagacctgaatgttcttctggccctctaagggagacgctgcatcatgacaatacgaaatgatgacagtgaaagcaaaaacagatcagacctgtgctgtgtgaaacagacatggggtctcgctatgttgcccaggctggtctccaactcctgagctcaagcgattgttccgccttggcctcccaaagtgctggggtgacagctgtgagccaccgagaccaacctcagatcagacctttgacaaactctgctgtggacaaagcattctggtgaatgtcaactcatctgatcttcacaaaaccgtgtggaagaccagacaggcattattacactaatttatgcctaaggaaacagggagttaaatagtacaaatttaggatttctgatgctgtatctcgaaaaaaaagtagagaatatgagcctgaagaagaggccctgtaaagggtcccagattgatgggacaggctgagacaaacggaatcacttttccctggatagaactaaccctcaatggtaccccactctgcatggtgattactgaggggactgtcaattgtccagcgaacttgatggtaattctaggagaaaaaggaactaatgtaatgctgtcagcatagaaagatgggtgccaacgagcattccaaaaaggaggctctgttaattcggtttcgatcaacaagtatttgctgagtgtctattgtgtccggtcagtgctaaggcctgagaatttagaagtgaaacagacccggtttccacccatgccacagaccactccacacctggtctggagtgacactggagggccaggcaggcacaggacagtaacttcgatataaggcagcaagttccacggtggaaggaggtggaaggtgcagatgcacgtacacacaggggttcagggaggcctccctggaagaaatgaagcctgcgaggccctgaaggatcagtaaacagagaggcataaggggcaggagagtaagatgattatgctacatgtaccttattgtgaacccaggaggatttggcctctgtcataaaaggcccccctgtgggttcataaacctcaatttacaaattgtgctttatatatcagttccttataagtttggttagcgtaaattggtttcttagaacttgatcatccctgagtgaactcacaaattcaagtttcagaatgtgcaaacctaagaaacaaacctcatgcttgtggttgagacatcgcactgtcaacatcacaaattctcagcacctgaatgcctggtatactatcaacatatattgttttaaatatgtaaataatagctttctagttatagagagtttgtccctacatttttccacttaatttttacaatcccattcccctgatgaaacaaccccagcctgggcaacatggcaaaatcctgcctctacaaaaaatacaaaaattagctgggtgtggtggcgtgcacctgcagtcccgggtatttgggaggctgaagtgggagaatcacttgagcccgggaggcagaggttgcagtgagccaagattgtaccgctgcactccagcctgggagacagagggagactctgtcccacccacccccgcctcccaaaagaaaagaaaagaaagaaaagaaaatgaaaccaccaagactgggagaagataaatgacttgtctgtggtcatctggctaataagaggtagaatggggctgaaaaagttcggtgctcttcctgaagaatccataggtcagaaagcagcaccatctgacctgcagcaatagcagcaacgtggaaagctaatcaactgacctcaaaaccactctcagtgaggctctggatggattcagaaccccaggcctagcaaagtgaagttgataaagatgtaaaggagatcgaaaattcaccatttggagagagattagctaaagactgcaggtcggatggaaaattctttccatggttctcccacaggttcttccctcatttggaactcgtgtttaaaagtcacaaagaccctgagttgggccaaggtctcgttcttcttcactgtgggccttgcagtgcaacatggcagggcctcgttccaaatgtcactcttcagagcctaagaaaacaagtaactttagggacacacctgtcaaccggagctcccaaattgtacccccctaaacacataatgctgagcatagaaaaattccagctctgcagagcgttatacttagggaaaggggtcacagacaaggaatgctggcagggctcattacaaatatctttgctgctggaacatgtattgtttggctagaaggcgtaggcttctctcagagagaaggaatgtccaaaagtatttcagacagtaagagacattctctgagccagctacacagctctccttcaaaccaacgggtagcggcaagcagctgaactgaccagcgagctcgcaaaagcaagcttttttttttttttctccctaaataagacagcaagtgatgtgtcttggcttggtttagcaaattttaagatagttccctgatgaccccaagagccctcaggccccatggaagctggagctaatgcatcttcctccaagcatcatctgctctaccaggatctaagccccttcacgagggcagaaggtataaaggctgcactgtgcgggaaatgctatggcagcaaagacagccaaacacgccagaaataacaggcacatgaaggaaatgtttctgagacagctcaaaaattccgagaagagattatcccgactgtcccaggttctcagccctgtctatggtatgcagccccataccacagtcatttgtcaccgagtcctaactttgtcagaggcccctcctttcaggtctctcaggcaccacccagttctggccctcctcacccccgtgagccaggcgacatccaagcagccccacggtgcacccggctctgtgctgcattctctgaatgtccctgaaggccagggctgttgtattctctacccactctctgtctagtatgggagccactggccagatgtgttgactgaacacttaagatgcagtaagtgtgaccaagaaactgggtttgtcattttatttcattttagttaatttaaatttaagtttaattagctacatgaggctatcagctgtggtataggacagcagagctccggaagcttttggcctggtgagaagaatcaggacaagcgcctccctggcctctcgcccactctgcacagccgctaaccattgctctcatgacattctttcccagccccagaacttttagccatgtgacatcatctattgattagagtccaaacttcttgtgctaactctctatgggttgccacaattagccattgtatgtcgttaacctaaatttcattcatctgctatgtcctgaccttaggggcttagaatatagttagaaaacagtatttcagaataaaaaaccattcttgtattacctctcgcactattccccctgttctccatgcttcgcattctctgttctatccccagctatagcactgtccccataaagcctactgtggttctcggctcatggtgtccttcctcccatctgcctcccgacgtcatgcctgtcttccagtgtttatgccttctccaggaagctttctcttgtggccctcgctgtgagctatagctcctccctttcaacatcttctagcacctcctcttactgtgcaggtgaggacactgaggcttaagggttaagtcacttgctcaaggtcacatacagtcggtcctctgtatccgcgggttcctcatcagtggattcaaccaactacagacagaaaatacagtatttgagggatgctgaactctttgaattagtgggttctgcgggtgcttaagcatccatggattttgttatcctcggcaaaggcgggggtcctgaaaccaatccccttggatactgaaggaaagaccacccttagtgataggaacctaggaacccaagttccctcatttccaaatcgtgttccctgacccacttatttactaactagtggtgaagccatcttcctgccagtatattttaacttcacaatgggatgtgagggccaggatgcacatgctttttaaatctccctctgtgcttgacatacagtagattgaaagtaagtgctggtagatacactggccaagctgtgctcttctctgaagtcagtattccaggagtaactcaccctggtcatctctgtgccctgggcacactgggcactccccacacacaggttgaacctggcaaataagactcacagcatcatgccacgtgcgagttaaagccacctggaggtcaggtcaggtcttcctgacaactgagtgcttcaaataacacaacagcagctaagttccccacatcaccttgagtgtctggagagctaggcctatgacttctctgtctcaggatccctctcagtgcccagaaaacagtggacatcaataaatgtaacaccaataacatcttcgttgagcgctatgctaagcacatcaggtatgttaactcatttattccccagtgtccatctctcagtgttttatacatacgggaactgaggctcaattagccgagcgtggtgtcgtgctcctgtaatcccagctattgggaggcacaagaatcactcgaacccaggagatggaggttgcagtgagccgagattgtgccacagcactgcaacagagtaagactccgtcttaaaaaaacaaaaaaacagaaaacaaaacaaacaaacactgaggctcagggaggttaagtcacctgcccaagttcatgagaccaaggagccgggaagcaggaaggggaaggcaggagtgtaactctgaaacctctgctcttaggcactggctttcagctgaactgatacctctggaaaacagtctcaaaaaagtccacttctcctcccaacaattcagacctaaaaaccatttggcggggaagggcagggcaagcttctgagttggggagggggtgtgggatcccaagctgaggtgtctgttggcaagcagggtgcaaagggcatctgtgcagggagggggctgcaagggagacagagactgctcacaggcaaggaatgaaatattaaacattaatgttaatattaatatttataattaatatatttatgatatatagcatatatacatattatattaattaattataactatattaatataattaattataactatattaatataatcaattataactatattaatataatcgattataactatattaatataatcgattataactatattaatataatcgattataactatattaatataatcgattatattaatattaatataatcgattatattaatattaatataatcgattatattaatattaatataatcaatattaacaaatatatactatataatataaataatacctaagtttatataatatgcataatgttaatatttattaatatttcagggacaatgggagtcatgaatatggagagacaaaactagaatgaaccccaaggtgctgcatcagaattgaaggtaccagtctgaactcatagttttcaacctattgaaataaatatagatgcacgtgtgtgtgtatgcacgtacatacaaatgttccctaattctgcccattgagaggcctgtggttagcaacaccccaacagcaataagcagacctagcttggctcctaaatttcattttccactaaaaggaaccagagccccttggataaaggactgattccacaggtgggtagggagcatctgttgccagaaagcaagaaagcacttaaagaatgatgtggacatgtcaaagggacacagaagccagcctggatgagatcccactggccctaactgtccacaaggacaatttgagcaaggatgtcaacaatttaagagcagattataaaccactgaataaaacagaaaaatacaaagaattgaaacggacattgatggcagacaggatattaacataattttaaagtatctctccaaggaatgcttctgaatgatgaaggggaaaagaataactgtacagtggaaaagcctggtaaaacccaccttagtgaccaaagtgaatgtcaccatagtgggacaaaaggaaatcaagtgccaccttatgggattcaacgaggacgcagcatcccttgggtgatgttccagccaaatacacgtgcccggtggaatcacacaagaacatcagacacactcacactgagggacactctgcaaactgacagtactgggcacaaacatgtccaggtcatggtcgaccgcagtggctcatgcctgtaatcccagcattttgggaggctgaggtgggcggatcacttgaggtcaggggttcgagaccagcctggccaacatggcaacaccctattctctactaaaaatacaaaaattagccgagcgtggtggagcatgcccgtaatcccagctacttggggcgctaaggcacaagaatcgcttgaacccgggaggtggaggttgcagcgagctgagatatcaccgctgcactccagcttgggcgacagagtgagtttccaactcaaaaaataaaaaaataaaataaaatccaggccacaagagtcaaagaaagactgaggaaggttccagactgcaggagagccaagagacaggataactagatgcaatgggcagtcctgaattggatcttttgttatgaaggacaacgctgggacatatggtgactcttgaatggggttagaggactagacggtgggaatgcatcagagtcagtgtcccgcgtggatggctgtgttgcggttctgtgggagaatgccctggtctgtattccaagggtaatggagtagcaggttgacaaattactttcaaatggttcaaaaaagaaagttcttttcactgtacttgcaattcttatgtaagctggaaattatctcaaaattaacgagaattttttatcgacgtagtattttacatatttatggaaaacatgtaagtatttgttacatgcataaactgtgtaatgaccaagtcagagtatctggggtatccatgaccttgagtattaatcatttgtatgtgttgggagcattacaagttttcgagttaccaatttttttttttttcctttgagacagggtcttactctgtcgcccaggctggagtgcagtgggacgaccacggctcacgcagcacagcctccacctcccaggctcaagcgatccttccacctcaaccacccaagtagctgggactacaggtgtgtgctgccacccccagctaattttttaatttttttgtagagacagggtctcactatgctgccagggctggtactgaactcctaggctcaagagatcctcccacctcggtctcccaaagtgctgggatcataggcatgagccaccatacccagccaaattttttaaagttattttttaaatctccacttaattcgattttggtaaaacacgacctgtaatttttctttatcggtaggtaataaaagcttcagatgattttactgatcactggtatgggcatatttcatgactttgccctttcatctcttgcatagttttaccctcaccaagcaagaccttccctgcctcagcactgtttgccctcttcgtgttttccagaacagaagtggccctgtttcgtgcccagagcagaagagaacgatgaagagctctgctctcccaggtcttcctggtctgtgtgtgtccaggttttgagggcctctcacatacacggctctggaccacgtaagatctaattttagcattttcctgctcggagaccacaatgtttggaacagcaggggctgacctgcccgtgcaggcctcctattgtgaagggcacgcgaagccaggataccgcagccctgcaggatgtgactcagcatcctgtctcagtgctggggcggccagcagctctggcaccaagtgctgctgctgacctcacctcttaagaccacaaatacccagggtaattggtgggataggcatgcagcatcagctctccctgttaagacaacttgcttgtccatccattatgctgggcttccttgtgaacaccacaggtatctatcaggaagagttcttccgaggaactgatctgctggtattttcaggacaccaagaatcaagagattggtcttgtttctctctttgctttgactaccaggaaactcaaagtcagatctgtggccaaattctggtaaccataccaatgctatgtcatgtattacatgtacaaaccttccccttacttcatcttattttcttctgctttcttcgtgtcccgattttctcactaatgttacattctattgttctctatgaatgttgtaaggtgtttcaaatcctttttggagggcactactgtagatacaacacacattacccctgagggataaggactctttttgactccacacagaatccctggcatttggcaaagaacccatatttaggcactaaatacacatgggctgaatggaaaaagccaatagctaagtaaaaaccacctccattaccatattgtttcacaagaggttcttttcccttccatctcatgaggtggggcctggtcaggagtccccagggcctgggaattaggttccttagggagccttcttgctgtaggggcagccaacaggtcagtggccttgactccagacctaaagagccactcctagactcccagctgcaacagacacagcgtggcacgggtgggcctggccactggggaagtgacaagtgatttccagatgctgcagccagcctggctctttccagaccacactgaaggccccttcctgtgggaattctgatggggcccagatttggggaaacacgcctcgaggactcttggcaagtgcgtgccaggcctggaccaggaatgacttctgtgggcacagggagagaccaggcatttcctaacacaggaccttgaacagccttctctgaaacaaagtctttctaaaaatagcttcaaaagtaaccattcaagaaaagaaagaaaaaaaaaactgtaaaagtaaaggcactcaagaatgatatttcccagataaaagcctggcacaggtttcagaggaacttgcaggaaaacaggtcaaggctgggtttttcctcttaggtgtcacttggttaacattggtctttggaggggaacaagtgcggcaggaagggctggcactgaaaatgatggccactgggtataggccagggccagacactgtacacagaacaagactctctggaggcctcaggagggccctgagaggaggaaggcaggtggtgggcccagggtcagacatgcaagtgagctaagtggcaaggccgatgccccatccagaagccccgctctgaccacacgcaggctctcccggcatgtcctcatttatgcggcagtctcttgtatctcactgcaattctgcccccacactgcaggctggccagcgtggcttcctcataagcacatcaccctgcatcccgacactgactacacccacaaagcaggagcccccgcaccctccagcccaatcgctcagttcgctttgaaaatggctcctctcgggggctgggcgcagtggctcatgcctgtaatcccagcactttgggaggccgaggtggttggattgcttgtggtcaggaattcaagaccagcctggccaacatggtgaaaccccatctctactaaaaatacaaaaaattcgccaggcatggtggtacaagcctatagtcctagctacccaagaggctgaggcaggagaatcacttgaacccaggaggcagaggttgcagttagccgagatcgtgccactgcactctagcctgggtgacggagcaagactgtctcaaaaaaaagaaaaaaaaaaagaaaatggctcctctggattttgattaatcctattttgattaatcctggtttctcattttcagccttccttgaagcagcatgacccatctggatgtcctcctcatctcaggaattttctaataagctgtctaaatccagagatccgaccacagaacaatgaatgccaaagatgagttctaaagatgcgagtactttctttctaaacggacgctgctttgtgtatggctctgctcctgggggcagacgcggcaggctaagccctgcggaggaggaggtgagtcccagcagagggtcacttcctctcagtagcccggctggttttctccactgcagggtcagaccatagccctgacccagctagacccccataagcgcatgaccttgctctcaccgtgggaataaaactcgtgatagtcagttacaaatacacagcaaatgatgagcagcacaatataaacacagatctagattggtgggtctgaggactcattcttaaatttggaggccatcacctaatcttgtcttttcactttacatagcaggagacagggacccagagaagtgaagaggcgttgccttaggttgcacagcagatgacgcctctcaagatggaccctaggttgtctgactccgtctcacagctttgccccatttatcatgaagatgaacgctggtaacactgctacctacgagctgagcttgcacgcacattcctggtgtgtacatgcatgcgtgcacgctcacgcaatgtgctaagtgcacaggaaggagaccagagccctgaggcgttcttttgaagtctaagtactggtgtttcgaaagtttaatgaaacctactagactctgagcaaaattcgttttacgttaaccttaatgaaaagtttaattaagttctgacagaattaactcttcacgtctctgtcctcatttgtccccattctagaatgagttttctaattaaaaaaaatatatagggccgggtgcagtggctcacgcctgtaatcccagcactttgggaggccgaggcgagtggatcacctgaggtcaggagttcgagaccaacctggccaacatggtgaaaccccgtctctactaaaaatacgaaaaattagctaggggtggtggcgcatgcctgtaatcccagctactcgggaggctgaggcaggagaatcactggaaccctggaggcagaggttgcagtgagccaagatcgtgccactgcactccagcctggtgacagagcaagtactcccatccccccccacaaaaaaaaagtatatatgtgtgtgtgtgtatatatatatatatagctaggcacagtggctcatgcctggaatcccagcactttgggaggccgatgtgggcagatcacttgagtccaggagttcaagatcagcctgggcaacacagtgagaccctgtctctaccaaaaatacaaggtggtgtgcacctgtggtcccagctacttgggaggctgaggtgggaggaccaattgagcccaggaggtcggggctgcagtgagctgtaatcatgccactgtactccagtctgggcaacagagcaagactctgtctcaaaaagaagaaaagagagagagagggaaaaaaaattgaaggcaaattctgattttcaaatcaaacgttccaacaaactgcagaaataaaacccgagttaaaccaaaaggaacagccaaacagcacaatgaccccaatgtttaaatatgccccaatgtttaaaagtgggagtcaatgggaggccactacctacaaggccacaggggttagggcaggactcaggtccctgaatcacagcagcctgcattcaaaccctggctcaggcctcccaccagcctcgtggaactggtttcctaaaatgaggagagtccctactttgcaggcttgtgacaacaagatgacagcaagtgcaaaagttccaagcccagagcctgcagcctgcagaagctggcctcattaccacccggatgttctccgggctgcagcacatgaaggggatacgtgacaatccctgctttaagtacagctcagggagttgacgggacctgcccaagcacatagtgatgccgctaatggctcaccaggaagaatggactgcaaagcctggttcttctgataaactccattctgtctcccagtgtgggttctgatgcatagggaggaggaaaagacagtgcttggattttggggtgaagagcacaggttttggagtcaatgagacatggagtatgagggtctcagctctaccgtttactactaaataaaaacaggccactgacctctctggggtttagtcttctcctccagggaatgggaattcaaatgtccttacagggttttcacaaagattaactgaaataatgcacacaaggcaatcacagagtggagtatgggtgctcccttttctctcctccatccctgctttattttttcgcctgggcacttaccaacacacgattattgcgcttgtttattttatttactgtcttgtctcctcaacagaatgtcagcttccagagcaggaatttttattttgtttgttgctatattcccagcccctaaaacagggcttggcacacagtaggagctcaaaaaatatttgttgaatgaatagctcacaagcagacagatgaggacagaggggtcttgagactgatctaacagcaccgatattactaaactgcaacggaggcaacggtgggaagaatttctctgtcctttgtttcctgaaagtccaagaccacttttagttgctcaacaggaaacaatactcaacttacaagacctctagggcctatccagggcaaactgggcactgtgaggcaggaggtcaggcagccctgtccctagggtggctcacggtctagtgggcagggccagcttcttcatatgtgctcagaggggccccgtgcttggtttaatactctgttggtgccatcttgaaattcttaataatgtttgttgttgttgtttgtttgttggtttgagacagagtctcactctgtcgcccagggtggaatgcagtggtgtgatctcagctcactgtaacctccacctcccgggttccagtgattctcctgcctcagcctcccaagtagctgggattacaggcacgcgccaccatactcggctaatttttgtatttttagcagagacagggtttcaccatgttgcccaggatggtctcaaactcctgacctcaagtgatccgcccgcctcggcctcccaaagtgctaggattacaggcgtgagccactgagcccagcctcttaataatgtttttaaaaggggctctcccatgttcattttgcactgggcttcacaaattacgcagccagtcctgcattacaggaaatatttctgtacctaagtacatatactacaaagcaagtaccaaacaccaaggaaacactaaggagagaaaaacgcctgtgagaagaaaaaggaagacacgaatcattcccaacagaagctgttaccatgaaggaagtacgggcaggggcatttgttgaatgtctactatgggagaaggggttcgcatcatgagcacatttaattctgacaaccaccctacaagctgtgtactatactggccatttgaaactaaggcctgcccgagatcatataatagcctaggaggtgacaaaggacagacacaggagccaaacccatgcccatccctccctaagtccaaaatcatagaaaaaaaaaaataagaatcaacatgggcggttatttttaaggccagcatgttcaaggtgggggcaaatccaagagacactaagcctcagagcatgaacaagcatgtgggtgctgagtggaggggaccagtgtttaccagggtgatgtcagactctgcaaggctcgctccccgtgtttctggtctcttcccatgagcaccaggcaccccttaccatccccaaactaggcacatctgtaacgctgaatggaagcctacttgtttacatgtgttctatgttagactgggggcatccctagaacacacacagattgactggtgggcagaattctgctaggtgcatgcacccgagtgagcctttctctttgaatgtgggagggaccagagaacaagatgggagagctgttcccttaattaggctgtgctgcacattaaaggcggtaagacagtcattccagtgataacaatctgtcataagaccctacagaagcagactctcctgttggccttgaagaagcaagcaccacgaattctccacagctgcaagaaaatgaattcaggccaggcgtggtggctcacgcctgtaatcccagcactttgggaggctgaggcgggtggatcacctgaagtcaggagtttgagaccagcctgaccaatacggtgaaaccccatctctactaaaaatacaaaaatttgcagggtacacctacagtcccagctactcgggaggctgagacaggacaaaaatttgcagggtacacctacagtcccagctactcgggaggctgagacaggacaaaaatttgcagggtacacctacagtcccagctactcgggaggctgagacaggagaattacttgaacccaggaggcagaggctgcagtgagccgagatcgcaccactgcactccagcctgggcaacagagcagaaaaaaaaaaaaaaaagtaaaaaaaaaaagaaaatgaattcagccaagaaccacgtgagcttaaaagaggaccctggggttcagacaagacctcagccccggccagcaagccttgtgagttcccgaacagagaacccagctataccgtgtccagattcctgacccatggaagctgtgagataataaacatggcctgggtgcggtggctggccaggcatgatggctcatgcctgtaatcccagcactttgggaggccaaggcgggcagatcacctgagttcaggtgctcgagaccaacctgcccgacatgatgaaaccctgtctctactaaaaatacaaaattggccaggtgtggtggtatgcgcctgtaatcccagatacttgggaggctgaggcaggagaatcgcttgaacccgggaggcggaggttgcagcgagctgagattgcgccattgcactctagcctgagcaacgtgagcgaaactccatttcaaatttaaacaaaataaacatatattgtttaactgttaagttagtggtaacttgtcatgcagcaggcaatgactgatacagtaacctatgcacacatccatctccagtacggacacagaacttggatgcacggggtgcatgacacctcttggcaggacttaactggacagacaagcaacaaagacaataaagcccaggctaagatggactgccaagggcagggaggaaccccagagtgtggacaggtgcaaaagtaggggtgttcaatgaagaggggaagcatggtctgcagggcaatgacatgccaacccccatccactctgacactgtaggggagggggtgaaggcaaaaccacacttcaaaaggctgtagggagaatggggtccctgggggacttccaagtggagaccaaaaggggaagggagtgcggagagaaaggcagaggagtcagggagttcacagtttaccactgaaaccaaataaaacagaagagacaaaatcctgcagctcgctctggcccaaacctttgctagggcaggcaatcacaaatgagcaaattataataattctaatgaccacgttcccgcaattgacttggaaatgctggattaaaaaaaaaaaacttcactcctgatccacaccctggggacaatattatctccccagtgtcctacctagcccacaactacttatgtctcatgccagactgagccagctcccgggatggcaagggagccaggagctgctgccagcagggccatctgctcaccaattcccacagtctgaacggcacagcttccaaagagggactacgagcggccagcagcagcctgcacatgcagaaggcaggggagagcgagggaaatggatctatactgctctgctgcaatcatctgcatgctgggtgtgagatgatcagttcttgagacacttcccagaaggccttcagaaatactgtctgagttacaacactgcttcctccaagtctgtattcttatttgcatcttataggaatgtagccgggtaaaggaggaaggctgcttcaagtcaaagggcatccatggtgggcgccctctcaggcctggacccagcacctgcaggagtcggcccctttaattctcctctgccgtgaactaacactgcacatcagcaatactttgtgaagaccgagcacagcaaccaagcccactgtggacctgattctaggcaaggaactttttttttttttgagacagggtcttgctgtcacccaggatggagtgcagcggcacaatctcagctcactgcagtctcgacctcctgggttcaagtgattctcctacctcaacctacttgagtagctgggactacaggcgtgcgccaccatgcccagctaatttttctattttttttgcggagatggggtcttgccatgttgtgtaggctggtctcaaactacggggctcaaagcaatccactcaccttggcttcccaaagtattgggattacaggcgtgagccactggggctggcctagacaaggaacatcacacaactactccacaaccctgaaaggtcacagtgtcatccctgttttataggtggaacaattgagacccacagagctgtaagaacacacaaaggtaaaggaactcagccaccagcacaggagccagatgccaaacttaggtctgcctgactcagaaccccactgccttccctctacacctggctgtttctcctacatgtctggaatttactgcagggtcaaaggttcatccatttaaactgttcacttttatcaacttacttattttgagacagagtctcgctctgttgcccaggctggagtgcagtgacgcaaactcggctcactgcaacctcccgggttcaaacaattctcctgcctcagcctcccgagtagctgggattacaggagcgcaccaccaggccggctgatttttgtatttttagtagaaagggggtttcaccatgttggccaggctggtctcgaactccggagctccagtgatccgtcccgccttggccttccaaagtgctgggattagagatgtgagccaccgtgcccagccatttaaactgtttaaatgctacacaaaggcagagaaatgaggccgtcactaagggatttgagagcagttagggatacaacaagggcacacagacctgcattgtaaggcgggtgtggcacctgtcacagatagggatgccaggggctccctgctttctctgaagagagggaaatcacaaatatctggggcaggcgcacttttagctggtcatgaggactacagccaggtgaaaaggaactggcctagggaacgtgtgtgacgggggagcagggagtagtcccaatggactggaaaaggcacatgcgagaggggagggtggaaaggccaccaacgccggtgacgctggagctcagaaaagactccgaggaccagaaggaagaagcatcaagggaccagggggtgatatgccaaggtagagaggatgggtctgaggtgttcctctgtgacgggacagaagagatgtgaggacctgaagaggcgccaccagggaacttgagaaaaagaaggcaggtggctcggaagacccgatccatgtgaccctgcaatttattggatatggatgaatcagagctgactttttcgatgacccaaaagatgaaactattaattaaggctagacggaggggagaaaagagagagaaataccctgctacctccagtttttctccctacagcacctcctagagatgaggtgatggcagctcaccttcaggatccattggaaacaaagagaaatctctccctaattctcctgaccccaaacagaaagcaaccaactattccataattttcttctctaggagagattcagagagaagaggcctgaaaatgcaaattaacacacggatgtgaatgagtttcagtcaaagcttaaaccaggacacaacttttcttgtgtagcgaggaaggctaggaggcaggatggtgtcctgtgcctaaaaaggtgagtgtgacgtcaaggtggctgaagaggggtttaaaagggcacctagggggacaagcccagagcccagcatcccaccctaaatgagaacacaggtctccaactccagcccagggtctccctgtggtcacaatggtgttggggacctgctgacagtggcacggaaggactctcggtggtggtcagaatgacccaacatcccaggaggcacctgccaccagttggcatgagtccttggtgctggccctggcgctgctgctaatccaccccagtggacttaggcatgctccctcacctgtatgtccaagacagctaattcaacagtactactaacctggtccccagaaaggcggcagagtaggatcaacttggcattagaggtctcgcttcaaatacaggatttcccaattccaatcttgggcctcagccaccaaccgggaaaacccccctccaagggctgttttgagaatatggaaagctgccaaagctttatttcccatccctttgtaaggtcccctgcagctcccaactagaagagaaagggaccttttattagcaagaaaagggccaggtgcagtgactgtcatcacgcctgtaatcccagcactttgggaggctgaggtgggaggatcacttaagcccaagagtttgagaccagcctcaacaacacagtgtgatctcatctcttcaaaacacatttaaaaaaattagccaggtgtggtggcgcacgcctgtagtcccagctacttgggaggctgaggtgggaagatagcttggccccaggagttcaaggctgcagtgagctatgatcacaccattgcactccggcctggacaacaaagaccctgtctctaaaaaataaaaataaaaagttttaattttaaaaaaggttaaatactaatgagaaaggtccacatacaaattttcatgtcactgctatttataagttcttattaactgaaaacactatgctataagctattaaaggtaactaaaaaataaaaatagctctttgccccaaagacagtctaagtgacagagctcagtactcactcatcaacaacagccctgagagagacaagagatggaagagattccaaccccaagaaagggctggaggggagccaggggaggaggcatgggggaggggacctccaagagggagctaggggagggcagatggggaggagagcccagaagggagctgggggaggggaacatgggggaggggagcatgggggaggggagctccaagggggagcatgggggaggggagatggggaggggagctctaggagggagctgggggaggggagatggggaggggagctccaggaaggagctgggggaggggagcattggggaggggagatgggggaggggagctccaagagtgagctgggggaggggagatggggaggggagctccaggagggagctgggggaggggagatggggaggggagctccaggagggagctgggggaaaagggcttggggagggtagctcaatgacgggacgtggggatggggagctaaggaggagatctgggagaggggagcttggggaggggagatgggggaggggagatgggggaggaaagctggggaaggggatctgggagagggaagcttggggagaggatctagggaggggagctgggggtggggagatggggaggagaaatgggagaggagcttggggagggggatctagggaggaaatctggggcaagggagctgagggaggggaacttggggaggggatttagggaggggagctgggggaaggggagctcagagaggggacttcagggaggggagatgggagaggggattggggagggatggttagggatgggatctagggaggggatttgggggagggaagcttggggaggggatctgggggaggggagtggggaagagagatggggagtcgggggaggggaacctggagggagggatctggggaaggggattttggggaggagaacaggtggagagaggagctggtggggagggcagttgggggcaggcatctgggggagatttggggggaagggaagctgggcgcccacaggagccgctgtgaggtgggcaagcccctctttcagttcctcctcgacagtcagtctccagacttccactccacccctccctgcttccacccagacagtctgatctgcaactcggcccatgactgcccccattgggaatccagctgcttctagcctgggaaccctgacgtgggccctgacctgaccaatcaaaaaccccagggtgatggagcaaatgtgtcctgtatcttgagcataacattaaaagtgaggacccagcagaagtcccccagcgaggacccagaaataaggaatctctttgattcttgcaggctagtgtttccctacccacataatctttagaaatcatgtgtgccgtaataaaagtgagtatttcccctcccttcactcaagcacacagaaacatcggagaaaagctgagcatatttctaccagttctgcatatgagtttgaccagaacaccctgctgtcggtaatgaatggttgaccccaatttctgaacacatatttccttttccaattaattttccttcccctcatgagataaaacagactatttttttttaaagaacaatattcctgaaaatttatttactttttttaaaactatgaggtcagagtttaagactggctccttggtatgaaggaatacatgatattaatataacaaagggctgaatcttccataaatcaacaaaacacccaaacaaaggcagaacttaatttttggcaaagaaaaaacaaaaatgtttttggtgtccattagtgaatacatcagctgaggactgccatcttggaatcttttaaatgagcagagctaaagatttctcataagcacaattaaagcaccctgaattgatacctttagggggttgagtatctgtttcaaatcagcaaagtgcttaccgcaaaaggaacaccttaccaaaagcaagatgaaaaagtgagggcagagtgtcatgattattacttttttttttaagcagaagaatagtctgcaagaaaatacataaaaatgctcaagttaggccgggcacagtagctcatgactgtaatcccagtacttcaggaggccaagcaggaagatgtcttgaggccaggagtccaagaccagcctgggcaacacagcaagaccttgtctctattagaaaataataagttaccaaaaaatgctcaaaatggtaatgtaagggtggtagaataatggcaattattttccttcttttccaagcgttatataatattattaaagtggctagacatatgtatggattttaaagcacttcagttttatgtgttttaggtataatttctaaagcactaaaaaattggcatattcttttttttttttttttaagacggagttttgctctgtcgccaggctggagtgcagtggcgcaatcttggctcactctgcctcccgggttcaagcgattcccccgcctcagccccccaagtagctgggactacaagcacacactaccacgcccggttaattttttctgtttttttagtagagacagggtttcaccatgttggccaggatggtctcgatctcctgaccttgtgatccacccgcctcagcctcccaaagtgctggaattacaggcgtgacccaccgcacccagcccaaaattggcatattctttttgaacgttttccctttgggagaggaacaagagcattccttacctgcttgggagaaagacttaggaacaagaattgaaagtctgcttacctgaggtttaattttcgatcttcttcgctgccagcctccaactacagagaaagaaagagaatatcacaccacaggcaccactgtcaacacgcctcgggcggcagtctcacattttctaccccggtactggaaaaagataaagatatccaggaaacctagctacttctaaacagccgtgccctttcctcaccaatcccggtctgtcccttggagtcatttccgtgggggaattttcaggtttccaaatgttgacccacattcctgccgcagtccaggggatggagtcctgttagctcaacatttcctatctggtgttgttacccagcacggtcttttagccctcagccctcaactttccgaggttgttctggaccttatcctgtttttctcttttaaggggagggggtcatgtttaaagagaatccacttcctccgcagagccaggcaataacagctgagtgatgaacaccattttcaaaaaaccaacccaggcaagacttgcacagtggaaggtggccaggaatcaggccgtctgtttgtgggtcttgaaagctcttgatggttctcgaaaagacttaaacatttgatacgaaacatcctaggctatcggtttatttatataaatgcaagaaagagatatttaatattttctgaaatctaaaaggccacgagtttgggctccagaagtacctatgacttatttttattttttttctttcagagagcaaactgaaaataagaaggaaacacatacacaccccccaaacaactccgcaccgctgggacttggcatgttttttatgttgcacagaggcgcccattgaatgggaaagagaaacctggaaagctgtgatggctgggagagatgcagggctgatcgaggacagaaatgaggcaggagccaagggcgaaggaaaaagggtccagagataatgtagggaggggcctgggcagcaagggacacccaccaggaggtggcaacttcaaccaagaatgagtacaccagcccggcgcagtggctcacacctggaatcccagcactgcaggaggccgaggtgggcggatcacctgaggtcaggagttcgagaccagcctagccaacaaggtgacacgctgtctctactaaaaatacaaaaattagccaggcacggtgacatgtacctgtaatcccagctacctgggaggctgaggcaggagaatcacttgaacccaggaggcggaggttgcagtgagccgagattgcaccactgcactccagcctggtgaaagagcaatacttcgtttcaaaaaaaaaaaaaaagcgtacaccagagggcctgggagtccctacatcatattaagatgaagtacatacaagattctgcagaggcacctaccccacactgaaggagaagtggaaagagcagggaaggcttcaatgaccacaaaacaagtcacaagagcaacaaattgacaaagagtatgttggggtctaacccagtggttctcaatgcggagcaagttctctcaacaggagacattggaagatgtctggaggcatttttctggaggtcactactggcacctagtgggtagaggacaggatcccacaacacacaggatggtccccccacaagagagaatgttctggtcccaagtatcaacagtgggttgagaaactctggtccaatccaaaaaagtgcctggagatctgcccatgaaaattagtcactttgaatgtttctcagaaataacaatgttatgatccattcctgaaaattattgatttatctattcttgtgctctgcctgttcacacaaaggaactgagataagattcacaggaaaatgacataagtgacataatcaaagactgggaaaaaaagaaaattaaaagagaaaagagagccttaggactgagggctgtgatcccccgtttcccacgccggcagcaggcctggctgtgtcaggaaagcactgccctaagtgtctgactcattatgaagttgcaatttgaagagtgatgacgtgacttgggggtacggacttcacaatcatttaactctcggtcactctctgaggttctcagatgaaaggccatctcaggtcagttattccagggaaactacatctgccaaggaacacatgaaagaggtaattcagtccttttagatgagccagggcccacacacaggaagcaactcaagcgagggcggaccagggcagaaccggcctggcctaggtctcctgaccccatacacacttgctgtctccatcccaccttgcttctcacctcaacacatctgaacgagggccttgccttcgggaaacatcccagcgcattcaaagccaagcaatgaatgctgcagctttgctatgatcaaataaaagctgcctgagttttactttatgtttatcagggtcattggcacttggtaaaaataatgctttatataataattgaaaatgtatctagtagacacaacacaaatgtccaacaaaatgtggtacatccatacaatggagtattatacagccatgaaaaggaatgaagtactgccacatgctacaatatgcatgaactttgaaaacgtgatgctgagtcaaagaagccagacacaaaaggccacacagggtgtgattccatttatataaaatgtccagaataagcaaatccatagatacagaaagtagattagtggttgcctaaggttagggagaatggggcagggggaggctgcaggtgagggctaacgggtacagggttcctattttggggaggatgaaaatgttctggaattagatggtggtggttgcacaatctcatgtatatactaaaaaccactgaattgtacactttaaaatggcaaatttatggtatgtaactaaaaaataataagaccttaaaatgcgtaagacaagaacagattaggttgcaagtaactctaggaacatggggttttgaatcagaaatctgggctgacaggttcagcctgaagccaacctctccccttacctcacataaacttttgtgatgagaacctgagattaagtgcataaattgccagacagcagtgaccggaacagaaaacagcccctcctcgctgtgggaaggaaaggcgctcctgcaacctaacttctcagtagcaggctattgatcgccagtgttcttttgcctctaatcagcgtgtagagggggattactagaaccttctgtgtatagataactcatgaatggcctctcctctccaaggagggggctgtgaaggttcaacttcccagccactctgaaaatgtccctgccaatcccagcaaaacaaggctgaagaactaccctaccaggagacagggctgtcaagccaaatgcaaacattattctcttgtctctctcagacacacaaacctcccccgtgttatcagtcaacttcccccactccctcccacaaagaaaggggctgaagagcccagatgctggctgcggaacttcctgggcctgggaccgcagggccgctcctccagtcttctctaaacacagctaagggtctgcaggcggacactcagccttgttataggtaagagtttagaccagaggccttgacgggttcttcaagagatggtgggcaagattgcgcgaccagagggtcatccctgcagctacagagggctgacctgctcagaggcccaaggccccagcctaggacaagccaggccaaccctgcaggctaagagggcaacagtgccctcaatcaaccccagaggaaaaagtggccaggcaaacggacctgggccacacacagacccacaaaaacgcgcacagtgccaggacacgcaacccaggaatgcacctatgcaatcacccagaatgggtcacagccacacagaaagatagatgcacataaacacacaggcctgagtgatgttacagaaaggaaaagccagactaaggctgcacgcacagacgtgaaacacagccacacagagcccacagcacgctcggtcaccgtcacacagtgacacgggcacgcctacagacagaactccagaggcggcaggcggggaaacaatctcacacgtttgtaggggcactcccagatgcctgtctcacgctggcacagtccccggtacggcaggtcagcaacagtcacatctcacatcgcacagccaggcatacaggcaaagggcctagaactaccccggccacaggtctcagaaccagcggctcacgcagtcacccaatcaagggtcccagttgcacatccagtcacccctggaccctggtcacactgcagagtcactcacaaatgggagtcccgacagacgcacagtcctccccagacagaggtcaacccaagatgggggtcacacctgaaatcacagtccccacacaatcacgaggtcacatctgcacacacagtctctgcacagtcacccttaggggtcacaacgcacacagtctccgcctaacaggggtcaccccaagatgggggtaaccccctgatgtgggtcacagcgcacacacagtatcctgcagacactcccagaggggtcgcactgcatacacagtccctgcaaagtcgccccccgataggggtcacaccgcacacaaagtccccgcacagctccccaagacagggtcacatcgcacacagtcctcgcacggtcaccccggtccggctgcccggctctgttcctacggcggggccccgaggagcccgcgcagccgcccccctgccccgcacgcgcggccccagctccggcggcctcggcgcggcgtccggcggcccaggccgggcgcggcgagcccggggctcacctcgctgttgctggccgaggaggaggcggcgctgggcgtgggcgagcgctgcagggtcaccagggccatggctgcggcgcggtgcgagggcgccacagacgtctcgagctagagccgccaccgccaccgccgcccgggccgggcccggggcctcctggagccgcgcgcgggcggccgggccgagccgggccgggcccgcccctccccctcggcgtcgccaccgcccccgcccccagctcccgcctcccgcgccggcgcgcgcaggcctcagtgcgcggagtgggcggggaagcgggcagggcgggacgaggaggcgcgcgtgcgcgggggccctgagggctgcccgaggcctcggctggtcgatcacgtccctcgcgcgcccgacacacgcgcccccgcccgcgcgccccgctatcaggcctgggactcgggggcgcgcgcgccgcccggagcccgtacgccccaggggccctgcccgctgctctgcctggggaaactgaggcccggcgaccgtgcagacaggactgtacagcgaccaggaaataaaagacgtcctggggccgggcgcggtggctcacgcctgtaatcccagcactttgggaggctgaggcgggcggattacgaggtcaagagatcgagaccatcctggccaacatggtgaaaccccgtctctactaaaaagacaaaaattagctgggcgcagtggtgcgcgcctgtagtcccagctactcgggaggctgaggcaagagaatcgcttgaatctgggaggcggaggttgcaatgagctgagatcgcgccactgcactccagcctgggcgacagagcgagactcggtctcaaaaaaacaaaaaacaaaaaacaaaaacagtaagcaaaatagattcgcctgattttgcagaggttaatcaagttattaggcacgtttttaaaaaagtattttgctaatctttttcaatgaattctttctgggtgttctgaaacccagccaactccttggaggtcagggaaggcttcccagaagagctttattctgaggcttgggcttgagcataagcaggattaacaggtgaaagaacagagagacagctctccaagcaggggggatcagcgtgccctgaagcaggaagaagtttgtcaaccggaggccagcactcagggaagggaagaggggaggaatggctggagtctccatcctctctggaaagatcgctccggctgctgcgtggatgagggaccacggggcagagggctgagggagaccagggaggaggctgctgctgttgtcccggggagaggtgaccagttatggggatggagaggggaacatggaataagataccaagaaggcaattctggcttgacttagtagtaggaaacttttcttttagccaaaatctcatctcccggctcccacccccaacctctgcatgttgcacaagcactcgcaaacgcagtggtcccagcctgccccgcagcttagcaaatttgtcttactgcccaacaggaaacccacgcagcctcctggattcttccccgtccctccctctgtcctggggctgtgacctcctccatgttattcacagggtctcagcacgattcatctcaaaggtgattctagtggggggcactgtagcttctacggagcgtttctaagaggggatttgtgggaatgtttgtggttgtcttgctgatggagggggagagctcctggcatttagagtgcaagagccttggatgctaaatgtcttccaatgcactggacagtctccccaacaagaattgctccattcccacaaaatgtttcctgggtgaaaaacccatttatagtaatttgaagccagaacctaactccatttcatgcatcaacactagtcttccttccttccttccttccttccttccttccttcctgccttccttccttccttcctctctttctctcactttttttctgaaacagggtctcactcccgtcacccaggctgaagtgcaatgtcacaatcatagctcactgcagcctccatctcccaggctcaaatcatcctcctgcttcagtctcctgagtacaacgggtacacaccaccacacccagctcctttaaaaaaaaagtttaactatgttgcccaggcaatcctcctgcttccgccttccaaagtgctgggattacagacagaagccaccatggctagcctggtattttttactgaattttcagaaaggtgactatgttgaaaccctgtctctcctaaaaatacaaaaaattagccaggcatggtggcgggcacctataatctcagctactcaggaggctgaggcaggagaatcacttgaacccgggaggcagaggttgcagcaatctgagatcgtgccactgcactccagcctgtgtgacacagcaagacagagagaaagagagaagggaagggaggggaggggaggggagaggaggggagaggaggggagaggaggggaggggagaggaggggaggggagaggaggggaggggaggggaggggagaggaggggagaggaggggaggggagaggaggggaggggagaggaggggaggggaggggaggggacgggagaggaggggaggggagggaaaggaagggaaaatacactttgttttgcttgagagttttgtcaagagttgttcatccatccttagggaaaaggaggtaatggatggcaacgcctctgctaatattagagcatcccacacaaggtgcccacaactgtagctgcactctaggtagacagacagtcataggtacttaaatgtcaaatataagggaaaattgtggacaaaattcagttgagtagagaatattttatttctcaaatccaagcacattgattattggcaggcccatgcttctgagatgcccctgtgtcctctmagggagtagtggctgagcatttccacattgtaatgcatgttgtttcattatgatttatttttcttttatgtctctcttacattagttttcaaatttgagagtttgagaatcccctggagaaaatacagattgctagaccccacctcccagagtttcgaattcacaaggtttgctgtagggctggaaaatttgcacgtctaacaaattcacaggcaatgctgatgcttctgtctggggacgacagtctgagaactactgcctatacaaatgcaatggcctcttcaccaagaaattcctacctagatctgatcctggtacccgtccgtggcccccaatcctaatccccctgctctggccggcctgcttttccactcaccccaacttttttggaggcagtctccaccccttctcacttcctcttagagctgagagcccttttcttccccacaactaactcttgctagaaatcacctccaaaaagctttccctgccccttaagcagtgtcatttccaggatctcgtagccctcaccctacccttaaacacacagcaagtgtcagtctgccttatcataatgggtccatctctctgtcttgtcccattaccgtagagccaggaacggtccctaagaaaagcctcaggaatcaggctgggaccagcgtgagggtgcaaaatgtaagagggtgcccccaaaaactcaatgattaagataaatagtattttaatgcaatattttagaaaatcaaaattaatgccaaatccatgatgaataaaatatttttaaaatttgctttttttttttttttttaattgagacagagtcttgctctgttgcccaggctggagtgcagtgtggcacaatctctgcctcttgggttcaagcagttctcctgcctcagcctcccgagtagctgggattacagacccccaccaccatgaccggctaatttttgtatttttagtagagatggggtttcaccatgttggccaggctggtctcaaattcctgaaatcagtgatctgcctgcctcggcctcccaaaatgctgggattacaggtgtgagccactgcacctggtcaaaatatttacaaaaattttttaagagccaaggtctcattctgtcacccaggactgggtgtagtggtgcaatcctagctcacttcagccttgaactctgggctcaagccatcctcctgcctctgcctccggagtacctgagactacaggtgtacaccaccacgcctggctgactttatttttgccagaaactgggtgttgctatgttgcccaggctggtttcaaactcctggaggcactcaatcccccgaccttggcctcccaaagctttgggattaccggcatgagccaccacacctggccaaagtatcaaatttttaagtaaaattggcatcagtattgtgtcactgattcttccacttacttcagacttcagtgtagctcagcaaagcacttttattgatcctgtctttatttgattcttttacaactttggccattctaaagccttttgtgaaaatggcctgtggttcagctgggcatggtggcgtgcacctgtaatcccagctactcgggaggctgtggcaggagaatcgcctgaaaccaggaggtggaggctgcagtgggctgagatcgtgccacttttgacactctgtctcaaaaaaaaaaaaaaaaaaaaaaaaggaagcctgtcggcttgactccagtagcctctgatggggtggagtggacaaggggaagtgaaagctcccaggcctcagtcagggcaggtcccaagaagccctgagcatggaggaggggaacaatccagtagaggcagctctgaagttttctcccatgcattagagccctttccaatcagtatcatgatttttcatcatataatagtttatttaatcatctttgacctcctccttgtagtcccagctcacttttgtaactaataaaaaacagtgagttattgagctatttgctctctgctaaggcacaatgcaaagtgctttgtgagtgtgtgggggacatgatttattaacatgtgactgtccccccacttatactccaagatcacctcctccaggaagccttccttgccccgtggctgggttaggcaccccttctctgtgctcctacagcccctgtgcattagtgacaatggcattgtggatctgccctaggcccatttctgggttgggacactttaggtacattcattcttgtcaccctgtgattctcatttcatgggtgaggaaattgatgcacagagtggttaaggcactggccccaagttatgtaactaaggagtggtgaacctggttcacccatgtttttctgctttagaactcaggcaaagacaggttcttccaggacagcctcagaaagtgttggtgcaaattaggttggtgcaaaagtaattgcggtttttgtcattttttttttttttaatggtgcaaaagtaattgcggttttgtcattaatgaccaactattataagtaatagttccctttttttttttttttgagatggaatcttgctctgttgcccaggctggagtgcagtggcttgatcttggctctctgcaaactccgcttcctgggttcaagtgattctcctgcctcagcctcccaagtagctgggattacaggtgcccacccccatgcccagctaatttttgtatttttagtagaaacggggtttcaccatgttggccaggctggtcccgaactcctgacctcaagtgatccacccacctcggcctccccaaagtgctgggattacaggtgtgagccactgcacctggccagtagtttgcctgttaaagcaaataacttgtaatttctccttaattattcattccaaaatgatattcagaggtaataaagctctgataggctgaataatggcctgcaaagatgtccatattccaaatccctagaatccctgcctatgttaccttgcatgctaagagggttttacagatgtgattaaactcaggatgtttagatggggaaattttcctggaggaggcccaagaggtcctaatgtaatcacaagggtccttataagagggaggtgagaaggtcagagtcagtagtaagagatgtgacaacggaactgagggattagagtgaaggaagaggccacaatccaaggaatgcaggcagttgctaaaagtggaaaaacaccaaaaaatgaattctcctttcagagcctccagaaagaatggagccctgctgatatctttttcttttcttttttgagttagggtcttgctcacagagctgtcacccaggctggagtgcagtggcatcatcatagctcacagcagcctcgacctccagggctcaagggattctcccacctcagcctcctgagtagctgcgactacagacacacaccactatgcccggttgactttttttaattattattatactttaagttctggggtacatgtgcagaatgtgcaggcttgttacataggtatacacgtgccatggtggtttgctgcacccatcaacccgtcatctgcattagatatttctcctaatgttatccctcccctggcccaccaccccctgactggccccggtgtgtgatgttccccatgcccggttgatttttaagggttttgtttgtttgtttgtttttttagagacgagggtctcagctgggtgcagtggctcatgcctgtaattccagcactttgggaggtaaggcgggcagattgcttcagcccaggagttcaagaccagcctgggcaacatggcgaaaccaaaaaatgcaaaaaattaactgggcatggtggcacatgcctgaggctgaggtgggagtatcgtctgagcctgggagatcaaggctgcagtgagccatgatcatgccactgtgctccagcctggttgatggggtgagaccctgtgtctaaaaaataaaagaaatgaaggtcttgctgtgtttcctaggctgttcttgaactcctaggctcaagcaatcctcctgcctcagccaccccagttgcttggattacaggcacaagccaccatgtccaatcctggcaacgtcttgattttagacttctgatctctacaattgcaagagaataaatttatgttgttttaagccacgaaatctctgggaatttgttacagcagccatacgaaatgaatataaaactcaacctccatttgggctttaaaaaacatatcattataatgccattacccagtatattccaggtgcttcccaagcgttgtgtcattttctcattcactcaactcatccaataaactatgtttgttgctctcctgggcactagtctaggaatctgggttccatcagtgaacaaaatggaatcactgcccttgaagagcattcaatcaagtgggaaatatagtaaaaatatatatatatgcaaatatgtttaaaatcatatgtggtaaatatattgcatttaaatgaattaataggccgggcacggtggctcatgcctgtaatcccagcactttgggaggccgaggccagtggatcacttgaggccaggagttcgagaccagcctggccaacatggcgaaaccccgtctctactaaaagtacaaaaattagccagttgtggtggtgggtgcctgtaatcccaggtactcgggaggctgaggcacaaaaatcgcttgaactgagggggtgcggaggttgcagtgagccgagatcatgccactgcactccagcctgggtgacagagtgagactgtctcaaaataataataataataattaattaaatgaattaatattggtaagggtccttagaacaagataggcactgatatgtgtcaaataaatgaaatatgatgtccaatcatgaaaaagcttgggagaaaaacaaagcaggctaagggcagagtaatggaggaggccacttagacaaatggtcagggaagcttctgggtgaggtgatatttgagcagaggaatcaccatgacagcaccaccagggaggtgtagaaaccctgggatctgcctggttcattcaaactggcctccccactaaggaactgtgaggtactttttctgagacccattttctttctgtctgtgtcacccaggctggagcgcagtggcgcgatctcggctcactgcaacctcctccccccaggctcaagtgatcctcccacctcagcctcctgagtagctaggattacaggtgtgtgccaccatacccagctaatttttgtatttttagtagagtcggcgtttcaccatgttggccaggccaggctgccaccttggcttcctacagtgctgggattacaggtgtgagccttcagacccagccgagacccactgtctttctctgtaaaattgatatgaaagtgatagtgctcggccgggcatagtggctcacgcctgtaatcccagcactttgggaggccaaggtgggcagataacctgaggtcaggagttcaagaccagcctgtccaagacggtgaaaccctgtctctactgaaaatacaaaaattagccaggtgtggtggtgggtgcctataatctcagctactcaggaggctgaggcaggagaatcgcttgaacccaggaagcagaggttacagtgagtcgaggtcccgccacttcactccagcctggacaacaaagcaagactccatctcaaaaaaaaaaaaaaaaagaaaagaaaagaaaagaaagtggtagtgctgacctcagagcttggttgtgtcaattgaacagcatactatgcaggaaaggcagagcgtgctgtcctatttactaatagtacctaaggtattgggttgaattgtgtccccacaaaattcactagtccctgtgaatgggaccttatttggaaatgaggtctttgcagctgatcaagttaagatgaggtcattagggcggggccctattcgcatatgactgtgtccgtatgaaaagggggaaatttgctgggcgcggtagctcatgcctataatcccagcactttgggagaccaaggcgggtggatcacctgaggtcaggagttcgagaccagcctgacaaacatggagaaaccctgtctctattacaaatacaaaattagccaggcgtggtggtgcatgtctgtaatcccagctacttcggaggctgaggcaggagaatcacttgaacccggggggtggaggttgcagtgaactgagattgcgccattgcactccagcctgggcaacaagagcgaaactgcatctcaaaaaataaacaaacaaacaaataaataaataaataataaaaggggaaatttggacccagagccaaggggaaaatgcttcctgaaggttgtagttgtgctgccacaagccaaagagcacccgagatggtcagcaaaccaccagagctaggagtgagaagtgaggagcagatttgcgtggccttctgaagaaaccagcaactcgatttcagagttccaggctccagaactgagagagtaaatgcctgtggtttaagcctcccagtttgtggcactttgttacagcagccacaggaaaggaacgcatctaacatgatcatttcatcagctgcagaaaatgaggctcagagcaaggctagggtttgaacccaggccaactagaccccagaccacatacatgggttgttggcctttctagcctgggaggtgacagtttggatgttccactatttgcagggaacggtgttcagaggactcaaagcttctgccacctgggccagggtgtccagtgttagatatggaagtcaggtatctggggcttctgacaaagcatctctctgggtgggtcaataggcaccagcagccaggcagttgagggatctctgcccctgtgcggagttggctgaagcctcccttcctctacccatcccttcatttaacctgcttgccagatcaaggtgttccctggcctctgccaggggtgtattcacctgaatttgccttttattcactatgatcacaagcaacacactgacccttgctgggcctcagaatctcaactcttggtggggtgcggtggcccatgctggtaatcccagcactgggaggccaaggtgggtgaatcacttgaggccacgagttagagaccagcctggccaacatggcaaaaacctgtctctactaaaaatacaaaaattagccaggcatggtggcatgcacctgtagtcccagctactcaggaggctgatgcacaagaatcacttgaatccaggagatggaggttgcaatgagccaagatcacaccactgcactccagcctgggtgacggagtgagactctgtctcaaaaaacaaacaaaagaatctcaactcttaatatggaatgattaatgtcctataagaaagcattggttagatcaattgggtatatgcatgtgatatccctcctagatagctctcagtggtcctcatctcctgatattcatgccctgtggagtctcttcacacaatacatagaactgacctgtgtaaccactaggatatcacagatactacagcatgtggcttctgaggctaggggtcataaaagacactgaagctgctgtcttgctgtctcttggatttctcattctgggggaatccagctaccatgtcatggggacatttaggacatttaagcagccaaagagagagggccgcatggcaagaaactgaggtctcctgccaatgaccagcactaacctattgtcatgtgaatgcaccaccttgaaaatggatcctccagccccagtcaggcctttatttatttatttacttattaattgagaccgggtctcattctgtctcccaggctggagtacagtggcaccatcttggctcgctgtaacctctgcctcctgggttcaagcgattctcattcttcagcctcccgagtagctgggattacaggcgtgcgctaccatgcccagctagtttttttgtatttttagtagagacagggtttcgccatgttgcccaggctggtctcaaactcctggcctccagtgatctgcctatctcggacccccaaagtgctgagattacaggcaagagccattgtgccaggccccccattcaagccttcagatgagatcacagccatggccaacatctggagtgcaacctcatgagacactctgagccagagctgcccagctgagctgcttccagattccaggcccaaagaaaatgtatgagataataaatgtttattgttttaagctgctaaattttaaggtaacttgttatgcagcaatagataacttttatatgctgccataaaaatattataaaaccatgcactagtacagaaagatttttataaaatattaagtggaagaaaagaaaagcaggccaccaaacagcgtaggacagtagaccccatttttgaaagaaaaatgtgaagagttaaaaaactctaccaaaaggggaaaaaaaagagggcatcaatggagagatggagaagctttgtttttggatgggaagactcagtatggtagagttaacaacctctcgaaattaaattaaatggaaatgctattgaaatcccaacttgattcttttgagtgtagggatctttacaacagataactggacaagctaacatttattgggtatatatgtgcgttgcatcacgtgacagtcactgtttcatcttaattccaccataggagaaagtccctctttatttaatttttctgagagtaaagtactgctattacctgttccccttcccattttacttaggaggtttcaagaggggacttgtctgagatcctggaaaccgtggaggtgagatgacatcaagcatgtttgatatttacatgtgtgcccttgggcctcctgccacatggcctccccactgtgccctggtttccctaagtaccagcccaaggacacatggataggaaaggtggagctggggcaccagcccagtctgcctgactccagagtccctggtcttaatcactaaaccaccccagaaaagtaaccgtgggagaagagacctgcaaactaggaaaaagaagattaaagggaaggaatctgttctgctagatattaaaacatatgacaaagctgtagaaattaaaacagaatggggccgggtgcagcagcttatgcctgtaatcccagcactttgggaggccaaggtgagtggatcacctgaggtcaggagttctagatcagtgtgaccaatatggtaaaaccctgtctctactaaaagtataaaaattagctgggcatagtggtgtgcacctgtagtcccagctactctgcaggctgagccaggagaattacttgaacctgggaggcagaggttgcagtgagccaagctcacactactacactccagcctgggctacagagcgagactccagttcaaaaaaaaaaaaaaagaaagaaaaaaaaagaagaagaaaaaaaaaaaccgggcgcagtggctcatgcctgtaatcccagcactttgggaggccgaggtgggtggatcacctgaggtcaggaattcaagaccagcctggccaacatggtgaaaccctgtctctactaaaaacacaaaatcagcagggtgtggtgctgcatgcgtataatcccagctacttgggagactgaggcaagagaatcccttgaacctggaaggcagaggttgcagtgaaccaagactgcgccactgcactccagcctgggcaacaagagcaaaattccatctcaaaaacaaaacaaaacaaaaacaaaaacaaaaacaaacatagaatgggtgctggcctagaaagccacaaacagatgaatggagcacaacattaagtccaggaataaactcaaacacacaggaaagtctggtgaacgataataggaggtaggtatctgtgaagcgtggagtaagagtgagttactcaaccaacagttctgctgccactggctagcaagtaaatgcggatccctaccccactgctagtctccaaaattaattccaaatgagtcagttaaatgtttaaaaaaccttcaaaagttgccaggcatggtggctcacgcctgtaatcccagcactttgggaggccgaggcgggtggatcacctgaggtcaggagttctagatcagtgtgaccaatatagcaaaaccccacctctactaaaaacacaaaaattagctgggcatggtcgagggcgcctatcgtcccagctactcaggaggctgagccaggagatttactagaacccaggaggcagaggttgcagtgggccaagatcacaccacccacactccagcctgggcaacagagtgagactcgttctcagaaaaaaaaaaaaaaaaaaccttcaaaagttatagaaagtctgtgtgagtaatttttaatatgaagcaagggagagagatgaagcagggtttctaaacatgactatagccatataaaagtatgttataaagctgggcgtggtggctcacgcctgtaatcccagcactttgggaggctgaggcgggtggatcacctgaggtcaggagtttgagaccagcctgaccaacatggagaaaccccgtctctactaaaaatacaaaaactagctgggtatggtggcgcatgtctgtaatcccagctcctcaggaggctgaggcaggagaattgcttgaagtcgggaggtggaggttgcagtgagccgagatcgcaccattgcactccagcctgggcaacaagagcgaaactccgactcaaaaaaaaaaaatgttataaaaccacacaccactataaagaaatgataatgcaaaaatcataaaggacgaaaaaaaaaaggaaatagatttaactacaaaaaagtttttgttttgcttttgtttttttagagttagagtcttgttctttttcccaggctggtacaatcatagctcactgccaccttgaactcttgggctcaagcaatcctcctgcctctgaaactgcgtttgcaaaaattataactgagaaaacgatgacagtgaaagagatctgacctaactgactccatcttgcttctaacctccaagctgtccgtgttcattcctgggtgtaggccaaactaactttgggaggaatttagtttatagtttaactttgtcaaagtttaactaagatgttaatagcccattttccaaaacaaacccctttcctgcctggggactagactgcctttgcaggactaacaaattattatagctaccagattagaaattatggtttaggagtcatgcagctgaagcctacaagattctgaatctcccaaattgctcctggagataacatcaccattgtaaaacctaagatcagtgcttgacatattttgcagacctcgcactcgatggatcagctggcactacccaaatggataaacaggctcatctgatctgtggtccccacccagaaactgacccagcataagaggaccgcttcaactcctataactttgtctccaacctgaacaatcaacactcccctactttctgaccccctacccaccaaattacccttaaaaaccttagccaggcgcagtggctcatgcctgtaatcccagcactttgggaggctaaggcaggcggatcacctgaggtcagggttcgagaccaaccatggccaacatagtgaaaccccatctctactaaaaatacaaaattagccaggtgtggtagtgtgcgcctgtaatcccagctactcaggaggctgaggcaggagaatcgcctgaacccgggacacagaggtggcagtgagccaagatcactccactgcactccagcctgtatgacaagagcaaaactcggtctcaaaaacaaaacaaaacaaaaaacccacagaaaaaaaccctgaaccatgatcctaaactctttcactattgcagttcccctgacttgatacattggctctgtctaggcagcgggcaaggataacccattgggcagttgcacctcagcctcctgagtagctgggattacaaatgcaagccacagctaaaaaaattttcaaacctttgtaggacagacaaattggggaaaacatttgcaacaaagggatgatacacatacatataaagagttctttcaaggctgggcacagtggctcacgcctgtaatcccagcactttgggaggccgaggcaggcagatcacgaggtcaggagttcaagaccagcctggccaatatggtgaaaccccatctgtactaaaaatacaaaaattagccgggtgtggtggcatgcgcctgtaatcccagttactcaggaggctgaggcaggagaattgcttgaacccgggaagcagagattgcagtgagccgagatcgcaccactgcactccagcctgggtgacagagtgagactccatctcaaaaaaaaaaaaaaaaaaaaagagttatttcaaattaataagaaaaataaccaacacaattcaatagaaaaatggggaaaaaagaataggcactttacaaagaaataaatacaaagcccagtggacatgaaactttgccatctccttagcagggcttggagtaagatggggagaaggaaggatgcaaaatttaaggaggctgtcactctcagggccatgtaagtacaaagtgggcatatgagggtaagtgcctccttaaatgtgtaaattgctagagccctgctggatggcagtctggcaaaatggatcaatattttaaatgtacaaaccctggcacaatgattccatttttaggaactgaccttatggaaacgatcaggcaagtgtgccaagaaacacatctaggatgtttttaatgtcgacaaattagaaatgacaggtaaattcaaccctacggactgactttaaaaattgttacatctggctgggcatggtggctcacgcctgtaatcccagcattttgggagaccaacatgggaggatcgcttgagcccaggagttcaagaccagtctgggcaacatagggagaccccgtcgctacaaaaaaaaaaaaaagtaaaaattagccaggttggtggtgcatgcctgtagttctaactactcaggaggctgaggagggaggatcacttgagccctagaggtcaagactacagtgaactgtgattgcgccactgtactccagcctgggcaatagagtgaggccctgtctcaaaaaagaaaaaaaaatgttacatccaggtacattggcatcctgtgtaaaaaggatgccatcctgtagtcccagctgcttgggaggctgaggcaggagaatcgtttgagcccaggaattcgaggcttcagtgagctatgttcacaccactgcacttcagcctaggcaacagagcaagactttgtcaataaattaaaagaaaaataaaaagtacgtcactgttctatagtggtcgtggaaagaggctcagggtatgccatattggtacaagtgaacagaggaaccaacatatgtcacatgatactatttttgccacctgcccgtgtttatgttcacatttggaaatatttgcccaaagtaatggtccctatttcctggtggtgggattaattgcaggggattcttactttcttctttatgcctgctgcataaatacttgaaatccttaaatactgcttaatacttgaaaaagtgattaaagctaattttgtctgagaaagagagtgggagttaacctgttattctgtaacttcctggccccaccagggttgactcctgcagagcattctccaggtaaatgtttttgccctggcctgactgtatttcagaactaccaggaggtcgttttgtttatcaaccacccagtggggtcaaaaagacccttaacttctacaattccagccaaataaacagaagttgctttcgaaagtctagggcctcccattactaggatcagtgagtttaggacttcagggtagtggaaagggccttggtcccacagagctgtctcagggcacttaaatttccctaagtgtaaaatggacagcttcaaccgtatcagtgtttctcacctttctcttttcttttcttttgagacagggtcttgctctgttacccaggctggagtgcaatggcaagatctcagctcagtgccgcctcaaccacccaggctaatcaatccttctacctcagcctcccaagtaactgggactacaggcctgtgccaccatgcttggctaattttttgtagagatggggtttcatcatgttgcccaggctggtatcaaacgcctgggctcaagagatcctcctgccccagcctctcaaagtgctgcgattacaggcgtgagccactgtgcctggctttttcttaaactcactctcctttttaataaagataaaattcttacacccttcctagtgggtacctttctccttattccaatagccgagaagatactgtggaactttactttctgtagattatatcacgaaaacaatagttgtcccccaagctcattttccaaaattaaataataattctaagtatgcttgtttgtacacagtacaggactttctgaagccacaggccacctccagtcctggtcactgatgcctggggtccttctctggctctcaattaaaagctatagtgtagtgactgagtaccccagttctgggacacaacctgggtgagggtcgccaggtaaaatacagggcgttctgggggaggtggcccacgcctgtaatcctagcactttgggaggccaaagtgggaggatcaggagttcaagaccagcctggccaacatggcaaacaatgtctatactaaaaataaaaaaattagcctggtgcagtggcacatgcctataatgccagctacttgggaggctgaggcacaagaatcacttgaaccagggaggcggagtttgcagtgagccaagaccacgccactgcactccagcctgggcaacagagcgagaccctatctcaaaaaaaaaaaaaatatagatacacacacacacacacacacacacacacacacacacacacacacacacatatggtgtcctggaatctatttcctagatctggcaaccctaacctagttcacatttgggcctctgcttccaggcagtgtgactataagcacagtctgtctttccttttttctttgtctcaccctctttcttcttctttccttcttccctccttgcctgcctgctttctccttctttcatttttcttcctccctttcctcccctccactccctcctccttccttcctttattccttacttcctctctccttttctctctctctttcttccctaattgtgtcaagtgcatcaatcttaattttaaatatgcagcttgatgaatttttacatatgcataaactcctgcaaccactacccagattaaggagcacgtttccagcatcccaggaaattttctcatgcctcttgctggtcagtatctcccccagaggtaaccactcttctcacagcctgttattgtcaattaattttgtatgttcttgaatttcataaaagtggaagtatgcaatatgagctcttaagtgtctggctgcttcttcttaacctaatgactgagattcattcaggttgctatatataacagtattttcccttttcattgctgtataatattccattgtgtgaatttttttttggaggggggagttttgtttcctgaaaacaccacaatttgtttatccatcctctgtctcatagatatttggttgtttccagtttggggtgtaaattcaaaataaaatcctaagggtccactaaatgaacacccttcttggcaaagggaaccccagaaaaactttaaaaactttgtttccagccatgatgagacaggaggtcaggcacaccacattacactcccttccttccttttgtggtttagatacaagaaaagatcagcatcaatgctaaaatagagggctgagtatggtgactcacacctgtaatcccagtcccctgggagactgaggaaggcagatcacttgaggccagaagttcgagaccagcctgggcaacatggtgaaactctgtctctacaaaataaaataaaataaaataaaataattagccaggcacggtggtgcgtgtcctgtggtcccagctactggggaggctgaggtgagaggatcgcttgagcccaggaagcagaggctgcagtgagtcatgatctttccactgcactccagcatgggtaatagagtgagactctgtctcaaaaaaaaaaaaaaaagagagagagattataagactgacagaacagactttttgtggcaataagataccaaattataaacacagcctaaggccatgtcaggcaagggttaagtcaggtgcccctactcttaaggaataaactatgttctaattatgttacaagatttttctttttctctagcagcgaaacaagcactggcctcagaagaagcaatattaaaacagttacaactcatctagcacacagacacccaactgacaccctgttcctccagtcataacaacaactacagctttgattgaacaagagactgagtttggtaactttctcctaataaaaagatcactgactatggactgcttctggtggggttacgaaaccgcaacctcatgtgcctgcatttcctgaaaagacattttgatgtgtaggttctaattgtaatacattgattgattgattgatcaattgattgattgagatagggtcttactctgttgcccaggctggagtgcagtggcacgatcacaactcactgcaacctctgcctcctgggctcaagcaatcctcccacctcagcctcccaagtagctgggactacaggtgcacgcaactgcgcccggctactttttgtattttttgtagagacaggggtttcgccatgttgcccaagctggtctcaaactcctgggctcaagcgatccacccaccttggactccaaaagtgctagtattataggcatgagccaccatggctggcctaattgtaatacatttaaatgttaagtctccaccccaaagtgaacatgggttgtatgttacatgcacatttgttcatacacatgtgttggggccaccttcataaatattcatagcttctcctgtaacctgctggatatatcattcagccaaccccttcagcacaaagctcctaacccaacccctcctccttcaaagtgcccgtctctgttcttggtaggaggcatacttcccaggccatggactggtcaccttgtgggctataaccccttataagaaataagatttcttctcctctctgaatttacacatttgtgattttttttttttttttttttagttaacaggggctatgaacattcttacagaagccttttgattgatgtgtgttttcatttatcttgggtatatatataggcgtgggcatgatagatattaggatagccatctttaacttcagtggatgctggagcaagtttctgaatttcaactctgaagtggggatgataataacagcacctgccttacagggctgtttcgagattcaaagagaaaatctgggtaaggcagggtgcggtggctcacgcctataatcccaccactttgggaggccaaggtgggcagatcacctgaggtcaggagttcaagaccagcctggccaacatggtgaaaccctgtctctactaaaaatagaaaaacaatgagccaggtgaggtggtatgtgcctgtaaacccagccactcgggagtctgaggcaggagaattgcttgaatctgggaggcagatgttgcagtgagttgagatggcaccactgcactccagcctgggcgacagagtgagactctgtctcagaaaaaaataaaaaagaaaaaaagaaaatccaggtatttagaattggtacaccgcaatttacaaaacgtaaattattgctgtgatggcagtggggagcatgaagatattggactaacttttatgaatgttcaagtgctcccatgatgaattaaacacacagggaactttataagggccatatgttatataagtgatacatgactattgtattaaaattcaaactagttagatataaagtaaaaagtgggtttcaccctatccattttttattattgaagaaaaaaaaatatgtcatagcgtggtggcttatgcctgtaatcccaaccctttgggaggtcggggtgggatgattgcttgaggccaggagtttgagaccagcttgggcaaaatagcaagaccctgtctttacaaaaagtaagtaatttggctgggtgttatggcatgcatctgtagtcctggctaggctgaagcagaaggattgcttgagcgcaggagttcaaggcgccactgcactctggcctgggtgacagagtgagatcctctctctctctctctctcttttttttttttttttttttgttttttgagactgggtctcactctgtcacccaggctagagtgcagtggcttgatcttggttcactgcaagctccgcctcccagttcaagtgattttcttgcctcagcctcccgagtagctgagattacggacatgtgccaccacggccggctaatttttgtatttttagtagagatagggtttcaccaacatgttagccaggctggtctcaaacgcctaacctcaagtgatccatccacctcggcctcccaaagtgctgcgattacaggcaagagccactgcgcctggcctgaccctgtctgttatcttttctttttctttttttttgttttcttttttttttttagacagagtatcgctctgtagcccaggctggagtgtgcagtggtgccatcttggctcactgctacctccacccaccaggttcaagcaattctcctgcctcagcctcctgtgtagccaggattacaggcacaccccaccactcctggctgattttttgtaattttagtagagacggggtttcgccatgttggccaggctggtctcgaactcctgacctcaggtgatccacccaccatggcctcccaaagtgtcagaattacaggtgtgaggcactgtgcccagccgaccctcttttaaaaaaggaaaaaatactatgcagtgagtattttgcatgcattttcttatttcatcttcgtctttttatttgatgatactaaaggcaggtgttagaggctggattgctaaagctgacccaaagaatgcctccctcagggctggttggtccctctctctcaggcctcagtcttcccatctgtacagtgaggtgcctgcagatctctgggctctaaaaatcacagctccatgtttatccctggcagaggaagggcctggagtcctgctgcttgcgtctctgggatacgggagcaaagagccacgcatcctcatggcccacacaggcgtcacctccagtctctccttggcctcatctccccagcgtcctggaatggcatcgggctggcccagggagcccctgtcctgtgcctctcctttcccctcaggggctgccaggctgaccacccccaccgcaggccaggcctacagtgccccatggaacgtcctgaccctcccccagggtggcagcaggaagaaggaagaaaggggatcctctccagctggccagagagacagaccttcttgtgctcatcaaccctccaagaatgcctgccctccctccttcccccaaggcctgtccacaggggcttgagatcagccagaaaagtcaggcaacttttcagggactgggagcgaggtctcccggccgggcctgggtccagtctctgtgggcagtgcagtgccgagccccacccctcaagccgtgccctgtccatagctccagactttgaccctgcactccagtccgggctggcggacagagggctggaaacaagacgctccagaatcaggagcttcccctcaggaaatagcatcctgtgtccccgcactgcagttgtctggtctctccagcagtttggtacttccggtgagtggcagatgcacctttgagctggggacaggggttgggagaggggagaggcaaaggatttcatgtcctcccaatgtcaaagacagggctcaacattacagcctaaggcaggtgacaggaaaggagagatccagcctctcaaacatccagcagagagaccataggtaagtgatttttccctccccaagcctcagtttcttcacctggaacatggggatcataactcccctcttacagcgtgagtctgagtgttaaaagaggtggtgcatgtaaagtgcttagagcagatctaggcacatagcaagtactcaaatggtagttattattatttttggtgggggagttggtaggctggttctcaaacttttatagcttctgttccatttcaaggataaactctgcaaataacttcatgagaagtagccgtgtggtgcaaccagggagaactaattatgttcattcaaatgcctcatctctggcttactgatttttttttttaaaaagaagtctttcatattctttgctatgggcacatagcaatcaaaggcatcagctgtctcagattgccttctaggggacaagggaggtcctaggcagataaatgcaagactgaaagacaagcagaaagcatcaagtggcaactgcatgccaactgcctaaatatttttttggagcagtgcagaaagcgccgatagaactgggtctaggtccgaatgctgtcccatactgactgcgtaaccttgggtgggtgacttctcctccctaaacctcagtcccagcctccagaatgagggcggtaaccttccctacttcctagagcagttgagaggattgagaggattatgtcggtactgcatctacaggtgtctggcaagtggcagagaccaaaatacattggttcccttcctgctccacacttacacagacattctaatcacacacacacacacacacacacacacacacacacacacaaatataataatcccagctgtttgcatcttctgggatacatactccaagcttgctgggttgaagtaatgatgtaaaacagaggagaacggcaacactaataaaaacatcagcaacaacacgaaaatgtccaaccgaataactgagctgggtgcgtttaagtccaaaagctcattacctacacgcatgaatgattttacctaaggctggatctgccacatctgacaatctgtctctggcttgtcatgaggacctcatgcatttattttgtattttaaaacacacacacacacacacacacacacacacacacgttgctataatcagtgtcaactttgactcatatcttgaatttttttaaaaaaagataattgacttaggactcacacttttttccttttaaattttttttttttttttttttttgacagagtttcactcttgtcacctgggctggagtgcaatggcatgatttctgcccactgcaatctccacctcccaggttcaagggattctcctgcctcggcctcccgagtagctggaatttcaggcgtgcaccaccatgccaagctaatttttttgtatttttgtagagacagggtttcaccatattggccaggctggtcttgaactcccgacctcaagtgatctgccagcctcgacctcccaaagtgctggaattaaagacgtgagccactgtgcccggcctttttgattttccattctattcctaccaacactctaaaaattcctacaggcattttattttattttattttatcttattttatattatattttatgtttgaaatgcaggactctgaagcttcagctgttcctatttaccggcttgattctcagatttttcaaaccatgtgatttactggcaagcatggcatttaagcacctaggcttatgagtcaggctggcctgggctctgcctctcaccacctgggtgtccaggagctgatattccagtgaggagacaataaggcaaggagctttgtcagctctcataaaagtttatagatgaggtcgggcatggtggctcacgcctgtaatcctagcactttgagagtctgaggccagcaaatcacctgaggtcagaagtttgagaccagcctggccaacatggtgaaaccttgtctctactaaaaatacaaaaattagccaggcatgttggtgcatgcctgtaatcccagctactcaggaggctgaggcaggagaatcacctgaacccgggaggcagagtctgcagtgagccaagattgtaccattgcactccagcctgggcgacaagagtgaaaattcctgctcgaaaaaataaagtttatagatgaggaaactgaggttcgattaggattaaccaactcatcctggtttgcctgggactctgatgcactgacttttagtctgaaagtctgcatcctgggaggaccctcagccctgggcaagctggggaggttggtcaccctcactcagtcaagttgagcaacttgcccagggttacatggctggtgtgtgcccaagtcaggctgcgaacctgggtctgtctgactctcagcctgggccatactgtctcttagattcttcatggagaattaggaaaaatacagaaagccctttattcctctgccttctcattgttaacatataaaaatggtcaagcgggcgggtgcagtggcacacacctgaaagcccagcgctttgggaggctgaggggggaggattgcttgagcctaggaattggaggtggcagtgagctatgattgtgccactgcactccagcctgggtgacagagtgagaccttgtctcttaaaaaaaagaaaaagagtggtcagctctccggaaattatgcagacagtcaaaaagcccagagaggggaattaacttagccaaggtcgcacagcaaggcagaagtgaagccaggtctgactctgcctttctcttctcctctttttttttttgaggcagaatttcgctctgttgcccagactggaatgcagtggtgcgaactcgactcgctgcaacctctgctgcccaggttcaagcgattctcctgcctcagcctcccgagtagctgggattacaggcgcctgccaccgcgcctggctaatttttgtagttttcagtagagatggggtttcaccatcttggccagactggtcttgaagtcctgacctcgtgatccacccgcctcggcctcccgaggtattgggattacaggcgtaagccactgcagctggtcctccctctctccttttgttcctgcaatgtctttgttctatgtgatttttcaaaatgctaggagacaggaaggaggctgctgtgtgttgagggcctactctgtgccaggcgtggtaccaagaacttttgctaaacttcttatttaatccttaaaatgaccctgtgagattgggattaaccctgttttgcagatgaagagcttgtgtctccagaggcaaagtatgggggaagagggaagagagaagaccaagggtccctgagaggggctgtcacctaagccccagtatccaagctcgggctcgaagctggaaggagaattgcctagaggaacgatacctttctgtttgttggttctatctccaacttggcttctgaaaccccaacagagtccagttcttgtgggctggagccgttttccctcctttataaaactaggccatattaagaatgtcccgctgtccagggccacaggcccgagttgccaggagctgaggtctgcgggaggagagttgtgagtgaagaggagggaaagttgaatttggctcttctgggcacaaataattctcttgttctgcctcagcaggagcctgcagaatatttccctgctgtgcgggcttaagtagcttcaaggttaaaagctggtaggccttctaaacttctcagggcccaatcagccctgtgccccaaggcaggtggagttctgtgctggaagaccaagttctgaggccagacactgcgtctgtcatgctcatagctgcatttgctagctgccagcctggcacatggtaggtgtgcattaagcgtgtgttgagtttactcaaattgaaattaagtcacagctgtaccatttaactggctgtgtgacttcaggtaagtcacatcacctctctgaaccacagtttcctcctctgtaagacgggactgataacagcagcccctacctcatgagagtgttgggagacttggatgaatggatgcttgtgaagcacttagtgccggggccagctggctcacagtaggtgctccacaaatgtcagtatattacttcttttgcatcaggcagcttgttaaatttgttacgtttggcatcttgttcaatttcccatccatcccctcaagcataggttattagaggttgaagcatcttgcccaaagttaacggccagtaggtggcagagctgagtcctgaagccagagcccatcgcactaaccaccggcctacccagcctacagttggtcgtgccctctgctgggtcttttctattcccagcccagaaactgggtgtctggggacgctccccagagaaagttgcatcattcaccagccgtgtgactgtggccaagtctcggtcacttctccatacctcagtgtttccatttgcaaaacgggaacaatgatattccttcctcctaggggtcatcgggaaggtcaaatataaaaagggcttggtggtgtctggcaccttctaagccttcagtggatggtggcaatggcgctaaggatgatggagatgatggtgatgatggtgtgcctcaacccttccttcccacaggctgctgcaatgcgtgtggtggtgattggagcaggagtcatcgggctgtccaccgccctctgcatccatgagcgctaccactcagtcctgcagccactggacataaaggtctacgcggaccgcttcaccccactcaccaccaccgacgtggctgccggcctctggcagccctacctttctgaccccaacaacccacaggaggcgtgagtgagggtcacatagggtagcctggggtgcccatggacctaagtctgcagagggagtcagggttcccatcaccaagagcaagccccttgtggaagctactgatctagcataaaataaagaaaatgccaggcgtggtggttcacgcctttaatcctagcactttgggaggtcgaggtgggaggatcacttgaggccaggagttccagatcagcctgggcaacgtggtgaaaccccatctctaccaaaaatacaaaaaattagccgggcatggtggcgcacacctgtaatcccagctactcgggaggctgaggcaggaaaaccatttgagcctaggaggtgaaggtggcagtgagctgagattccgccactgcactcgtgacagagtgagactctgtttcaaaaagaaaaaaataaagaaaagattcataaatattaagccccttgctctgtgccagatactaggaggctttgtctcgtcttccctaaactgggtgcctgtcaataccacatgattggtgaatctggaaaacttcctctgttttaatttatacatttttatttattttttgagattgtgtttcactcttgtcgcccagactggagtgcaatggcgtgatcctggctcactgcatcctctgcctcccaggttcaagcgattctcctgcctcagcttcccaagtaactgggattacaggcatctgccaccacgcctggctaatttttgtatttttagtagagatggagtttcatgttggccagactggtctcgaactcctgacctcaagtgatctgcccaccttgacctcccaaagtgctgggattacaggcatgagccatcatgccttgccaaattttatctttttaaatagagatagggtctcactatgttgcccgggctggtcttgaactcctgggctcaagtgatctgccctccttggcctcccaaaagtgctggaattacaggcatgagccatcatgccttgccaaattttatctttttaaatagagatagggtctcactatgttgcccgggctggtcttgaactcctgggctcaagtgatctgccctccttggcctcccaaagtgctgggattacaggtgtgagcccttgcacccagctgaatctagaaaacttctaagtgggtgaacatctaagtgggtggatggatgcacagatttatcaaataaattgcaaaggtcattatggtagtttagaaactgccagatggttcagcaaatggaacacccaatgaatagcagctcaaacagattaaaaaaaaatttttaagaggcatcctgtcacccaggctgaagtgcagtgacatgatcatagctcattgcagccttgacctcctgggctcaagtgatcctcccacctcagcctcccaagtagcgaggacacacatgcatgctatcatgcctggataattttctttattttttgtagagccagggtcttcctatgttacccaggcttgtctcaaactcctgacctcaagtgaccctcctgcctcagcctcctgaagagctgggattataggcatgagccactgcacccagccagaattttaattcacacagctgtaaaaaaccaatgattctgatcagtgggcagtgattcggggcccaggctccttccatctaatggctctgccgtttttccacgtgcttttgaggtcacctcaatgtcaccattcacatgggctggtgactgcagaaggatcatgcaggaccacacgtgcagagtctttagagtcccttggccagaaacaagtcacacagtcacatttagctgcgagagggtctaggaaatgtaggtgagctgtgtgcccagggggaggaggaaaagttgtaggagcggcaagtcgatgtctgccaccagtgtttacaaggaggggtgcttgcagccagactgaacagtgtggctcataatccccaaagccaggtcaaggacttcactgaaactcatcagccatgtaatcccatgctggaggtgcactccatatggttatgatggggcatccttcattccctctcttctttattctattaatggggaaatattggaaaatttaggagggagaagacccaaggcatttggggagttgcaggagtgaacgtggtggatttctgggttttggacacaccccaagctcctgatcatgccacagccccatgccagctgacctaaggttttttgcccagctcagggcattgggtgatcgaactcttcatgacccttccagggactggagccaacagacctttgactatctcctgagccatgtccattctcccaacgctgaaaacctgggcctgttcctaatctcgggctacaacctcttccatgaagccattccggtgggtgaacagttcttgaccatgagggatgagcacccagggctggggtagtgagggtgggtgcagcagagccttaatcacagatgagggcggggtgctttgagtctcgtaggcaacagactcctgggttcaaaacaggtttggtttaaattctatttttgctttgaaaattatttttgttttacattttgctgttaaattggcagaggacaaagaatcttctgatgcccaggggaaactagcctttgattagcatggctaaaatacaaacatgttctgcagtgacgggcacttggtgctgaagccaaaaggtttcaagtgcccctgaaggtcccaaggctttttatcaagaaggaataaaatactcatcaaagcaaaaactgccaaagcatttattatgtgccaggtccagtcctaactaatttacagctagcgactaatttaattctctttataaccgggaggtaagggctgtccttatcctcacttaataaatgagaaaacggaggctccaagaaatggagtaacttgcccaaggccacaragctcgccagtggcagagctgggatttgaacccaggccatctgtgactccatggtgtccagtgtgctaacaggaacagcacagccctgggacggtttgctcaggctccttggagagggtggtctggcgctgtgcccagagccccgtgccagctctcaaggttcattcaacctttggcactgtgctaagggctttatccacattatctgttacctttcatgggaccaagagtatttttttttttttgagacagggtctcactgtattgcccaggctggagtgcattggcatgatctcggctcactgcaacctctgcttcctgggttcaagccattctcctgtctcagcctcctgagtaattgggattacaggtgcgcactaccacgcctggctaatttttgtatttttaagagatggggtttcactatgacggccgggctggtctcgaactcctgacctcaagtgatctgcctgccttggcctcccaaagtgctgggattacaggcgtgaaccactgcacccggccaagagtgattattaactccatgatacagacaaggaaactgagtctcagagaattcaagtagcaagtgatgaggctggggtctctgacactatgctctgctgtctgacactatgctctgttgctttctctcatccccggggactctcactgtttctgctttctctcccctatttctgacttttcccctataactcaccctcggtcttactcttacccttaccataaataggggttaagaacatgaactctggaactaagctgtatgggttaaaatctcaacaccaccatttattagctgtgtaatcttagacaagttatttaatctttctaagcctcaattggtccatctgtaaactggggaaagaatagcatccaccccaatggcttcttgtgaagattaaatggaccagtataagaaaatgcttggaacagtgccttatatgcacttagcattacataagtctctgtcattatcatttttttttttttttgagatgaagtctcgctctgtggcccaggctggagtgcagcggcacaatttcggcttactgcaacctccagctcccagattcaagcaattctcctgcctcaatctcctgagtatctaggattacaggcatgcaccaccatatcttgctaatttttgtattattatttagtataaacagggtttcaccatgttggccagactggtctggagctcctgacctcaggtgatccacccatctcagcttcccaaagtgctgggattacaggtgtgagccacctcgcctggcccattatcattattattgacttccatcccacccagtgccccctttgtccttcctcttcaggacccttcctggaaggacacagttctgggatttcggaagctgacccccagagagctggatatgttcccagattacgggtgagtttattgtcacaggcaaaggggactggggcctgacgagttagcagacctgtccagaaggcagcagagggtagaggcaccagatttcctgtcctacccaggccctggtaccctggtctcctggtccttggtccagctccttcagagaggctacccactcaaacctggccttgggctgggaaggtagggggtatgaaatcacagatctcaagcccagaagctccatatcaccatattgttttgtagatgaagatactgagtttcagagaggctaagtgacttcctaaggtcacacagccaagtggccaaactgggattccaaccagtctgtatgaccccacacccctcctttcttttctctacagcctgatgcctctctggtcttctcctcaccccaccccacaccacacctgaatcccctctacgaatgcacattcaatctccacttgcattttccaatgtcagatatggccttttctgatagaaaaattttccttgcattgagctcaaaaccacgtccccccttgaacttcacgtagtggtcctggcactaccctttgggcccacagaacaacattgctcccacctccatttcacagccttcaaatatagctctgatttttacctttatttccaccttttgcttactgtgactctagctatggctggttccacacaagcctaattctggagggaaagaactatctacagtggctgactgaaaggtgagattttaagcttcactttgagggaggtacctcccagagaccaagttgtagtggaagatggttcgtgggcttccctcagcatggactaacccccaggtttgaagaatacccttaggcctggtgtgggagctatccttggtcctgatcaccgctgggcacagaggcaatggatcctgagcctagctgagcatcagaaccacctgggcagctgtttacacatgatgtccattaacaacctctttcaaatccctaatgtttgtgtaatagttttagatgtattcttttaaggtttccagatacatttacatcatctgcaaaacataagctgccttttatttttatctccctctctctttttttttcttctctaactgctttggccaatacctctagaacaatgttactcatacagatgatagtggatgtctttgacttgttcctcatgttaataggaatcttgcagtgttctaaattagcaaacactcacttgagagatacattggtatttatattcacatacattcatattaagggagattccataccttttttgtgtgtgtgagatggagtctcgctctgtcacccaggctggagtgcagtggtgcgatcttggctcactgcaagctctgcctcctgggttcatgtcattctcctgcctcagcctcctgagtagctgcgactacaggtgcctgccaccaccacacctggctaattttttgtatttttagtagagatgggttttcaccatgttagccaggatggtctcaatctcctgacctcgtaatctgcccgcctcggcctcccaaaatgctgggattacaggtgtcagccaccacgcccagcctgattccatacattttttatatattactgttttttaaagatttttaggccaggcatggtggttcatacctgtaatcctagcactttgagaggccgaggtgggcagatcacttgagcccaggagttcaagaccagcctgggcaacatggcaaaaccctgtctctacagaaaaattcaaaaatcagccaggtataatggtgcatgcctgtagtcacagctacttaggaggctgaggtgggaggatggctttatcccgggaaggagaggctgcagtgagctgtgatcatgccactgcactccagcctggggggacagggcgagaccctgtctcaaaaaaaaaaaaaaagattaaaaaaatatggaatatatagtggcttttatcagatgacctcagaagattttttttaaatgtagattttaggaccccactctatacctgctgaattagaacttctgggataaggttcataaatttgctttttttcatttttttgagacaaaatcttactttgtcacccaggctggagtgggatgtagtggtatgaacacaactcacagcagcctcaacttcctgggctcaaatgatcctcccacctcagcctccaaagtagctgggaccacatgcatgtgccacaatgcctatctaatttttaaatatttttgtagagatagggtctcactatgttgcccaggctggtctcaaacccctgggctcaagcaatcttcctgcctcagcctcccaaagtgctgggattacaggcgtgagcaaacaggcctagcaaaaatttgcattttaagaagcttcctggcgattctaattatcagccatgtttgggaatcattgtactaagacatggctatttctcctaacctggggacacatgacccttgtccagtcttttccaggaaaaacatgccctcaagatgtttttctatcttgaggaaatgatggaaatgagatagttccaagggtatgcttcaccttctttttggcttatttcctgttctttggatgtttctagtgtatttctttctttcttcttttttttttttttttttttgagacagagtcttgctctgtcacccaggctggagtgcagtggcgcaatctcggctcactgcaagctccgcctcctgggttcatgcctttctcctgcctcagcctcccgagtagcttggaatacaggcgcctgccaccacgtctggctaattttttttttgtattgttagtagagacggggtttcaccgtgttagccaggatggtctcaatctcctgacctcgtgatccgcccacctcggcctcccaaagtactgggattacaggcgtgagccaccgcgcctcgctgtttctagtgtatttctaatcgtgatagatgtttttcctatgggatgtttaaaaggagggtggatgtcctcagcccacctccctcctcatgcccggcttctgacaaaggggaatttggcactggtacaactctccccttctctactctgaatctcattgcctttgctgttacaaagcaatgtggtggtcataggaagtgctgggggctaagaggcctgggtttgagttccaactccatcattgactcactctatggccttcagcaaggcccttcccccactccatctgcccaacaaggggcttggaccatctctggtttctcaaaggagattttgtggaccaccagtccagtaggtgctcatgagctgatttgatgacacagccatcttctcaagcagcatcctgtgcaactaacgtccgcagaaggttgtttggggaaaggtccctgtgccacccttcttggtgggatgggggcagatagctgaatactgggctttttgatgtgtttgatcatcccaggttaactgagaggggagtgaagttcttccagcggaaagtggagtcttttgaggaggtgagttgcagggctgatgcggtggatggggcagggaagaagtagggaggcctctgcttcttgctgctgagtcgggggctcccttctcaggctcctagggtccccacaggcctgcctcagcacccttgccccagaagcactcaggtattctgaagggaggaagtctctgccttcatgttggtagtgggaacaaaggaacactgggatcatggtggccattaggagctgatttatatctgagactcaatgagttttgggtctagagagctggccgcattttctcagtgtcagctgcactccaaggtcagaacttggttgcttcctagccctaccgacatctgtgttggtctttctgcaaagtccaggccctcagctgactcacctctaaagaagcaccaccaccaataataatgacaggaaaagccaccatctccaggcaccagcaaaaagagctttactgtatggcttcattcaatcccagcatctaaaaccctgcttggcacaaggaaggcgctccgtacatgtagctactagtgctatgtcatgaagactaacctgctctggtcaggccctgatggacaccgaagatacatggtcgacccaatgcagtcctcattctcagtcattcactcaggaacaatagtagcgtcttgcaatgtgtgtgtcccttaacttactcgtggtgagagtcactggggctgggttggggagcttaggggctcacgatgcgtgcttgagatgagatcatctcatctgtagacagagctggggttccaacgtgtcttctgcaaatgtcttggcagagtagaaggcaagagaataaagttaaaaggagtcagaaggagaaagagaactctctctgcttcctttctgacttcttttgggaggttccaggaagatttcccccatccaaagaactgttttacaaccacttttatattcagagttgtgcaggagcctcataacagcctatgaacagccatgggcagcctcattttacaggggcagctgagaattaaggaggtaaccagacattttcaaggtcacacgtcagataaatggcagtatgaaaatttgaagccaggcccctctgattcctcattgagacctctccccactgttcatcagggagtagacagattgagggtagaagaaaggggaagagagaacaggggataccagggtcttccccacctttcatcccccactaccctgttggttgctaccaggtggcaagagaaggcgcagacgtgattgtcaactgcactggggtatgggctggggcgctacaacgagaccccctgctgcagccaggccgggggcagatcatgaaggtgagtgtgagggtgagacccctaccttttgttaataggaagatcattctgcatgcttatttcatccctcaagatcatggacaaatcaggaacatctgttagaggaaccccccggactgcagggaattgacatgtaaaaaaaacaaacctgtcccacccccattgctctctttcaggatttcctcttgatcgtgaagcatgcatgtatgcgcttgtacctatgtgggagcagcatatgcctgtattgcaataaaaatagcaaacattagagtgtttaccaagcgcgagatacagtcctaagcactttattgtgtttattattattattaattattaattgtgttattattattatcattgttattattatttttgagacagggtatcactccattgcccaggttagagtgcagtatcttgatcatggctcactgtagccttgacctcccaggctcccaccttagcctactgagtagctgagactacaagcgcatgccaccaccatgctcagccaatttttttattttttgtagagaaaggatttcaccatattgctcaggctggtctcaaactcctgggctcaagtgatccccccaccttggcctgtcaaagtgctgggattacaggcgtgagccaccacgctcagcctattgtgttaattaatttagtgatggccacagcccttcgagctgggtactaccatatcgttattgtcatcttacagatgaagaaattgaggcacagaggagttaagtaacaggcacaagttcacacggtagtacgcagtgcaattgggattggaatccaggcaacctggcttaagagcctgtgcgtgcaagcattgttccatgcctcctcttgctgtgtgtgtgcatatgagggtatgtgtgtgtgcatgtatgtgtgtgtgtgtatgtaagggtatgtgtgcatatgtgtgtgtgcatgtgtgagggtgtgtgtgcatgtgtgagggtgtgtgtgcatgtgtgtgagggtgtgtgcatatgtgatggtgtgtgcacatatgtgagggtgtgtgtgcatgtgtgtgagggtgtgtgcatatgtgtgatggtgtgtgtgcacgtatgtgggggtgattgtgcatgtatgtgagggtgtatgtgcatatgtgtgatggtgtgcgtgcatgcacaccatgtgaggggtatgtgtgtgtgcatgtgtgtgaaggtgtgtgcgcatgtgagagtgtatgtacgtgtgtgatggtgtgtgtgtgtgagggtatgtatgcatgtgtgtgagggtatgtgtgtgcatgtgtgtgagggtgtgtgcatgtatgtaagggtgtgtgtacatgcatgtgtgtgaggggtatatgtgtggatgcatgtgagggtgtgtgtgtgcatgtgtgtgagggtgtgtgtctgagggtgtgtgtgtgcatgtgcacctgtgagtgttcataggtgtgcaggtgtgtgtgcttctgtgtgtaggggtgcgtgtgtgtgttcctaatgtgggctgatgggtgtaacaaccaaatgagtgactgaagcataagtctcaaatcatcgaggtttatggagccagcttgagggcgcacccaggaaaaacgcgagtcacagatgcacctgtgactcctttttccaaagaggttctcaggagatttagtctttatacattttctttaaaaaaaaaaaagtgagagaagggtgtagcagcgagagaatgattgcatacttgtgaaactttagttagtgcccagtaaatctacattttacataagatgaaggtttgggccaggcgtggtgactcacacctgtaatcccagcactttgggaggctgaggcaggtggatcacgaggtcaggagttcgagaccagcctggccaacgtggtgaaaccccatctctactaaaaatacaaaaaattagctgggtgtggtggcgggtgcctgtaatcccagctactcgggaggctgacgcaggagaatcgcttgaacccgggaggcagaggctgcagtgagccgagactacaccactgcactccagcctggcaacagagcgaggctgtctcaaaaaaaaaaaaaaaaaaaaaaattgaaggtttgaaggaaaaaggaatggaggaagttctgtatctgggaagataagcttgtcattgatgttatcagtgtggagtctgttgaaagggctggtttctgcttaccccttagggaagaaagcctaactttggtcaggtcattgagggaggggatataatgagacgtgtcggacctcccttccccccgcagctgtgaactcagctccaaggtttctctggggctcctggggccaagagggggtctgttcagtcggttggggacttagaattttatttttatttctcatgtgtatgcatttacatgtgtgtactggtgcttttcttcggacatgtgggtgaggagaaacaatgcttcagggagcaggggtggctgccaattagggcagctcttcctgcaagaggcaagcagtcaggtgcagacttgggccatagtgtcatgagaggtcttataaggaatcagcctggccactcttgtcaggacatctggccacagaggggagcaagggcagccacattgactcacctccgctgatgagactttcctgccctgaatcaacaggtggacgccccttggatgaagcacttcattctcacccatgacccagagagaggcatctacaattccccgtacatcatcccagggtaaaattggactgttctcgggcagaagagtggtccccttcatgccctcttcatgaccctgctgcctcccccaagctccttactccctgcagttgttccctttcaatgtttttatgtacttagctattttttattattattttttgagacagagtttcactcttattgcccaggctggagtgtaatggtgcgatcttggctcactgcaacctctgcctcccaggttcaagcaattatcctgcctcagcctcccaagtagctgagattacaggtgcccaccaccacatccagctaattttttgtatttttagtagagacagggtttcaccatgttggccaggctggtcttgaactcctgactcaggtgatccacctacccttgcctcccaaagtgctgggattacaggcgtgagccaccgtgcctggccccctttcaatgtttttagtgagtttgagctactgaatccctgggaaggcagactcagcctcgactgaggtctaccgtgaacattcttttggatgacaatagtggtgatgctggagacaaaggcagtggatgtaatgtggtgacactaaaagtggtatgtaggtggctcacgcctgtaatcccagcactttgcgaggccaatgtgggaggatttcttgagcccaggagttcaagaccagcttgggcaacatggcaagaccccgtctctacaaaaatacaaaaattagccgggcgtgatggtgtatgcctatggtcccagctattcgagaggctgagatgggaggattgcttgaacctgggaggttgaggatgcagtgagccatgttcacaccactgtactccagcttgggccacagagcgagaccccatctcaaaaaaaaaaaaaagtggtgtgaatggcaataatgggagtgggaatgggaatggtgattggggctgatggtgatgataatgttaacggtggagatgacaatgtcactgaaaccagtggtggtgttcatgggatgacaatattgttgatagcggaatggtggtattagggataatattgtattgatggggaagacagcgttcatgggggtggtgattagcgtaagagttgtagagtggtgatgttaatggaggtggtctggtgctgatgaggagatcaatgttgatgaaggtgtgattgggagtggggatggtagctggtgctgatggaaatgacactatcaatgatgttaatactgtagcagagctgacagtctcaaaggcaatgttaataacatggttgcaccaaccatgttatctcaatggcgatgttactggtgtcgtggagatgacaatatcaatggcaatgttagtggtggtggtgaaatgatgaatgcagttggtggtgatgacctattaatgatagtagcaaagacaatgttgttgatggagatgacaacattgatggaagtggtgatggaagagttcgttgttggtgttgatggtgatgacagtggcaattgaggtagtgatggtggtggtgttagcagaggtgacaaggttgatggtaatgacctttattcatctcagagccttcattttccttcatccttgaccctcctcatttgtatctaggacccagacagttactcttggaggcatcttccagttgggaaactggagtgaactaaacaatatccaggaccacaacaccatttgggaaggctgctgcagactggagcccacactgaaggtaaggtagggaggagtagcagtgccctaaaccaaggtcgtgggagcttggtaatgaggacacttcaggacgggaagatgccaccgctgggataactgggcaaattaattccagcaagggatgtggaacataacagaatttgataatgtacagggaagttcttgctatgggctaatgaatcctgtctggccatggctgagagcccttggttttcacatttgtctgcgagtgatgatgacagtagtgatggtgatgaggatgagttggtactgatggtgaggaaaatgctgagaatggtaatagtgatggtgataaggtggtgacagttgttaaaattatggtggtggctgatggtgagggtagtggttgatgatggaattggtggaaaggtggaagcagtaatggtaatgatgttggtagctgataaagatggtgttggtggtagtggtgattgataaagatgactgtgattatattagtggtggtggtgatgagattctaaaagctaactccctactacctaaaaatggcagcaggaaaaaaaaatccagaaatgagtgatcagcacttttctttccagaatgcaagaattattggtgaacgaactggcttccggccagtacgcccccagattcggctagaaagagaacagcttcgcactggaccttcaaacacagaggtatgctcccatggcaaggaaagtaatgccctcttccactcctcagatggctctggcattttcagggarcagtcatgtctgatctcaagttccacacaggctccatagcaggcaggggcagtggtggctaatatcccctcctctataaatggggaaactgaggctcaatgatggttaaggacctgctcaaggttacatagaggggcagtggtgatgttaatggaggtggtgctgatgagatcaatgttgataatggtgtgactgggagtggggatggtagctggtgctgatggaaatgacactatcaagtatgttagtaccacagcagaggtgacgatctcaaaggcagtgttaacatggctgcactaactgtctcattggcaatattaatcgtgtggcagagatgacagtatcaatggcagtgttaatgatggtggtgaaatggtgaatggggttggttttctaaagtctgtggtcaaataacaggaaaatgtgtacttactggatgtgtacttcgtgtcagacacagcagcaagtccattacatgaatgaccttattaaatctcctctggagctctttgggatagggacagttctccctatgcttcggatgaggaaactggggtgaattaagaggtgaagtcacttgcccaagtcagaccactggtggaaggcagggctgggatgtgatttgaatttgactccaaggctatttccagatatccattttgtggctgccccatcatctcttgcaactgttccagggggtccccaccattccaccccggtgccaagagaagctcaggtggcatctggctttgcccaggactcttcgggaggctcctgagtcttccagggcagaagagcttcatctattctttccactgtccctctcggacctggccaccttctctcttgcctctcctaggtcatccacaactatggccatggaggctacgggctcaccatccactggggatgtgccctggaggcagccaagctctttgggagaatcctggaagaaaagaaattgtccagaatgccaccatcccacctctgaagactccagtgactgctgcctccccccacaagaactcccttctcccctcagccaatgaatcaatgtgctccttcataagccattgcttctccctcacttctttcctcaaagaagcatgaggtgagagaaagccacaaagtcagtgcctggagaagggttcagcccaacatggggcccctctcatcactgaaatccctctaccttctctgggtctggcattataaagaacagctgaggctgtcattccatgagtcttcagaagaaaggacagctcagaaaatcaaagaggccaactgcccagagccacagaaaatggaggataattgaggctaagtaacctgattacaagttgtactaacatattaaaggttctgaaaagtcctgcagcaaagacaactatctgatgttgtttaacccagtgcttgctaaacctatctggctatggaactcttttgcccagagcacccatgaatgccatgacacaaatctgagaaaatgctggaacagattttgttgtatctgttgtgtttgttgtaggaggttatacatacaactggggtgtggagggggcagagaggtgaggcactgaactagtaacacatggtgtttgttccacatctagaattccaaatggcatcagctattcaccgagtggccccatgagcaccacgtaacctttgaggaggggccactggagggatcatcccacaaggaaccccttcatagagaactgttttagtccattttctgttgcttataacagaatatctgaaactggagattttttttttttttttttgagacaggatctcactctgtcacccaggctggtgtgcagtggcatgattttggctcactgcaacctccgcctcccaggctcaaatgatcctccctcctcagccacccgagtagctgggactacaggcgcttgctaccatgcccagctaattttgtgtgtgtgtgtgtgtgtgtgtgtgttttgtagagagtgttttgtagagactgggtttggccatgttgtccaggctggcgttgaactcctgggatcaagtgatcctcctgcctcagcctccaaagtggtgggattataggcataagccaccacgcctggcggaaactgtggaattaatagagaaaaggaatttatttattaccgttatagagtctgagaagtccaaggttgaggggccacatctggtgagagccttctctctggctggtgcagaggtggggactctctgcagagtcccagggaggcttagggcatcacgtggtgagggggctgattgtgctaatgtgctagctcagctctgtcccttgtcttagaaagccaccattttccttcccaagatgacccattaatccattaacctaataacccattaattgataaatggattaatccatttatgagagcagcgctcttaggatccaatcacctcttaaaggcgccacctctccagaccaccactaaggtggtggactaaggactaagtctcaacgtgagttttggcagggacgtttaagcaatagcaagaactaaactcaccaagca

SEQ ID NO: 2 - DNA, 1573 bp, Homo sapiens
  5′UTR 1..143
  CDS 144..1187
  3′UTR 1188..1573
  polyA_signal 1549..1554
tgcactccagtccgggctggcggacagagggctggaaacaagacgctccagaatcaggagcttcccctcaggaaatagcatcctgtgtccccgcactgcagttgtctggtctctccagcagtttggtacttccggctgctgcaatgcgtgtggtggtgattggagcaggagtcatcgggctgtccaccgccctctgcatccatgagcgctaccactcagtcctgcagccactggacataaaggtctacgcggaccgcttcaccccactcaccaccaccgacgtggctgccggcctctggcagccctacctttctgaccccaacaacccacaggaggcggactggagccaacagacctttgactatctcctgagccatgtccattctcccaacgctgaaaacctgggcctgttcctaatctcgggctacaacctcttccatgaagccattccggacccttcctggaaggacacagttctgggatttcggaagctgacccccagagagctggatatgttcccagattacggctatggctggttccacacaagcctaattctggagggaaagaactatctacagtggctgactgaaaggttaactgagaggggagtgaagttcttccagcggaaagtggagtcttttgaggaggtggcaagagaaggcgcagacgtgattgtcaactgcactggggtatgggctggggcgctacaacgagaccccctgctgcagccaggccgggggcagatcatgaaggtggacgccccttggatgaagcacttcattctcacccatgacccagagagaggcatctacaattccccgtacatcatcccagggacccagacagttactcttggaggcatcttccagttgggaaactggagtgaactaaacaatatccaggaccacaacaccatttgggaaggctgctgcagactggagcccacactgaagaatgcaagaattattggtgaacgaactggcttccggccagtacgcccccagattcggctagaaagagaacagcttcgcactggaccttcaaacacagaggtcatccacaactatggccatggaggctacgggctcaccatccactggggatgtgccctggaggcagccaagctctttgggagaatcctggaagaaaagaaattgtccagaatgccaccatcccacctctgaagactccagtgactgctgcctccccccacaagaactcccttctcccctcagccaatgaatcaatgtgctccttcataagccattgcttctccctcacttctttcctcaaagaagcatgaggtgagagaaagccacaaagtcagtgcctggagaagggttcagcccaacatggggcccctctcatcactgaaatccctctaccttctctgggtctggcattataaagaacagctgaggctgtcattccatgagtcttcagaagaaaggacagctcagaaaatcaaagaggccaactgcccagagccacagaaaatggaggataattgaggctaagtaacctgattacaagttgtactaacatattaaaggttctgaaaagtcctgca

SEQ ID NO: 3 - DNA, 1691 bp, Homo sapiens
  5′UTR 1..143
  CDS 144..1187
  3′UTR 1188..1691
tgcactccagtccgggctggcggacagagggctggaaacaagacgctccagaatcaggagcttcccctcaggaaatagcatcctgtgtccccgcactgcagttgtctggtctctccagcagtttggtacttccggctgctgcaatgcgtgtggtggtgattggagcaggagtcatcgggctgtccaccgccctctgcatccatgagcgctaccactcagtcctgcagccactggacataaaggtctacgcggaccgcttcaccccactcaccaccaccgacgtggctgccggcctctggcagccctacctttctgaccccaacaacccacaggaggcggactggagccaacagacctttgactatctcctgagccatgtccattctcccaacgctgaaaacctgggcctgttcctaatctcgggctacaacctcttccatgaagccattccggacccttcctggaaggacacagttctgggatttcggaagctgacccccagagagctggatatgttcccagattacggctatggctggttccacacaagcctaattctggagggaaagaactatctacagtggctgactgaaaggttaactgagaggggagtgaagttcttccagcggaaagtggagtcttttgaggaggtggcaagagaaggcgcagacgtgattgtcaactgcactggggtatgggctggggcgctacaacgagaccccctgctgcagccaggccgggggcagatcatgaaggtggacgccccttggatgaagcacttcattctcacccatgacccagagagaggcatctacaattccccgtacatcatcccagggacccagacagttactcttggaggcatcttccagttgggaaactggagtgaactaaacaatatccaggaccacaacaccatttgggaaggctgctgcagactggagcccacactgaagaatgcaagaattattggtgaacgaactggcttccggccagtacgcccccagattcggctagaaagagaacagcttcgcactggaccttcaaacacagaggtcatccacaactatggccatggaggctacgggctcaccatccactggggatgtgccctggaggcagccaagctctttgggagaatcctggaagaaaagaaattgtccagaatgccaccatcccacctctgaagactccagtgactgctgcctccccccacaagaactcccttctcccctcagccaatgaatcaatgtgctccttcataagccattgcttctccctcacttctttcctcaaagaagcatgaggtgagagaaagccacaaagtcagtgcctggagaagggttcagcccaacatggggcccctctcatcactgaaatccctctaccttctctgggtctggcattataaagaacagctgaggctgtcattccatgagtcttcagaagaaaggacagctcagaaaatcaaagaggccaactgcccagagccacagaaaatggaggataattgaggctaagtaacctgattacaagttgtactaacatattaaaggttctgaaaagtcctgcagcaaagacaactatctgatgttgtttaacccagtgcttgctaaacctatctggctatggaactcttttgcccagagcacccatgaatgccatgacacaaatctgagaaaatgctggaa

SEQ ID NO: 4 - DNA, 2620 bp, Homo sapiens
  5′UTR 1..1155
  CDS 1156..1818
  3′UTR 1819..2620
gaaacccacgcagcctcctggattcttccccgtccctccctctgtcctggggctgtgacctcctccatgttattcacagggtctcagcacgattcatctcaaagcagcgaaacaagcactggcctcagaagaagcaatattaaaacagttacaactcatctagcgcacagacacccaactgacaccctgttcctccagtcataacaacaactacagctttgattgaacaagagactgagtttggtaactttctcctaataaaaagatcactgactatggactgcttctggtggggttacgaaaccgcaacctcatgtgcctgcatttcctgaaaagacattttgatgtaggaagggcctggagtcctgctgcttgcgtctctgggatacgggagcaaagagccacgcatcctcatggcccacacaggcgtcacctccagtctctccttggcctcatctccccagcgtcctggaatggcatcgggctggcccagggagcccctgtcctgtgcctctcctttcccctcaggggctgccaggctgaccacccccaccgcaggccaggcctacagtgccccatggaacgtcctgaccctcccccagggtggcagcaggaagaaggaagaaaggggatcctctccagctggccagagagacagaccttcttgtgctcatcaaccctccaagaatgcctgccctccctccttcccccaaggcctgtccacaggggcttgagatcagccagaaaagtcaggcaacttttcagggactgggagcgaggtctcccggccgggcctgggtccagtctctgtgggcagtgcagtgccgagccccacccctcaagccgtgccctgtccatagctccagactttgaccctgcactccagtccgggctggcggacagagggctggaaacaagacgctccagaatcaggagcttcccctcaggaaatagcatcctgtgtccccgcactgcagttgtctggtctctccagcagtttggtacttccgatgaagagcttgtgtctccagaggcaaagtatgggggaagagggaagagagaagaccaagggtccctgagaggggctgtcccctaagccccagtatccaagctcgggctcgaagctggaaggagaattgcctagaggctgctgcaatgcgtgtggtggtgattggagcaggagtcatcgggctgtccaccgccctctgcatccatgagcgctaccactcagtcctgcagccactggacataaaggtctacgcggaccgcttcaccccactcaccaccaccgacgtggctgccggcctctggcagccctacctttctgaccccaacaacccacaggaggcggactggagccaacagacctttgactatctcctgagccatgtccattctcccaacgctgaaaacctgggcctgttcctaatctcgggctacaacctcttccatgaagccattccggacccttcctggaaggacacagttctgggatttcggaagctgacccccagagagctggatatgttcccagattacggctatggctggttccacacaagcctaattctggagggaaagaactatctacagtggctgactgaaaggttaactgagaggggagtgaagttcttccagcggaaagtggagtcttttgaggaggtggcaagagaaggcgcagacgtgattgtcaactgcactggggtatgggctggggcgctacaacgagaccccctgctgcagccaggccgggggcagatcatgaaggacccagacagttactcttggaggcatcttccagttgggaaactggagtgaactaaacaatatccaggaccacaacaccatttgggaaggctgctgcagactggagcccacactgaagaatgcaagaattattggtgaacgaactggcttccggccagtacgcccccagattcggctagaaagagaacagcttcgcactggaccttcaaacacagaggtcatccacaactatggccatggaggctacgggctcaccatccactggggatgtgccctggaggcagccaagctctttgggagaatcctggaagaaaagaaattgtccagaatgccaccatcccacctctgaagactccagtgactgctgcctccccccacaagaactcccttctcccctcagccaatgaatcaatgtgctccttcataagccattgcttctccctcacttctttcctcaaagaagcatgaggtgagagaaagccacaaagtcagtgcctggagaagggttcagcccaacatggggcccctctcatcactgaaatccctctaccttctctgggtctggcattataaagaacagctgaggctgtcattccatgagtcttcagaagaaaggacagctcagaaaatcaaagaggccaactgcccagagccacagaaaatggaggataattgaggctaagtaacctgattacaagttgtactaacatattaaaggttctgaaaagtcctgcagcaaagacaactatctgatgttgtttaacccagtgcttgctaaacctatctggctatggaactcttttgcccagagcacccatgaatgccatgacacaaatctgagaaaatgctggaa

SEQ ID NO: 5 - DNA, 1576 bp, Homo sapiens
  5′UTR 1..143
  CDS 144..380
  3′UTR 381..1576
tgcactccagtccgggctggcggacagagggctggaaacaagacgctccagaatcaggagcttcccctcaggaaatagcatcctgtgtccccgcactgcagttgtctggtctctccagcagtttggtacttccggctgctgcaatgcgtgtggtggtgattggagcaggagtcatcgggctgtccaccgccctctgcatccatgagcgctaccactcagtcctgcagccactggacataaaggtctacgcggaccgcttcaccccactcaccaccaccgacgtggctgccggcctctggcagccctacctttctgaccccaacaacccacaggaggcgacccttcctggaaggacacagttctgggatttcggaagctgacccccagagagctggatatgttcccagattacggctatggctggttccacacaagcctaattctggagggaaagaactatctacagtggctgactgaaaggttaactgagaggggagtgaagttcttccagcggaaagtggagtcttttgaggaggtggcaagagaaggcgcagacgtgattgtcaactgcactggggtatgggctggggcgctacaacgagaccccctgctgcagccaggccgggggcagatcatgaaggtggacgccccttggatgaagcacttcattctcacccatgacccagagagaggcatctacaattccccgtacatcatcccagggacccagacagttactcttggaggcatcttccagttgggaaactggagtgaactaaacaatatccaggaccacaacaccatttgggaaggctgctgcagactggagcccacactgaagaatgcaagaattattggtgaacgaactggcttccggccagtacgcccccagattcggctagaaagagaacagcttcgcactggaccttcaaacacagaggtcatccacaactatggccatggaggctacgggctcaccatccactggggatgtgccctggaggcagccaagctctttgggagaatcctggaagaaaagaaattgtccagaatgccaccatcccacctctgaagactccagtgactgctgcctccccccacaagaactcccttctcccctcagccaatgaatcaatgtgctccttcataagccattgcttctccctcacttctttcctcaaagaagcatgaggtgagagaaagccacaaagtcagtgcctggagaagggttcagcccaacatggggcccctctcatcactgaaatccctctaccttctctgggtctggcattataaagaacagctgaggctgtcattccatgagtcttcagaagaaaggacagctcagaaaatcaaagaggccaactgcccagagccacagaaaatggaggataattgaggctaagtaacctgattacaagttgtactaacatattaaaggttctgaaaagtcctgcagcaaagacaactatctgatgttgtttaacccagtgcttgctaaacctatctggctatggaactcttttgcccagagcacccatgaatgccatgacacaaatctgagaaaatgctggaa

SEQ ID NO: 6 - DNA, 1345 bp, Homo sapiens
  5′UTR 1..113
  CDS 114..959
  3′UTR 960..1345
  polyA_signal 1321..1326
gaaacccacgcagcctcctggattcttccccgtccctccctctgtcctggggctgtgacctcctccatgttattcacagggtctcagcacgattcatctcaaaggctgctgcaatgcgtgtggtggtgattggagcaggagtcatcgggctgtccaccgccctctgcatccatgagcgctaccactcagtcctgcagccactggacataaaggtctacgcggaccgcttcaccccactcaccaccaccgacgtggctgccggcctctggcagccctacctttctgaccccaacaacccacaggaggcggactggagccaacagacctttgactatctcctgagccatgtccattctcccaacgctgaaaacctgggcctgttcctaatctcgggctacaacctcttccatgaagccattccggtggcaagagaaggcgcagacgtgattgtcaactgcactggggtatgggctggggcgctacaacgagaccccctgctgcagccaggccgggggcagatcatgaaggtggacgccccttggatgaagcacttcattctcacccatgacccagagagaggcatctacaattccccgtacatcatcccagggacccagacagttactcttggaggcatcttccagttgggaaactggagtgaactaaacaatatccaggaccacaacaccatttgggaaggctgctgcagactggagcccacactgaagaatgcaagaattattggtgaacgaactggcttccggccagtacgcccccagattcggctagaaagagaacagcttcgcactggaccttcaaacacagaggtcatccacaactatggccatggaggctacgggctcaccatccactggggatgtgccctggaggcagccaagctctttgggagaatcctggaagaaaagaaattgtccagaatgccaccatcccacctctgaagactccagtgactgctgcctccccccacaagaactcccttctcccctcagccaatgaatcaatgtgctccttcataagccattgcttctccctcacttctttcctcaaagaagcatgaggtgagagaaagccacaaagtcagtgcctggagaagggttcagcccaacatggggcccctctcatcactgaaatccctctaccttctctgggtctggcattataaagaacagctgaggctgtcattccatgagtcttcagaagaaaggacagctcagaaaatcaaagaggccaactgcccagagccacagaaaatggaggataattgaggctaagtaacctgattacaagttgtactaacatattaaaggttctgaaaagtcctgca

SEQ ID NO: 7 - protein, 347 aa, Homo sapiens
MRVVVIGAGVIGLSTALCIHERYHSVLQPLDIKVYADRFTPLTTTDVAAGLWQPYLSDPNNPQEADWSQQTFDYLLSHVHSPNAENLGLFLISGYNLFHEAIPDPSWKDTVLGFRKLTPRELDMFPDYGYGWFHTSLILEGKNYLQWLTERLTERGVKFFQRKVESFEEVAREGADVIVNCTGVWAGALQRDPLLQPGRGQIMKVDAPWMKHFILTHDPERGIYNSPYIIPGTQTVTLGGIFQLGNWSELNNIQDHNTIWEGCCRLEPTLKNARIIGERTGFRPVRPQIRLEREQLRTGPSNTEVIHNYGHGGYGLTIHWGCALEAAKLFGRILEEKKLSRMPPSHL

SEQ ID NO: 8 - protein, 220 aa, Homo sapiens
MRVVVIGAGVIGLSTALCIHERYHSVLQPLDIKVYADRFTPLTTTDVAAGLWQPYLSDPNNPQEADWSQQTFDYLLSHVHSPNAENLGLFLISGYNLFHEAIPDPSWKDTVLGFRKLTPRELDMFPDYGYGWFHTSLILEGKNYLQWLTERLTERGVKFFQRKVESFEEVAREGADVIVNCTGVWAGALQRDPLLQPGRGQIMKDPDSYSWRHLPVGKLE

SEQ ID NO: 9 - protein, 78 aa, Homo sapiens
MRVVVIGAGVIGLSTALCIHERYHSVLQPLDIKVYADRFTPLTTTDVAAGLWQPYLSDPNNPQEATLPGRTQFWDFGS

SEQ ID NO: 10 - protein, 281 aa, Homo sapiens
MRVVVIGAGVIGLSTALCIHERYHSVLQPLDIKVYADRFTPLTTTDVAAGLWQPYLSDPNNPQEADWSQQTFDYLLSHVHSPNAENLGLFLISGYNLFHEAIPVAREGADVIVNCTGVWAGALQRDPLLQPGRGQIMKVDAPWMKHFILTHDPERGIYNSPYIIPGTQTVTLGGIFQLGNWSELNNIQDHNTIWEGCCRLEPTLKNARIIGERTGFRPVRPQIRLEREQLRTGPSNTEVIHNYGHGGYGLTIHWGCALEAAKLFGRILEEKKLSRMPPSHL

SEQ ID NO: 11 - DNA, 456 bp, Homo sapiens
  allele 152: 99-16105-152  polymorphic base A or G
cgctttgttgtattctttgttatttatccattttgccaaattatctgcaagtagaaatatcgaaataagaagctctttagcaatttactttggatattggttttcttttgaaggacagttattaaaatagcttgtaggattactcattttcrtttttcttctttttaaatataaagcaatgtcatcacttttttccctgtattatatttctcctcaataattgatatgctacattaaaggaacacaaaatggtcttaattatgcaataatgatcaaggcaaagagtgtttcctgggaactaatggttgcctgagaggaggtgatggcttgaggtccagctggttattaagccgcaggaaatgctgcaggccaagatttgtattatttctctgagatgaaaatgaacccaaaaaaaggcaaaatgggtttttctccactaatgggtaaaatgaactc

SEQ ID NO: 12 - DNA, 463 bp, Homo sapiens
  allele 215: 99-5919-215  polymorphic base A or G
tttctcttgactacagcaatgcagatttcaattctgccattgaattcccagacatattcgtcatccccattttcatcccccaccaccctgccattttcttcgtgttaacttgttttcctgactcacagaaatcaccttttcctgtatacatttttaggatgtcagactttattctaatgatttctcctagttgccccccaaaattgtattctacrgtgtgattttaaagctgaattttcaagatgatatttcatatctatattttcacaagcttttcttctatgaatgttattgtcagctgtcagggtgtgagatggtacttgatactacattctttccaagctgttgcctgaatcggtttaagacaaagtcattactaggctgtaaactgttgctctgcaaaattgagcagcacgtatttaaccactcatacttcttagctctccaacactttgagtcrata

SEQ ID NO: 13 - DNA, 742 bp, Homo sapiens
  5′UTR 1..46
  CDS 47..508
  3′UTR 509..742
  polyA_signal 718..723
  allele 21: 8-135-112   polymorphic base C or T
tcatctctgcttcacaatgcygatgatttagctgggaggacccaaaatgctggaaaagctgatgggtgctgattmtctccagcttttcagatccagatatacattgggtaaaatctacttcataggttttcaaargagcattcttctgagcaaatctgaaaactctctaaactctattgcaaaggagacagaakaaggaagagagacggtaacaaggaaagaargatggaagagaaggcatgaggacggctatttggaaatggcacagaggcatttacagagatcattatgtccttgggtctcttaccttcctcagccctatgcagagcttgaagaagtaagcagccatgttggaaaagtcttcatggcaagaaactatgagttccttgmctatgaggcctctaargaccgcaggcagcctctagaacgaatgtggacctgcaactacaaccagcaaaaagaccagtcatgcaaccacaaggaaataacttctaccaaagctgaatgagtttggaagcagattcttcccagccaatccttctgatgacaatgtagtctggccaacatcttcactggamtctgacggactctgtgtctgggacccagctgataacacgtggtgatgggattgtatttgcaaytctctggtcagtaagtgataaaatgccatttctatgcacccacctggcctgtgtgactgggagaatytctctttttattaawtgtgcttcaagttttaaca

SEQ ID NO: 14 - protein, 153 aa, Homo sapiens
  VARIANT 10: Xaa=Ser or Tyr
MLEKLMGADXLQLFRSRYTLGKIYFIGFQXSILLSKSENSLNSIAKETEXGRETVTRKEXWKRRHEDGYLEMAQRHLQRSLCPWVSYLPQPYAELEEVSSHVGKVFMARNYEFLXYEASKDRRQPLERMWTCNYNQQKDQSCNHKEITSTKAE

SEQ ID NO: 15 - DNA, 476 bp, Homo sapiens
catgaggacggctatttggaaatggcacagaggcatttacagagatcattatgtccttgggtctcttaccttcctcagccctatgcagagcttgaagaagtaagcagccatgttggaaaagtcttcatggcaagaaactatgagttccttgcctatgaggcctctaaggaccgcaggcagcctctagaacgaatgtggacctgcaactacaaccagcaaaaagaccagtcatgcaaccacaaggaaataacttctaccaaagctgaatgagtttggaagcagattcttcccagccaatccttctgatgacaatgtagtctggccaacatcttcactggactctgacggactctgtgtctgggacccagctgataacacgtggtgatgggattgtatttgcaactctctggtcagtaagtgataaaatgccatttctatgcacccacctggcctgtgtgactgggagaatctctctt

SEQ ID NO: 16 - protein, 89 aa, Homo sapiens
HEDGYLEMAQRHLQRSLCPWVSYLPQPYAELEEVSSHVGKVFMARNYEFLAYEASKDRRQPLERMWTCNYNQQKDQSCNHKEITSTKAE

SEQ ID NO: 17 - DNA, 1633 bp, Homo sapiens
ttggggtccattgcaacccgaggcgagactagagttcccaagcgagaagggaagaggcagtgggtgcacgtggaaggcggacagagggctggaaacaagacgctccagaatcaggagcttcccctcaggaaatagcatcctgtgtccccgcactgcagttgtctggtctctccagcagtttggtacttccggctgctgcaatgcgtgtggtggtgattggagcaggagtcatcgggctgtccaccgccctctgcatccatgagcgctaccactcagtcctgcagccactgcacataaaggtctacgcggaccgcttcaccccactcaccaccaccgacgtggctgccggcctctggcagccctacctttctgaccccaacaacccacaggaggcggactggagccaacagacctttgactatctcctgagccatgtccattctcccaacgctgaaaacctgggcctgttcctaatctcgggctacaacctcttccatgaagccattccggacccttcctggaaggacacagttctgggatttcggaagctgacccccagagagctggatatgttcccagattacggctatggctggttccacacaagcctaattctggagggaaagaactatctacagtggctgactgaaaggttaactgagaggggagtgaagttcttccagcggaaagtggagtcttttgaggaggtggcaagagaaggcgcagacgtgattgtcaactgcactggggtatgggctggggcgctacaacgagaccccctgctgcagccaggccgggggcagatcatgaaggtggacgccccttggatgaagcacttcattctcacccatgacccagagagaggcatctacaattccccgtacatcatcccagggacccagacagttactcttggaggcatcttccagttgggaaactggagtgaactaaacaatatccaggaccacaacaccatttgggaaggctgctgcagactggagcccacactgaagaatgcaagaattattggtgaagcaactggcttccggccagtacgcccccagattcggctagaaagagaacagcttcgcactggaccttcaaacacagaggtcatccacaactatggccatggaggctacgggctcaccatccactggggatgtgccctggaggcagccaagctctttgggagaatcctggaagaaaagaaattgtccagaatgccaccatcccacctctgaagactccagtgactgctgcctccccccacaagaactcccttctcccctcagccaatgaatcaatgtgctccttcataagccattgcttctccctcacttctttcctcaaagaagcatgaggtgagagaaagccacraagtcagtgcctggagaagggttcagcccaacatggggcccctctcatcactgaaatccctctaccttctctgggtctggcattataaagaacagctgaggctgtcattccatgagtcttcagaagaaaggacagctcagaaagtcaaagaggccaactgcccagagccacagaaaatggaggataattgaggctaagtaacctgattacaagttgtactaacatattaaaggttctgaaaagtcctgcaaaa

SEQ ID NO: 18 - protein, 347 aa, Homo sapiens
MRVVVIGAGVIGLSTALCIHERYHSVLQPLDIKVYADRFTPLTTTDVAAGLWQPYLSDPNNPQEADWSQQTFDYLLSHVHSPNAENLGLFLISGYNLFHEAIPDPSWKDTVLGFRKLTPRELDMFPDYGYGWFHTSLILEGKNYLQWLTERLTERGVKFFQRKVESFEEVAREGADVIVNCTGVWAGALQRDPLLQPGRGQIMKVDAPWMKHFILTHDPERGIYNSPYIIPGTQTVTLGGIFQLGNWSELNNIQDHNTIWEGCCRLEPTLKNARIIGEATGFRPVRPQIRLEREQLRTGPSNTEVIHNYGHGGYGLTIHWGCALEAAKLFGRILEEKKLSRMPPSHL

SEQ ID NO: 19 - DNA, 1200 bp, Homo sapiens
atggacacagcacggattgcagttgtcggggcaggtgtggtggggctctccacggctgtgtgcatctccaaactggtgccccgatgctccgttaccatcatttcagacaagtttactccagataccaccagtgatgtggcagccggaatgcttattcctcacacttatccagatacacccattcacacgcagaagcagtggttcagagaaacctttaatcacctctttgcaattgccaattctgcagaagctggagatgctggtgttcatttggtatcaggttggcagatatttcagagcactccgactgaagaagtgccattctgggctgacgtggttctgggatttcgaaagatgactgaggctgagctgaagaaattcccccagtatgtgtttggtcaggcttttacaaccctgaaatgtgaatgccctgcctacctcccgtggttggagaaaaggataaagggaagtggaggctggacactcactcggcgaatagaagacctgtgggaacttcatccgtcctttgacatcgtggtcaactgttcaggccttggaagcagacagcttgcaggagactcaaagattttccctgtaaggggccaagtcctccaagttcaggctccctgggtggagcattttatccgagatggcagtgggctgacatatatttatcctggtacatcccatgtaaccctaggtggaactaggcaaaaaggggactggaatctgtccccggatgcagaaaatagcagagagattctttcccgatgctgtgctctggagccctccctccacggagcctgcaacatcagggagaaggtgggcttgaggccctacaggccaggcgtgcgactgcagacagagctccttgcgcgagatggacagaggctgcctgtagtccaccactatggccatgggagtgggggcatctcagtgcactggggcactgctctggaggccgccaggctggtgagcgagtgtgtccatgccctcaggacccccattcccaagtcaaacctgtagatgacataaaatgacagcaaagagactgagagactgttgatcaaagcacagaacaggttcaaataacttttccactgcatgaaagtttaattagacatttctttgttttcaacattagaagtggtgtaacatgtaagctgagcacggtagcatgcctatagtcccagctacttg

SEQ ID NO: 20 - DNA, 1023 bp, Homo sapiens
atggacacagcacggattgcagttgtcggggcaggtgtggtggggctctccacggctgtgtgcatctccaaactggtgccccgatgctccgttaccatcatttcagacaagtttactccagataccaccagtgatgtggcagccggaatgcttattcctcacacttatccagatacacccattcacacgcagaagcagtggttcagagaaacctttaatcacctctttgcaattgccaattctgcagaagctggagatgctggtgttcatttggtatcagggataaagggaagtggaggctggacactcactcggcgaatagaagacctgtgggaacttcatccgtcctttgacatcgtggtcaactgttcaggccttggaagcagacagcttgcaggagactcaaagattttccctgtaaggggccaagtcctccaagttcaggctccctgggtggagcattttatccgagatggcagtgggctgacatatatttatcctggtacatcccatgtaaccctaggtggaactaggcaaaaaggggactggaatctgtccccggatgcagaaaatagcagagagattctttcccgatgctgtgctctggagccctccctccacggagcctgcaacatcagggagaaggtgggcttgaggccctacaggccaggcgtgcgactgcagacagagctccttgcgcgagatggacagaggctgcctgtagtccaccactatggccatgggagtgggggcatctcagtgcactggggcactgctctggaggccgccaggctggtgagcgagtgtgtccatgccctcaggacccccattcccaagtcaaacctgtagatgacataaaatgacagcaaagagactgagagactgttgatcaaagcacagaacaggttcaaataacttttccactgcatgaaagtttaattagacatttctttgttttcaacattagaagtggtgtaacatgtaagctgagcacggtagcatgcctatagtcccagctacttg

SEQ ID NO: 21 - protein, 341 aa, Homo sapiens
MDTARIAVVGAGVVGLSTAVCISKLVPRCSVTIISDKFTPDTTSDVAAGMLIPHTYPDTPIHTQKQWFRETFNHLFAIANSAEAGDAGVHLVSGWQIFQSTPTEEVPFWADVVLGFRKMTEAELKKFPQYVFGQAFTTLKCECPAYLPWLEKRIKGSGGWTLTRRIEDLWELHPSFDIVVNCSGLGSRQLAGDSKIFPVRGQVLQVQAPWVEHFIRDGSGLTYIYPGTSHVTLGGTRQKGDWNLSPDAENSREILSRCCALEPSLHGACNIREKVGLRPYRPGVRLQTELLARDGQRLPVVHHYGHGSGGISVHWGTALEAARLVSECVHALRTPIPKSNL

SEQ ID NO: 22 - protein, 282 aa, Homo sapiens
MDTARIAVVGAGVVGLSTAVCISKLVPRCSVTIISDKFTPDTTSDVAAGMLIPHTYPDTPIHTQKQWFRETFNHLFAIANSAEAGDAGVHLVSGIKGSGGWTLTRRIEDLWELHPSFDIVVNCSGLGSRQLAGDSKIFPVRGQVLQVQAPWVEHFIRDGSGLTYIYPGTSHVTLGGTRQKGDWNLSPDAENSREILSRCCALEPSLHGACNIREKVGLRPYRPGVRLQTELLARDGQRLPVVHHYGHGSGGISVHWGTALEAARLVSECVHALRTPIPKSNL

SEQ ID NO: 23 - DNA, 47 bp, Artificial Sequence
  oligonucleotide 24-1443-126
tacggcttagtaagttggagaacyaggatcagaagacaggtctgcct

SEQ ID NO: 24 - DNA, 47 bp, Artificial Sequence
  oligonucleotide 24-1457-52
tctgagatgcccctgtgtcctctmagggagtagtggctgagcatttc

SEQ ID NO: 25 - DNA, 47 bp, Artificial Sequence
  oligonucleotide 27-93-181
cccagctctgccactggcgagctytgtggccttgggcaagttactcc

SEQ ID NO: 26 - DNA, 47 bp, Artificial Sequence
  oligonucleotide 24-1461-256
gatggctctggcattttcagggarcagtcatgtctgatctcaagttc

(19) United States
(12) Patent Application Publication
(10) Pub. No.: US 2003/0185754 A1
(43) Pub. Date: Oct. 2, 2003

(54) Treatment of CNS disorders using D-amino acid oxidase and D-aspartate oxidase antagonists

(75) Inventors: Daniel Cohen, Paris (FR); Ilya Chumakov, Vaux-le-Penil (FR)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(73) Assignee: GENSET, S.A., Paris (FR)

(21) Appl. No.: 10/051,681

(22) Filed: Jan. 16, 2002

Related U.S. Application Data
(60) Provisional application No. 60/261,883, filed on Jan. 16, 2001.
(60) Provisional application No. 60/305,445, filed on Jul. 13, 2001.
(60) Provisional application No. 60/333,881, filed on Nov. 19, 2001.

(51) Int. Cl. A61K 49/00

(52) U.S. Cl. 424/9.2; 800/3

(57) ABSTRACT

Compounds that are antagonists of D-amino acid oxidase and D-aspartate oxidase, methods of treating CNS disorders including bipolar disorder, psychosis and schizophrenia using the compounds, and pharmaceutically acceptable compositions that contain the antagonists are disclosed.

### RELATED APPLICATIONS

This application claims priority from U.S. Provisional Patent Application Serial Nos. 60/261,883, filed Jan. 16, 2001; 60/305,445, filed Jul. 13, 2001; 60/___,___, filed Oct. 22, 2001; and 60/333,881 filed Nov. 19, 2001, which disclosures are hereby incorporated by reference in their entireties.

### FIELD OF THE INVENTION

This invention provides means to identify compounds useful in the treatment of CNS-related disorders such as schizophrenia, bipolar disorder, depression and other mood disorders, means to determine the predisposition of individuals to said disorders, as well as means for the disease diagnosis and prognosis of said disorders. More specifically, this invention relates to means of treating said disorders using antagonists of D-amino acid oxidase (DAO) and D-aspartate oxidase (DDO).

### BACKGROUND

Advances in the technological armamentarium available to basic and clinical investigators have enabled increasingly sophisticated studies of brain and nervous system function in health and disease. Numerous hypotheses both neurobiological and pharmacological have been advanced with respect to the neurochemical and genetic mechanisms involved in central nervous system (CNS) disorders, including psychiatric disorders and neurodegenerative diseases. However, CNS disorders have complex and poorly understood etiologies, as well as symptoms that are overlapping, poorly characterized, and difficult to measure. As a result future treatment regimes and drug development efforts will be required to be more sophisticated and focused on multigenic causes, and will need new assays to segment disease populations, and provide more accurate diagnostic and prognostic information on patients suffering from CNS disorders.

Neurological Basis of CNS Disorders

Neurotransmitters serve as signal transmitters throughout the body. Diseases that affect neurotransmission can therefore have serious consequences. For example, for over 30 years the leading theory to explain the biological basis of many psychiatric disorders such as depression has been the monoamine hypothesis. This theory proposes that depression is partially due to a deficiency in one of the three main biogenic monoamines, namely dopamine, norepinephrine and/or serotonin.

In addition to the monoamine hypothesis, numerous arguments tend to show the value in taking into account the overall function of the brain and no longer only considering a single neuronal system. In this context, the value of dual specific actions on the central aminergic systems including second and third messenger systems has now emerged.

Endocrine Basis of CNS Disorders

It is furthermore apparent that the main monoamine systems, namely dopamine, norepinephrine and serotonin, do not completely explain the pathophysiology of many CNS disorders. In particular, it is clear that CNS disorders may have an endocrine component; the hypothalamic-pituitary-adrenal (HPA) axis, including the effects of corticotrophin-releasing factor and glucocorticoids, plays an important role in the pathophysiology of CNS disorders.

In the hypothalamus-pituitary-adrenal (HPA) axis, the hypothalamus lies at the top of the hierarchy regulating hormone secretion. It manufactures and releases peptides (small chains of amino acids) that act on the pituitary, at the base of the brain, stimulating or inhibiting the pituitary's release of various hormones into the blood. These hormones, among them growth hormone, thyroid-stimulating hormone and adrenocorticotrophic hormone (ACTH), control the release of other hormones from target glands. In addition to functioning outside the nervous system, the hormones released in response to pituitary hormones also feed back to the pituitary and hypothalamus. There they deliver inhibitory signals that serve to limit excess hormone biosynthesis.

CNS Disorders

Neurotransmitter and hormonal abnormalities are implicated in disorders of movement (e.g. Parkinson's disease, Huntington's disease, motor neuron disease, etc.), disorders of mood (e.g. unipolar depression, bipolar disorder, anxiety, etc.) and diseases involving the intellect (e.g. Alzheimer's disease, Lewy body dementia, schizophrenia, etc.). In addition, these systems have been implicated in many other disorders, such as coma, head injury, cerebral infarction, epilepsy, alcoholism and the mental retardation states of metabolic origin seen particularly in childhood.

CNS disorders can encompass a wide range of disorders, and a correspondingly wide range of genetic factors. Examples of CNS disorders include neurodegenerative disorders, psychotic disorders, mood disorders, autism, substance dependence and alcoholism, mental retardation, and other psychiatric diseases including cognitive, anxiety, eating, impulse-control, and personality disorders. Disorders can be defined using the Diagnosis and Statistical Manual of Mental Disorders fourth edition (DSM-IV) classification.

Even when considering just a small subset of CNS disorders, it is evident from the lack of adequate treatment for and understanding of the molecular basis of the psychotic disorders schizophrenia and bipolar disorder that new targets for therapeutic invention and improved methods of treatment are needed. For both schizophrenia and bipolar disorder, all the known molecules used for the treatment have side effects and act only against the symptoms of the disease. There is a strong need for new molecules without associated side effects and directed against targets which are involved in the causal mechanisms of schizophrenia and bipolar disorder. Therefore, tools facilitating the discovery and characterization of these targets are necessary and useful.

The aggregation of schizophrenia and bipolar disorder in families, the evidence from twin and adoption studies, and the lack of variation in incidence worldwide, indicate that schizophrenia and bipolar disorder are primarily genetic conditions, although environmental risk factors are also involved at some level as necessary, sufficient, or interactive causes. For example, schizophrenia occurs in 1% of the general population. But, if there is one grandparent with schizophrenia, the risk of getting the illness increases to about 3%; one parent with Schizophrenia, to about 10%. When both parents have schizophrenia, the risk rises to approximately 40%.

Identification of Schizophrenia Susceptibility gene on Chromosome 13q31-q33.

The identification of genes involved in a particular trait such as a specific central nervous system disorder, like schizophrenia, can be carried out through two main strategies currently used for genetic mapping: linkage analysis and association studies. Linkage analysis requires the study of families with multiple affected individuals and is now useful in the detection of mono- or oligogenic inherited traits. Conversely, association studies examine the frequency of marker alleles in unrelated trait (T+) individuals compared with trait negative (T−) controls, and are generally employed in the detection of polygenic inheritance.

Genetic link or “linkage” is based on an analysis of which of two neighboring sequences on a chromosome contains the least recombinations by crossing-over during meiosis. To do this, chromosomal markers, like microsatellite markers, have been localized with precision on the genome. Genetic link analysis calculates the probabilities of recombinations on the target gene with the chromosomal markers used, according to the genealogical tree, the transmission of the disease, and the transmission of the markers. Thus, if a particular allele of a given marker is transmitted with the disease more often than chance would have it (recombination level between 0 and 0.5), it is possible to deduce that the target gene in question is found in the neighborhood of the marker. Using this technique, it has been possible to localize several genes demonstrating a genetic predisposition of familial cancers. In order to be able to be included in a genetic link study, the families affected by a hereditary form of the disease must satisfy the “informativeness” criteria: several affected subjects (and whose constitutional DNA is available) per generation, and at best having a large number of siblings.

Results of previous linkage studies supported the hypothesis that chromosome 13 was likely to harbor a schizophrenia susceptibility locus on 13q32 (Blouin J L et al., 1998, Nature Genetics, 20:70-73; Lin M W et al., 1997, Hum. Genet., 99(3):417-420). These observations suggesting the presence of a schizophrenia locus on the chromosome 13q32 locus had been obtained by carrying out linkage studies. Linkage analysis had been successfully applied to map simple genetic traits that show clear Mendelian inheritance patterns and which have a high penetrance, but this method suffers from a variety of drawbacks. First, linkage analysis is limited by its reliance on the choice of a genetic model suitable for each studied trait. Furthermore, the resolution attainable using linkage analysis is limited, and complementary studies are required to refine the analysis of the typical 20 Mb regions initially identified through this method. In addition, linkage analysis has proven difficult when applied to complex genetic traits, such as those due to the combined action of multiple genes and/or environmental factors. In such cases, too great an effort and cost are needed to recruit the adequate number of affected families required for applying linkage analysis to these situations. Finally, linkage analysis cannot be applied to the study of traits for which no large informative families are available.

Novel schizophrenia gene: g34872 (sbg1).

More recently, instead of using linkage studies, a novel schizophrenia and bipolar disorder related gene referred to as the g34872 gene located on the chromosome 13q31-q33 locus was identified using an alternative method of conducting association studies. This alternative method involved generating biallelic markers (primarily single nucleotide polymorphisms (SNPs)) in the region of interest, identifying markers in linkage disequilibrium with schizophrenia, and conducting association studies in unrelated schizophrenia and bipolar disorder case and control populations.

In summary, a BAC contig covering the candidate genomic region was constructed using 27 public sequence-tagged site (STS) markers localised on chromosome 13 in the region of 13q31-q33 to screen a 7 genome equivalent proprietary BAC library. From these materials, new STSs were generated allowing construction of a dense physical map of the region. In total, 275 STSs allowed identification of 255 BACs that were all sized and mapped by in situ chromosomal hybridisation for verification. New biallelic markers were generated by partial sequencing of insert ends from subclones of some of the BAC inserts localized to the human chromosome 13q31-q33 region. In a first phase of the analysis, a first set of 34 biallelic markers on 9 different BACs across the chromosome 13q31-q33 candidate locus were analysed in schizophrenic cases and controls, thereby identifying a subregion showing an association with schizophrenia. Following this first analysis, further biallelic markers were generated as described above in order to provide a very high density map of the target region. A minimal set of 35 BACs was identified and fully sequenced which resulted in several contigs including a contig of over 900 kb comprising sequences of the target region.

These biallelic markers were used in association studies in order to refine a particular subregion of interest, which contained a candidate schizophrenia gene, g34872. The biallelic markers were genotyped in several studies carried out in different populations to confirm the association with the subregion. Association studies were first performed on two different screening samples of schizophrenia cases and controls from a French Canadian population comprising 139 cases and 141 controls, and 215 cases and 241 controls, respectively, as well on bipolar disorder cases and controls from an Argentinian population. The results obtained after several studies using this population indicated a genomic region of about 150 kb showing a significant association with schizophrenia. This association was then confirmed in separate studies using cases and controls from a U.S. schizophrenia population, as well as in further samples from the Argentinian and French Canadian populations.

The approximately 150 kb genomic region associated with schizophrenia was found to contain the candidate gene g34872. In addition to characterizing the intron-exon structure of the g34872 gene, a range of mRNA splicing variants including tissue specific mRNA splicing variants were identified, and the existence of the mRNA was demonstrated. Subsequently, a peptide fragment derived from the g34872 polypeptide product, the amino acid sequence of which is shown in SEQ ID No 5, demonstrated a decrease in locomotor movement frequency, and an increase in stereotypy when injected intraperitoneally in mice. Further discussion of the identification of the g34872 gene is provided in co-pending U.S. patent application Ser. No. 09/539,333 titled “Schizophrenia associated genes, proteins and biallelic markers” and co-pending International Patent Application No. PCT/IB00/00435, both filed Mar. 30, 2000 and which disclosures are hereby incorporated by reference in their entireties.

g34872 Interacting Proteins and Schizophrenia.

There is a strong need to identify genes involved in schizophrenia and bipolar disorder. There is also a need to identify genes involved in the g34872 pathway and genes whose products functionally interact with the g34872 gene products. These genes may provide new intervention points in the treatment of schizophrenia or bipolar disorder and allow further study and characterization of the g34872 gene and related biological pathway. The knowledge of these genes and the related biological pathways involved in schizophrenia will allow researchers to understand the etiology of schizophrenia and bipolar disorder and will lead to drugs and medications which are directed against the cause of the diseases. There is also a great need for new methods for detecting a susceptibility to schizophrenia and bipolar disorder, as well as for preventing or following up the development of the disease. Diagnostic tools could also prove extremely useful. Indeed, early identification of subjects at risk of developing schizophrenia would enable early and/or prophylactic treatment to be administered. Moreover, accurate assessments of the eventual efficacy of a medicament as well as the patent's eventual tolerance to it may enable clinicians to enhance the benefit/risk ratio of schizophrenia and bipolar disorder treatment regimes.

The present invention thus relates to any gene encoding for proteins which interact with g34872 polypeptides, herein referred to as g34872 binding partners. By yeast 2-hybrid technology, the inventors have cloned several g34872 binding partners. The inventors demonstrate that D-amino acid oxidase is included in the group of said g34872 binding partners. Knowledge of g34872 binding partner permits the development of medicaments for the treatment of CNS disease mediated by genes selected from the group comprising g34872, D-amino acid oxidase and any other g34872 binding partners. Furthermore, knowledge of g34872 binding partners provides a means for the detection of g34872, g34872-binding partners, g34872-binding partners complexes or interactions between g34872 and its binding partners.

g34872 Interacting Proteins and Schizophrenia: D-amino Acid Oxidase.

D-Amino acid oxidase (DAO) was one of the first enzymes to be described and the second flavoprotein to be discovered in the mid 1930s. DAO converts D-amino acids into the corresponding .alpha.-keto acids. It does this by catalyzing the dehydrogenation of D-amino acids to their imino counterparts and a reduced flavin-product complex. The reduced flavin is then (re)oxidized by dioxygen to yield FADox and H2O2, whereas the imino acid spontaneously hydrolyzes to the keto acid and NH4+. Although DAO is present in most organisms and mammalian tissues, its physiological role in vertebrates has been unclear. DAO oxidizes: D-Met, D-Pro, D-Phe, D-Tyr, D-Ile, D-Leu, D-Ala and D-Val. D-Ser, D-Arg, D-His, D-norleucine and D-Trp are oxidized at a low rate. D-Ornithine, cis-4-hydroxy-D-proline, D-Thr, D-Trp-methyl ester, N-acetyl-D-Ala and D-Lys are oxidized at a very low rate. D-Asp, D-Glu and their derivatives, Gly and all the L-amino acids are not oxidized (or are at a rate which is undetectable). D-Aspartate oxidase (DDO) oxidizes only D-Asp, D-Glu and their following derivatives: D-Asn, D-Gln, D-Asp-dimethyl-ester and N-methyl-D-Asp.

CNS disorders are a type of neurological disorder. CNS disorders can be drug induced; can be attributed to genetic predisposition, infection or trauma; or can be of unknown etiology. CNS disorders comprise neuropsychiatric disorders, neurological diseases and mental illnesses; and include neurodegenerative diseases, behavioral disorders, cognitive disorders and cognitive affective disorders. There are several CNS disorders whose clinical manifestations have been attributed to CNS dysfunction (i.e., disorders resulting from inappropriate levels of neurotransmitter release, inappropriate properties of neurotransmitter receptors, and/or inappropriate interaction between neurotransmitters and neurotransmitter receptors). Several CNS disorders can be attributed to a cholinergic deficiency, a dopaminergic deficiency, an adrenergic deficiency and/or a serotonergic deficiency. CNS disorders of relatively common occurrence include presenile dementia (early onset Alzheimer's disease), senile dementia (dementia of the Alzheimer's type), Parkinsonism including Parkinson's disease, Huntington's chorea, tardive dyskinesia, hyperkinesia, mania, attention deficit disorder, anxiety, dyslexia, schizophrenia, psychosis, bipolar disorder, depression and Tourette's syndrome.

Neurotransmitter and hormonal abnormalities are implicated in disorders of movement (e.g. Parkinson's disease, Huntington's disease, motor neuron disease, etc.), disorders of mood (e.g. unipolar depression, bipolar disorder, anxiety, etc.) and diseases involving the intellect (e.g. Alzheimer's disease, Lewy body dementia, schizophrenia, etc.). In addition, neurotransmitter and hormonal abnormalities have been implicated in a wide range of disorders, such as coma, head injury, cerebral infarction, epilepsy, alcoholism and the mental retardation states of metabolic origin seen particularly in childhood.

Schizophrenia

In developed countries schizophrenia occurs in approximately one per cent of the adult population at some point during their lives. There are an estimated 45 million people with schizophrenia in the world, with more than 33 million of them in the developing countries. Moreover, schizophrenia accounts for a fourth of all mental health costs and takes up one in three psychiatric hospital beds. Most schizophrenia patients are never able to work. The cost of schizophrenia to society is enormous. In the United States, for example, the direct cost of treatment of schizophrenia has been estimated to be close to 0.5% of the gross national product. Standardized mortality ratios (SMRs) for schizophrenic patients are estimated to be two to four times higher than the general population and their life expectancy overall is 20% shorter than for the general population.

The most common cause of death among schizophrenic patients is suicide (in 10% of patients) which represents a 20 times higher risk than for the general population. Deaths from heart disease and from diseases of the respiratory and digestive system are also increased among schizophrenic patients.

Schizophrenia comprises a group of psychoses with either ‘positive’ or ‘negative’ symptoms. Positive symptoms consist of hallucinations, delusions and disorders of thought; negative symptoms include emotional flattening, lack of volition and a decrease in motor activity.

A number of biochemical abnormalities have been identified and, in consequence, several neurotransmitter based hypotheses have been advanced over recent years; the most popular one has been “the dopamine hypothesis,” one variant of which states that there is over-activity of the mesolimbic dopamine pathways at the level of the D₂ receptor. However, researchers have been unable to consistently find an association between various receptors of the dopaminergic system and schizophrenia.

Bipolar Disorder

Bipolar disorders are relatively common disorders, occurring in about 1.3% of the population, and have been reported to constitute about half of the mood disorders seen in psychiatric clinics with severe and potentially disabling effects. Bipolar disorders have been found to vary with gender depending of the type of disorder; for example, bipolar disorder I is found equally among men and women, while bipolar disorder II is reportedly more common in women. The age of onset of bipolar disorders is typically in the teenage years and diagnosis is typically made in the patient's early twenties. Bipolar disorders also occur among the elderly, generally as a result of a neurological disorder or other medical conditions. In addition to the severe effects on patients' social development, suicide completion rates among bipolar patients are reported to be about 15%.

Bipolar disorders are characterized by phases of excitement and often depression; the excitement phases, referred to as mania or hypomania, and depressive phases can alternate or occur in various admixtures, and can occur to different degrees of severity and over varying duration. Since bipolar disorders can exist in different forms and display different symptoms, the classification of bipolar disorder has been the subject of extensive studies resulting in the definition of bipolar disorder subtypes and widening of the overall concept to include patients previously thought to be suffering from different disorders. Bipolar disorders often share certain clinical signs, symptoms, treatments and neurobiological features with psychotic illnesses in general and therefore present a challenge to the psychiatrist to make an accurate diagnosis. Furthermore, because the course of bipolar disorders and various mood and psychotic disorders can differ greatly, it is critical to characterize the illness as early as possible in order to offer means to manage the illness over a long term.

The costs of bipolar disorders to society are enormous. The mania associated with the disease impairs performance and causes psychosis, and often results in hospitalization. This disease places a heavy burden on the patient's family and relatives, both in terms of the direct and indirect costs involved and the social stigma associated with the illness, sometimes over generations. Such stigma often leads to isolation and neglect. Furthermore, the earlier the onset, the more severe are the effects of interrupted education and social development.

The DSM-IV classification of bipolar disorder distinguishes among four types of disorders based on the degree and duration of mania or hypomania as well as two types of disorders which are evident typically with medical conditions or their treatments, or to substance abuse. Mania is recognized by elevated, expansive or irritable mood as well as by distractability, impulsive behavior, increased activity, grandiosity, elation, racing thoughts, and pressured speech. Of the four types of bipolar disorder characterized by the particular degree and duration of mania, DSM-IV includes:

bipolar disorder I, including patients displaying mania for at least one week;

bipolar disorder II, including patients displaying hypomania for at least 4 days, characterized by milder symptoms of excitement than mania, who have not previously displayed mania, and have previously suffered from episodes of major depression;

bipolar disorder not otherwise specified (NOS), including patients otherwise displaying features of bipolar disorder II but not meeting the 4 day duration for the excitement phase, or who display hypomania without an episode of major depression; and

cyclothymia, including patients who show numerous manic and depressive symptoms that do not meet the criteria for hypomania or major depression, but which are displayed for over two years without a symptom-free interval of more than two months.

The remaining two types of bipolar disorder as classified in DSM-VI are disorders evident or caused by various medical disorder and their treatments, and disorders involving or related to substance abuse. Medical disorders which can cause bipolar disorders typically include endocrine disorders and cerebrovascular injuries, and medical treatments causing bipolar disorder are known to include glucocorticoids and the abuse of stimulants. The disorder associated with the use or abuse of a substance is referred to as “substance induced mood disorder with manic or mixed features”.

Diagnosis of bipolar disorder can be very challenging. One particularly troublesome difficulty is that some patients exihibit mixed states, simultaneously manic and dysphoric or depressive, but do not fall into the DSM-IV classification because not all required criteria for mania and major depression are met daily for at least one week. Other difficulties include classification of patients in the DSM-IV groups based on duration of phase since patients often cycle between excited and depressive episodes at different rates. In particular, it is reported that the use of antidepressants may alter the course of the disease for the worse by causing “rapid-cycling”. Also making diagnosis more difficult is the fact that bipolar patients, particularly at what is known as Stage III mania, share symptoms of disorganized thinking and behavior with bipolar disorder patients. Furthermore, psychiatrists must distinguish between agitated depression and mixed mania; it is common that patients with major depression (14 days or more) exhibit agitiation, resulting in bipolar-like features. A yet further complicating factor is that bipolar patients have an exceptionally high rate of substance, particularly alcohol abuse. While the prevalence of mania in alcoholic patients is low, it is well known that substance abusers can show excited symptoms. Difficulties therefore result for the diagnosis of bipolar patients with substance abuse.

Depression

Depression is a serious medical illness that affects 340 million people worldwide. In contrast to the normal emotional experiences of sadness, loss, or passing mood states, clinical depression is persistent and can interfere significantly with an individual's ability to function. As a result, depression is the leading cause of disability throughout the world with an estimated cost of $53 billion each year in the United States alone.

Symptoms of depression include depressed mood, diminished interest or pleasure in activities, change in appetite or weight, insomnia or hypersomnia, psycho-motor agitation or retardation, fatigue or loss of energy, feelings of worthlessness or excessive guilt, anxiety, inability to concentrate or act decisively, and recurrent thoughts of death or suicide. A diagnosis of unipolar major depression (or major depressive disorder) is made if a person has five or more of these symptoms and impairment in usual functioning nearly every day during the same two-week period. The onset of depression generally begins in late adolescence or early adult life; however, recent evidence suggests depression may be occurring earlier in life in people born in the past thirty years.

The World Health Organization predicts that by the year 2020 depression will be the greatest burden of ill-health to people in the developing world, and that by then depression will be the second largest cause of death and disability. Beyond the almost unbearable misery it causes, the big risk in major depression is suicide. Within five years of suffering a major depression, an estimated 25% of sufferers try to kill themselves. In addition, depression is a frequent and serious complication of heart attack, stroke, diabetes, and cancer. According to one recent study that covered a 13-year period, individuals with a history of major depression were four times as likely to suffer a heart attack compared to people without such a history.

Depression may also be a feature in up to 50% of patients with CNS disorders such as Parkinson's disease and Alzheimer's disease.

Low levels of the dopamine metabolite HVA are found in the CSF in patients with depression. In addition, dopamine agonists produce a therapeutic response in depression.

Presently, antidepressants are designed to address many of the symptoms of depression by increasing neurotransmitter concentration in aminergic synapses. Distinct pharmacologic mechanisms allow the antidepressants to be separated into seven different classes. The two classical mechanisms are those of tricyclic antidepressants (TCAs) and monoamine oxidase inhibitors (MAOIs). The most widely prescribed agents are the serotonin selective reuptake inhibitors (SSRIs). Three other classes of antidepressants, like the SSRls, increase serotonergic neurotransmission, but they also have additional actions, namely dual serotonin and norepinephrine reuptake inhibition; serotonin-2 antagonism/reuptake inhibition; and alpha₂ antagonism plus serotonin-2 and -3 antagonism. The selective norepinephrine and dopamine reuptake inhibitors define a novel class of antidepressant that has no direct actions on the serotonin system.

For CNS disorders such as schizophrenia, bipolar disorder, depression and other mood disorders, all the known molecules used for treatment have side effects and act only against the symptoms of the disease. There is a strong need for new molecules without associated side effects or reduced side effects which are directed against targets that are involved in the causal mechanisms of such CNS disorders. It would be desirable to provide a useful method for the prevention and treatment of such CNS disorders by administering a DAO antagonist compound to a patient susceptible to or suffering from such a disorder. Alternatively, it would be desirable to provide a useful method for the prevention and treatment of such CNS disorders by administering a DDO antagonist compound to a patient susceptible to or suffering from such a disorder.

For CNS disorders such as Parkinson's Disease, Alzheimer's Disease, and other neurodegenerative disorders there are limited numbers of pharmaceutical compositions available for treatment and the known molecules used for treatment have side effects and act only against the symptoms of the disease. There is a strong need for new molecules without associated side effects or reduced side effects which are directed against targets that are involved in the causal mechanisms of such CNS disorders. It would be desirable to provide a useful method for the prevention and treatment of such CNS disorders by administering a DAO activator compound to a patient susceptible to or suffering from such a disorder. Alternatively, it would be desirable to provide a useful method for the prevention and treatment of such CNS disorders by administering a DDO activator compound to a patient susceptible to or suffering from such a disorder.

The pharmaceutical compositions of the present invention are useful for the prevention and treatment of such CNS disorders.

Treatment

As there are currently no cures for CNS disorders such as schizophrenia, bipolar disorder, depression and other mood disorders, the objective of treatment is to reduce the severity of the symptoms, if possible to the point of remission. Due to the similarities in symptoms, schizophrenia, depression and bipolar disorder are often treated with some of the same medicaments. Both diseases are often treated with antipsychotics and neuroleptics.

For schizophrenia, for example, antipsychotic medications are the most common and most valuable treatments. There are four main classes of antipsychotic drugs which are commonly prescribed for schizophrenia. The first, neuroleptics, exemplified by chlorpromazine (Thorazine), has revolutionized the treatment of schizophrenic patients by reducing positive (psychotic) symptoms and preventing their recurrence. Patients receiving chlorpromazine have been able to leave mental hospitals and live in community programs or their own homes. But these drugs are far from ideal. Some 20% to 30% of patients do not respond to them at all, and others eventually relapse. These drugs were named neuroleptics because they produce serious neurological side effects, including rigidity and tremors in the arms and legs, muscle spasms, abnormal body movements, and akathisia (restless pacing and fidgeting). These side effects are so troublesome that many patients simply refuse to take the drugs. Besides, neuroleptics do not improve the so-called negative symptoms of schizophrenia and the side effects may even exacerbate these symptoms. Thus, despite the clear beneficial effects of neuroleptics, even some patients who have a good short-term response will ultimately deteriorate in overall functioning.

The well known deficiencies in the standard neuroleptics have stimulated a search for new treatments and have led to a new class of drugs termed atypical neuroleptics. The first atypical neuroleptic, Clozapine, is effective for about one third of patients who do not respond to standard neuroleptics. It seems to reduce negative as well as positive symptoms, or at least exacerbates negative symptoms less than standard neuroleptics do. Moreover, it has beneficial effects on overall functioning and may reduce the chance of suicide in schizophrenic patients. It does not produce the troubling neurological symptoms of the standard neuroleptics, or raise blood levels of the hormone prolactin, excess of which may cause menstrual irregularities and infertility in women, impotence or breast enlargement in men. Many patients who cannot tolerate standard neuroleptics have been able to take clozapine. However, clozapine has serious limitations. It was originally withdrawn from the market because it can cause agranulocytosis, a potentially lethal inability to produce white blood cells. Agranulocytosis remains a threat that requires careful monitoring and periodic blood tests. Clozapine can also cause seizures and other disturbing side effects (e.g., drowsiness, lowered blood pressure, drooling, bed-wetting, and weight gain). Thus it is usually taken only by patients who do not respond to other drugs.

Researchers have developed a third class of antipsychotic drugs that have the virtues of clozapine without its defects. One of these drugs is risperidone (Risperdal). Early studies suggest that it is as effective as standard neuroleptic drugs for positive symptoms and may be somewhat more effective for negative symptoms. It produces more neurological side effects than clozapine but fewer than standard neuroleptics. However, it raises prolactin levels. Risperidone is now prescribed for a broad range of psychotic patients, and many clinicians seem to use it before clozapine for patients who do not respond to standard drugs, because they regard it as safer. Another new drug is Olanzapine (Zyprexa) which is at least as effective as standard drugs for positive symptoms and more effective for negative symptoms. It has few neurological side effects at ordinary clinical doses, and it does not significantly raise prolactin levels. Although it does not produce most of clozapine's most troubling side effects, including agranulocytosis, some patients taking olanzapine may become sedated or dizzy, develop dry mouth, or gain weight. In rare cases, liver function tests become transiently abnormal.

Outcome studies in schizophrenia are usually based on hospital treatment studies and may not be representative of the population of schizophrenia patients. At the extremes of outcome, 20% of patients seem to recover completely after one episode of psychosis, whereas 14-19% of patients develop a chronic unremitting psychosis and never fully recover. In general, clinical outcome at five years seems to follow the rule of thirds: with about 35% of patients in the poor outcome category; 36% in the good outcome category, and the remainder with intermediate outcome. Prognosis in schizophrenia does not seem to worsen after five years.

Whatever the reasons, there is increasing evidence that leaving schizophrenia untreated for long periods early in course of the illness may negatively affect the outcome. However, the use of drugs is often delayed for patients experiencing a first episode of the illness. The patients may not realize that they are ill, or they may be afraid to seek help; family members sometimes hope the problem will simply disappear or cannot persuade the patient to seek treatment; clinicians may hesitate to prescribe antipsychotic medications when the diagnosis is uncertain because of potential side effects. Indeed, at the first manifestation of the disease, schizophrenia is difficult to distinguish from bipolar manic-depressive disorders, severe depression, drug-related disorders, and stress-related disorders. Since the optimum treatments differ among these diseases, the long term prognosis of the disorder also differs the beginning of the treatment.

For both CNS disorders such as schizophrenia, bipolar disorder, depression and other mood disorder, known molecules used for the treatment have side effects and act only against the symptoms of the disease. There is a strong need for new molecules without associated side effects and directed against targets which are involved in the causal mechanisms of such CNS disorders. Therefore, tools facilitating the discovery and characterization of these targets are necessary and useful.

The aggregation of schizophrenia and bipolar disorder in families, the evidence from twin and adoption studies, and the lack of variation in incidence worldwide, indicate that schizophrenia, depression, and bipolar disorder are primarily genetic conditions, although environmental risk factors are also involved at some level as necessary, sufficient, or interactive causes. For example, schizophrenia occurs in 1% of the general population. But, if there is one grandparent with schizophrenia, the risk of getting the illness increases to about 3%; one parent with Schizophrenia, to about 10%. When both parents have schizophrenia, the risk rises to approximately 40%.

Consequently, there is a strong need to identify genes involved in such CNS disorders. The knowledge of these genes will allow researchers to understand the etiology of schizophrenia, depression, bipolar disorder and other mood disorders and could lead to drugs and medications which are directed against the cause of the diseases, not just against their symptoms.

There is also a great need for new methods for detecting a susceptibility to such CNS disorders as schizophrenia, depression and bipolar disorder, as well as for preventing or following up the development of the disease. Diagnostic tools could also prove extremely useful. Indeed, early identification of subjects at risk of developing such CNS disorders would enable early and/or prophylactic treatment to be administered. Moreover, accurate assessments of the eventual efficacy of a medicament as well as the patent's eventual tolerance to it may enable clinicians to enhance the benefit/risk ratio of treatment regimes for CNS disorders such as those for schizophrenia, depression, bipolar disorder or other mood disorders.

### SUMMARY OF THE INVENTION

The present invention stems from an identification of novel polymorphisms including biallelic markers located on human chromosome 13q31-q33 locus, an identification and characterization of novel schizophrenia-related genes located on human chromosome 13q31-q33 locus, and from an identification of genetic associations between alleles of biallelic markers located on human chromosome 13q31-q33 locus and disease, as confirmed and characterized in a panel of human subjects. The novel polymorphisms and the schizophrenia-associated gene sequences has been filed in U.S. patent application Ser. No. 09/539,333 and International Patent Application No. PCT/IB00/00435, which disclosures are hereby incorporated by reference in their entireties.

CNS disorders which can be treated in accordance with the present invention include presenile dementia (early onset Alzheimer's disease), senile dementia (dementia of the Alzheimer's type), Parkinsonism including Parkinson's disease, Huntington's chorea, tardive dyskinesia, hyperkinesia, mania, attention deficit hyperactivity disorder (ADHD), attention deficit disorder (ADD), anxiety disorders, dyslexia, phycotic disorders, schizophrenia, bipolar disorder, major depressive episodes, manic episodes, hypomanic episodes, depression, autistic diorders, substance abuse, excessive aggression, tic disorders and Tourette's syndrome. Preferred disorders of the present invention include schizophrenia, depression and bipolar disorder. Further preferred embodiments of schizophrenia and schizophreniform disorders include: schizophrenia (catatonic), schizophrenia (disorganized), schizophrenia (paranoid), schizophrenia (undifferential), schizophrenia (residual), schizophreniform disorder, brief reactive psychosis, schizoaffective disorder, induced psychotic disorder, schizotypal personality disorder, schizoid personality disorder, paranoid personality disorder and delusional (paranoid) disorder.

The present invention pertains to methods for providing treatment of CNS disorders to a subject susceptible to such a disorder, and for providing treatment to a subject suffering from a CNS disorder. In particular, the method comprises administering to a patient an amount of a DAO or DDO antagonist or inhibitor compound effective for providing some degree of reversal or amelioration of the progression of the CNS disorder, reversal or amelioration of the symptoms of the CNS disorder, and reversal or amelioration of the reoccurrence of the CNS disorder.

The present invention further pertains to methods for providing prevention of CNS disorders to a subject susceptible to such a disorder, and for providing treatment to a subject suffering from a CNS disorder. In particular, the method comprises administering to a patient an amount of a DAO or DDO antagonist compound effective for providing some degree of prevention of the progression of the CNS disorder (i.e., provide protective effects), prevention of the symptoms of the CNS disorder, and prevention of the reoccurrence of the CNS disorder.

The present invention further pertains to the genomic sequence of DAO, novel exons discovered in the DAO gene, novel polymorphic biallelic markers (SNPs) discovered in the DAO gene, methods of detecting persons susceptible to a CNS disorder, novel methods of antagonizing, inhibiting or reducing the activity of DAO, novel methods of agonizing, promoting, increasing the activity of DAO, and a novel composition which affects DAO activity. The present invention further pertains to nucleic acid molecules comprising the genomic sequences of a novel human gene encoding g34872 (sbg1) proteins, proteins encoded thereby, as well as antibodies thereto, as described in copending U.S. patent application Ser. No. 09/539,333 and International Patent Application No. PCT/IB00/00435, which disclosures are hereby incorporated by reference in their entireties. The invention also deals with the cDNA sequences encoding the g34872, DAO and DDO proteins, and variants thereof. Oligonucleotide probes or primers hybridizing specifically with a g34872, DAO, and DDO genomic or cDNA sequence are also part of the present invention, as well as DNA amplification and detection methods using said primers and probes.

A further object of the invention consists of recombinant vectors comprising any of the nucleic acid sequences described above, and in particular of recombinant vectors comprising a g34872, DDO, and DAO regulatory sequence or a sequence encoding a g34872, DDO, and DAO protein, as well as of cell hosts and transgenic non human animals comprising said nucleic acid sequences or recombinant vectors.

The invention also concerns to biallelic markers of the g34872, DAO and DDO gene and the use thereof. Included are probes and primers for use in genotyping biallelic markers of the invention.

An embodiment of the invention encompasses any polynucleotide of the invention attached to a solid support polynucleotide may comprise a sequence disclosed in the present specification; optionally, said polynucleotide may comprise, consist of, or consist essentially of any polynucleotide described in the present specification; optionally, said determining may be performed in a hybridization assay, sequencing assay, microsequencing assay, or an enzyme-based mismatch detection assay; optionally, said polynucleotide may be attached to a solid support, array, or addressable array; optionally, said polynucleotide may be labeled.

Finally, the invention is directed to drug screening assays and methods for the screening of substances for the treatment of schizophrenia, bipolar disorder or a related CNS disorder based on the role of g34872, DAO, or DDO nucleotides and polynucleotides in disease. One object of the invention deals with animal models of schizophrenia, including mouse, primate, non-human primate bipolar disorder or related CNS disorder based on the role of g34872, DAO, or DDO in disease. The invention is also directed to methods for the screening of substances or molecules that inhibit the expression of g34872, DAO, or DDO, as well as with methods for the screening of substances or molecules that interact with a g34872, DAO, or DDO polypeptide, or that modulate the activity of a g34872, DAO, or DDO polypeptide.

As noted above, certain aspects of the present invention stem from the identification of genetic associations between schizophrenia and bipolar disorder and alleles of biallelic markers of g34872 gene and the DAO gene. The invention provides appropriate tools for establishing further genetic associations between alleles of biallelic markers in the g34872 and DAO locus and either side effects or benefit resulting from the administration of agents acting on CNS disorders or symptoms such as schizophrenia, depression or bipolar disorder, or schizophrenia or bipolar disorder symptoms, includng agents like chlorpromazine, clozapine, risperidone, olanzapine, sertindole, quetiapine and ziprasidone.

The invention provides appropriate tools for establishing further genetic associations between alleles of biallelic markers of DAO and g34872 with a trait. Methods and products are provided for the molecular detection of a genetic susceptibility in humans to schizophrenia and bipolar disorder. They can be used for diagnosis, staging, prognosis and monitoring of this disease, which processes can be further included within treatment approaches. The invention also provides for the efficient design and evaluation of suitable therapeutic solutions including individualized strategies for optimizing drug usage, and screening of potential new medicament candidates.

A preferred embodiment of the invention includes a method of treating a central nervous system disorder in a patient in need thereof, the method comprising administering said patient an effective amount of a composition or compound comprising a DAO antagonist or inhibitor or a DDO antagonist or inhibitor.

Further preferred is a method of treating psychosis, the method comprising administering to a patient suffering therefrom a therapeutically effective amount of a composition or compound comprising a DAO antagonist or inhibitor or a DDO inhibitor or antagonist.

Further preferred is a method of treating schizophrenia, the method comprising administering to a patient suffering therefrom a therapeutically effective amount of a composition or compound comprising a DAO antagonist or inhibitor or a DDO inhibitor or antagonist.

Further preferred is a method of treating bipolar disorder, the method comprising administering to a patient suffering therefrom a therapeutically effective amount of a composition or compound comprising a DAO antagonist or inhibitor or a DDO inhibitor or antagonist.

A preferred embodiment of the invention includes a method of treating a central nervous system disorder in a patient in need thereof, the method comprising administering said patient an effective amount of a composition or compound comprising a DAO antagonist or inhibitor and a DDO antagonist or inhibitor.

Further preferred is a method of treating psychosis, the method comprising administering to a patient suffering therefrom a therapeutically effective amount of a composition or compound comprising a DAO antagonist or inhibitor and a DDO inhibitor or antagonist.

Further preferred is a method of treating schizophrenia, the method comprising administering to a patient suffering therefrom a therapeutically effective amount of a composition or compound comprising a DAO antagonist or inhibitor and a DDO inhibitor or antagonist.

Further preferred is a method of treating bipolar disorder, the method comprising administering to a patient suffering therefrom a therapeutically effective amount of a composition or compound comprising a DAO antagonist or inhibitor and a DDO inhibitor or antagonist.

A preferred embodiment of the invention includes a method of treating a central nervous system disorder in a patient in need thereof, the method comprising administering said patient an effective amount of a composition or compound comprising a g34872 antagonist or inhibitor.

Further preferred is a method of treating psychosis, the method comprising administering to a patient suffering therefrom a therapeutically effective amount of a composition or compound comprising a g34872 inhibitor or antagonist.

Further preferred is a method of treating schizophrenia, the method comprising administering to a patient suffering therefrom a therapeutically effective amount of a composition or compound comprising a g34872 inhibitor or antagonist.

Further preferred is a method of treating bipolar disorder, the method comprising administering to a patient suffering therefrom a therapeutically effective amount of a composition or compound comprising a g34872 inhibitor or antagonist.

A preferred embodiment of the invention includes a method of treating a central nervous system disorder in a patient in need thereof, the method comprising administering said patient an effective amount of a composition or compound comprising a DAO antagonist or inhibitor or a DDO antagonist or inhibitor in combination with a g34872 antagonist or inhibitor composition or compound.

Further preferred is a method of treating psychosis, the method comprising administering to a patient suffering therefrom a therapeutically effective amount of a composition or compound comprising a DAO antagonist or inhibitor or a DDO antagonist or inhibitor in combination with a g34872 antagonist or inhibitor composition or compound.

Further preferred is a method of treating schizophrenia, the method comprising administering to a patient suffering therefrom a therapeutically effective amount of a composition or compound comprising a DAO antagonist or inhibitor or a DDO antagonist or inhibitor in combination with a g34872 antagonist or inhibitor composition or compound.

Further preferred is a method of treating bipolar disorder, the method comprising administering to a patient suffering therefrom a therapeutically effective amount of a composition or compound comprising a DAO antagonist or inhibitor or a DDO antagonist or inhibitor in combination with a g34872 antagonist or inhibitor composition or compound.

A preferred embodiment of the invention includes a method of treating a central nervous system disorder in a patient in need thereof, the method comprising administering said patient an effective amount of a composition or compound comprising a combination of a DAO antagonist or inhibitor, a DDO antagonist or inhibitor, and a g34872 antagonist or inhibitor composition or compound.

Further preferred is a method of treating psychosis, the method comprising administering to a patient suffering therefrom a therapeutically effective amount of a composition or compound comprising a combination of a DAO antagonist or inhibitor, a DDO antagonist or inhibitor, and a g34872 antagonist or inhibitor composition or compound.

Further preferred is a method of treating schizophrenia, the method comprising administering to a patient suffering therefrom a therapeutically effective amount of a composition or compound comprising a combination of a DAO antagonist or inhibitor, a DDO antagonist or inhibitor, and a g34872 antagonist or inhibitor composition or compound.

Further preferred is a method of treating bipolar disorder, the method comprising administering to a patient suffering therefrom a therapeutically effective amount of a composition or compound comprising a combination of a DAO antagonist or inhibitor, a DDO antagonist or inhibitor, and a g34872 antagonist or inhibitor composition or compound.

A preferred embodiment of the invention includes a method of treating a central nervous system disorder in a patient in need thereof, the method comprising administering said patient an effective amount of a composition or compound comprising at least one of the following: a DAO antagonist or inhibitor, a DDO antagonist or inhibitor, or a g34872 antagonist or inhibitor composition or compound.

Further preferred is a method of treating psychosis, the method comprising administering to a patient suffering therefrom a therapeutically effective amount of a composition or compound comprising at least one of the following: a DAO antagonist or inhibitor, a DDO antagonist or inhibitor, or a g34872 antagonist or inhibitor composition or compound.

Further preferred is a method of treating schizophrenia, the method comprising administering to a patient suffering therefrom a therapeutically effective amount of a composition or compound comprising at least one of the following: a DAO antagonist or inhibitor, a DDO antagonist or inhibitor, or a g34872 antagonist or inhibitor composition or compound.

Further preferred is a method of treating bipolar disorder, the method comprising administering to a patient suffering therefrom a therapeutically effective amount of a composition or compound comprising at least one of the following: a DAO antagonist or inhibitor, a DDO antagonist or inhibitor, or a g34872 antagonist or inhibitor composition or compound.

It should be appreciated that compositions or compounds known in the art to be used in methods of treating a central nervous system disorder, a psychosis, a schizophrenic disorder, or a bipolar disorder in a patient in need thereof, which are known to or inherently act to inhibit or antagonize DAO, DDO or g34872 are preferentially excluded from the present invention.

A further preferred embodiment of the invention relates to methods to inhibit DAO activity. Furthermore, the invention relates to a method to treat schizophrenia by inhibiting DAO activity. Further preferred is a method to treat schizophrenia by inhibiting DAO activity using a composition comprising a ketimine to inhibit DAO activity.

Another preferred embodiment is directed to a method to inhibit DDO activity. Furthermore, the invention relates to a method to treat schizophrenia by inhibiting DDO activity. Further preferred is a method to treat schizophrenia by inhibiting DDO activity using a composition comprising a ketimine to inhibit DDO activity.

Another preferred embodiment of the invention relates to methods of inhibiting the interaction between DAO and g34872.

Another preferred embodiment of the invention relates to a method of inhibiting the interaction between g34872 and DDO.

Another embodiment of the invention relates to any polypeptide fragment of a DAO polypeptide of SEQ ID NOs: 7, 8, 9, 10, or 18 which antagonizes the interaction between said DAO polypeptide and a g34872 polypeptide of SEQ ID NO: 14, or fragment thereof. Further preferred is a fragment of a DAO polypeptide comprising amino acids 23-347 of SEQ ID NO: 7. Further preferred is a fragment of a DAO polypeptide comprising amino acids 227-347 of SEQ ID NO: 7. Further preferred is a fragment of a DAO polypeptide comprising amino acids 31-347 of SEQ ID NO: 7. Further preferred is a fragment of a DAO polypeptide comprising amino acids 51-347 of SEQ ID NO: 7. Further preferred is a fragment of a DAO polypeptide comprising amino acids 66-347 of SEQ ID NO: 7. Further preferred is a fragment of a DAO polypeptide comprising amino acids 101-347 of SEQ ID NO: 7. Further preferred is a fragment of a DAO polypeptide comprising amino acids 126-347 of SEQ ID NO: 7. Further preferred is a fragment of a DAO polypeptide comprising amino acids 146-347 of SEQ ID NO: 7. Further preferred is a fragment of a DAO polypeptide comprising amino acids 175-347 of SEQ ID NO: 7. Further preferred is a fragment of a DAO polypeptide comprising amino acids 180-347 of SEQ ID NO: 7. Further preferred is a fragment of a DAO polypeptide comprising amino acids 1-189 of SEQ ID NO: 7. Further preferred is a fragment of a DAO polypeptide comprising amino acids 1-205 of SEQ ID NO: 7. Further preferred is a fragment of a DAO polypeptide comprising amino acids 31-189 of SEQ ID NO: 7. Further preferred is a fragment of a DAO polypeptide comprising amino acids 31-205 of SEQ ID NO: 7. Further preferred is a fragment of a DAO polypeptide comprising amino acids 84-205 of SEQ ID NO: 7.

A further preferred embodiment of the invention relates to compositions which bind to a DAO polypeptide or fragment thereof. Further preferred are compositions which bind to a fragment of a DAO polypeptide comprising amino acids 23-347 of SEQ ID NO: 7. Further preferred are compositions which bind to a fragment of a DAO polypeptide comprising amino acids 227-347 of SEQ ID NO: 7. Further preferred are compositions which bind to a fragment of a DAO polypeptide comprising amino acids 31-347 of SEQ ID NO: 7. Further preferred are compositions which bind to a fragment of a DAO polypeptide comprising amino acids 51-347 of SEQ ID NO: 7. Further preferred are compositions which bind to a fragment of a DAO polypeptide comprising amino acids 66-347 of SEQ ID NO: 7. Further preferred are compositions which bind to a fragment of a DAO polypeptide comprising amino acids 101-347 of SEQ ID NO: 7. Further preferred are compositions which bind to a fragment of a DAO polypeptide comprising amino acids 126-347 of SEQ ID NO: 7. Further preferred are compositions which bind to a fragment of a DAO polypeptide comprising amino acids 146-347 of SEQ ID NO: 7. Further preferred are compositions which bind to a fragment of a DAO polypeptide comprising amino acids 175-347 of SEQ ID NO: 7. Further preferred are compositions which bind to a fragment of a DAO polypeptide comprising amino acids 180-347 of SEQ ID NO: 7. Further preferred are compositions which bind to a fragment of a DAO polypeptide comprising amino acids 1-189 of SEQ ID NO: 7. Further preferred are compositions which bind to a fragment of a DAO polypeptide comprising amino acids 1-205 of SEQ ID NO: 7. Further preferred are compositions which bind to a fragment of a DAO polypeptide comprising amino acids 31-189 of SEQ ID NO: 7. Further preferred are compositions which bind to a fragment of a DAO polypeptide comprising amino acids 31-205 of SEQ ID NO: 7. Further preferred are compositions which bind to a fragment of a DAO polypeptide comprising amino acids 84-205 of SEQ ID NO: 7.

A further preferred embodiment is directed to a method of treating schizophrenia, the method comprising administering to a patient suffering therefrom a therapeutically effective amount of a composition comprising a composition which binds to a DAO polypeptide, or a fragment thereof. Further preferred is a method of treating schizophrenia, the method comprising administering to a patient suffering therefrom a therapeutically effective amount of a composition comprising a composition which binds to a DAO polypeptide comprising amino acids 23-347 of SEQ ID NO: 7. Further preferred is a method of treating schizophrenia, the method comprising administering to a patient suffering therefrom a therapeutically effective amount of a composition comprising a composition which binds to a DAO polypeptide comprising amino acids 227-347 of SEQ ID NO: 7. Further preferred is a method of treating schizophrenia, the method comprising administering to a patient suffering therefrom a therapeutically effective amount of a composition comprising a composition which binds to a DAO polypeptide comprising amino acids 31-347 of SEQ ID NO: 7. Further preferred a method of treating schizophrenia, the method comprising administering to a patient suffering therefrom a therapeutically effective amount of a composition comprising a composition which binds to a DAO polypeptide comprising amino acids 51-347 of SEQ ID NO: 7. Further preferred is a method of treating schizophrenia, the method comprising administering to a patient suffering therefrom a therapeutically effective amount of a composition comprising a composition which binds to a DAO polypeptide comprising amino acids 66-347 of SEQ ID NO: 7. Further preferred is a method of treating schizophrenia, the method comprising administering to a patient suffering therefrom a therapeutically effective amount of a composition comprising a composition which binds to a DAO polypeptide comprising amino acids 101-347 of SEQ ID NO: 7. Further preferred is a method of treating schizophrenia, the method comprising administering to a patient suffering therefrom a therapeutically effective amount of a composition comprising a composition which binds to a DAO polypeptide comprising amino acids 126-347 of SEQ ID NO: 7. Further preferred is a method of treating schizophrenia, the method comprising administering to a patient suffering therefrom a therapeutically effective amount of a composition comprising a composition which binds to a DAO polypeptide comprising amino acids 146-347 of SEQ ID NO: 7. Further preferred is a method of treating schizophrenia, the method comprising administering to a patient suffering therefrom a therapeutically effective amount of a composition comprising a composition which binds to a DAO polypeptide comprising amino acids 175-347 of SEQ ID NO: 7. Further preferred is a method of treating schizophrenia, the method comprising administering to a patient suffering therefrom a therapeutically effective amount of a composition comprising a composition which binds to a DAO polypeptide comprising amino acids 180-347 of SEQ ID NO: 7. Further preferred is a method of treating schizophrenia, the method comprising administering to a patient suffering therefrom a therapeutically effective amount of a composition comprising a composition which binds to a DAO polypeptide comprising amino acids 1-189 of SEQ ID NO: 7. Further preferred is a method of treating schizophrenia, the method comprising administering to a patient suffering therefrom a therapeutically effective amount of a composition comprising a composition which binds to a DAO polypeptide comprising amino acids 1-205 of SEQ ID NO: 7. Further preferred is a method of treating schizophrenia, the method comprising administering to a patient suffering therefrom a therapeutically effective amount of a composition comprising a composition which binds to a DAO polypeptide comprising amino acids 31-189 of SEQ ID NO: 7. Further preferred is a method of treating schizophrenia, the method comprising administering to a patient suffering therefrom a therapeutically effective amount of a composition comprising a composition which binds to a DAO polypeptide comprising amino acids 31-205 of SEQ ID NO: 7. Further preferred is a method of treating schizophrenia, the method comprising administering to a patient suffering therefrom a therapeutically effective amount of a composition comprising a composition which binds to a DAO polypeptide comprising amino acids 84-205 of SEQ ID NO: 7.

A further preferred embodiment is directed to a method of treating bipolar disorder, the method comprising administering to a patient suffering therefrom a therapeutically effective amount of a composition comprising a composition which binds to a DAO polypeptide, or a fragment thereof. Further preferred is a method of treating bipolar disorder, the method comprising administering to a patient suffering therefrom a therapeutically effective amount of a composition comprising a composition which binds to a DAO polypeptide comprising amino acids 23-347 of SEQ ID NO: 7. Further preferred is a method of treating bipolar disorder, the method comprising administering to a patient suffering therefrom a therapeutically effective amount of a composition comprising a composition which binds to a DAO polypeptide comprising amino acids 227-347 of SEQ ID NO: 7. Further preferred is a method of treating bipolar disorder, the method comprising administering to a patient suffering therefrom a therapeutically effective amount of a composition comprising a composition which binds to a DAO polypeptide comprising amino acids 31-347 of SEQ ID NO: 7. Further preferred a method of treating bipolar disorder, the method comprising administering to a patient suffering therefrom a therapeutically effective amount of a composition comprising a composition which binds to a DAO polypeptide comprising amino acids 51-347 of SEQ ID NO: 7. Further preferred is a method of treating bipolar disorder, the method comprising administering to a patient suffering therefrom a therapeutically effective amount of a composition comprising a composition which binds to a DAO polypeptide comprising amino acids 66-347 of SEQ ID NO: 7. Further preferred is a method of treating bipolar disorder, the method comprising administering to a patient suffering therefrom a therapeutically effective amount of a composition comprising a composition which binds to a DAO polypeptide comprising amino acids 101-347 of SEQ ID NO: 7. Further preferred is a method of treating bipolar disorder, the method comprising administering to a patient suffering therefrom a therapeutically effective amount of a composition comprising a composition which binds to a DAO polypeptide comprising amino acids 126-347 of SEQ ID NO: 7. Further preferred is a method of treating bipolar disorder, the method comprising administering to a patient suffering therefrom a therapeutically effective amount of a composition comprising a composition which binds to a DAO polypeptide comprising amino acids 146-347 of SEQ ID NO: 7. Further preferred is a method of treating bipolar disorder, the method comprising administering to a patient suffering therefrom a therapeutically effective amount of a composition comprising a composition which binds to a DAO polypeptide comprising amino acids 175-347 of SEQ ID NO: 7. Further preferred is a method of treating bipolar disorder, the method comprising administering to a patient suffering therefrom a therapeutically effective amount of a composition comprising a composition which binds to a DAO polypeptide comprising amino acids 180-347 of SEQ ID NO: 7. Further preferred is a method of treating bipolar disorder, the method comprising administering to a patient suffering therefrom a therapeutically effective amount of a composition comprising a composition which binds to a DAO polypeptide comprising amino acids 1-189 of SEQ ID NO: 7. Further preferred is a method of treating bipolar disorder, the method comprising administering to a patient suffering therefrom a therapeutically effective amount of a composition comprising a composition which binds to a DAO polypeptide comprising amino acids 1-205 of SEQ ID NO: 7. Further preferred is a method of treating bipolar disorder, the method comprising administering to a patient suffering therefrom a therapeutically effective amount of a composition comprising a composition which binds to a DAO polypeptide comprising amino acids 31-189 of SEQ ID NO: 7. Further preferred is a method of treating bipolar disorder, the method comprising administering to a patient suffering therefrom a therapeutically effective amount of a composition comprising a composition which binds to a DAO polypeptide comprising amino acids 31-205 of SEQ ID NO: 7. Further preferred is a method of treating bipolar disorder, the method comprising administering to a patient suffering therefrom a therapeutically effective amount of a composition comprising a composition which binds to a DAO polypeptide comprising amino acids 84-205 of SEQ ID NO: 7.

A further preferred embodiment of the invention relates to compositions which bind to a g34872 polypeptide of SEQ ID NO: 14, or fragment thereof. Further preferred are compositions which bind to a g34872 polypeptide comprising amino acids 65-153 of SEQ ID NO: 14, or fragment thereof. Further preferred are compositions which bind to a polypeptide of SEQ ID NO: 16 or fragment thereof.

A further preferred embodiment is directed to a method of treating schizophrenia, the method comprising administering to a patient suffering therefrom a therapeutically effective amount of a composition comprising a composition which binds to a g34872 polypeptide of SEQ ID NO: 14, or fragment thereof. Further preferred is a method of treating schizophrenia, the method comprising administering to a patient suffering therefrom a therapeutically effective amount of a composition comprising a composition which binds to a g34872 polypeptide comprising amino acids 65-153 of SEQ ID NO: 14, or fragment thereof. Further preferred is a method of treating schizophrenia, the method comprising administering to a patient suffering therefrom a therapeutically effective amount of a composition comprising a composition which binds to a polypeptide of SEQ ID NO: 16 or fragment thereof.

A further preferred embodiment is directed to a method of treating bipolar disorder, the method comprising administering to a patient suffering therefrom a therapeutically effective amount of a composition comprising a composition which binds to a g34872 polypeptide of SEQ ID NO: 14, or fragment thereof. Further preferred is a method of treating bipolar disorder, the method comprising administering to a patient suffering therefrom a therapeutically effective amount of a composition comprising a composition which binds to a g34872 polypeptide comprising amino acids 65-153 of SEQ ID NO: 14, or fragment thereof. Further preferred is a method of treating bipolar disorder, the method comprising administering to a patient suffering therefrom a therapeutically effective amount of a composition comprising a composition which binds to a polypeptide of SEQ ID NO: 16 or fragment thereof.

A further preferred embodiment of the invention relates to any polypeptide fragment of a g34872 polypeptide of SEQ ID NO: 14 which antagonizes the interaction between said g34872 polypeptide or fragment thereof and a DAO polypeptide or fragment thereof. Further preferred is any fragment of g34872 which antagonizes the increase in DAO activity by a g34872 polypeptide. Further preferred is a fragment of a g34872 polypeptide comprising the amino acids of SEQ ID NO: 16.

A further preferred embodiment of the invention relates to compositions which antagonize the interaction between a g34872 polypeptide of SEQ ID NO: 14, or a fragment thereof, and a DAO polypeptide of SEQ ID NOs: 7-10 or 18, or a fragment thereof.

A further preferred embodiment of the invention relates to compositions which antagonize the interaction between a g34872 polypeptide of SEQ ID NO: 14, or a fragment thereof, and a DDO polypeptide of SEQ ID NOs: 21 or 22, or a fragment thereof.

A further preferred embodiment of the invention relates to compositions which antagonize the interaction between a g34872 polypeptide of SEQ ID NO: 14, or a fragment thereof, and a DDO polypeptide of SEQ ID NOs: 21 or 22, or a fragment thereof.

Another embodiment of the invention relates to methods of increasing the activity of DAO with a g34872 polypeptide or fragment thereof. Furthermore, the invention relates to methods of increasing the activity of DDO with a g34872 polypeptide or fragment thereof.

A further embodiment of the invention relates to methods of inhibiting the glycosylation of DAO.

A further embodiment of the invention relates to methods of enhancing the multimerization of DAO.

A further embodiment of the invention relates to methods of inhibiting translation of DAO.

A further embodiment of the invention relates to differential identification of DAO variants.

A preferred embodiment of the invention is directed to a composition or a compound which reduces, inhibits or antagonizes DAO activity. Further preferred, the composition or compound is a competitive inhibitor or antagonist of DAO activity. Further preferred, the composition or compound is a noncompetitive inhibitor or antagonist of DAO activity. Further preferred, the composition or compound is a uncompetitive inhibitor or antagonist of DAO activity. Further preferred, the composition or compound is an allosteric inhibitor or antagonist of DAO activity. Further preferred, the composition or compound is a reversible inhibitor or antagonist of DAO activity. Further preferred, the composition or compound is an irreversible inhibitor or antagonist of DAO activity.

A further embodiment is directed to a composition or compound which reduces, inhibits or antagonizes DDO activity. Further preferred, the composition or compound is a competitive inhibitor or antagonist of DDO activity. Further preferred, the composition or compound is a noncompetitive inhibitor or antagonist of DDO activity. Further preferred, the composition or compound is a uncompetitive inhibitor or antagonist of DDO activity. Further preferred, the composition or compound is an allosteric inhibitor or antagonist of DDO activity. Further preferred, the composition or compound is a reversible inhibitor or antagonist of DDO activity. Further preferred, the composition or compound is an irreversible inhibitor or antagonist of DDO activity. Further preferred are compositions or compounds which reduce, inhibit or antagonize the activity of DAO and DDO.

Further preferred is a method of treating a CNS disorder with a composition or compound which reduces, inhibits or antagonizes DAO activity. Further preferred is a method of treating a CNS disorder with a composition or compound which reduces, inhibits or antagonizes DDO activity. Further preferred is a method of treating a CNS disorder with a composition or compound which reduces, inhibits or antagonizes both DAO and DDO activity. Further preferred is a method of treating a CNS disorder with a composition or compound which reduces, inhibits or antagonizes either DAO and DDO activity. Further preferred is a method of treating a CNS disorder with a first composition or compound which reduces, inhibits or antagonizes DAO in combination with a second composition which reduces, inhibits or antagonizes DDO activity. Further preferred is a method of treating a CNS disorder with a composition or compound which reduces, inhibits or antagonizes DAO in combination with another composition. Further preferred is a method of treating a CNS disorder with a composition or compound which reduces, inhibits or antagonizes DAO in combination with another composition routinely used in the treatment of said CNS disorder. Further preferred is a method of treating a CNS disorder with a composition or compound which reduces, inhibits or antagonizes DAO in combination with another composition unrelated to the treatment of said CNS disorder. Further preferred is a method of treating a CNS disorder with a composition or compound which reduces, inhibits or antagonizes DDO in combination with another composition. Further preferred is a method of treating a CNS disorder with a composition or compound which reduces, inhibits or antagonizes DDO in combination with another composition routinely used in the treatment of said CNS disorder. Further preferred is a method of treating a CNS disorder with a composition or compound which reduces, inhibits or antagonizes DDO in combination with another composition unrelated to the treatment of said CNS disorder.

Preferred compositions or compounds of the invention which reduce, inhibit or antagonize DAO or DDO activity are selected from, but not limited to, the list comprising:

i. IRI, 2-oxo-3-pentynoate;

ii. CMI, Aminoguanidine (Guanylhydrazine; Carbamimidic hydrazide; Pimagedine; GER 11; Hydrazinecarboximidamide) or hydrochloride salt (Guanylhydrazine hydrochloride), bicarbonate salt, nitrate salt, sulfate (2:1) salt, sulfate (1:1) salt, and hemisulfate salt thereof;

iii. FI, benzoic acid;

iv. FI, sodium benzoate;

v. FI, 2-aminobenzoate;

vi. FI, 3-aminobenzoate;

vii. FI, 4-aminobenzoate (p-aminobenzoate, PABA, Vitamin Bx, Vitamin H1);

viii. CMI, Methylglyoxal bis(guanylhydrazone) ( also known as: Methyl GAG; Mitoguazone; 1,1′-((Methylethanediylidene)dinitrilo)diguanidine; Hydrazinecarboximidamide, 2,2′-(1-methyl-1,2-ethanediylidene)bis-; Pyruvaldehyde bis(amidinohydrazone); Megag; Mitoguazona [INN-Spanish]; Guanidine, 1,1′-((methylethanediylidene)dinitrilo)di-; 1,1′-((Methylethanediylidene)dinitrilo)diguanidine);

ix. CMI, Methylglyoxal bis(guanylhydrazone), dihydrochloride;

x. CMI, phenylglyoxal bis(guanylhydrazone) (PhGBG);

xi. CMI, glyoxal bis(guanylhydrazone) (GBG; Guanidine, 1,1′-(ethanediylidenedinitrilo)di-(8CI); Hydrazinecarboximidamide, 2,2′-(1,2-ethanediylidene)bis-(9CI));

xii. CMI, indole-propionic (IPA, 3-(3-Indolyl)propanoic acid);

xiii. CMI, 3-indole-acetic acid (Heteroauxin, IAA);

xiv. CMI, Indole-3-acetic acid Sodium salt;

xv. CMI, Indole-3-acetone;

xvi. CMI, Indole-3-acetamide;

xvii. CMI, Indole-3-acetyl-L-aspartic acid;

xviii. CMI, Indole-3-acetyl-L-alanine;

xix. CMI, Indole-3-acetylglycine;

xx. CMI, Indole-3-acetaldehyde Sodium Bisulfite Addition compound;

xxi. CMI, Indole-3-carboxylic acid;

xxii. CMI, Indole-3-pyruvic acid (3-(3-Indolyl)-2-oxopropanoic acid);

xxiii. FI, salicylic acid (2-Hydroxybenzoic acid);

xxiv. FI, salicylic acid Sodium Salt;

xxv. FI, Salicylic acid Potassium Salt;

xxvi. IRI, Dansyl chloride (5-(Dimethylamino)naphthalene-1-sulfonyl chloride);

xxvi. IRI, Dansyl fluoride (5-(Dimethylamino)naphthalene-1-sulfonyl fluoride);

xxviii. CMI, dansyl glycine;

xxix. CMI, Alanine tetrazole;

xxx. FI, benzoic tetrazole;

xxxi. CMI, tetrazole;

xxxii. CMI, Riboflavin 5′-pyrophosphate (RPP, 5-Phospho-alpha-D-ribosyl diphosphate, P-Rib-PP, P-RPP);

xxxiii. IRI, DL-propargylglycine (DL-PG, 2-Amino-4-pentynoic acid);

xxxiv. IRI, L-C-Propargylglycine;

xxxv. IRI, N-Acetyl-DL-propargylglycine;

xxxvi. FII, (±)-Sodium 3-hydroxybutyrate;

xxxvii. FI, Trigonelline Hydrochloride (1-Methylpyridinium-3-carboxylate);

xxxviii. FI, N-methylnicotinate;

xxxix. FI, Methyl 6-methylnicotinate;

xl. FI, Ethyl 2-methylnicotinate;

xli. CMI, Kojic acid (2-Hydroxymethyl-5-hydroxy-gamma-pyrone, 5-Hydroxy-2-hydroxymethyl-4-pyranone);

xlii. CMI, derivatives of kojic acid, such as: 6-(PYRROLIDINOMETHYL)-KOJIC ACID HYDROCHLORIDE, 6-(MORPHOLINOMETHYL)-KOJIC ACID, 6-(DIETHYLAMINOMETHYL)-KOJIC ACID Hydrochloride;

xliii. IRI, O-(2,4-dinitrophenyl)hydroxylamine;

xliv. CMI, 2,4-DINITROPHENYL GLYCINE;

xlv. CMI, Hydroxylamine Hydrochloride;

xlvi. IRI, Methyl-p-nitrobenzenesulfonate (Methyl 4-nitrobenzenesulfonate);

xlvii. FIV, Aminoethylcysteine-ketimine (AECK, Thialysine ketimine, 2H-1,4-Thiazine-5,6-dihydro-3-carboxylic acid, S-Aminoethyl-L-cysteine ketimine, 2H-1,4-Thiazine-3-carboxylic acid, 5,6-dihydro-);

xlviii. FIV, 1,4-thiazine derivatives;

xlix. CMI, 4-Phenyl-1,4-sulfonazan (Tetrahydro-4-phenyl-4H-1,4-thiazine 1-oxide, 4H-1,4-Thiazine, tetrahydro-4-phenyl-, 1-oxide);

l. CMI, Phenothiazine (Thiodiphenylamine, 10H-Phenothiazine, AFI-Tiazin, Agrazine, Antiverm, Dibenzo-1,4-thiazine);

li. CMI, 3,4-Dihydro-2H-1,4-thiazine-3,5-dicarboxylic acid (3,4-Dhtca, CAS#86360-62-5);

lii. CMI, Nifurtimox (Nifurtimox [BAN:INN], 1-((5-Nitrofurfurylidene)amino)-2-methyltetrahydro-1,4-thiazine-4,4-dioxide, 3-Methyl-4-(5′-nitrofurylidene-amino)-tetrahydro-4H-1,4-thiazine-1,1-dioxide, BAY 2502, 4-((5-Nitrofurfurylidene)amino)-3-methylthiomorpholine 1,1-dioxide, etc);

liii. FIV, 3-(1-Pyrrolidinylmethyl)-4-(5,6-dichloro-1-indancarbonyl)-tetrahydro-1,4-thiazine hydrochloride (R 84760; R 84761; Thiomorpholine, 4-((5,6-dichloro-2,3-dihydro-1H-inden-1-yl)carbonyl)-3-(1-pyrrolidinylmethyl)-, monohydrochloride, (R-(R*,S*))-);

liv. FIV, ketimine reduced forms;

lv. CMI, cystathionine;

lvi. FIII, cystathionine ketimine;

lvii. FIV, lanthionine ketimine;

lviii. FIV, thiomorpholine-2-carboxylic acid;

lix. CMI, thiomorpholine-2,6-dicarboxylic acid;

lx. FIV, TMDA (1,4-Thiomorpholine-3,5-dicarboxylic acid);

lxi. IRI, 1-chloro-1-nitroethane;

lxii. FI, anthranilate;

lxiii. FI, Ethyl 2-aminobenzoate (ethyl anthranilate);

lxiv. FI, Methyl 2-aminobenzoate (Methyl anthranilate);

lxv. FI, picolinate;

lxvi. FI, Ethyl picolinate (2-(Ethoxycarbonyl)pyridine, Ethyl 2-pyridinecarboxylate,;

lxvii. CMI, L-Leucine methyl ester, hydrochloride;

lxviii. CMI, L-leucine ([(S)-(+)-leucine]);

lxix. IRI, Fluorodinitrobenzene (1-Fluoro-2,4-dinitrobenzene, 2,4-DNFB, Benzene, 1-fluoro-2,4-dinitro-, VAN, etc);

lxx. IRI, Dinitrochlorobenzene (1-Chloro-2,4-dinitrobenzene, 1,3-Dinitro-4-chlorobenzene, etc);

lxxi. IRI, 1,2-cyclohexanedione;

lxxii. IRI, Allylglycine (D-Allylglycine, 4-Pentenoic acid, 2-amino-);

lxxiii. CMI, 2-amino-2,4-pentadienoate;

lxxiv. CMI, 2-hydroxy-2,4-pentadienoate;

lxxv. CMI, 2-amino-4-keto-2-pentenoate;

lxxvi. FII, 2-hydroxybutyrate;

lxxvii. FII, Sodium 2-hydroxybutyrate;

lxxviii. IRI, N-chloro-D-leucine;

lxxix. CMI, N-Acetyl-D-leucine;

lxxx. CMI, D-Leu (D-2-Amino-4-methylpentanoic acid);

lxxxi. IRI, D-propargylglycine; 2-Amino-4-pentynoic acid; D,L-Propargylglycine; L-2-Amino-4-pentynoic acid;

lxxxii. CMI, Progesterone (4-Pregnene-3,20-dione);

lxxxiii. CMI, FAD (Flavin adenine dinucleotide, 1H-Purin-6-amine, flavin dinucleotide, Adenosine 5′-(trihydrogen pyrophosphate), 5′-5′-ester with riboflavin, etc);

lxxxiv. CMI, 6-OH-FAD;

lxxxv. IRI, Phenylglyoxal (2,2-Dihydroxyacetophenone);

lxxxvi. IRI, Phenylglyoxal Monohydrate (2,2-Dihydroxyacetophenone monohydrate);

lxxxvii. FIII, Cyclothionine (Perhydro-1,4-thiazepine-3,5-dicarboxylic acid, 1,4-Hexahydrothiazepine-3,5-dicarboxylic acid, 1,4-Thiazepine-3,5-dicarboxylic acid, hexahydro-);

lxxxviii. CMI, alpha-alpha′-iminodipropionic (Alanopine; 2,2′-Iminodipropionic acid; L-Alanine, N-(1-carboxyethyl)-);

lxxxix. CMI, Meso-Diaminosuccinic acid (3-Aminoaspartic acid; Diaminosuccinic acid; CAS RN: 921-52-8); meso-2,3-Diaminosuccinic acid (CAS RN: 23220-52-2);

xc. CMI, Thiosemicarbazide (thiocarbamoyl hydrazide);

xci. CMI, Thiourea (Sulfourea; Thiocarbamide);

xcii. CMI, Methylthiouracil (4(6)-Methyl-2-thiouracil, 4-Hydroxy-2-mercapto 6-methylpyrimidine);

xciii. CMI, Sulphathiazole (N1-(2-Thiazolyl)sulfanilamide, 4-Amino-N-2-thiazolylbenzenesulfonamide);

xciv. CMI, Sulfathiazole Sodium Salt (4-Amino-N-2-thiazolylbenzenesulfonamide sodium salt);

xcv. CMI, Thiocyanate;

xcvi. FI, 3-METHYLBENZYL THIOCYANATE;

xcvii. CMI, methimazole (2-Mercapto-1-methylimidazole, 1-Methylimidazole-2-thiol);

xcviii. FII, Dicarboxylic hydroxyacids;

xcix. FII, 1,3-Acetonedicarboxylic acid (3-Oxoglutaric acid);

c. CMI, D-tartaric acid ([(2S,3S)-(−)-tartaric acid, unnatural tartaric acid]);

ci. CMI, L-tartaric acid ([(2R,3R)-(+)-tartaric acid, natural tartaric acid]);

cii. CMI, DL-tartaric acid;

ciii. potassium tartrate;

civ. FII, D-malic acid; [(R)-(+)-malic acid, (R)-(+)-hydroxysuccinic acid];

cv. FII, L-malic acid; [(S)-(−)-malic acid, (S)-(−)-hydroxysuccinic acid];

cvi. FII, DL-Malic acid (DL-hydroxysuccinic acid);

cvii. FII, Alpha-keto acids that are analogues of the amino acids alanine, leucine, phenylanaline, phenylglycine, tyrosine, serine, aspartate, etc and salts and derivatives thereof;

cviii. FII, pyruvic acid (2-Oxopropionic acid, alpha-Ketopropionic acid);

cix. FII, sodium pyruvate;

cx. FII, Pyruvic acid methyl ester (methyl pyruvate);

cxi. FI, Phenylpyruvic acid;

cxii. FII, Calcium phenylpyruvate (calcium pyruvate);

cxiii. FI, Phenylpyruvic acid Sodium salt (Sodium phenylpyruvate);

cxiv. FII, 4-hydroxyphenyl pyruvic acid;

cxv. FII, sodium alpha-ketoisovaleric acid (3-Methyl-2-oxobutyric acid Sodium salt, 3-Methyl-2-oxobutanoic acid sodium salt, a-Ketoisovaleric acid Sodium salt; Ketovaline Sodium salt);

cxvi. FI, benzoylformic acid (a-Oxophenylacetic acid, Phenylglyoxylic acid);

cxvii. FII, 4-methylthio-2-oxopentanoic acid;

cxviii. FII, 4-Methyl-2-oxopentanoic acid (4-Methyl-2-oxovaleric acid; alpha-Ketoisocaproic acid;;

cxix. FII, 4-methylthio-2-oxybutanoic acid;

cxx. FII, 2-oxybutanoic acid (hydroxybutyrate; 2-Hydroxybutyric acid; alpha-Hydroxy-n-butyric acid;

cxxi. FII, DL-alpha-Hydroxybutyric acid Sodium Salt (sodium(±)-2-Hydroxybutyrate);

cxxii. FII, Indole-3-pyruvic acid (alpha-Keto analogue of tryptophan);

cxxiii. The reaction product between cysteamine and bromopyruvate;

cxxiv. CMI, cysteamine (2-Aminoethanethiol; 2-Mercaptoethylamine);

cxxv. CMI, pantetheine;

cxxvi. CMI, S-adenosylmethionine;

cxxvii. IRI, Ethyl bromopyruvate;

cxxviii. IRI, Methyl bromopyruvate;

cxxix. IRI, Bromopyruvate; and

cxxx. CMI, 5-S-Cysteinyldopamine,

wherein IRI indicates Irreversible Inhibitor compositions; CMI indicates Competitive Inhibitor compositions not included in Formula I-IV compositions; FI indicates Formula I compositions as described herein; FII indicates Formula II compositions as described herein; FIII indicates Formula III compositions as described herein; and FIV indicates Formula IV compositions as described herein. It should be appreciated that Formula I-IV compositions are competitive, noncompetitive, uncompetitive or allosteric inhibitors of DAO or DDO.

Preferred compositions to be used in methods of the invention to reduce, inhibit, or antagonize DAO or DDO catalytic activity in vitro or in vivo are selected from the above list of compositons “i” through and including “cxxx”; more preferred are compositions selected from irreversible inhibitor compositions, Formula I compositions, Formula II compositions, Formula III compositions and Formula IV compositions; even more preferred are compositions selected from Formula I compositions, Formula II compositions, Formula III compositions and Formula IV; most preferred are compositions selected from Formula I and Formula IV. Further preferred compositions to be used in methods of the invention to reduce, inhibit, or antagonize DAO or DDO catalytic activity in vitro or in vivo are selected from the group comprising benzoate, aminoethylcysteine ketimine (AECK), and derivatives thereof.

In a further preferred embodiment, preferred compositions or compounds to be used in methods of the invention of treating a CNS disorder are selected from the above list of compositons “i” through and including “cxxx”; more preferred are compositions selected from irreversible inhibitor compositions, Formula I compositions, Formula II compositions, Formula III compositions and Formula IV compositions; even more preferred are compositions selected from Formula I compositions, Formula II compositions, Formula III compositions and Formula IV; most preferred are compositions selected from Formula I and Formula IV. Further preferred compositions to be used in methods of the invention of treating a CNS disorder are selected from the group comprising benzoate, aminoethylcysteine ketimine (AECK), and derivatives thereof.

A highly preferred compound or composition of the invention to reduce, inhibit or antagonize DAO or DDO activity is selected from the list comprising, but not limited to: Aminoethylcysteine-ketimine (AECK, Thialysine ketimine, 2H-1,4-Thiazine-5,6-dihydro-3-carboxylic acid, S-Aminoethyl-L-cysteine ketimine, 2H-1,4-Thiazine-3-carboxylic acid, 5,6-dihydro-); aminoethylcysteine (thialysine); cysteamine; pantetheine; cystathionine and S-adenosylmethionine.

Another preferred embodiment of the invention is directed to a compound or composition which reduces, inhibits or antagonizes the oxidation or degradation of an amino acid or derivative thereof. Another preferred embodiment of the invention is directed to a compound or composition which reduces, inhibits or antagonizes the oxidation or degradation an L-amino acid or derivative thereof. Another preferred embodiment of the invention is directed to a compound or composition which reduces, inhibits or antagonizes the oxidation or degradation of an D-amino acid or derivative thereof. Another preferred embodiment of the invention is directed to a compound or composition which reduces, inhibits or antagonizes the oxidation or degradation of glycine or derivative thereof. A further preferred embodiment of the invention is directed to a compound or composition which reduces, inhibits or antagonizes the oxidation or degradation of at least one D-amino acid selected from the list comprising: D-Met, D-Pro, D-Phe, D-Tyr, D-Ile, D-Leu, D-Ala, D-Val, D-Ser, D-Arg, D-His, D-norleucine, D-Trp, D-Ornithine, cis-4-hydroxy-D-proline, D-Thr, D-Trp-methyl ester, N-acetyl-D-Ala, D-Lys, D-Asp, D-Glu, D-Asn, D-Gln, D-Asp-dimethyl-ester and N-methyl-D-Asp. Further preferred is a composition which reduces, inhibits, or antagonizes the oxidation or degradation of D-serine. Further preferred is a composition or compound which reduces, inhibits or antagonizes the oxidation or degradation of D-Ser, N-methyl-D-Asp, D-Asp or Gly. A preferred compound or composition of the invention which reduces, inhibits or antagonizes the oxidation or degradation of an amino acid, or derivative thereof, is selected from the list including, but not limited to comprising: Aminoethylcysteine-ketimine (AECK, Thialysine ketimine, 2H-1,4-Thiazine-5,6-dihydro-3-carboxylic acid, S-Aminoethyl-L-cysteine ketimine, 2H-1,4-Thiazine-3-carboxylic acid, 5,6-dihydro-); aminoethylcysteine (thialysine); cysteamine; pantetheine; cystathionine and S-adenosylmethionine. A preferred compound or composition of the invention which reduces, inhibits or antagonizes the oxidation or degradation of D-Met, D-Pro, D-Phe, D-Tyr, D-Ile, D-Leu, D-Ala, D-Val, D-Ser, D-Arg, D-His, D-norleucine, D-Trp, D-Ornithine, cis-4-hydroxy-D-proline, D-Thr, D-Trp-methyl ester, N-acetyl-D-Ala, D-Lys, D-Asp, D-Glu, D-Asn, D-Gln, D-Asp-dimethyl-ester, N-methyl-D-Asp or Gly is selected from the list including, but not limited to comprising: Aminoethylcysteine-ketimine (AECK, Thialysine ketimine, 2H-1,4-Thiazine-5,6-dihydro-3-carboxylic acid, S-Aminoethyl-L-cysteine ketimine, 2H-1,4-Thiazine-3-carboxylic acid, 5,6-dihydro-); aminoethylcysteine (thialysine); cysteamine; pantetheine; cystathionine and S-adenosylmethionine. A preferred compound or composition of the invention which reduces, inhibits or antagonizes the oxidation or degradation of D-Ser is selected from the list including, but not limited to comprising: Aminoethylcysteine-ketimine (AECK, Thialysine ketimine, 2H-1,4-Thiazine-5,6-dihydro-3-carboxylic acid, S-Aminoethyl-L-cysteine ketimine, 2H-1,4-Thiazine-3-carboxylic acid, 5,6-dihydro-); aminoethylcysteine (thialysine); cysteamine; pantetheine; cystathionine and S-adenosylmethionine.

Another embodiment of the invention is directed to a composition which reduces, inhibits or antagonizes the oxidation of Reduced-Flavin Adenine Dinucleotide (Re-FAD). Another embodiment of the invention is directed to a composition which reduces, inhibits or antagonizes the reduction of Oxidized-Flavin Adenine Dinucleotide (Ox-FAD). A further embodiment is directed to a composition which reduces, inhibits or antagonizes the activity of flavokinase. A further embodiment is directed to a composition which reduces, inhibits or antagonizes the activity of FAD pyrophosphorylase. A further embodiment is directed to a composition which binds to or interacts with Re-FAD or Ox-FAD. A further embodiment is directed to a composition which binds to or interacts with flavokinase or FAD pyrophosphorylase.

A further preferred embodiment is directed to a composition or compound which increases, agonizes or promotes the activity of cystathionine beta-synthase. A preferred composition which increases, agonizes or promotes the activity of cystathionine beta-synthase comprises S-adenosylmethionine or homocysteine. Another preferred composition which increases, agonizes or promotes the activity of cystathionine beta-synthase is pyridoxine or derivative thereof.

A further preferred embodiment of the invention is directed to a method of screening for a composition which binds to or interacts with DAO, DDO, Re-FAD, Ox-FAD, flavokinase, FAD pyrophosphorylase, cystathionine beta synthase, L-amino acid oxidase, or glutamine transaminase. A further preferred embodiment of the invention is directed to a method of screening for a composition which reduces, inhibits or antagonizes the activity of DAO, DDO, flavokinase, FAD pyrophosphorylase, L-amino acid oxidase, or glutamine transaminase. A further preferred embodiment of the invention is directed to a method of screening for a composition which promote, increase, or agonize the activity of cystathionine beta synthase, L-amino acid oxidase, or glutamine transaminase.

Thus, in one aspect is provided a method of identifying a candidate molecule for the treatment of disease or for increasing the levels of or decreasing the degradation of amino acids, said method comprising: a) contacting a DAO, DDO, flavokinase, FAD pyrophosphorylase, L-amino acid oxidase, or glutamine transaminase polypeptide or a biologically active fragment thereof with a test compound; and b) determining whether said compound selectively binds to said polypeptide; wherein a determination that said compound selectively binds to said polypeptide indicates that said compound is a candidate molecule for the treatment of disease or for increasing the levels of or decreasing the degradation of amino acids.

Also provided is a method of identifying a candidate molecule for the treatment of disease or for increasing the levels of or decreasing the degradation of amino acids, said method comprising: a) contacting a DAO, DDO, flavokinase, FAD pyrophosphorylase, L-amino acid oxidase, or glutamine transaminase polypeptide or a biologically active fragment thereof polypeptide with a test compound; and b) determining whether said compound selectively inhibits the activity of said polypeptide; wherein a determination that said compound selectively inhibits the activity of said polypeptide indicates that said compound is a candidate molecule for the treatment of disease or for increasing the levels of or decreasing the degradation of amino acids.

In one aspect the invention discloses a method of identifying or assessing a candidate molecule for the treatment of a CNS disorder, said method comprising: (a) providing a test DAO-inhibitor or DDO-inhibitor compound; and (b) administering said compound to an animal model of schizophrenia, depression or bipolar disorder, wherein a determination that said compound ameliorates a representative characteristic of a CNS disorder in said animal model indicates that said compound is a candidate molecule for the treatment of a CNS disorder. Also encompassed is a method of identifying or assessing a candidate molecule for the treatment of a CNS disorder, said method comprising: contacting a DAO or DDO polypeptide or a biologically active fragment thereof with a test compound; (a) determining whether said compound (i) binds to said polypeptide, or (ii) inhibits the activity of said polypeptide; and (b) if said compound binds to said polypeptide or inhibits said polypeptide, administering said compound to an animal model of schizophrenia, depression or bipolar disorder, wherein a determination that said compound ameliorates a representative characteristic of CNS disorder in said animal model indicates that said compound is a candidate molecule for the treatment of a CNS disorder. Preferably said CNS disorder is psychotic disorder. Most preferably said CNS disorder is depression, bipolar disorder, or schizophrenia.

In further preferred embodiment, said animal model is a rat conditioned avoidance model, said representative characteristic is an avoidance response of the rat to mild shock, and said compound is a candidate molecule for the treatment of a CNS disorder if it is able to reduce the percentage of said avoidance responses by at least 50% without producing greater than 50% response failures.

In other further preferred embodiment, said animal model is a gerbil model of foot-tapping induced by an anxiogenic agent, said representative characteristic is anxiogenic agent-induced foot-tapping, and said compound is a candidate for the treatment of a CNS disorder if it is able to reduce the duration and/or intensity of said foot-tapping.

In other further preferred embodiment, said animal model is a gerbil model of foot-tapping evoked by aversive stimulation, said representative characteristic is aversive stimulation-evoked foot-tapping, and said compound is a candidate for the treatment of a CNS disorder if it is able to inhibit said foot-tapping.

In other further preferred embodiment, said animal model is a ferret model of emesis, said representative characteristic is cisplatin-induced retches and vomits, and said compound is a candidate for the treatment of a CNS disorder if it is able to reduce the number of said cisplatin-induced retches and vomits.

In other further preferred embodiment, said animal model is a guinea pig model of separation-induced vocalisation, said representative characteristic is separation-induced vocalisation, and said compound is a candidate for the treatment of a CNS disorder it is able to attenuate said separation-induced vocalisations.

In other further preferred embodiment, said animal model is a rodent model of behavioral activity assessment employing Omnitech Digiscan activity monitors, said representative characteristic is an aspect of locomotor activity, and said compound is a candidate for the treatment of a CNS disorder if it is able to reduce said aspect of locomotor activity. Preferably said rodent is rat or mouse. Preferably said compound is a candidate for the treatment of a CNS disorder if it is able to reduce said aspect of locomotor activity by at least 50%.

In other further preferred embodiment, said animal model is a rat model of amphetamine-stimulated locomotion, said representative characteristic is amphetamine-stimulated locomotion, and said compound is a candidate for the treatment of a CNS disorder if it is able to reverse said amphetamine-stimulated locomotion. Preferably said compound is a candidate for the treatment of a CNS disorder if it is able to reverse said amphetamine-stimulated locomotion by at least 50%.

In other further preferred embodiment, said animal model is a rat model of prepulse inhibition (PPI) of acoustic startle, said representative characteristic is diminished PPI, and said compound is a candidate for the treatment of a CNS disorder if it is able to increase said PPI.

In other further preferred embodiment, said animal model is a mouse model of apomorphine-induced climbing behavior, said representative characteristic is apomorphine-induced climbing behavior, and said compound is a candidate for the treatment of a CNS disorder if it is able to reduce said apomorphine-induced climbing behavior. Preferably, said compound is a candidate for the treatment of a CNS disorder if it is able to reduce said apomorphine-induced climbing behvavior by at least 50%.

In other further preferred embodiment, said animal model is a mouse model of 1-(2,5-dimethoxy-4-iodophenyl)-2-aminopropane (DOI)-induced head twitches and scratches, said representative characteristic is head twitches and scratches, and said compound is a candidate for the treatment of a CNS disorder if it is able to inhibit said DOI-induced head twitches and scratches. Preferably said compound is a candidate for the treatment of a CNS disorder if it is able to inhibit said DOI-induced head twitches and scratches by at least 50%.

Another mouse model is locomotor activity, stationary rod (Zic1−/+), acoustic startle response, and prepulse inhibition tests (Zic2kd/+) Ogura H, Aruga J, Mikoshiba K. Behav Genet. 2001 May;31(3):317-24 Another mouse model is the DBA/2 mouse model model wherein the representative characteristics are improvements in deficient sensory inhibition (Simosky J K, Stevens K E, Kem W R, Freedman R. (Biol Psychiatry Oct. 1, 2001;50(7):493-500). Another mouse model is the prepulse inhibition of startle in DBA/2J strain mice wherein the representative characteristics are improvements in prepulse inhibition of startle without disturbing the basal startle response (Olivier B, Leahy C, Mullen T, Paylor R, Groppi V E, Samyai Z, Brunner D. Psychopharmacology (Berl) 2001 July;156(2-3):284-90). Another model is the cannabinoid receptor knockout mice animal model wherein the representative characterists are improvements in the symptoms caused by the knockout (Fritzsche M. Psychopharmacology (Berl) 2001 May;155(3):299-309). Another model is the adenosine A(2A) receptor knockout mouse model for anxiety wherein the representative characterists are reductions in anxiety, aggressiveness in males and response to caffeine (Int J Neuropsychopharmcol Dec. 1, 1998;1(2):187-190). Another model is mouse D(1A) knockout model wherein the representative characterists are improvements in the brain metabolic response to ketamine. The test measures increases in 2-DG uptake in limbic cortical regions, hippocampal formation, nucleus accumbens, basolateral amygdala, and caudal parts of the substantia nigra pars reticulata(Miyamoto S, Mailnan R B, Lieberman J A, Duncan G E. Brain Res Mar. 16, 2001;894(2):167-80).the heterozygote reeler mouse model wherein the representative characterists are improvements in the dendritic spine and GABAergic defects described in schizophrenia (Costa E, Davis J, Pesold C, Tueting P, Guidotti A.

Curr Opin Pharmacol Feb. 2, 2002;2(1):56-62). Another mouse model are mice deleted for the DiGeorge/velocardiofacial syndrome region model wherein the representative characteristics are improvements in abnormal sensorimotor gating and learning and memory impairments (Paylor R, McIlwain K L, McAninch R, Nellis A, Yuva-Paylor L A, Baldini A, Lindsay E A, Hum Mol Genet Nov. 1, 2001;10(23):2645-50). Another mouse model is the behavioral abnormalities of Zic1 and Zic2 mutant mice model wherein the representative characteristics are improvements in impaired sensory inhibition characterized by diminished response of the hippocampal evoked potential to the second of closely paired auditory stimuli (500-m/sec interstimulus interval). Test experiments include the hanging, spontaneous

Also described is a method of identifying a candidate molecule for the treatment of disease or for increasing the levels of or decreasing the degradation of amino acids, said method comprising: a) providing a cell comprising a DAO, DDO, flavokinase, FAD pyrophosphorylase, L-amino acid oxidase, or glutamine transaminase polypeptide or a biologically active fragment thereof; b) contacting said cell with a test compound; and c) determining whether said compound selectively inhibits DAO, DDO, flavokinase, FAD pyrophosphorylase, L-amino acid oxidase, or glutamine transaminase activity; wherein a determination that said compound selectively inhibits the activity of said polypeptide indicates that said compound is a candidate molecule for the treatment of disease or for increasing the levels of or decreasing the degradation of amino acids.

Further provided is a method of identifying a candidate molecule for the treatment of disease or for increasing the levels of or decreasing the degradation of amino acids, said method comprising: a) contacting a cystathionine beta synthase, L-amino acid oxidase, or glutamine transaminase polypeptide or a biologically active fragment thereof polypeptide with a test compound; and b) determining whether said compound selectively increases the activity of said polypeptide; wherein a determination that said compound selectively increases the activity of said polypeptide indicates that said compound is a candidate molecule for the treatment of disease or for increasing the levels of or decreasing the degradation of amino acids.

Another embodiment is method of identifying a candidate molecule for the treatment of disease or for increasing the levels of or decreasing the degradation of amino acids, said method comprising : a) providing a cell comprising a cystathionine beta synthase, L-amino acid oxidase, or glutamine transaminase polypeptide or a biologically active fragment thereof; b) contacting said cell with a test compound; and c) determining whether said compound selectively increases cystathionine beta synthase, L-amino acid oxidase, or glutamine transaminase activity; wherein a determination that said compound selectively increases the activity of said polypeptide indicates that said compound is a candidate molecule for the treatment of disease or for increasing the levels of or decreasing the degradation of amino acids.

further preferred embodiment of the invention is directed to a method of antagonizing, reducing or inhibiting DAO activity in vitro. Further preferred is a method of antagonizing, reducing or inhibiting DAO activity in vivo. Further preferred is a method of antagonizing, reducing or inhibiting DAO activity in vitro or in vivo comprising the step of contacting DAO with a composition which reduces, inhibits or antagonizes the activity of DAO. A preferred activity of DAO to be inhibited is the oxidation of a substrate, preferably the substrate is a D-Amino Acid, preferably the D-amino acid is D-Ser, D-Asp, or N-methyl-D-Asp.

A further preferred embodiment of the invention is directed to a method of antagonizing, reducing or inhibiting DDO activity in vitro. Further preferred is a method of antagonizing, reducing or inhibiting DDO activity in vivo. Further preferred is a method of antagonizing, reducing or inhibiting DDO activity in vitro or in vivo comprising the step of contacting DDO with a composition which reduces, inhibits or antagonizes the activity of DDO. A preferred activity of DDO to be inhibited is the oxidation of a substrate, preferably the substrate is a D-Amino Acid, preferably the D-amino acid is D-Asp, D-Glu, D-Asn, D-Gln, D-Asp-dimethyl-ester or N-methyl-D-Asp.

Another embodiment of the invention is directed to compositions which increase the levels of at least one D-amino acid in vitro. Further preferred are compositions which increase the levels of at least one D-amino acid in vivo, preferably in tissues of mammals, further preferably in tissues of mice, rats, dogs, cows, pigs, apes, monkeys or humans. Still further preferred are compositions which increase levels of at least one D-amino acid in tissues of the central nervous system, preferably the brain or spinal cord. Still further preferred are compositions which increase levels of at least one D-amino acid in tissues of the brain, preferably the hippocampus, amygdala, substantia nigra, cerebellum, corpus callosum, caudate nucleus, cerebral cortex, thalamus, or pituitary gland. Other preferred tissues in which compositions of the invention increase levels of at least one D-amino acid include, but are not limited to the kidney, liver, adipose, muscle, and testis.

A preferred embodiment of the invention is directed to a use of a polypeptide of SEQ ID NO: 15, or a fragment thereof, in a method to increase DAO activity. Further preferred is a use of a polypeptide of SEQ ID NO: 15, or a fragment thereof, in a method to increase DDO activity. Further preferred is a use of a polypeptide of SEQ ID NO: 15, or a fragment thereof, in a method to decrease serine racemase activity.

A preferred embodiment of the invention is directed to a use of a polypeptide of SEQ ID NO: 15, or a fragment thereof, in a method of increasing production of compounds or compositions which are the product of a reaction involving DAO as a catalyst.

A preferred embodiment of the invention is directed to a method of screening for compositions or compounds that bind to g34872 polypeptides (SEQ ID NO: 15) or g34872 polynucleotides (SEQ ID NO: 14), or fragments thereof. Further preferred is a method of contacting g34872 polypeptides, or fragments thereof, with DAO thereby increasing DAO activity above a basal level. Further preferred is a method of reducing, inhibiting, antagonizing or blocking the interaction of DAO and g34872. Further preferred is a method of treating a CNS disorder by blocking the interaction of g34872 and DAO. Further preferred is a method of treating a CNS disorder with a compound or composition which reduces,blocks, inhibits or antagonizes the interaction between g34872 and DAO.

The preferred DAO polypeptides of the invention include polypeptides of SEQ ID NO: 7-10 and 19, and fragments thereof as well as polynucleotides that encode the same. The preferred DDO polypeptides of the invention include polypeptides of SEQ ID NO: 22 and 23, and fragments thereof, as well as polynucleotides that encode the same. Preferred DAO polynucleotides of the invention include SEQ ID NO: 2-6, and 18, and fragments thereof, as well as polypeptides encoded by the same. Preferred DDO polynucleotides of the invention include SEQ ID NO: 20 and 21, and fragments thereof, as well as polypeptides encoded by the same.

Preferred biallelic markers of DAO are described in SEQ ID NO: 1, as well as represented by 47-mers of marker 24-1443-126 (SEQ ID NO: 24), marker 24-1457-52 (SEQ ID NO: 26), and marker 24-1461-256 (SEQ ID NO: 29).

Another embodiment of the invention is directed at compositions which differentially bind to polypeptides of SEQ ID NO: 7. Another embodiment of the invention is directed at compositions which differentially bind to polypeptides of SEQ ID NO: 8. Another embodiment of the invention is directed at compositions which differentially bind to polypeptides of SEQ ID NO: 9. Another embodiment of the invention is directed at compositions which differentially bind to polypeptides of SEQ ID NO: 10. Further preferred are compositions which bind to polypeptides of SEQ ID NO: 10 but not to polypeptides of SEQ ID NO: 7, 8, or 9. Further preferred are compositions which bind to polypeptides of SEQ ID NO: 9 but not to polypeptides of SEQ ID NO: 7, 8, or 10. Further preferred are compositions which bind to polypeptides of SEQ ID NO: 8 but not to polypeptides of SEQ ID NO: 7, 9, or 10. Further preferred are compositions which bind to polypeptides of SEQ ID NO: 7 but not to polypeptides of SEQ ID NO: 8, 9, or 10. Further preferred are compositions which bind to polypeptides of SEQ ID NO: 8, 9, or 10 but not to polypeptides of SEQ ID NO: 7.

Another embodiment of the invention is directed to a composition which differentially binds to a monomeric polypeptide comprising SEQ ID NO: 7, 8, 9, 10, or 15, or a polypeptide fragment thereof. Further preferred is a composition which binds to a monomeric polypeptide of SEQ ID NO: 7, or a fragment thereof, but not to a homo- or hetero-multimeric form comprising at least a monomer of a polypeptide of SEQ ID NO: 7, or a fragment thereof. Further preferred is a composition which binds to a monomeric polypeptide of SEQ ID NO: 8, or a fragment thereof, but not to a homo- or hetero-multimeric form comprising at least a monomer of a polypeptide of SEQ ID NO: 8, or a fragment thereof. Further preferred is a composition which binds to a monomeric polypeptide of SEQ ID NO: 9, or a fragment thereof, but not to a homo- or hetero-multimeric form comprising at least a monomer of a polypeptide of SEQ ID NO: 9, or a fragment thereof. Further preferred is a composition which binds to a monomeric polypeptide of SEQ ID NO: 10, or a fragment thereof, but not to a homo- or hetero-multimeric form comprising at least a monomer of a polypeptide of SEQ ID NO: 10, or a fragment thereof. Further preferred is a composition which binds to a monomeric polypeptide of SEQ ID NO: 15, or a fragment thereof, but not to a homo- or hetero-multimeric form comprising at least a monomer of a polypeptide of SEQ ID NO: 15, or a fragment thereof.

Another embodiment of the invention is directed to a composition which binds to a multimeric polypeptide comprising at least one polypeptide of SEQ ID NO: 7, 8, 9, 10, or 15, or a fragment thereof. Further preferred is a composition which binds to a homo- or hetero-multimeric form comprising at least one monomer of a polypeptide of SEQ ID NO: 7, or a fragment thereof, but does not bind to a monomeric polypeptide of SEQ ID NO: 7, or a fragment thereof. Another embodiment of the invention is directed to a composition which binds to a homo- or hetero-multimeric form comprising at least one monomer of a polypeptide of SEQ ID NO: 8, or a fragment thereof, but does not bind to a monomeric polypeptide of SEQ ID NO: 8, or a fragment thereof. Another embodiment of the invention is directed to a composition which binds to a homo- or hetero-multimeric form comprising at least one monomer of a polypeptide of SEQ ID NO: 9, or a fragment thereof, but does not bind to a monomeric polypeptide of SEQ ID NO: 9, or a fragment thereof. Another embodiment of the invention is directed to a composition which binds to a homo- or hetero-multimeric form comprising at least one monomer of a polypeptide of SEQ ID NO: 10, or a fragment thereof, but does not bind to a monomeric polypeptide of SEQ ID NO: 10, or a fragment thereof. Another embodiment of the invention is directed to a composition which binds to a homo- or hetero-multimeric form comprising at least one monomer of a polypeptide of SEQ ID NO: 15, or a fragment thereof, but does not bind to a monomeric polypeptide of SEQ ID NO: 15, or a fragment thereof.

Another embodiment of the invention is directed at compositions which differentially bind to polynucleotides of SEQ ID NO: 2. Another embodiment of the invention is directed at compositions which differentially bind to polynucleotides of SEQ ID NO: 3. Another embodiment of the invention is directed at compositions which differentially bind to polynucleotides of SEQ ID NO: 4. Another embodiment of the invention is directed at compositions which differentially bind to polynucleotides of SEQ ID NO: 5. Another embodiment of the invention is directed at compositions which differentially bind to polynucleotides of SEQ ID NO: 6. Further preferred are compositions which bind to polynucleotides of SEQ ID NO: 6 but not to polynucleotides of SEQ ID NO: 2, 3, 4, or 5. Further preferred are compositions which bind to polynucleotides of SEQ ID NO: 5 but not to polynucleotides of SEQ ID NO: 2, 3, 4, or 6. Further preferred are compositions which bind to polynucleotides of SEQ ID NO: 4 but not to polynucleotides of SEQ ID NO: 2, 3, 5, or 6. Further preferred are compositions which bind to polynucleotides of SEQ ID NO: 3 but not to polynucleotides of SEQ ID NO: 2, 4, 5, or 6. Further preferred are compositions which bind to polynucleotides of SEQ ID NO: 2 but not to polynucleotides of SEQ ID NO: 3, 4, 5, or 6. Further preferred are compositions which bind to polynucleotides of SEQ ID NO: 3, 4, 5, or 6 but not to polynucleotides of SEQ ID NO: 2.

A further preferred embodiment of the invention is directed to a genomic sequence comprising polynucleotides of SEQ ID NO: 1. Further preferred are methods to genotype regions of the polynucleotides of SEQ ID NO: 1.

An embodiment of the invention is directed to a purified or isolated nucleic acid comprising the sequence of SEQ ID NO: 1 or complement thereof. Further preferred is a purified or isolated nucleic acid comprising at least 10 consecutive nucleotides of the sequence of SEQ ID NO: 1 or complement thereof. Still further preferred is a nucleic acid comprises at least 15 consecutive nucleotides of the sequence of SEQ ID NO: 1 or complement thereof.

An another embodiment of the invention is directed to a purified or isolated nucleic acid comprising at least 10 consecutive nucleotides of the sequence of SEQ ID NO: 1, or complement thereof, of one or more exons. Further preferred is a purified or isolated nucleic acid of SEQ ID NO: 1, or complement thereof, comprising the sequence of at least 10 consecutive nucleotides from nucleotides 40389 to 40670 of SEQ ID NO: 1, or complement thereof. Also preferred is a purified or isolated nucleic acid of SEQ ID NO: 1, or complement thereof, comprising the sequence of at least 10 consecutive nucleotides from nucleotides 42666 to 42778 of SEQ ID NO: 1, or complement thereof. Also preferred is a purified or isolated nucleic acid of SEQ ID NO: 1, or complement thereof, comprising the sequence of at least 10 consecutive nucleotides from nucleotides 43416 to 43519 of SEQ ID NO: 1, or complement thereof. Also preferred is a purified or isolated nucleic acid of SEQ ID NO: 1, or complement thereof, comprising the sequence of at least 10 consecutive nucleotides from nucleotides 61159 to 61402 of SEQ ID NO: 1, or complement thereof. Also preferred is a purified or isolated nucleic acid of SEQ ID NO: 1, or complement thereof, comprising the sequence of at least 10 consecutive nucleotides from nucleotides 64050 to 64711 of SEQ ID NO: 1, or complement thereof. Also preferred is a purified or isolated nucleic acid of SEQ ID NO: 1, or complement thereof, comprising the sequence of at least 10 consecutive nucleotides from nucleotides 68126 to 68261 of SEQ ID NO: 1, or complement thereof. Also preferred is a purified or isolated nucleic acid of SEQ ID NO: 1, or complement thereof, comprising the sequence of at least 10 consecutive nucleotides from nucleotides 84906 to 85541 of SEQ ID NO: 1, or complement thereof.

A further preferred embodiment of the invention is directed to a purified or isolated nucleic acid comprising the sequence of SEQ ID NO: 2 or complement thereof. A still further preferred embodiment of the invention directed to a purified or isolated nucleic acid comprising the sequence of SEQ ID NO: 3 or complement thereof. Another further preferred embodiment of the invention directed to a purified or isolated nucleic acid comprising the sequence of SEQ ID NO: 4 or complement thereof. Another further preferred embodiment of the invention directed to a purified or isolated nucleic acid comprising the sequence of SEQ ID NO: 5 or complement thereof. Another further preferred embodiment of the invention directed to a purified or isolated nucleic acid comprising the sequence of SEQ ID NO: 6 or complement thereof. Another further preferred embodiment of the invention directed to a purified or isolated nucleic acid comprising the sequence of SEQ ID NO: 14 or complement thereof. Another further preferred embodiment of the invention directed to a purified or isolated nucleic acid comprising the sequence of SEQ ID NO: 16 or complement thereof. Another further preferred embodiment of the invention directed to a purified or isolated nucleic acid comprising the sequence of any one of the sequences of SEQ ID NO: 18, 20, or 21, or complement thereof.

Another embodiment of the invention is directed to a purified or isolated nucleic acid comprising at least 10 consecutive nucleotides of at least one of the sequences of SEQ ID NO: 2-6, or complement thereof. Further preferred is a purified or isolated nucleic acid comprising at least 15 consecutive nucleotides of at least one of the sequences of SEQ ID NO: 2-6, or complement thereof.

Another embodiment of the invention is directed to a purified or isolated nucleic acid comprising at least 10 consecutive nucleotides of the sequence of SEQ ID NO: 14, or complement thereof. Further preferred is a purified or isolated nucleic acid comprising at least 15 consecutive nucleotides of the sequence of SEQ ID NO: 14, or complement thereof.

Another embodiment of the invention is directed to a purified or isolated nucleic acid encoding the polypeptide of SEQ ID NO: 7. Further preferred is a purified or isolated nucleic acid encoding at least 10 consecutive amino acids of the polypeptide of SEQ ID NO: 7. Still further preferred is a purified or isolated nucleic acid, wherein said nucleic acid encodes at least 15 consecutive amino acids of the polypeptide of SEQ ID NO: 7.

Another embodiment of the invention is directed to a purified or isolated nucleic acid encoding the polypeptide of SEQ ID NO: 8. Further preferred is a purified or isolated nucleic acid encoding at least 10 consecutive amino acids of the polypeptide of SEQ ID NO: 8. Still further preferred is a purified or isolated nucleic acid, wherein said nucleic acid encodes at least 15 consecutive amino acids of the polypeptide of SEQ ID NO: 8.

Another embodiment of the invention is directed to a purified or isolated nucleic acid encoding the polypeptide of SEQ ID NO: 9. Further preferred is a purified or isolated nucleic acid encoding at least 10 consecutive amino acids of the polypeptide of SEQ ID NO: 9. Still further preferred is a purified or isolated nucleic acid, wherein said nucleic acid encodes at least 15 consecutive amino acids of the polypeptide of SEQ ID NO: 9.

Another embodiment of the invention is directed to a purified or isolated nucleic acid encoding the polypeptide of SEQ ID NO: 10. Further preferred is a purified or isolated nucleic acid encoding at least 10 consecutive amino acids of the polypeptide of SEQ ID NO: 10. Still further preferred is a purified or isolated nucleic acid, wherein said nucleic acid encodes at least 15 consecutive amino acids of the polypeptide of SEQ ID NO: 10.

Another embodiment of the invention is directed to a purified or isolated nucleic acid encoding the polypeptide of SEQ ID NO: 15. Further preferred is a purified or isolated nucleic acid encoding at least 10 consecutive amino acids of the polypeptide of SEQ ID NO: 15. Still further preferred is a purified or isolated nucleic acid, wherein said nucleic acid encodes at least 15 consecutive amino acids of the polypeptide of SEQ ID NO: 15.

Another embodiment of the invention is directed to a purified or isolated nucleic acid encoding the polypeptide of SEQ ID NO: 17. Further preferred is a purified or isolated nucleic acid encoding at least 10 consecutive amino acids of the polypeptide of SEQ ID NO: 17. Still further preferred is a purified or isolated nucleic acid, wherein said nucleic acid encodes at least 15 consecutive amino acids of the polypeptide of SEQ ID NO: 17.

Another embodiment of the invention is directed to a purified or isolated nucleic acid encoding the polypeptide of SEQ ID NO: 19. Further preferred is a purified or isolated nucleic acid encoding at least 10 consecutive amino acids of the polypeptide of SEQ ID NO: 19. Still further preferred is a purified or isolated nucleic acid, wherein said nucleic acid encodes at least 15 consecutive amino acids of the polypeptide of SEQ ID NO: 19.

Another embodiment of the invention is directed to a purified or isolated nucleic acid encoding the polypeptide of SEQ ID NO: 22. Further preferred is a purified or isolated nucleic acid encoding at least 10 consecutive amino acids of the polypeptide of SEQ ID NO: 22. Still further preferred is a purified or isolated nucleic acid, wherein said nucleic acid encodes at least 15 consecutive amino acids of the polypeptide of SEQ ID NO: 22.

Another embodiment of the invention is directed to a purified or isolated nucleic acid encoding the polypeptide of SEQ ID NO: 23. Further preferred is a purified or isolated nucleic acid encoding at least 10 consecutive amino acids of the polypeptide of SEQ ID NO: 23. Still further preferred is a purified or isolated nucleic acid, wherein said nucleic acid encodes at least 15 consecutive amino acids of the polypeptide of SEQ ID NO: 23.

A further preferred embodiment of the invention is directed at the biallelic markers.

### BRIEF DESCRIPTION OF THE DRAWINGS

FIG. 1 demonstrates the activity of yeast expressed recombinant g34782 and DAO polypeptides.

FIG. 2 demonstates the activity of bacterial expressed recombinant g34872 and DAO polypeptides.

FIG. 3 demonstrates the in vitro activation of purified DAO by g34872 using D-serine as a substrate.

FIG. 4 demonstrates the dose dependent affect of g34872 on DAO activity.

FIG. 5 demonstrates the kinetics of the interaction between g34872 and DAO.

FIG. 6 is a table demonstrating the results of a DAO biallelic marker association analysis between French Canadian schizophrenia cases and controls.

### BRIEF DESCRIPTION OF THE SEQUENCES PROVIDED IN THE SEQUENCE LISTING

SEQ ID NO: 1 genomic sequence of DAO;

SEQ ID NO: 2 DAO cDNA;

SEQ ID NO: 3 novel cDNA with Exons U 2 3 4 5 6 7 8 9 10 11 Long;

SEQ ID NO: 4 novel cDNA with Exons B C Ulong V 2 3 4 5 6 7 9 10 11 Long;

SEQ ID NO: 5 novel cDNA with Exons U 24 5 6 7 8 9 10 11 Long;

SEQ ID NO: 6 novel cDNA with Exons B 2 3 7 8 9 10 11;

SEQ ID NO: 7 polypeptide of DAO from cDNA of SEQ ID NO: 2 and 3;

SEQ ID NO: 8 polypeptide of DAO from cDNA of SEQ ID NO: 4;

SEQ ID NO: 9 polypeptide of DAO from cDNA of SEQ ID NO: 5;

SEQ ID NO: 10 polypeptide of DAO from cDNA of SEQ ID NO: 6;

SEQ ID NO: 11-12 polynucleotides comprising g34872 biallelic markers 99/16105-152 and 99/5919-215;

SEQ ID NO: 13 polynucleotides of g34872, including polymorphisms;

SEQ ID NO: 14 polypeptides of g34872, wherein the amino acid at position 10 is tyrosine or serine, the amino acid at position 30 is lysine or arginine, the amino acid at position 50 is glutamate or a premature stop, the amino acid at position 60 is arginine or glycine, and the amino acid at position 115 is aspartate or alanine;

SEQ ID NO: 15 g34872 polynucleotide encoding polypeptide of SEQ ID NO: 16 used in 2-Hybrid experiments;

SEQ ID NO: 17 polynucleotide of DAO encoding polypeptide of SEQ ID NO: 18;

SEQ ID NOs: 19 and 20 polynucleotides of DDO encoding polypeptides of SEQ ID NOs: 21 and 22, respectively; and

SEQ ID NOs: 23-26 polynucleotides comprising DAO biallelic markers 24-1443/126, 24-1457/52, 27-93/181, and 24-1461/256, respectively, noting polymorphic base at position 24.

The g34872 genomic sequence and biallelic markers are described in SEQ ID NO: 1 of U.S. patent application Ser. No:09/539,333 and Internation Patent Application No:PCT/IB00/00435, which disclosures are hereby incorporated by reference in their entireties.

In accordance with the regulations relating to Sequence Listings, the following codes have been used in the Sequence Listing to indicate the locations of biallelic markers within the sequences and to identify each of the alleles present at the polymorphic base. The code “r” in the sequences indicates that one allele of the polymorphic base is a guanine, while the other allele is an adenine. The code “y” in the sequences indicates that one allele of the polymorphic base is a thymine, while the other allele is a cytosine. The code “m” in the sequences indicates that one allele of the polymorphic base is an adenine, while the other allele is an cytosine. The code “k” in the sequences indicates that one allele of the polymorphic base is a guanine, while the other allele is a thymine. The code “s” in the sequences indicates that one allele of the polymorphic base is a guanine, while the other allele is a cytosine. The code “w” in the sequences indicates that one allele of the polymorphic base is an adenine, while the other allele is an thymine.

### DETAILED DESCRIPTION OF THE INVENTION

The present invention relates to methods for providing prevention of a CNS disorder to a subject susceptible to such a disorder, and for providing treatment to a subject suffering from a CNS disorder. In particular, the method comprises administering to a patient an amount of a DAO or DDO antagonist compound effective for providing some degree of prevention or amelioration of the progression of the CNS disorder (i.e., provide protective effects), amelioration of the symptoms of the CNS disorder, and amelioration of the reoccurrence of the CNS disorder.

CNS disorders which can be treated in accordance with the present invention include presenile dementia (early onset Alzheimer's disease), senile dementia (dementia of the Alzheimer's type), Parkinsonism including Parkinson's disease, Huntington's chorea, tardive dyskinesia, hyperkinesia, mania, attention deficit hyperactivity disorder (ADHD), attention deficit disorder (ADD), anxiety disorders, dyslexia, phycotic disorders, schizophrenia, bipolar disorder, major depressive episodes, manic episodes, hypomanic episodes, depression, autistic diorders, substance abuse, excessive aggression, tic disorders and Tourette's syndrome. Preferred disorders of the present invention include schizophrenia and bipolar disorder. Further preferred embodiments of schizophrenia and schizophreniform disorders include: schizophrenia (catatonic), schizophrenia (disorganized), schizophrenia (paranoid), schizophrenia (undifferential), schizophrenia (residual), schizophreniform disorder, brief reactive psychosis, schizoaffective disorder, induced psychotic disorder, schizotypal personality disorder, schizoid personality disorder, paranoid personality disorder and delusional (paranoid) disorder.

The identification of genes involved in a particular trait such as a specific central nervous system disorder, like schizophrenia, can be carried out through two main strategies currently used for genetic mapping: linkage analysis and association studies. Linkage analysis requires the study of families with multiple affected individuals and is now useful in the detection of mono- or oligogenic inherited traits. Conversely, association studies examine the frequency of marker alleles in unrelated trait (T+) individuals compared with trait negative (T−) controls, and are generally employed in the detection of polygenic inheritance.

In the present application, additional biallelic markers located in the DAO gene associated with schizophrenia are disclosed. The identification of these biallelic markers in association with schizophrenia has allowed for the further definition of the chromosomal region suspected of containing a genetic determinant involved in a predisposition to develop schizophrenia and has resulted in the identification of novel gene sequences disclosed herein which are associated with a predisposition to develop schizophrenia. Furthermore, biallelic markers in the g34872 gene, previously described, as well as in the DAO gene presently described can be used alone or in combination to determine individuals at risk for developing a CNS disorder. Moreover, biallelic markers in the g34872 gene, previously described, as well as in the DAO gene presently described can be used alone or in combination to determine individuals who will benefit from the treatment described by the present invention. Additionally, the sequence information provides a resource for the further identification of new genes and markers in those regions. Additionally, the sequences comprising the the schizophrenia-associated genes are useful, for example, for the isolation of other genes in putative gene families, the identification of homologs from other species, treatment of disease and as probes and primers for diagnostic or screening assays as described herein. Furthermore, the identified polymorphisms are used in the design of assays for the reliable detection of genetic susceptibility to schizophrenia and bipolar disorder. They are also used in the design of drug screening protocols to provide an accurate and efficient evaluation of the therapeutic and side-effect potential of new or already existing medicament or treatment regime.

Definitions

The term “treat” or “treating” means to ameliorate, alleviate symptoms, eliminate the causation of the symptoms either on a temporary or permanent basis, or to prevent or slow the appearance of symptoms of the named disorder or condition.

The dose of the compound is that amount effective to prevent occurrence of the symptoms of the disorder or to treat some symptoms of the disorder from which the patient suffers. By “effective amount”, “therapeutically effective amount” “therapeutic amount” or “effective dose” is meant that amount sufficient to elicit the desired pharmacological or therapeutic effects, thus resulting in effective prevention or treatment of the disorder. Prevention of the disorder is manifested by delaying the onset of the symptoms of the disorder to a medically significant extent. Treatment of the disorder is manifested by a decrease in the symptoms associated with the disorder or an amelioration of the reoccurrence of the symptoms of the disorder. A therapeutically effective amount of a compound of the present invention can be easily determined by one skilled in the art by administering a quantity of a compound to an individual and observing the result. In addition, those skilled in the art are familiar with identifying individuals having a CNS disorder readily able to identify individuals who suffer from the CNS disorder.

The terms “antagonist” and “inhibitor” are considered to be synomous and can be used interchangeably throughout the disclosure. The “antagonist” compounds of the invention may be administered together with a typical or atypical anti-CNS disorder drug, such as an antipsychotic drug. Typical antipsychotics include: haloperidol, fluphenazine, perphenazine, chlorpromazine, molindone, pimozide, trifluoperazine and thioridazine, thiadiazole, oxadiazole and others. Atypical antipsychotics include: clozapine, risperidone, olanzapine, sertindole, M100907, ziprasidone, seroquel, zotepine, amisulpride, iloperidone, phenelzine and others. Typical antidepressant and anti-anxiety agents include: heterocyclic antidepressants (TCAs, tetracyclics, and the like), SSRIs, mixed serotonin and norepinephrine reuptake inhibitors, dopamine reuptake inhibitors and MAOIs. The antagonists may also be used to treat individuals for whom the above drugs are contraindicated. The present invention also provides a method for the treatment or prevention of schizophrenia, bipolar disorder, or other CNS disorders without concomitant therapy with other antipsychotic, antidepressant, anti-anxiety, or other drugs, in a patient who is non-responsive. The antipsychotic, antidepressant, anti-anxiety, or other drugs may be administered at a subtherapeutic doses, i.e., at a lower dose than the dosage that is typically used for treatments with the above drugs alone. Drugs used for the treatment of schizophrenia, bipolar disorder, depression, and other CNS disorders, that are either recognized as a DAO or DDO inhibitor or that inherently act as an inhibitor of DAO or DDO are specifically excluded from the definition of DAO or DDO “antagonist” and may be specifically excluded from the present invention. Further, any molecule, compound or drug disclosed herein may be specifically excluded from the invention.

“Alkyl” means a branched or unranked saturated hydrocarbon chain containing 1 to 8 carbon atoms, such as methyl, ethyl, propyl, iso-propyl, butyl, iso-butyl, tert-butyl, n-pentyl, n-hexyl, and the like, unless otherwise indicated.

“Alkoxy” means the group —OR wherein R is alkyl as herein defined. Preferably, R is a branched or unbranched saturated hydrocarbon chain containing 1 to 3 carbon atoms.

“Halo” means fluoro, chloro, bromo, or iodo, unless otherwise indicated.

“Phenyl” includes all possible isomeric phenyl radicals, optionally monosubstituted or multi-substituted with substituents selected from the group consisting of alkyl, alkoxy, hydroxy, halo, and haloalkyl.

Preferred heteroaryl rings include pyrrole, furan, thiophene, pyridine, pyrimidine, pyridazine, pyrazine, triazole, tetrazole, pyrazole, imidazole, isothiazole, thiazole, isoxazole and oxazole. Preferred “heteroaryl fused to phenyl” rings indole, isoindole, benzofuran, benzothiophene, quinoline, isoquinoline, quinoxaline, quinazoline, benzotriazole, indazole, benzimidazole, benzothiazole, benzisoxazole, and benzoxazole. It is assumed that “heteroaryl fused to phenyl” rings are included when using the term heteroaryl rings. The term “saturated or partially unsaturated heterocycloalkyl ring” means a saturated or partially unsaturated (but not aromatic, or fully saturated) heterocycle having 5-7 ring atoms, and containing 1-3 heteroatoms selected from N, O, or S. Preferred saturated or partially unsaturated heterocycloalkyl rings include piperidine, piperazine, morpholine, tetrahydropyran, thiomorpholine, or pyrrolidine.

The term “pharmaceutically acceptable salt” refers to salts of the subject compounds which posses the desired pharmacological activity and which are neither biologically nor otherwise undesirable. The salts can be formed with inorganic acids such as acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, fumarate, glucoheptanoate, glycerophosphate, hemisulfate heptanoate, hexanoate, hydrochloride hydrobromide, hydroiodide, 2-hydroxyethanesulfonate, lactate, maleate, methanesulfonate, 2-naphthalenesulfonate, nicotinate, oxalate, thiocyanate, tosylate and undecanoate. Base salts include ammonium salts, alkali metal salts such as sodium and potassium salts, alkaline earth metal salts such as calcium and magnesium salts, salt with organic bases such as dicyclohexylamine salts, N-methyl-D-glucamine, and salts with amino acids such as arginine, lysine, and so forth. Also, the basic nitrogen-containing groups can be quarternized with such agents as lower alkyl halides, such as methyl, ethyl, propyl, and butyl chloride, bromides and iodides; dialkyl sulfates like dimethyl, diethyl, dibutyl and diamyl sulfates, long chain halides such as decyl, lauryl, myristyl and stearyl chlorides, bromides and iodides, aralkyl halides like benzyl and phenethyl bromides and others. Water or oil-soluble or dispersible products are thereby obtained. Furthermore, pharmaceutical and pharmaceutically acceptable compositions are described infra.

The compounds of this invention possess asymmetric centers and thus can be produced as mixtures of stereoisomers or as individual stereoisomers. The individual stereoisomers may be obtained by using an optically active starting material, by resolving a racemic or non-racemic mixture of an intermediate at some appropriate stage of the synthesis, or by resolution of the compound of formula (I). It is understood that the individual stereoisomers as well as mixtures (racemic and non-racemic) of stereoisomers are encompassed by the scope of the present invention. The compounds of this invention possess at least one asymmetric centers and thus can be produced as mixtures of stereoisomers or as individual R- and S-stereoisomers. The individual enantiomers may be obtained by resolving a racemic or non-racemic mixture of an intermediate at some appropriate stage of the synthesis. It is understood that the individual R- and S-stereoisomers as well as mixtures of stereoisomers are encompassed by this invention.

“Isomers” are different compounds that have the same molecular formula.

“Stereoisomers” are isomers that differ only in the way the atoms are arranged in space.

“Enantiomers” are a pair of stereoisomers that are non-superimposable mirror images of each other.

“Diastereoisomers” are stereoisomers which are not mirror images of each other. “Racemic mixture” means a mixture containing equal parts of individual enantiomers. “Non-racemic mixture” is a mixture containing unequal parts of individual enantiomers or stereoisomers.

“Substituted Alkyls” include carboxyalkyls such as acetyl, aminoalkyls, dialkylaminoalkyls, hydoxyalkyls and mercaptoalkyls, alkylsilyl.

The present invention relates to compounds of Formulae I-VI including , but not limited to the specific examples presented herein. Further, any of these compounds may take the form of a pharmaceutically acceptable salt.

It should be appreciated that the compounds of the invention described herein can be synthesized by an artisan skilled in the art of organic chemistry.

The term “psychotic condition” as used herein means pathologic psychological conditions which are psychoses or may be associated with psychotic features. Such conditions include, but are not limited to the psychotic disorders which have been characterized in the DSM-IV-R, Diagnostic and Statistical Manual of Mental Disorders, Revised 4th Ed. (1994), including schizophrenia and acute mania. The DSM-IV-R was prepared by the Task Force on Nomenclature and Statistics of the American Association, and provides clear descriptions of diagnostic categories. The skilled artisan will recognize that there are alternative nomenclatures, nosologies, and classification systems for pathologic psychological conditions and that these systems evolve with medical scientific progress.

The term “schizophrenia” encompasses, or alternatively may be specifically limited to, Schizophrenia, Schizophreniform Disorder, Schizoaffective Disorder, Delusional Disorder, Brief Psychotic Disorder, Psychotic Disorder Due to a General Medical Condition, Psychotic Disorder Not Otherwise Specified, or described elsewhere herein. The symptoms of these disorders are in large part as defined in the Diagnostic and Statistical Manual of Mental Disorder, fourth edition (DSMIV). The sections of the DSMIV that relate to these disorders are hereby incorporated by reference.

The term “bipolar disorder” as used herein refers to a condition characterized as a Bipolar Disorder, in the DSM-IV-R. Diagnostic and Statistical Manual of Mental Disorders, Revised, 3rd Ed. (1994) as catagory 296.xx. To further clarify, Applicants contemplate the treatment of both bipolar disorder I and bipolar disorder II as described in the DSM-IV-R. The term further includes cyclothymic disorder. Cyclothymic disorder refers to an alternation of depressive symptoms and hypomanic symptoms. The skilled artisan will recognize that there are alternative nomenclatures, nosologies, and classification systems for pathologic psychological conditions and that these systems evolve with medical scientific progress.

As used herein, the term “non-responsive” in relation to major depressive disorder means patients who have not had a reasonable clinical response (e.g. a 50% reduction in Hamilton Depression Scale (HAM-D) from a patient's baseline score after treatment with one or more clinical courses of conventional antidepressants).

A “major depressive episode” is defined as at least two weeks of depressed mood or loss of interest, which may be accompanied by other symptoms of depression. The symptoms must persist for most of the day (i.e. for at least two thirds of the patients' waking hours), nearly every day (i.e. for at least ten out of fourteen days) for at least two consecutive weeks. A “depressed mood” is often described by the patient as feeling sad, hopeless, helpless or worthless. The patient may also appear sad to an observer, for example, through facial expression, posture, voice and tearfulness. In children and adolescents, the mood may be irritable. A “loss of interest” is often described by the patient as feeling less interested in hobbies or not feeling any enjoyment in activities that were previously considered to be pleasurable.

A major depressive episode may be accompanied by other symptoms of depression including significant weight loss when not dieting or weight gain (e.g. a change of more than 5% body weight in one month), or decrease or increase in appetite; insomnia or hypersomnia; psychomotor agitation or retardation; fatigue or loss of energy; feelings of worthlessness or excessive or inappropriate guilt; diminished ability to think or concentrate; or indecisiveness; and recurrent thoughts of death, recurrent suicidal ideation with or without a specific plan, or a suicide attempt.

A “manic episode” is defined by a distinct period during which there is an abnormally and persistently elevated, expansive, or irritable mood. This period of abnormal mood must last at least 1 week (or less if hospitalization is required). The mood disturbance must be accompanied by at least three additional symptoms from a list that includes inflated self-esteem or grandiosity, decreased need for sleep, pressure of speech, flight of ideas, distractibility, increased involvement in goal-directed activities or psychomotor agitation, and excessive involvement in pleasurable activities with a high potential of painful consequences. If the mood is irritable (rather than elevated or expansive), at least four of the above symptoms must be present. The disturbance must be sufficiently severe to cause marked impairment in social or occupational functioning or to require hospitalization, or it is characterized by the presence of psychotic features.

A “hypomanic episode” is less severe than a manic episode. The symptoms of a hypomanic episode are generally the same as those which define a manic episode, except that delusions and hallucinations are not present and the episode is not severe enough to cause marked impairment of social and occupational functioning or to require hospitalisation of the individual.

The term “autistic disorder” as used herein means a condition characterized as an Autistic Disorder in the DSM-IV-R as category 299.xx, including 299.00, 299.80, and 299.10, preferably 299.00.

The term “anxiety disorder” includes, but is not limited to, obsessive-compulsive disorder, psychoactive substance anxiety disorder, post-traumatic stress disorder, generalized anxiety disorder, anxiety disorder NOS, and organic anxiety disorder.

The term “substance abuse” as used herein means the undesired physical and/or psychological dependence on a drug. The term refers to dependence on a substance such as cocaine, psychedelic agents, marijuana, amphetamines, hallucinogen, phencyclidine, benzodiazepines, alcohol and nicotine.

The term “attention deficit hyperactivity disorder and “ADHD” as used herein mean a condition or disorder characterized by a persistent pattern of inattention, hyperactivity, impulsivity, or any combination thereof.

The term “excessive aggression” as used herein refers to a condition characterized by aggression that is so excessive that it interferes with the individual's daily functions, relationships, and may threaten the safety of the individual, for example in a situation in which violent suicide is contemplated. The excessive aggression which may be treated using the method claimed herein is independent of a psychotic condition and not directly related to the consumption of a drug or other substance.

A tic is a sudden, rapid recurrent, nonrhythmic, stereotyped motor movement or vocalization, experienced as irresistible but suppressible for varying lengths of time. Common simple motor tics include eye blinking, neck jerking, shoulder shrugging, facial grimacing, and coughing. Common simple vocal tics include throat clearing, grunting, sniffing, snorting, and barking. Common complex motor tics include facial gestures, grooming behaviors, jumping, touching, stamping, and smelling an object. Common complex vocal tics include repeating words or phrases out of context, coprolalia (use of socially unacceptable words, frequently obscene) palilalia (repeating one's own sounds or words), and echolalia(repeating the last heard sound, word or phrase). The term “tic disorder” as used herein means includes tic disorders featuring one or more motor tics and one or more tic and more vocal tics, and vocal tics. Examples include Transient Tic Disorder, Tourette's Disorder, Chronic Vocal Tic Disorder, and Tic Disorder not otherwise specified as described by DSM-IV-R.

The terms “comprising”, “consisting of”, or consisting essentially of” have distinct meaning and each term may be substituted for another herein to change the scope of the invention.

As used interchangeably herein, the term “oligonucleotides”, and polynucleotides” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. Although the term “nucleotide” is also used herein to encompass “modified nucleotides” which comprise at least one modifications (a) an alternative linking group, (b) an analogous form of purine, (c) an analogous form of pyrimidine, or (d) an analogous sugar, for examples of analogous linking groups, purine, pyrimidines, and sugars see for example PCT publication No. WO 95/04064, the disclosure of which is incorporated herein by reference. However, the polynucleotides of the invention are preferably comprised of greater than 50% conventional deoxyribose nucleotides, and most preferably greater than 90% conventional deoxyribose nucleotides. The polynucleotide sequences of the invention may be prepared by any known method, including synthetic, recombinant, ex vivo generation, or a combination thereof, as well as utilizing any purification methods known in the art.

The term “purified” is used herein to describe a polynucleotide or polynucleotide vector of the invention which has been separated from other compounds including, but not limited to other nucleic acids, carbohydrates, lipids and proteins (such as the enzymes used in the synthesis of the polynucleotide), or the separation of covalently closed polynucleotides from linear polynucleotides. A polynucleotide is substantially pure when at least about 50%, preferably 60 to 75% of a sample exhibits a single polynucleotide sequence and conformation (linear versus covalently close). A substantially pure polynucleotide typically comprises about 50%, preferably 60 to 90% weight/weight of a nucleic acid sample, more usually about 95%, and preferably is over about 99% pure. Polynucleotide purity or homogeneity may be indicated by a number of means well known in the art, such as agarose or polyacrylamide gel electrophoresis of a sample, followed by visualizing a single polynucleotide band upon staining the gel. For certain purposes higher resolution of can be provided by using HPLC or other means well known in the art. A polypeptide is substantially pure when at least about 50%, preferably 60 to 75% of a sample exhibits a single polypeptide sequence. A substantially pure polypeptide typically comprises about 50%, preferably 60 to 90% weight/weight of a protein sample, more usually about 95%, and preferably is over about 99% pure. Polypeptide purity or homogeneity is indicated by a number of means well known in the art, such as polyacrylamide gel electrophoresis of a sample, followed by visualizing a single polypeptide band upon staining the gel. For certain purposes higher resolution can be provided by using HPLC or other means well known in the art. The term purified may also is used herein to describe a chemical composition of the invention which have been separated from other compounds.

The term “isolated” requires that the material be removed from its original environment (e.g., the natural environment if it is naturally occurring). For example, a naturally-occurring polynucleotide or polypeptide present in a living animal is not isolated, but the same polynucleotide or DNA or polypeptide, separated from some or all of the coexisting materials in the natural system, is isolated. Such polynucleotide could be part of a vector and/or such polynucleotide or polypeptide could be part of a composition, and still be isolated in that the vector or composition is not part of its natural environment.

The term “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified.

The terms “trait” and “phenotype” are used interchangeably herein and refer to any clinically distinguishable, detectable or otherwise measurable property of an organism such as symptoms of, or susceptibility to a disease for example. Typically the terms “trait” or “phenotype” are used herein to refer to symptoms of, or susceptibility to schizophrenia or bipolar disorder; or to refer to an individual's response to an agent acting on schizophrenia or bipolar disorder; or to refer to symptoms of, or susceptibility to side effects to an agent acting on schizophrenia or bipolar disorder.

The term “allele” is used herein to refer to variants of a nucleotide sequence. A biallelic polymorphism has two forms. Typically the first identified allele is designated as the original allele whereas other alleles are designated as alternative alleles. Diploid organisms may be homozygous or heterozygous for an allelic form.

The term “heterozygosity rate” is used herein to refer to the incidence of individuals in a population, which are heterozygous at a particular allele. In a biallelic system the heterozygosity rate is on average equal to 2Pₐ(1−Pₐ), where Pₐ is the frequency of the least common allele. In order to be useful in genetic studies a genetic marker should have an adequate level of heterozygosity to allow a reasonable probability that a randomly selected person will be heterozygous.

The term “genotype” as used herein refers the identity of the alleles present in an individual or a sample. In the context of the present invention a genotype preferably refers to the description of the biallelic marker alleles present in an individual or a sample. The term “genotyping” a sample or an individual for a biallelic marker involves determining the specific allele or the specific nucleotide(s) carried by an individual at a biallelic marker.

The term “mutation” as used herein refers to a difference in DNA sequence between or among different genomes or individuals which has a frequency below 1%.

The term “haplotype” refers to a combination of alleles present in an individual or a sample on a single chromosome. In the context of the present invention a haplotype preferably refers to a combination of biallelic marker alleles found in a given individual and which may be associated with a phenotype.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A polymorphism may comprise a substitution, deletion or insertion of one or more nucleotides. A single nucleotide polymorphism is a single base pair change. Typically a single nucleotide polymorphism is the replacement of one nucleotide by another nucleotide at the polymorphic site. Deletion of a single nucleotide or insertion of a single nucleotide, also give rise to single nucleotide polymorphisms. In the context of the present invention “single nucleotide polymorphism” preferably refers to a single nucleotide substitution. Typically, between different genomes or between different individuals, the polymorphic site may be occupied by two different nucleotides.

The terms “biallelic polymophism” and “biallelic marker” are used interchangeably herein to refer to a polymorphism having two alleles at a fairly high frequency in the population, preferably a single nucleotide polymorphism. A “biallelic marker allele” refers to the nucleotide variants present at a biallelic marker site. Typically the frequency of the less common allele of the biallelic markers of the present invention has been validated to be greater than 1%, preferably the frequency is greater than 10%, more preferably the frequency is at least 20% (i.e. heterozygosity rate of at least 0.32), even more preferably the frequency is at least 30% (i.e. heterozygosity rate of at least 0.42). A biallelic marker wherein the frequency of the less common allele is 30% or more is termed a “high quality biallelic marker.” All of the genotyping, haplotyping, association, and interaction study methods of the invention may optionally be performed solely with high quality biallelic markers.

The location of nucleotides in a polynucleotide with respect to the center of the polynucleotide are described herein in the following manner. When a polynucleotide has an odd number of nucleotides, the nucleotide at an equal distance from the 3′ and 5′ ends of the polynucleotide is considered to be “at the center” of the polynucleotide, and any nucleotide immediately adjacent to the nucleotide at the center, or the nucleotide at the center itself is considered to be “within 1 nucleotide of the center.” With an odd number of nucleotides in a polynucleotide any of the five nucleotides positions in the middle of the polynucleotide would be considered to be within 2 nucleotides of the center, and so on. When a polynucleotide has an even number of nucleotides, there would be a bond and not a nucleotide at the center of the polynucleotide. Thus, either of the two central nucleotides would be considered to be “within 1 nucleotide of the center” and any of the four nucleotides in the middle of the polynucleotide would be considered to be “within 2 nucleotides of the center”, and so on. For polymorphisms which involve the substitution, insertion or deletion of 1 or more nucleotides, the polymorphism, allele or biallelic marker is “at the center” of a polynucleotide if the difference between the distance from the substituted, inserted, or deleted polynucleotides of the polymorphism and the 3′ end of the polynucleotide, and the distance from the substituted, inserted, or deleted polynucleotides of the polymorphism and the 5′ end of the polynucleotide is zero or one nucleotide. If this difference is 0 to 3, then the polymorphism is considered to be “within 1 nucleotide of the center.” If the difference is 0 to 5, the polymorphism is considered to be “within 2 nucleotides of the center.” If the difference is 0 to 7, the polymorphism is considered to be “within 3 nucleotides of the center,” and so on. For polymorphisms which involve the substitution, insertion or deletion of 1 or more nucleotides, the polymorphism, allele or biallelic marker is “at the center” of a polynucleotide if the difference between the distance from the substituted, inserted, or deleted polynucleotides of the polymorphism and the 3′ end of the polynucleotide, and the distance from the substituted, inserted, or deleted polynucleotides of the polymorphism and the 5′ end of the polynucleotide is zero or one nucleotide. If this difference is 0 to 3, then the polymorphism is considered to be “within 1 nucleotide of the center.” If the difference is 0 to 5, the polymorphism is considered to be “within 2 nucleotides of the center.” If the difference is 0 to 7, the polymorphism is considered to be “within 3 nucleotides of the center,” and so on.

The term “upstream” is used herein to refer to a location which, is toward the 5′ end of the polynucleotide from a specific reference point.

The terms “base paired” and “Watson & Crick base paired” are used interchangeably herein to refer to nucleotides which can be hydrogen bonded to one another be virtue of their sequence identities in a manner like that found in double-helical DNA with thymine or uracil residues linked to adenine residues by two hydrogen bonds and cytosine and guanine residues linked by three hydrogen bonds (See Stryer, L., Biochemistry, 4th edition, 1995).

The terms “complementary” or “complement thereof” are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

The terms “DAO gene ”, when used herein, encompasses genomic, mRNA and cDNA sequences encoding any D-amino acid oxidase proteins of the invention, including the untranslated regulatory regions of the genomic DNA.

The terms “g34872 gene ”, when used herein, encompasses genomic, mRNA and cDNA sequences encoding any g34872 protein, including the untranslated regulatory regions of the genomic DNA.

The terms “DDO gene”, when used herein, encompasses genomic, mRNA and cDNA sequences encoding any D-aspartate oxidase protein, including the untranslated regulatory regions of the genomic DNA.

As used herein the term “13q31-g33-related biallelic marker” relates to a set of biallelic markers residing in the human chromosome 13q31-q33 region. The term 13q31-q33-related biallelic marker encompasses all of the biallelic markers disclosed in Table 6b of U.S. patent application Ser. No. 09/539,333 and international application PCT/IB00/00435, which disclosures are incorporated by reference in their entireties, and any biallelic markers in linkage disequilibrium therewith ,as well as any biallelic markers disclosed in Table 6c (of same U.S. patent application Ser. No. 09/539,333 and international application PCT/IB00/00435) and any biallelic markers in linkage disequilibrium therewith. The preferred chromosome 13q31-q33-related biallelic marker alleles of the present invention include each one the alleles described in Tables 6b (of same U.S. patent application Ser. No. 09/539,333 and international application PCT/IB00/00435) individually or in groups consisting of all the possible combinations of the alleles listed.

As used herein the term “Region D-related biallelic marker” relates to a set of biallelic markers in linkage disequilibrium with the subregion of the chromosome 13q31-q33 region referred to herein as Region D. The term Region D-related biallelic marker encompasses the biallelic markers A1 to A242, A249 to A251, A257 to A263, A269 to A270, A278, A285 to A299, A303 to A307, A324, A330, A334 to A335, A346 to A357 and A361 to A489 disclosed in Table 6b and any biallelic markers in linkage disequilibrium with markers A1 to A242, A249 to A251, A257 to A263, A269 to A270, A278, A285 to A299, A303 to A307, A324, A330, A334 to A335, A346 to A357 and A361 to A489, of U.S. patent application Ser. No. 09/539,333 and international application PCT/IB00/00435, which disclosures are incorporated by reference in their entireties.

As used herein the term “sbg1-related biallelic marker” relates to a set of biallelic markers in linkage disequilibrium with the sbg1 gene or an sbg1 nucleotide sequence. The term sbg1-related biallelic marker encompasses the biallelic markers A85 to A219 disclosed in Table 6b and any biallelic markers in linkage disequilibrium therewith, of U.S. patent application Ser. No. 09/539,333 and international application PCT/IB00/00435, which disclosures are incorporated by reference in their entireties.

As used herein the term “g34665-related biallelic marker” relates to a set of biallelic markers in linkage disequilibrium with the g34665 gene or an sbg1 nucleotide sequence. The term g34665-related biallelic marker encompasses the biallelic markers A230 to A236 disclosed in Table 6b and any biallelic markers in linkage disequilibrium therewith.

The term “polypeptide” refers to a polymer of amino acids without regard to the length of the polymer; thus, peptides, oligopeptides, and proteins are included within the definition of polypeptide. This term also does not specify or exclude prost-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

As used herein, the term “non-human animal” refers to any non-human vertebrate, birds and more usually mammals, preferably primates, farm animals such as swine, goats, sheep, donkeys, and horses, rabbits or rodents, more preferably rats or mice. As used herein, the term “animal” is used to refer to any vertebrate, preferable a mammal. Both the terms “animal” and “mammal” expressly embrace human subjects unless preceded with the term “non-human”.

As used herein, the term “antibody” refers to a polypeptide or group of polypeptides which are comprised of at least one binding domain, where an antibody binding domain is formed from the folding of variable domains of an antibody molecule to form three-dimensional binding spaces with an internal surface shape and charge distribution complementary to the features of an antigenic determinant of an antigen., which allows an immunological reaction with the antigen. Antibodies include recombinant proteins comprising the binding domains, as wells as fragments, including Fab, Fab′, F(ab)₂, and F(ab′)₂ fragments.

As used herein, an “antigenic determinant” is the portion of an antigen molecule, in this case an sbg1 polypeptide, that determines the specificity of the antigen-antibody reaction. An “epitope” refers to an antigenic determinant of a polypeptide. An epitope can comprise as few as 3 amino acids in a spatial conformation which is unique to the epitope. Generally an epitope comprises at least 6 such amino acids, and more usually at least 8-10 such amino acids. Methods for determining the amino acids which make up an epitope include x-ray crystallography, 2-dimensional nuclear magnetic resonance, and epitope mapping e.g. the Pepscan method described by Geysen et al. 1984; PCT Publication No. WO 84/03564; and PCT Publication No. WO 84/03506.

A complete description of “Variants and Fragments”, “Identity Between Nucleic Acids Or Polypeptides”, “Stringent Hybridization Conditions”, “DNA Constructs that Enables Directing Temporal and Spatial Expression of sbg1 Nucleic Acid Sequences in Recombinant Cell Hosts and in Transgenic Animals” are fully detailed in co-pending U.S. patent application Ser. No. 09/539,333 titled “Schizophrenia associated genes, proteins and biallelic markers” and co-pending International Patent Application No. PCT/IB00/00435, both filed Mar. 30, 2000 and which disclosures are hereby incorporated by reference in their entireties.

Genomic Sequences of g34872 and DAO polynucleotides

Particularly preferred g34872 nucleic acids of the invention include isolated, purified, or recombinant polynucleotides comprising, consisting essentially of, or consisting of a contiguous span of at least 12, 15, 18, 20, 25, 30, 35, 40, 50, 60, 70, 80, 90, 100, 150, 200, 500, or 1000 nucleotides of nucleotide positions 213818 to 243685 of U.S. patent application Ser. No. 09/539,333 SEQ ID No: 1, or the complements thereof (U.S. patent application Ser. No. 09/539,333 and international application PCT/IB00/00435, which disclosures are incorporated by reference in their entireties).

DAO polynucleotides of the invention are described in SEQ ID NO: 1 of the present invention. Particularly preferred nucleic acids of the invention include isolated, purified, or recombinant polynucleotides comprising, consisting essentially of, or consisting of a contiguous span of at least 12, 15, 18, 20, 25, 30, 35, 40, 50, 60, 70, 80, 90, 100, 150, 200, 500, or 1000 nucleotides of nucleotide positions 6000-86600 of SEQ ID No: 1. Nucleic acids of the invention encompass DAO nucleic acid from any source, including primate, non-human primate, mammalian and human DAO nucleic acids.

Further preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 1 or the complements thereof, wherein said contiguous span comprises a DAO related biallelic marker. Optionally, said biallelic marker is selected from the group comprising 24-1443/126, 24-1457/52, or 24-1461/256. Preferably, said biallelic marker is 24-1461/256.

It should be noted that nucleic acid fragments of any size and sequence may also be comprised by the polynucleotides described in this section.

Thus, the invention embodies purified, isolated, or recombinant polynucleotides comprising a nucleotide sequence selected from the group consisting of the exons of the DAO gene (SEQ ID NO: 1), or a sequence complementary thereto. Preferred are purified, isolated, or recombinant polynucleotides comprising at least one exon of the DAO gene, or a complementary sequence thereto or a fragment or a variant thereof. Also encompassed by the invention are purified, isolated, or recombinant nucleic acids comprising a combination of at least two exons of the DAO gene selected from the group consisting of exons Z, A, B, C, Ulong, U, V, Z, 2, 3, 4, 5, 6, 7, 8, 9, 10, 11, and 11 long, wherein the polynucleotides are arranged within the nucleic acid in the same relative order as in SEQ ID NO: 1.

Particularly preferred nucleic acids of the invention include isolated, purified, or recombinant polynucleotides comprising a contiguous span of at least 12, 15, 18, 20, 25, 30, 35, 40, 45, 50, 55, 60, 65, 70, 75, 80, 90, 100 or 200 nucleotides of SEQ ID No 1. or the complements thereof.

Another object of the invention consists of a purified, isolated, or recombinant nucleic acid that hybridizes with an DAO nucleotide sequence of SEQ ID NO: 1, or a complementary sequence thereto or a variant thereof, under the stringent hybridization conditions as defined above.

The present invention further embodies purified, isolated, or recombinant polynucleotides comprising a nucleotide sequence selected from the group consisting of the introns of the DAO gene (SEQ ID NO: 1), or a sequence complementary thereto.

In other embodiments, the present invention encompasses the DAO gene as well as DAO genomic sequences consisting of, consisting essentially of, or comprising the sequence of nucleotide positions of SEQ ID No 1, a sequence complementary thereto, as well as fragments and variants thereof.

The invention also encompasses a purified, isolated, or recombinant polynucleotide comprising a nucleotide sequence of DAO having at least 70, 75, 80, 85, 90, or 95% nucleotide identity with SEQ ID NO: 1 or a complementary sequence thereto or a fragment thereof.

These nucleic acids, as well as their fragments and variants, may be used as oligonucleotide primers or probes in order to detect the presence of a copy of a gene comprising an g34782, DAO or DDO nucleic acid sequence in a test sample, or alternatively in order to amplify a target nucleotide sequence within an g347982, DAO or DDO nucleic acid sequence or adjoining region.

Additional preferred nucleic acids of the invention include isolated, purified, or recombinant DAO polynucleotides comprising a contiguous span of at least 12, 15, 18, 20, 25, 30, 35, 40, 45, 50, 55, 60, 65, 70, 75, 80, 90, 100 or 200 nucleotides of SEQ ID NO: 1, or the complements thereof, wherein said contiguous span comprises at least one biallelic marker. Optionally, said contiguous span comprises an DAO-related biallelic marker. It should be noted that nucleic acid fragments of any size and sequence may also be comprised by the polynucleotides described in this section. Either the original or the alternative allele may be present at said biallelic marker.

Yet further nucleic acids of the invention include isolated, purified, or recombinant polynucleotides comprising a contiguous span of at least 12, 15, 18, 20, 25, 30, 35, 40, 50, 60, 70, 80, 90, 100, 150, 200 or 500 nucleotides, to the extent that said span is consistent with the nucleotide position range, of SEQ ID NO: 1, wherein said contiguous span comprises at least 1, 2, 3, 5, or 10 of the following nucleotide positions of SEQ ID No 1: 215820 to 215941, 216661 to 217009, 230409 to 290721, 231272 to 231411, 234202 to 234321, 240528 to 240567, 240528 to 240827 and 240528 to 240996, or the complements thereof, as well as polynucleotides having at least 70, 75, 80, 85, 90, or 95% nucleotide identity with said span, and polynucleotides capable of hybridizing with said span.

The present invention also comprises a purified or isolated nucleic acid encoding an DAO protein having the amino acid sequence of any one of SEQ ID NOs: 7-10 or a peptide fragment or variant thereof.

While this section is entitled “Genomic Sequences of sbg1,” it should be noted that nucleic acid fragments of any size and sequence may also be comprised by the polynucleotides described in this section, flanking the genomic sequences sbg1 on either side or between two or more such genomic sequences.

DAO cDNA Sequences

The expression of the DAO gene has been shown to lead to the production of several mRNA species. Several cDNA sequences corresponding to these mRNA are set forth in SEQ ID NOs: 2-6.

The invention encompasses a purified, isolated, or recombinant nucleic acid comprising a nucleotide sequence selected from the group consisting of SEQ ID NOs: 2-6, complementary sequences thereto, splice variants thereof, as well as allelic variants, and fragments thereof. Moreover, preferred polynucleotides of the invention include purified, isolated, or recombinant DAO cDNAs consisting of, consisting essentially of, or comprising a nucleotide sequence selected from the group consisting of SEQ ID NOs: 2-6. Particularly preferred nucleic acids of the invention include isolated, purified, or recombinant polynucleotides comprising a contiguous span of at least 8, 12, 15, 18, 20, 25, 30, 35, 40, 50, 60, 70, 75, 80, 100, 200 or 500 nucleotides, to the extent that the length of said contiguous span is consistent with the length of the SEQ ID NOs: 2-6, or the complements thereof.

It should be noted that nucleic acid fragments of any size and sequence may also be comprised by the polynucleotides described in this section.

The invention also pertains to a purified or isolated nucleic acid comprising a polynucleotide having at least 70, 80, 85, 90 or 95% nucleotide identity with a polynucleotide selected from the group consisting of SEQ ID NOs: 2-6, advantageously 99% nucleotide identity, preferably 99.5% nucleotide identity and most preferably 99.8% nucleotide identity with a polynucleotide selected from the group consisting of SEQ ID NOs: 2-6, or a sequence complementary thereto or a biologically active fragment thereof.

Another object of the invention relates to purified, isolated or recombinant nucleic acids comprising a polynucleotide that hybridizes, under the stringent hybridization conditions defined herein, with a polynucleotide selected from the group consisting of SEQ ID NOs: 2-6, or a sequence complementary thereto or a variant thereof or a biologically active fragment thereof. The DAO cDNA forms of SEQ ID NOs: 2-6 are further described in the sequence listing.

Primers used to isolate the particular DAO cDNAs or for genotyping are listed in SEQ ID NO: 1. Biallelic markers for DAO, and genotyping primers thereof, are listed in SEQ ID NOs: 1, 24, 26, and 29. Polynucleotides of g34872 are listed in SEQ ID NO: 14 and 16. g34872 biallelic marker 99-16105-152 of SEQ ID NO: 12and g34872 biallelic marker 99-5919-215 of SEQ ID NO: 13 are listed and primers to make are described therein. cDNA of g34872 is listed in SEQ ID NO: 14 and polynucleotides used in 2-hybrid experiments are listed in SEQ ID NO: 16.

The present inventors have also identified novel exons and variations in cDNA sequence as obtained from various tissues and these are listed as Exons 11 long, Z, A, B, C, and UL of SEQ ID NO: 1, and in polynucleotides of SEQ ID NOs: 2-6. Novel forms of DAO polypeptides are listed in SEQ ID NO: 8-10.

These variants represent rare and novel forms of DAO which are preferably used to screen for compositions to use in methods of treating CNS disorders.

It should be noted that nucleic acid fragments of any size and sequence may also be comprised by the polynucleotides described in this section, flanking the genomic sequences of g34872, DAO and DDO on either side or between two or more such genomic sequences.

DAO and DDO Antagonists

The term “antagonist” as used herein refers to the inhibition of enzymatic reaction whereby DAO or DDO converts a D-amino acid substrate into the corresponding .alpha.-keto acid. The antagonists may be specified as either competitive, non-competitive, uncompetitive, allosteric, or irreversible inhibitors of DAO or DDO enzymatic activity. The term “activity” or “enzymatic activity” of DAO or DDO refers to the enzymatic reaction above. Antagonists may be specified in terms of the degree of inhibition of DAO or DDO activity. Preferred antagonists reduce DAO or DDO activity by at least 10%, 20%, 30%, 40%, 50%, 60%, 70%, 80%, 90%, or 95%. Inhibitory effect may also be specified as an inhibition constant or Kᵢ(M) values. Preferred antagonists have a Kᵢ(M) with a numeric value less than 5×10⁻², 1×10⁻², 5×10⁻³, 1×10⁻¹, 5×10⁻¹, 1×10⁻⁴, 5×10⁻⁵, 1×10⁻⁶, 5×10⁻⁷, 1×10⁻⁷. It is noted that there is an inverse relationship between the Kᵢ(M) numeric value and the inhibitory effect, i.e., as the Kᵢ(M) value decreases, the inhibitory effect increases. Antagonists may also be specified in terms of their specificity for DAO or DDO. Therefore, included in the present invention are antagonists that inhibit DAO or DDO activity but do not inhibit other human flavoproteins (p-Hydroxybenzoate hydroxylase, cholesterol oxidase and glucose oxidase)or has a Kᵢ(M) numeric value for other human flavoproteins greater than 1×10⁻², 5×10⁻². It should be appreciated from the definition that the generic terms “antagonist” and “inhibitor” can be used interchangeably to indicate any composition which inhibits DAO or DDO activity as defined above. In addition, specific types of antagonists or inhibitors can be set forth independently as described in the specification, for example a competitive inhibitor.

Over 200 inhibitors of DAO and DDO have been studied to date. DAO and DDO antagonists may be selected from the compositions presented supra, or other antagonists known in the art, or made using the methods described herein, or known in the art. Alternatively, DAO and DDO antagonists can be purchased from commercial suppliers. A non-limiting list of compounds useful in accordance with the invention is provided in Table I. DAO and DDO antagonists are further comprise the families of compositions selected from the groups comprising: Competitive Inhibitor compositions, Irreversible Inhibitor compositions, Formula I, Formula II, Formula III, Formula IV, Formula V, and Formula VI compositions, and subgroups thereof, as presented herein. Further preferred representative compositions of the Formulae I-VI, and subgroups thereof, include, but are not limited to the detailed description infra.

Formula I compositions, or pharmaceutically acceptable salts thereof, are represented by the structure comprising:

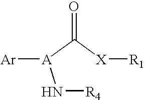

wherein:

a) A is alkyl such as methyl, ethyl, propyl or butyl; branched chain alkyl such as isobutyl, isopropyl, isopentyl or cycloalkyl such as cyclopropyl, cyclopentyl or cyclohexyl. Such groups may themselves be substitued with C₁-C₆ alkyl, halo, hydroxyl or amino;

X is O or N;

c) Ar is an aromatic mono-, bi- or tricyclic fused heterocyclic ring, wherein the ring is either unsubstituted or substituted in one to five position(s) with hydrogen, halogen, hydroxyl, —CN, COR₂, —CONR₂R₃, —S(O)ₙR₂, —OPO(OR₂)R₃, —PO(OR₃)R₃, —OC(O)NR₂R₃, —COOR₂, —CONR₂R₃, —SO₃H, —NR₂R₃, —NR₂COR₃, —NR₃COOR₃, —SO₂NR₂R₃, —N(R₂)SO₂R₃, —NR₂CONR₂R₂, —SO₂NHCOR₂, —CONHSO₂R₂, —SO₂NHCN, —OR₁, C₁-C₆ straight or branched chain alkyl or alkenyl, or C₁-C₆ branched or straight chain alkyl or alkenyl which is substituted with one or more, halogen, hydroxyl, amino, carboxy, carboxamide, nitrile, nitro, alkoxy, trifluoromethyl, sulfur, sulfonate, phosphonate, phosphate, Ar¹, N₃ or a combination thereof and wherein the heterocyclic ring contains 1-6 heteroatom(s) selected from the group consisting of O, N, S, and a combination thereof;

d) R₄ is H, alkyl, Ar¹, O, substituted alkyl;

e) R¹ is (C₁-C₆)alkyl, Ar¹, (C₁-C₄)alkoxycarbonylmethyl, substituted alkyl;

f) R₂ and R₃ are each, independently, hydrogen, C₁-C₆ straight or branched chain alkyl or alkenyl, or C₁-C₆ branched or straight chain alkyl or alkenyl which is substituted with one or more, halogen, hydroxyl, amino, carboxy, carboxamide, nitrile, nitro, alkoxy, trifluoromethyl, sulfur, sulfonate, phosphonate, phosphate, Ar¹, or N₃; and

g) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 3-7 members; and wherein the heterocyclic ring contains 1-6 heteroatom(s) selected from the group consisting of O, N, S, and a combination thereof.

Further preferred Formula I compositions, or pharmaceutically acceptable salts thereof, are Formula Ia compositions, or pharmaceutically acceptable salts thereof, comprising the structure:

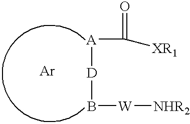

wherein:

a) A and B consist of C or N and D may contain 0-2 members consisting of C or N;

b) W is C₁-C₄ alkyl such as (CH₂)ₙ, branched chain alkyl;

c) n is 0-4. Further, when n=0 it is assumed that —NHR₂ is covalently bound to B;

d) X is O or N;

e) R₂ is H, alkyl, Ar¹, or O substituted alkyl;

f) R¹ is (C₁-C₆)alkyl, Ar¹, (C₁-C₄)alkoxycarbonylmethyl, or substituted alkyl;

g) Ar is an aromatic mono-, bi- or tricyclic fused heterocyclic ring, wherein the ring is either unsubstituted or substituted in one to six position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, C₃-C₆ cycloalkyl or a combination thereof;

wherein the individual ring sizes are 5-6 members; and wherein the heterocyclic ring contains 1-6 heteroatom(s) selected from the group consisting of O, N, S, and a combination thereof, and

h) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof, wherein the individual ring sizes are 3-7 members; and wherein the heterocyclic ring contains 1-6 heteroatom(s) selected from the group consisting of O, N, S, and a combination thereof.

Further preferred Formula Ia compositions, or pharmaceutically acceptable salts thereof, are Formula Ib compositions, or pharmaceutically acceptable salts thereof, comprising the structure:

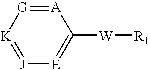

wherein:

a) A, G, K, J, E are members of a six membered carbo or heterocyclic aromatic ring, wherein the heterocyclic ring contains 1-6 heteroatom(s) selected from the group consisting of C, N and a combination thereof;

b) A, G, K, J, E may each independently be unsubstituted or substituted with hydrogen, halogen, hydroxyl, —CN, COR₂, —CONR₂R₃, —S(O)ₙR₂, —OPO(OR₂)OR₃, —PO(OR₃)R₃, —OC(O)NR₂R₃, —COOR₂, —CONR₂R₃, —SO₃H, —NR₂R₃, —NR₂COR₃, —NR₃COOR₃, SO₂NR₂R₃, —N(R₂)SO₂R₃, —NR₂CONR₂R₂, —SO₂NHCOR₂, —CONHSO₂R₂, —SO₂NHCN, —OR₁, C₁-C₆ straight or branched chain alkyl or alkenyl, or C₁-C₆ branched or straight chain alkyl or alkenyl which is substituted with one or more halogen, hydroxyl, amino, carboxy, carboxamide, nitrile, nitro, alkoxy, trifluoromethyl, sulfur, sulfonate, phosphonate, phosphate, Ar¹, or N₃;

c) R₁ is CN, COR₂, —CONR₂R₃, —S(O)ₙR₂, —OPO(OR₂)OR₃, —PO(OR₃)R₃, —OC(O)NR₂R₃, —COOR₂, —CONR₂R₃, —SO₃H, —NR₂R₃, —NR₂COR₃, —NR₃COOR₃, —SO₂NR₂R₃, —N(R₂)SO₂R₃, —NR₂CONR₂R₂, —SO₂NHCOR₂, —CONHSO₂R₂, —SO₂NHCN, SCN, COCO₂H, C₁-C₆ straight or branched chain alkyl or alkenyl, or C₁-C₆ branched or straight chain alkyl or alkenyl which is substituted with one or more halogen, hydroxyl, amino, carboxy, carboxamide, nitrile, nitro, alkoxy, trifluoromethyl, sulfur, sulfonate, phosphonate, phosphate, Ar¹, or N₃;

d) W is N, (CH₂)ₓ, or —NCH₂;

e) x=0-4;

f) n=0-2;

g) R₂ and R₃ are each, independently, hydrogen, C₁-C₆ straight or branched chain alkyl or alkenyl, or C₁-C₆ branched or straight chain alkyl or alkenyl which is substituted with one or more halogen, hydroxyl, amino, carboxy, carboxamide, nitrile, nitro, alkoxy, trifluoromethyl, sulfur, sulfonate, phosphonate, phosphate, Ar¹, or N₃; and

h) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 5-6 members; and wherein the heterocyclic ring contains 1-6 heteroatom(s) selected from the group consisting of O, N, S, and a combination thereof.

Specific examples of Formulae I, Ia, and Ib compositions, or pharmaceutically acceptable salts thereof, include, but not limited to, the list comprising:

a) Benzoic acid;

b) 2-Aminobenzoate;

c) 3-Aminobenzoate;

d) 4-Aminobenzoate;

e) Salicylic acid;

f) N-Methylnicotinate;

g) Methyl-6-methylnicotinate;

h) Ethyl-2-methylnicotinate;

i) Anthranilate;

j) Ethyl-2-aminobenzoate;

k) Methyl-2-aminobenzoate;

l) Picolinate;

m) Ethyl-2-pyridinecarboxylate;

n) 3-Methylbenzyl thiocyanate;

o) Phenyl pyruvic acid;

p) Phenylglyoxilic acid;

q) 1-Methyl pyridinium-3-carboxylate;

r) Befloxatone; (5R)-5-(Methoxymethyl)-3-[4-[(3R)-4,4,4-trifluoro-3-hydroxybutoxy]phenyl]-2-oxazolidinone;

s) Bupropion; 1-(3-Chlorophenyl)-2-[(1,1-dimethylethyl)amino]-1-propanone;

t) Cotinine; 1-Methyl-5-(3-pyridinyl)-2-pyrrolidinone;

u) Duloxetine; (γS)—N-Methyl-γ-(1-naphthalenyloxy)-2-thiophenepropanamine;

v) Fenpentadiol; 2-(4-Chlorophenyl)-4-methyl-2,4-pentanediol;

w) Fluvoxamine; (E)-5-Methoxy-1-[4-(trifluoromethyl)phenyl]-1-pentanone O-(2-aminoethyl)oxime;

x) Iproclozide; 4-(Chlorophenoxy)acetic acid 2-(1-methylethyl)hydrazide;

y) Iproniazid; 4-Pyridinecarboxylic acid 2-(1-methylethyl)hydrazide;

z) Levophacetoperane; α-Phenyl-2-piperidinemethanol acetate;

aa) Rolipram; 4-[3-(Cyclopentyloxy)-4-methoxyphenyl]-2-pyrrolidinone;

bb) Tranylcypromine; (1R,2S)-rel-2-Phenylcyclopropanamine; and

cc) Milnacipran; (1R,2S)-rel-2-(Aminomethyl)—N,N-diethyl-1-phenylcyclopropanecarboxamide.

Formula II compositions, or pharmaceutically acceptable salts thereof, are represented by the structure comprising:

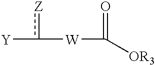

wherein:

a) W=(CH₂)ₙ;

b) n=0-5;

c) Z is O or hydroxyl;

d) Y═H, Ar¹, R₄(CH₂)ₓ, R₁S(CH₂)ₓ—, R₁SO(CH₂)ₓ—, R₁SO₂(CH₂)ₓ—, R₁SO₃(CH₂)ₓ—, HNR₁SO₂(CH₂)ₓ—, R₁R₂N(CH₂)ₓ, R₁O(CH₂)—, CF₃, or OH;

e) x=0-6;

f) R₁, R₂ and R₃ are each independently hydrogen, C₁-C₆ straight or branched chain alkyl or C₁-C₆ branched or straight chain alkyl substituted with one or more halogen, hydroxyl, amino, carboxy, carboxamide, nitrile, nitro, alkoxy, trifluoromethyl, sulfur, sulfonate, phosphonate, phosphate, or Ar¹;

g) R₄ is halogen, CN, N₃, C₁-C₆ straight or branched chain alkyl or C₁-C₆ branched or straight chain alkyl substituted with one or more halogen, hydroxyl, nitro, alkoxy, trifluoromethyl, sulfonate, phosphonate, phosphate, Ar¹, —COR₁, —COOR₁, —CONR₁R₂, CN, —NR₁, —NR₁R₂, —SR₁, —SO₂NHCN, or N₃; and

h) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 5-6 members; and wherein the heterocyclic ring contains 1-6 heteroatom(s) selected from the group consisting of O, N, S, and a combination thereof.

Further preferred Formula II compositions, or pharmaceutically acceptable salts thereof, are Formula IIa compositions, or pharmaceutically acceptable salts thereof, comprising the structure:

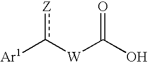

wherein:

a) Y is Ar¹;

b) Z is a carbonyl or hydroxyl;

c) W is (CH₂)ₙ wherein (n=0, 1, or 2) and R₃═H; and

d) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 5-6 members; and wherein the heterocyclic ring contains 1-6 heteroatom(s) selected from the group consisting of O, N, S, and a combination thereof.

Specific examples of Formula II and IIa compositions, or pharmaceutically acceptable salts thereof, include, but are not limited to, the list comprising:

a) 2-Oxo-propionic acid;

b) 5-Guanidiono-2-oxo-pentanoic acid;

c) 2-Oxo-succinamic acid;

d) 2-Oxo-succinic acid;

e) 3-Mercapto-2-oxo-propionic acid;

f) 3-(1H-Imidazol-4-yl)-2-oxo-propionic acid;

g) 3-Methyl-2-oxo-pentanoic acid;

h) Oxo-acetic acid;

i) 4-Carbamoyl-2-oxo butyric acid;

j) 2-Oxo-pentanedioic acid;

k) 4-Methyl-2-oxo-penatanoic acid;

l) 6-Amino-2-oxo-hexanoic acid;

m) 4-Methylsulfanyl-2-oxo-butyric acid;

n) 2-Oxo-3-phenyl propionic acid;

o) 3-Hydroxy-2-oxo-propionic acid;

p) 3-Hydroxy-2-oxo-butyric acid;

q) 3-(1H-Indol-3-yl)-2-oxo-propionic acid;

r) 3-(4-Hydroxy-phenyl)-2-oxo-propionic acid;

s) 3-methyl-2-oxo-butyric acid;

t) 2-Hydroxy butyric acid;

u) 3-Hydroxy byutyric acid; and

v) 3-Oxoglutaric acid.

Formula III compositions, or pharmaceutically acceptable salts thereof, are represented by the structure comprising:

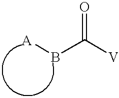

wherein:

a) A and B taken together, form a 5-8 membered saturated or partially unsaturated heterocyclic ring containing at least one additional O, S, SO, SO₂, NH, or NR¹ heteroatom in any chemically stable oxidation state;

b) V is O, OR₁, NR₂, NR₁R₂, CHR₁R₂, CH₂R₃, CHR₃R₄, or CH₂N₃;

c) R₁ and R₂ are independently hydrogen, C₁-C₆ straight or branched chain alkyl or C₁-C₆ branched or straight chain alkyl substituted with one or more halogen, hydroxyl, amino, carboxy, carboxamide, nitro, alkoxy, trifluoromethyl, sulfur, sulfonate, phosphonate, or Ar¹;

d) R₃ and R₄ are either halogen, C₁-C₆ straight or branched chain alkyl or C₁-C₆ branched or straight chain alkyl substituted with one or more hydroxyl, amino, carboxy, carboxamide, nitro, alkoxy, trifluoromethyl, sulfur, sulfonate, phosphonate, Ar¹, —OC(O)R₁, —COOR₁, —CONR₁R₂, CN, NR₁, NR₁R₂, SR₁, SO₂NHCN, or N₃; and

e) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 5-6 members; and wherein the heterocyclic ring contains 1-6 heteroatom(s) selected from the group consisting of O, N, S, and a combination thereof.

Specific examples of Formula III compositions include, but are not limited to, Cystathionine ketimine and Cyclothionine.

Further preferred Formula III compositions, or pharmaceutically acceptable salts thereof, are Formula IV compositions, or pharmaceutically acceptable salts thereof, represented by the structure comprising:

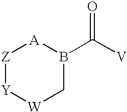

wherein:

a) W—Y-Z-A-B comprise a six membered saturated or partially saturated carbocyclic or heterocylic ring, wherein the heterocyclic ring contains heteroatom(s) selected from the group consisting of O, N, S, and any combination thereof;

b) B is either C, CH or N;

c) A, W, Y, Z are each independently CH₂, CHR₃, CR₃R₄, O, S, SO, SO₂, NH, NR₁, NR₁R₂, or C═O;

d) V is O, OR₁, NR₂, NR₁R₂, CHR₁R₂, CH₂R₃, CHR₃R₃, or CH₂N₃;

e) R₁ and R₂ are independently hydrogen, C₁-C₆ straight or branched chain alkyl or C₁-C₆ branched or straight chain alkyl substituted with one or more, halogen, hydroxyl, amino, carboxy, carboxamide, nitrile, nitro, alkoxy, trifluoromethyl, sulfur, sulfonate, phosphonate, phosphate, or Ar¹;

f) R₃ and R₄ are each independently halogen, —OC(O)R₁, —COOR₁, —CONR₁R₂, CN, —NR₁, —NR₁R₂, —SR₁, —SO₂NHCN, N₃, C₁-C₆ straight or branched chain alkyl or C₁-C₆ branched or straight chain alkyl substituted with one or more halogen, hydroxyl, nitro, alkoxy, trifluoromethyl, sulfonate, phosphonate, Ar¹, —OC(O)R₁, —COOR₁, —CONR₁R₂, CN, —NR₁, —NR₁R₂, —SR₁, —SO₂NHCN, or N₃; and

g) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof, wherein the individual ring sizes are 5-6 members; and wherein the heterocyclic ring contains 1-6 heteroatom(s) selected from the group consisting of O, N, S, and any combination thereof.

Specific examples of Formula IV compositions, or pharmaceutically acceptable salts thereof, include, but are not limited to, Aminoethylcysteine-ketimine (2H-1,4-thiazine-5,6-dihydro-3-carboxylic acid), Thiomorpholine-2-carboxylic acid, Lanthionine ketimine, and 1,4-Thiomorpholine-3,5-dicarboxylic acid.

Formula V compositions, or pharmaceutically acceptable salts thereof, are represented by the structure comprising:

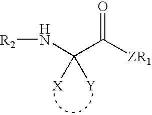

wherein:

a) Z is O or NH;

b) R¹ is (C₁-C₆)alkyl, Ar¹, or (C₁-C₄)alkoxycarbonylmethyl;

c) X, Y, independently of one another, are H, Ar¹, (C₁-C₆)alkyl (which can be interrupted or substituted by heteroatoms, such as N, P, O, S or Si, it being possible for the heteroatoms themselves to be substituted by (C₁-C₃)alkyl once or several times), (C₂-C₆)alkenyl, (C₁-C₆) haloalkyl,or halogen. When X and Y are each carbon they may be covalently joined to form a saturated or partially unsaturated carbocyclic compound of 3-8 members consisting independently of C, N, O, and S, further wherein ring members may themselves be unsubstituted or substituted with halo, hydroxyl, carboxy, nitro, trifluoromethyl, C₁-C₆ straight or branched chain alkyl or alkenyl, C₁-C₄ alkoxy, C₁-C₄ alkenyloxy, phenoxy, benzyloxy, amino, substituted alkyl, Ar¹, or a combination thereof;

d) R₂ is H, alkyl, Ar¹, or O substituted alkyl; and

e) Ar¹ is a mono-, bi- or tricyclic, carbo- or hetero cyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 3-7 members; and wherein the heterocyclic ring contains 1-6 heteroatom(s) selected from the group consisting of O, N, S, and any combination thereof.

Further preferred Formula V compositions, or pharmaceutically acceptable salts thereof, are Formula Va compositions, or pharmaceutically acceptable salts thereof, comprising the structure:

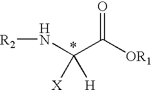

wherein:

a) *=asymmetric center and

b) R¹═(C₁-C₆)alkyl, Ar¹, (C₁-C₄)alkoxycarbonylmethyl and

c) X is H, (C₁-C₆)alkyl (which can be interrupted or substituted by heteroatoms, such as N, P, O, S or Si, it being possible for the heteroatoms themselves to be substituted by (C₁-C₃) alkyl once or several times), (C₂-C₆)alkenyl, (C₁-C₆) haloalkyl, halogen, or Ar¹;

d) R₂ is H, alkyl, Ar¹, or O substituted alkyl;

e) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 3-7 members; and wherein the heterocyclic ring contains 1-6 heteroatom(s) selected from the group consisting of O, N, S, and any combination thereof.

Further preferred Formula V compositions, or pharmaceutically acceptable salts thereof, include Formula Vb compositions, or pharmaceutically acceptable salts thereof, comprising the structure:

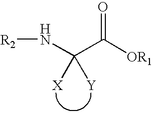

wherein:

a) X and Y are each carbon;

b) X and Y are connected by a saturated or partially saturated ring of 3-8 carbons and such a ring may itself be substituted in one to five position(s) with halo, hydroxyl, carboxy, amino, nitro, cyano, trifluoromethyl, C₁-C₆ straight or branched chain alkyl or alkenyl, C₁-C₄ alkoxy, C₁-C₄ alkenyloxy, or substituted alkyl groups;

c) R¹ is (C₁-C₆)alkyl, Ar¹, or (C₁-C₄)alkoxycarbonylmethyl;

d) R₂ is H, alkyl, Ar¹, or O substituted alkyl; and

e) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 3-7 members; and wherein the heterocyclic ring contains 1-6 heteroatom(s) selected from the group consisting of O, N, S, and any combination thereof.

Further preferred Formula Vb compositions include rings joining X and Y which comprise 3-6 members.

Further preferred Formula V compositions, or pharmaceutically acceptable salts thereof, are Formula Vc compositions, or pharmaceutically acceptable salts thereof, comprising the structure:

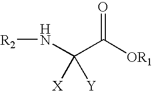

wherein:

a) X, Y, independently of one another, are H, Ar¹, (C₁-C₆)alkyl (which can be interrupted or substituted by heteroatoms, such as N, P, O, S or Si, it being possible for the heteroatoms themselves to be substituted by (C₁-C₃)alkyl once or several times), (C₂-C₆)alkenyl, (C₁-C₆) haloalkyl, or halogen such as naphthyl or phenyl;

b) R₂ is H, alkyl, Ar¹, or O substituted alkyl; and

c) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 3-7 members; and wherein the heterocyclic ring contains 1-6 heteroatom(s) selected from the group consisting of O, N, S, and any combination thereof.

Formula VI compositions, or pharmaceutically acceptable salts thereof, are represented by the structure comprising:

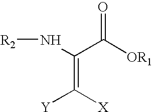

wherein:

a) R¹ is (C₁-C₆)alkyl, Ar¹, or (C₁-C₄)alkoxycarbonylmethyl;

b) R₂ is H, alkyl, Ar¹, or O substituted alkyl;

c) Y is H, Ar¹, (C₁-C₆)alkyl (which can be interrupted or substituted by heteroatoms, such as N, P, O, S or Si, it being possible for the heteroatoms themselves to be substituted by (C₁-C₃)alkyl once or several times), (C₂-C₆)alkenyl, (C₁-C₆) haloalkyl, or halogen; and

d) X is alkyl or phenyl.

In further preferred Formula VI compositions, Y is Ar¹. In still further preferred Formula VI compositions, Y is phenyl, naphthyl, 3-formylindole, imidazole, or pyrazole.

Additional compounds of the present invention include but are not limited to: (irreversible or suicide inhibitors): 2-Oxo-pentynoate, Dansyl chloride, Dansyl Fluoride, Propargylglycine, N-Acetyl-propargylglycine, O-(2,4-dinitrophenyl)hydroxylamine, 1-Chloro-2-nitroethane, 1,2-cyclohexadione, Allylglycine, N-chloro-D-leucine, Phenylglyoxal, Ethyl bromopyruvate, Methyl bromopyruvate, and Bromopyruvate. Further compounds include, but are not limited to: Methylglyoxal bis(guanylhydrazone), Hydrazinecarboximidamide, Pyruvaldehyde bis(amidinohydrazone), 3-(3-Indolyl)propanoic acid, 3-indole-acetic acid, Indole-3-acetone, Indole-3-acetamide, Indole-3-acetyl-L-aspartic acid, Indole-3-acetyl-L-alanine, Indole-3-acetylglycine, Indole-3-carboxylic acid, Indole-3-pyruvic acid, dansyl glycine, Alanine tetrazole, tetrazole, Riboflavin 5′-pyrophosphate, 5-Hydroxy-2-hydroxymethyl-4-pyranone, Hydroxylamine Hydrochloride, Tetrahydro-4-phenyl-4H-1,4-thiazine 1-oxide, Phenothiazine, 3,4-Dihydro-2H-1,4-thiazine-3,5-dicarboxylic acid, Nifurtimox (1-((5-Nitrofurfurylidene)amino)-2-methyltetrahydro-1,4-thiazine-4,4-dioxide), 2-amino-2,4-pentadienoate, 2-amino-4-keto-2-pentenoate, N-Acetyl-D-leucine, D-Leu (D-2-Amino-4-methylpentanoic acid, Progesterone (4-Pregnene-3,20-dione, FAD (Flavin adenine dinucleotide), 6-OH-FAD (Flavin adenine dinucleotide), N-(1-carboxyethyl)-L-Alanine, 3-Aminoaspartic acid, thiocarbamoyl hydrazide, 5-S-Cysteinyldopamine , phosphatidyl serine, 4-Hydroxy-2-mercapto 6-methylpyrimidine, 4-Amino—N-2-thiazolylbenzenesulfonamide, Thiocyanate, 2-Mercapto-l-methylimidazole, tartaric acid, 2-Aminoethanethiol, S-adenosylmethionine, and Thiourea.

The non-limiting listing of antagonists listed herein, or in Table I, may be altered or derivatized utilizing methods known in the art to produce one or more of the following compounds: a) a prodrug; b) a compound with greater enzymatic activity; c) a compound with more specificity for DAO or DDO; d) a compound with lower toxicity; or e) a compound lacking unwanted side effects. Methods for measuring DAO or DDO activity are well known in the art and may be performed using methods disclosed herein or disclosed in a reference cited herein. All of the references cited below for the exemplary DAO or DDO antagonists are incorporated by reference herein in their entireties.

Exemplary DAO or DDO Antagonists

a) 2-oxo-3-pentynoate; (Biochemistry May 4, 1999; 38 (18):5822-8).

b) Aminoguanidine; (J Neurochem 1998 March;70(3): 1323-6).

c) Benzoate (benzoic acid) and salts thereof (e.g., sodium benzoate); (Neurosci Lett Nov. 8, 1996;218(3):145-8).

d) o-, m-, and p-aminobenzoate (J. Biochem. (Tokyo) 1976 November, 80(5): 1101-1108)

e) Methylglyoxal bis(guanylhydrazone) (MGBG); phenylglyoxal bis(guanylhydrazone) (PhGBG); glyoxal bis(guanylhydrazone) (GBG); (Anticancer Drug Des Oct. 11, 1996;11(7):493-508).

f) Alpha-keto acids that are analogs of the amino acids alanine, valine, leucine, phenylanaline, phenylglycine, tyrosine, tryptophan; serine, aspartate; etc. (e.g., pyruvic acid, alpha-ketoisovaleric acid, 4-methylthio-2-oxopentanoic acid, 4-methylthio-2-oxybutanoic acid, phenylpyruvic acid, indol-3-pyruvic acid, benzoylformic acid, 4-hydroxyphenyl pyruvic acid, and salts and derivatives thereof), indole-propionic, 3-indole-acetic acid, salicylic acid, and salts and derivatives thereo (Enzyme Microb Technol Apr. 18, 1996;18(5):379-82).

g) Dansyl chloride; (FEBS Lett Arp. 24, 1995;363(3):307-10).

h) Alanine tetrazole and benzoic tetrazole; (Res Commum Chem Pathol Pharmacol 1994 February;83(2):209-22).

i) Riboflavin 5′-pyrophosphate (RPP); (Anal Biochem May 1, 1992;202(2):348-55).

j) D-propargylglycine (D-PG); (J Biochem (Tokyo) 1991 January;109(1):171-7).

k) D,L-beta-hydroxybutyrate; (J Histochem Cytochem 1991 January;39(1):81-6).

l) Trigonelline, i.e., N-methylnicotinate; (J Biochem (Tokyo) 1990 May;107(5):726-31).

m) Kojic acid and salts thereof; (J Biol Chem Feb.15, 1989;264(5):2509-17).

n) O-(2,4-dinitrophenyl)hydroxylamine; (Biochemistry Mar. 24, 1987;26(6):1717-22).

o) Benzoate; (D'Silva, C., Willams, C. H., Jr., & Massey, V. (1986) Biochemistry 25, 5602-5608).

p) Methyl-p-nitrobenzenesulfonate; (J Biol Chem May 10, 1984;259(9):5585-90).

q) Aminoethylcysteine-ketimine (2H-1,4-thiazine-5,6-dihydro-3-carboxylic acid); 1,4-thiazine derivatives, ketimine reduced forms (thiomorpholine-2-carboxylic acid and thiomorpholine-2,6-dicarboxylic acid); (Biochim Biophys Acta Oct. 17, 1983;748(1):40-7).

r) The reaction product between cysteamine and bromopyruvate; (J Appl Biochem 1983 August-October;5(4-5):320-9).

s) 1-chloro-1-nitroethane; (J Biol Chem Jan. 25, 1983;258(2):1136-41)

t) Benzoate; anthranilate; picolinate; L-leucine; (J Biol Chem Sep. 10, 1982;257(17):9958-62).

u) Fluorodinitrobenzene; (Nishino, T., Massey, V., and Williams, C. H., Jr. (1980) J Biol Chem 255, 3610-3616).

v) 1,2-cyclohexanedione; (Eur J Biochem 1981 October; 119(3):553-7).

w) Allylglycine; 2-amino-2,4-pentadienoate; 2-hydroxy-2,4-pentadienoate; (Biochemistry 1978 December 26;17(26):5620-6).

x) 2-amino-4-keto-2-pentenoate; (Biochemistry Dec. 26, 1978;17(26):5613-9).

y) D,L-2-hydroxybutyrate; (J Cell Biol 1978 April;77(1):59-71).

z) P-aminobenzoate; (J Bichem (Tokyo)1976 November;80(5):1073-83).

aa) N-chloro-D-leucine; (J Biol Chem Oct. 10, 1976;251(19):6150-3).

bb) D-propargyglycine; (Biochemistry Jul. 13, 1976;15(14):3070-6).

cc) D-2-amino-4-pentynoic acid (D-propargylglycine); (J Biochem (Tokyo) 1975 July; 78(1):57-63).

dd) Progesterone (Biochim Biophys Acta Jan. 12, 1978;522(1):43-8).

ee) Long chain, medium chain and short chain free fatty acids (Biochem Int 1990 December;22(5):837-42).

ff) 6-OH-FAD (Biochim Biophys Acta Apr. 12, 1999;1431(1):212-22).

gg) Phenylglyoxal, L-tartrate (Eur J Biochem Apr. 1, 1992;205(1): 127-32).

hh) Cyclothionine, TMDA, alpha-alpha′-iminodipropionic (Physiol Chem Phys Med NMR 1986;18(1):71-4).

ii) Inhibitors disclosed in J Histochem Cytochem 1990 September;38(9):1377-81.

jj) Meso-Diaminosuccinic aci(Eur J Biochem 1981 July;117(3):635-8).

kk) Thiosemicarbazide, thiourea, methylthiouracyl, sulphathiazole, thiocyanate, and methimazole (Endocrinol Exp 1976; 10(4):243-51).

ll) Dicarboxylic hydroxyacids (Enzymologia Dec. 31, 1967;33(6):325-30). mm) Malic and tartaric acid (Boll Soc Ital Biol Sper Oct. 31, 1966;42(20):1455-7).

It should be appreciated that DAO and DDO antagonists of the invention include the compounds listed above and throughout the specification, as well as the salts and derivatives thereof these compounds.

Methods of Screening for Compounds Modulating DAO or DDO Expression and/or Activity

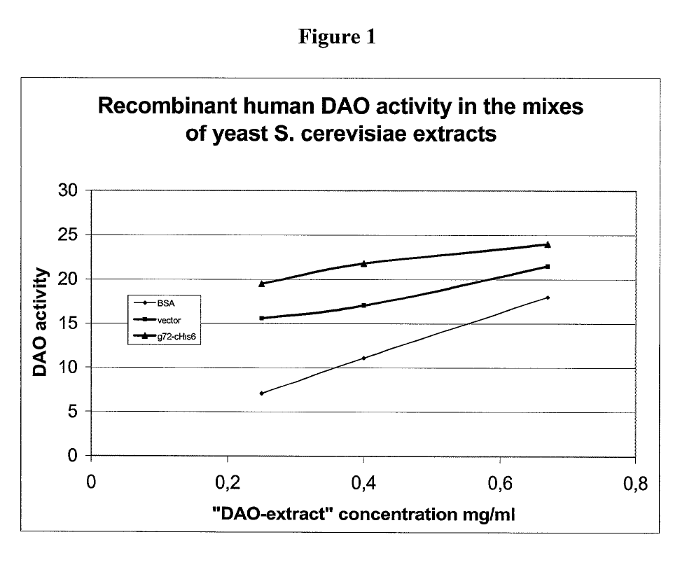

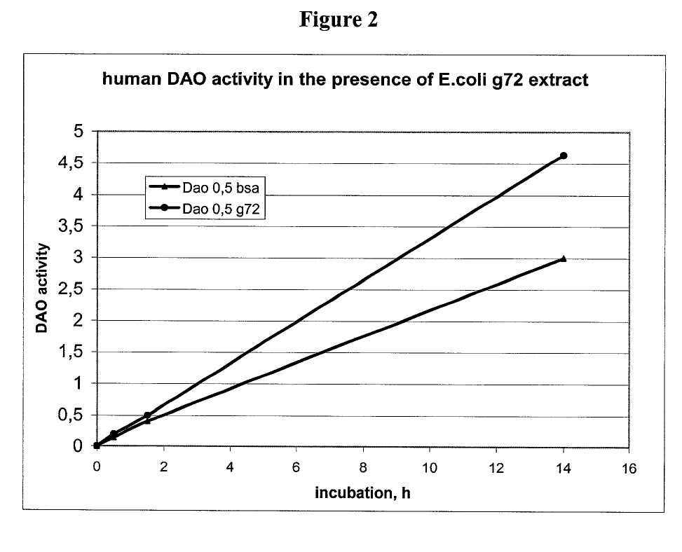

Methods that can be used for testing antagonistic compounds for their ability to inhibit or decrease the activity of a DAO or DDO polypeptide or inhibit or decrease the expression of a DAO or DDO gene product (mRNA or polypetpide) are well known in the art. Suitable DAO and DDO polypeptides useful for methods of screening include both recombinant DAO and DDO or DAO and DDO polypeptides purified from tissue (e.g., hog kidneys). Preferred DAO and DDO polypeptides, and polynucleotides useful to make said polypeptides, are the human DAO and DDO sequences of FIGS. 1 and 2. Preferred antagonists of the present invention are antagonists of the polypeptides of FIGS. 1 and 2. Further preferred antagonists of the present invention inhibit the oxidative deamination of D-amino acids. Further preferred antagonists of the present invention inhibit the oxidative deamination of D-Serine or D-Aspartate. The assays described herein and known in the art for measuring DAO or DDO enzymatic activity can be performed either in vitro or in vivo.

Antagonists according to the present invention include naturally occurring and synthetic compounds and small molecules. Antagonists of the present invention may either block binding of DAO or DDO to either its cofactor, FAD, or substrate, or block enzymatic activity, e.g., oxidative deamination of D-amino acids. Whether any candidate antagonist of the present invention can enhance or inhibit DAO or DDO activity is determined using well known methods in the art for measuring DAO or DDO activity. One method for screening involves contacting a sample comprising a DAO or DDO polypeptide with a test compound and assaying DAO or DDO activity in the presence of a substrate. The level of DAO or DDO activity is compared to a sample that does not contain the test compound, whereby a decreased DAO or DDO level of activity over the standard indicates that the candidate compound is an antagonist of DAO or DDO. DAO or DDO activity can be measured as an isolated or purifed enzyme or in a biological sample comprising cells or tissue expressing DAO or DDO.

Alternatively, one of skill in the art can identify compounds that inhibit expression of a DAO or DDO gene product (mRNA or polypeptide). Cells expressing DAO or DDO (e.g., liver, kidney, or brain cells) are incubated in the presence and absence of the test compound. By measuring the expression level of a DAO or DDO gene product in the presence and absence of the test compound or the level of DAO or DDO activity in the presence and absence of the test compound, compounds can be identified that suppress expression of a DAO or DDO gene product. Alternatively, constructs comprising a DAO or DDO regulatory sequence operably linked to a reporter gene (e.g. luciferase, chloramphenicol acetyl transferase, LacZ, green fluorescent polypeptide, beta galactosidase, etc.) can be introduced into host cells and the effect of the test compounds on expression of the reporter gene detected. Cells suitable for use in the foregoing assays include, but are not limited to, cells having the same origin as tissues or cell lines in which the polypeptide is known to be expressed (e.g., kidney, liver and brain). The quantification of the expression of a DAO or DDO polypeptide may be realized either at the mRNA level (using for example Northen blots, RT-PCR, preferably quantitative RT-PCR with primers and probes specific for the DAO or DDO mRNA of interest) or at the polypeptide level (by measuring DAO or DDO enzymatic activity or by using polyclonal or monoclonal antibodies in immunoassays such as ELISA or RIA assays, Western blots, immunochemistry).

In other aspects, an assay is a cell-based assay in which a cell which expresses a DAO or DDO protein or biologically active portion thereof is contacted with a test compound and the ability of the test compound to inhibit, activate, or increase DAO or DDO activity determined. Determining the ability of the test compound to inhibit, activate, or increase DAO or DDO activity can be accomplished by monitoring the bioactivity of the DAO or DDO protein or biologically active portion thereof. Preferably, amino acid oxidation is monitored. The cell, for example, can be of mammalian origin, bacterial origin or a yeast cell. For example, in some embodiments, the cell can be a mammalian cell, bacterial cell or yeast cell.

The test compounds of the present invention can be obtained using any of the numerous approaches in combinatorial library methods known in the art, including: biological libraries; spatially addressable parallel solid phase or solution phase libraries; synthetic library methods requiring deconvolution; the one-bead one-compound library method; and synthetic library methods using affinity chromatography selection. The biological library approach is used with peptide libraries, while the other four approaches are applicable to peptide, non-peptide oligomer or small molecule libraries of compounds (Lam, K. S. (1997) Anticancer Drug Des. 12:145, the disclosure of which is incorporated herein by reference in its entirety).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233, the disclosures of which are incorporated herein by reference in their entireties.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devin (1990) Science 249:404-406); (Cwirla et al. (1990) Proc. Natl. Acad. Sci. 87:6378-6382); (Felici (1991) J. Mol. Biol. 222:301-310); (Ladner supra.), the disclosures of which are incorporated herein by reference in their entireties.

Determining the ability of the test compound to inhibit DAO or DDO activity can also be accomplished, for example, by coupling the DAO or DDO protein or biologically active portion thereof with a radioisotope or enzymatic label such that binding of the DAO or DDO protein or biologically active portion thereof to its cognate target molecule can be determined by detecting the labeled DAO or DDO protein or biologically active portion thereof in a complex. Preferably, a DAO or DDO ‘target molecule’ is a molecule with which a DAO or DDO protein binds or interacts in nature, such that DAO or DDO-mediated function is achieved. In one example, a DAO target molecule is a g34872 polypeptide. For example, compounds (e.g., DAO or DDO protein or biologically active portion thereof) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product. The labeled molecule is placed in contact with its cognate molecule and the extent of complex formation is measured. For example, the extent of complex formation may be measured by immuno precipitating the complex or by performing gel electrophoresis.

It is also within the scope of this invention to determine the ability of a compound (e.g., DAO or DDO protein or biologically active portion thereof) to interact with its cognate target molecule without the labeling of any of the interactants. For example, a microphysiometer can be used to detect the interaction of a compound with its cognate target molecule without the labeling of either the compound or the target molecule. McConnell, H. M. et al. (1992) Science 257:1906-1912, the disclosure of which is incorporated herein by reference in its entirety. A microphysiometer such as a cytosensor is an analytical instrument that measures the rate at which a cell acidities its environment using a light-addressable potentiometric sensor (LAPS). Changes in this acidification rate can be used as an indicator of the interaction between compound and receptor.

In a preferred embodiment, the assay comprises contacting a cell which expresses or which is reponsive to a DAO or DDO protein or biologically active portion thereof, with a target molecule to form an assay mixture, contacting the assay mixture with a test compound, and determining the ability of the test compound to inhibit or increase the activity of the DAO or DDO protein or biologically active portion thereof, wherein determining the ability of the test compound to inhibit or increase the activity of the DAO or DDO protein or biologically active portion thereof, comprises determining the ability of the test compound to inhibit or increase a biological activity of the DAO or DDO expressing cell (e.g., determining the ability of the test compound to inhibit or increase transduction, protein:protein interactions, substrate binding).

In another embodiment, an assay is a cell-based assay comprising contacting a cell expressing a DAO or DDO target molecule (i.e. a molecule with which DAO or DDO interacts) with a test compound and determining the ability of the test compound to modulate (e.g. stimulate or inhibit respectively) the activity of the DAO or DDO target molecule. Determining the ability of the test compound to modulate the activity of a DAO or DDO target molecule can be accomplished, for example, by determining the ability of the DAO or DDO protein to bind to or interact with the DAO or DDO target molecule.

Determining the ability of the DAO or DDO protein to bind to or interact with a DAO or DDO target molecule can be accomplished by one of the methods described above for determining direct binding. In a preferred embodiment, determining the ability of the DAO or DDO protein to bind to or interact with a DAO or DDO target molecule can be accomplished by determining the activity of the target molecule. For example, the activity of the target molecule can be determined by contacting the target molecule with the DAO or DDO protein or a fragment thereof and measuring induction of a cellular second messenger of the target (i.e. intracellular Ca2+, diacylglycerol, P3, etc.), detecting catalytic/enzymatic activity of the target an appropriate substrate, detecting the induction of a reporter gene (comprising a target-responsive regulatory element operatively linked to a nucleic acid encoding a detectable marker, e.g., luciferase), or detecting a target-regulated cellular response, for example, signal transduction or protein:protein interactions.

In other preferred embodiments, an assay of the present invention is a cell-free assay in which a DAO or DDO protein or biologically active portion thereof is contacted with a test compound and the ability of the test compound to bind to the DAO or DDO protein or biologically active portion thereof is determined. Binding of the test compound to the protein can be determined either directly or indirectly as described above. In a preferred embodiment, the assay includes contacting the DAO or DDO protein or biologically active portion thereof with a known compound which binds DAO or DDO (e.g., a DAO or DDO target molecule) to form an assay mixture, contacting the assay mixture with a test compound, and determining the ability of the test compound to interact with a DAO or DDO protein, wherein determining the ability of the test compound to interact with a DAO or DDO protein comprises determining the ability of the test compound to preferentially bind to DAO or DDO or biologically active portion thereof as compared to the known compound.

In another embodiment, the assay is a cell-free assay in which a DAO or DDO protein or biologically active portion thereof is contacted with a test compound and the ability of the test compound to modulate (e.g., inhibit the activity of the DAO or DDO or activate the activity of the DAO or DDO) the activity of the protein or biologically active portion thereof is determined. Determining the ability of the test compound to modulate the activity of a protein can be accomplished, for example, by determining the ability of the protein to bind to a target molecule by one of the methods described above for determining direct binding. This can also be accomplished for example using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705, the disclosures of which are incorporated herein by reference in their entireties. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a DAO or DDO protein can be accomplished by determining the ability of the DAO or DDO protein to further modulate the activity of a downstream effector a DAO or DDO target molecule. For example, the activity of the effector molecule on an appropriate target can be determined or the binding of the effector to an appropriate target can be determined as previously described.

In yet another embodiment, the cell-free assay involves contacting a DAO or DDO protein or biologically active portion thereof with a known compound which binds the DAO or DDO protein to form an assay mixture, contacting the assay mixture with a test compound, and determining the ability of the test compound to interact with the DAO or DDO protein, wherein determining the ability of the test compound to interact with the DAO or DDO protein comprises determining the ability of the DAO or DDO protein to preferentially bind to or modulate the activity of a DAO or DDO target molecule.

The cell-free assays of the present invention are amenable to use of both soluble and/or membrane-bound forms of isolated proteins (e.g. DAO or DDO proteins or biologically active portions thereof or molecules to which DAO or DDO targets bind). In the case of cell-free assays in which a membrane-bound form an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl—N-methylglucamide, decanoyl-N-methylglucamide, Triton™ X-100, Triton™ X-114, Thesit™, Isotridecypoly(ethylene glycol ether)n,3-(3-cholamidopropyl)dimethylamminio-1-propane sulfonate (CHAPS), 3-(3-cholamidopropyl)dimethylamminio-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either a DAO or DDO protein or a target molecule to facilitate separation of complexed from uncomplexed forms of one or both of the proteins, as well as to accommodate automation of the assay. Binding of a test compound to a DAO or DDO protein, or interaction of a DAO or DDO protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/DAO or DDO fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or DAO or DDO protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of DAO or DDO binding or activity determined using standard techniques.

Other techniques for immobilizing proteins on matrices can also be used in the screening assays of the invention. For example, either a DAO or DDO protein or a DAO or DDO target molecule can be immobilized utilizing conjugation of biotin and streptavidin. Biotinylated DAO or DDO protein or target molecules can be prepared from biotin—NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with DAO or DDO protein or target molecules but which do not interfere with binding of the DAO or DDO protein to its target molecule can be derivatized to the wells of the plate, and unbound target or DAO or DDO protein trapped in the wells by antibody conjugation. Methods for detecting such complexes, in addition to those described above for the GST-immobilized complexes, include immunodetection of complexes using antibodies reactive with the DAO or DDO protein or target molecule, as well as enzyme-linked assays which rely on detecting an enzymatic activity associated with the DAO or DDO protein or target molecule.

In yet another aspect of the invention, the proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300, the disclosures of which are incorporated herein by reference in their entireties), to identify other proteins which bind to or interact with the DAO or DDO proteins, and/or are involved in the activity of the DAO or DDO proteins.

The two-hybrid system is based on the modular nature of most transcription factors, which consist of separable DNA-binding and activation domains. Briefly, the assay utilizes two different DNA constructs. In one construct, the gene that codes for a DAO or DDO protein or a fragment thereof is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a DAO or DDO-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the DAO or DDO protein.

This invention further pertains to novel agents identified by the above-described screening assays and to processes for producing such agents by use of these assays. Accordingly, in one embodiment, the present invention includes a compound or agent obtainable by a method comprising the steps of any one of the aformentioned screening assays (e.g., cell-based assays or cell-free assays). For example, in one embodiment, the invention includes a compound or agent obtainable by a method comprising contacting a cell which expresses a DAO or DDO target molecule with a test compound and the determining the ability of the test compound to bind to, or modulate the activity of, the DAO or DDO target molecule. In another embodiment, the invention includes a compound or agent obtainable by a method comprising contacting a cell which expresses a DAO or DDO target molecule with a DAO or DDO protein or biologically-active portion thereof, to form an assay mixture, contacting the assay mixture with a test compound, and determining the ability of the test compound to interact with, or modulate the activity of, the DAO or DDO target molecule. In another embodiment, the invention includes a compound or agent obtainable by a method comprising contacting a DAO or DDO protein or biologically active portion thereof with a test compound and determining the ability of the test compound to bind to inhibit the activity of, the DAO or DDO protein or biologically active portion thereof. In yet another embodiment, the present invention included a compound or agent obtainable by a method comprising contacting a DAO or DDO protein or biologically active portion thereof with a known compound which binds the DAO or DDO protein to form an assay mixture, contacting the assay mixture with a test compound, and determining the ability of the test compound to interact with, or modulate the activity of the DAO or DDO protein.

Antagonist compounds that inhibit DAO or DDO activity or inhibit expression of a DAO or DDO gene product can also be identified using in vivo screens. In these assays, the test compound is administered (e.g. IV, IP, IM, orally, or otherwise), to the animal, for example, at a variety of dose levels. The effect of the test compound on DAO or DDO activity or gene product expression is determined by comparing the levels of DAO or DDO activity or gene product expression, respectively, in the tissues of test and control animals that express DAO or DDO. Suitable test animals include, but not limited to, rodents (e.g., mice and rats) and primates. Humanized non-human animals, such as humanized mice, can also be used as test animals, that is, animals in which the endogenous polypeptide is ablated (knocked out) and the homologous human polypeptide added back by standard transgenic approaches. Such animals express only the human form of a polypeptide.

In vivo assays also include animal models for CNS disorders. These models include, but are not limited to: conditioned avoidance behavior in rats model; gerbil foot-tapping model; ferret emesis model; separation-induced vocalization model; behavioral activity assessment of mice and rats in the omnitech digiscan animal activity monitors; blockade of amphetamine-stimulated locomotion in rats model; prepulse inhibition (PPI) of acoustic startle in rats model; inhibition of apomorphine-induced climbing behaviour model; and the DOI-induced head twitches and scratches model as described herein and known in the art.

Other antagonists of the present invention include antisense and triple helix tools to inhibit expression of a DAO or DDO gene product. In antisense approaches, nucleic acid sequences complementary to a DAO or DDO mRNA or genomic sequence are hybridized to the DAO or DDO mRNA or genomic DNA intracellularly, thereby blocking the expression of the DAO or DDO polypeptide encoded by the mRNA. The antisense nucleic acid molecules to be used in DAO or DDO therapy may be either DNA or RNA sequences. Preferred methods using antisense polynucleotide according to the present invention are the procedures described by Sczakiel et al.(l995), which disclosure is hereby incorporated by reference in its entirety. Other preferred antisense polynucleotides according to the present invention are sequences complementary to either a sequence of DAO or DDO mRNAs comprising the translation initiation codon ATG or a sequence of DAO or DDO.

It is preferable that the antisense polynucleotides comprise sequences complementary to a DAO or DDO initiation codon (ATG) or genomic DNA containing a splicing donor or acceptor site. It is also preferable that the antisense polynucleotides of the invention have a 3′ polyadenylation signal that has been replaced with a self-cleaving ribozyme sequence, such that RNA polymerase II transcripts are produced without poly(A) at their 3′ ends, these antisense polynucleotides being incapable of export from the nucleus, such as described by Liu et al.(1994), which disclosure is hereby incorporated by reference in its entirety. The DAO or DDO antisense polynucleotides may also comprise, within the ribozyme cassette, a histone stem-loop structure to stabilize cleaved transcripts against 3′-5′ exonucleolytic degradation, such as the structure described by Eckner et al.(1991), which disclosure is hereby incorporated by reference in its entirety.

The antisense nucleic acids should have a length and melting temperature sufficient to permit formation of an intracellular duplex having sufficient stability to inhibit the expression of the DAO or DDO mRNA in the duplex. Strategies for designing antisense nucleic acids suitable for use in DAO or DDO therapy are disclosed in Green et al., (1986) and Izant and Weintraub, (1984), the disclosures of which are incorporated herein by reference.

In some strategies, antisense molecules are obtained by reversing the orientation of the DAO or DDO coding region with respect to a promoter so as to transcribe the opposite strand from that which is normally transcribed in the cell. Another approach involves transcription of DAO or DDO antisense nucleic acids in vivo by operably linking DNA containing the antisense sequence to a promoter in a suitable expression vector.

Alternatively, oligonucleotides which are complementary to the strand normally transcribed in the cell may be synthesized in vitro. Thus, the antisense nucleic acids are complementary to the corresponding mRNA and are capable of hybridizing to the mRNA to create a duplex. The antisense sequences may also contain modified sugar phosphate backbones to increase stability and make them less sensitive to RNase activity. Examples of modifications suitable for use in antisense strategies include 2′ O-methyl RNA oligonucleotides and polypeptide-nucleic acid (PNA) oligonucleotides. Further examples are described by Rossi et al., (1991), which disclosure is hereby incorporated by reference in its entirety.

Various types of antisense oligonucleotides complementary to the sequence of the DAO or DDO cDNA or genomic DNA may be used. For example, stable and semi-stable antisense oligonucleotides described in International Application No. PCT WO94/23026, hereby incorporated by reference, can be used. In these molecules, the 3′ end or both the 3′ and 5′ ends are engaged in intramolecular hydrogen bonding between complementary base pairs. These molecules are better able to withstand exonuclease attacks and exhibit increased stability compared to conventional antisense oligonucleotides.

In yet another method of using antisense technology to inhibit expression of a DAO or DDO polypeptide, the covalently cross-linked antisense oligonucleotides described in International Application No. WO 96/31523, hereby incorporated by reference, is used. These double- or single-stranded oligonucleotides comprise one or more, respectively, inter- or intra-oligonucleotide covalent cross-linkages, wherein the linkage consists of an amide bond between a primary amine group of one strand and a carboxyl group of the other strand or of the same strand, respectively, the primary amine group being directly substituted in the 2′ position of the strand nucleotide monosaccharide ring, and the carboxyl group being carried by an aliphatic spacer group substituted on a nucleotide or nucleotide analog of the other strand or the same strand, respectively.

The antisense oligodeoxynucleotides and oligonucleotides disclosed in International Application No. WO 92/18522, incorporated by reference, may also be used. These molecules are stable to degradation and contain at least one transcription control recognition sequence which binds to control polypeptides and are effective as decoys therefor. These molecules may contain “hairpin” structures, “dumbbell” structures, “modified dumbbell” structures, “cross-linked” decoy structures and “loop” structures.

Further, the cyclic double-stranded oligonucleotides described in European Patent Application No. 0 572 287 A2, hereby incorporated by reference may be used. These ligated oligonucleotide “dumbbells” contain the binding site for a transcription factor and inhibit expression of the DAO or DDO under control of the transcription factor by sequestering the factor.

Use of the closed antisense oligonucleotides disclosed in International Application No. WO 92/19732, hereby incorporated by reference, is also an alternative. Because these molecules have no free ends, they are more resistant to degradation by exonucleases than are conventional oligonucleotides. These oligonucleotides may be multifunctional, interacting with several regions which are not adjacent to the target mRNA.

The appropriate level of antisense nucleic acids required to inhibit DAO or DDO expression may be determined using in vitro expression analysis. The antisense molecule may be introduced into the cells by diffusion, injection, infection or transfection using procedures known in the art. For example, the antisense nucleic acids can be introduced into the body as a bare or naked oligonucleotide, oligonucleotide encapsulated in lipid, oligonucleotide sequence encapsidated by viral polypeptide, or as an oligonucleotide operably linked to a promoter contained in an expression vector. The expression vector may be any of a variety of expression vectors known in the art, including retroviral or viral vectors, vectors capable of extrachromosomal replication, or integrating vectors. The vectors may be DNA or RNA.

The antisense molecules are introduced onto cell samples at a number of different concentrations preferably between 1×10⁻¹M to 1×10⁻⁴M. Once the minimum concentration that can adequately control DAO or DDO expression is identified, the optimized dose is translated into a dosage suitable for use in vivo. For example, an inhibiting concentration in culture of 1×10⁻⁷ translates into a dose of approximately 0.6 mg/kg bodyweight. Levels of oligonucleotide approaching 100 mg/kg bodyweight or higher may be possible after testing the toxicity of the oligonucleotide in laboratory animals. It is additionally contemplated that cells from the vertebrate are removed, treated with the antisense oligonucleotide, and reintroduced into the vertebrate.

In a preferred application of this invention, the polypeptide encoded by the DAO or DDO is first identified or the enzymatic activity measured, so that the effectiveness of antisense inhibition on translation can be monitored using techniques that include but are not limited to antibody-mediated tests such as RIAs and ELISA, functional assays, or radiolabeling, and assays to measure DAO or DDO activity.

An alternative to the antisense technology that is used according to the present invention to inhibit expression of a DAO or DDO gene product comprises using ribozymes that will bind to a DAO or DDO target sequence via their complementary polynucleotide tail and that will cleave the corresponding DAO or DDO RNA by hydrolyzing its target site (namely “hammerhead ribozymes”). Briefly, the simplified cycle of a hammerhead ribozyme comprises (1) sequence specific binding to the target DAO or DDO RNA via complementary antisense sequences; (2) site-specific hydrolysis of the cleavable motif of the target DAO or DDO strand; and (3) release of cleavage products, which gives rise to another catalytic cycle. The construction and production of hammerhead ribozymes is well known in the art and is described more fully in Haseloff and Gerlach, Nature 20334:585-591 (1988). Indeed, the use of long-chain antisense polynucleotide (at least 30 bases long) or ribozymes with long antisense arms are advantageous. A preferred delivery system for antisense ribozyme is achieved by covalently linking these antisense ribozymes to lipophilic groups or to use liposomes as a convenient vector. Preferred antisense ribozymes according to the present invention are prepared as described by Rossi et al, (1991) and Sczakiel et al.(1995), the specific preparation procedures being referred to in said articles being herein incorporated by reference.

The DAO or DDO genomic DNA may also be used to inhibit the expression of the DAO or DDO based on intracellular triple helix formation. Triple helix oligonucleotides are used to inhibit transcription from a genome. They are particularly useful for studying alterations in cell DAO or DDO activity. The DAO or DDO cDNAs or genomic DNA or a fragment of those sequences, can be used to inhibit DAO or DDO expression in individuals having a CNS disorder associated with expression of a particular DAO or DDO. Similarly, a portion of the DAO or DDO genomic DNA can be used to study the effect of inhibiting DAO or DDO transcription within a cell. Traditionally, homopurine sequences are considered the most useful for triple helix strategies. However, homopyrimidine sequences may also be used to inhibit DAO or DDO expression. Such homopyrimidine oligonucleotides bind to the major groove at homopurine:homopyrimidine sequences.

To carry out DAO or DDO therapy strategies using the triple helix approach, the sequences of the DAO or DDO genomic DNA are first scanned to identify 10-mer to 20-mer homopyrimidine or homopurine stretches which could be used in triple-helix based strategies for inhibiting DAO or DDO expression. Following identification of candidate homopyrimidine or homopurine stretches, their efficiency in inhibiting DAO or DDO expression is assessed by introducing varying amounts of oligonucleotides containing the candidate sequences into tissue culture cells which express the DAO or DDO. Treated cells are monitored for altered DAO or DDO enzymatic activity or reduced DAO or DDO expression as described above.

The oligonucleotides which are effective in inhibiting DAO or DDO expression in tissue culture cells may then be introduced in vivo using the techniques and at a dosage calculated based on the in vitro results, as described for antisense polynucleotides.

In some embodiments, the natural (beta) anomers of the oligonucleotide units can be replaced with alpha anomers to render the oligonucleotide more resistant to nucleases. Further, an intercalating agent such as ethidium bromide, or the like, can be attached to the 3′ end of the alpha oligonucleotide to stabilize the triple helix. For information on oligonucleotides suitable for triple helix formation see Griffin et al. (Science 245:967-71, 1989), which is hereby incorporated by this reference.

Pharmaceutical and Physiologically Acceptable Compositions and Administration Thereof

The compounds and compositions for use in the invention can be prepared utilizing readily available starting materials and employing common synthetic methodologies well-known to those skilled in the art. Alternatively, compounds useful in the practice of the invention can be purchased from commercial vendors, such as Sigma Chemical Company (St. Louis, Mo.).

The relative activity, potency and specificity of a DAO or DDO antagonist can be determined by a pharmacological study in animals according to the method of Nyberg et al. [Psychopharmacology 119, 345-348 (1995)],described herein, or known in the art. The test provides an estimate of relative activity, potency and, through a measure of specificity, an estimate of therapeutic index. Other animal studies which may be used include, but are not limited to, studies involving conditioned avoidance, apomorphine induced climbing, blockade of 5-hydroxy-tryptophan-induced head twitching and other animal models disclosed herein or known in the art. Although the differential metabolism among patient populations can be determined by a clinical study in humans, less expensive and time-consuming substitutes are provided by the methods of Kerr et al. [Biochem. Pharmacol. 47, 1969-1979 (1994)] and Karam et al. [Drug Metab. Dispos. 24, 1081-1087 (1996)]. Similarly, the potential for drug-drug interactions may be assessed clinically according to the methods of Leach et al. [Epilepsia 37, 1100-1106 (1996)] or in vitro according to the methods of Kerr et al.[op. cit.] and Turner and Renton [Can. J. Physiol. Pharmacol. 67, 582-586 (1989)]. In addition, the relative activity, potency and specificity of a DAO or DDO antagonist may be tested using various in vitro assays.

The effective dose can vary, depending upon factors such as the condition of the patient, the severity of the symptoms of the disorder, and the manner in which the pharmaceutical composition is administered. For human patients, the effective dose of typical compounds generally requires administering the compound in an amount of at least about 1, often at least about 10, and frequently at least about 25 mg/24 hr./patient. For human patients, the effective dose of typical compounds requires administering the compound which generally does not exceed about 500, often does not exceed about 400, and frequently does not exceed about 300 mg/24 hr./patient. In addition, administration of the effective dose is such that the concentration of the compound within the plasma of the patient normally does not exceed 500 ng/ml, and frequently does not exceed 100 ng/ml.

The compounds and compositions of the present invention can be administered to a patient at dosage levels in the range of about 0.1 to about 1,000 mg per day. For a normal human adult having a body weight of about 70 kilograms, it is estimated that a dosage in the range of about 0.01 to about 100 mg per kilogram of body weight per day is sufficient. The specific dosage used, however, can vary. For example, the dosage can depend on a numbers of factors including the requirements of the patient, the severity of the condition being treated, and the pharmacological activity of the compound being used. The determination of optimum dosages for a particular patient is well-known to those skilled in the art. The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

In some embodiments, various combinations of DAO or DDO antagonists can be used in the practice of the invention. Thus, compositions containing more than one DAO antagonist can be used to in therapeutic methodologies according to the invention. Alternatively, compositions containing more than one DDO antagonist can be used in the disclosed methodologies. In yet another embodiment, combinations of at least one DAO antagonist and at least one DDO antagonist can be used in treatment methodologies disclosed herein.

Preferred compounds useful according to the method of the present invention have the ability to pass across the blood-brain barrier of the patient. As such, such compounds have the ability to enter the central nervous system of the patient. The log P values of typical compounds useful in carrying out the present invention generally are greater than 0, often are greater than about 1, and frequently are greater than about 1.5. The log P values of such typical compounds generally are less than about 4, often are less than about 3.5, and frequently are less than about 3. Log P values provide a measure of the ability of a compound to pass across a diffusion barrier, such as a biological membrane. See, Hansch, et al., J. Med. Chem., Vol. 11, p. 1 (1968). Alternatively, the compositions of the present invention can bypass the blood brain barrier through the use of compositions and methods known in the art for bypassing the blood brain barrier (e.g., U.S. Pat. Nos. 5,686,416; 5,994,392, incorporated by reference in their entireties) or can be injected directly into the brain. Suitable areas for injection include the cerebral cortex, cerebellum, midbrain, brainstem, hypothalamus, spinal cord and ventricular tissue, and areas of the PNS including the carotid body and the adrenal medulla. The compositions can be administered as a bolus or through the use of other methods such as an osmotic pump.

The compounds of the present invention can be administered to a patient alone or as part of a composition that contains other components such as excipients, diluents, and carriers, all of which are well-known in the art. The compositions can be administered to humans and animals either orally, rectally, parenterally (intravenous, by intramuscularly or subcutaneously), intracisternally, intravaginally, intraperitoneally, intravesically, locally (powders, ointments or drops), or as a buccal or nasal spray, or inhaled.

Compositions suitable for parenteral injection can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one customary inert excipient (or carrier) such as sodium citrate or dicalcium phosphate or (a) fillers or extenders, as for example, starches, lactose, sucrose, glucose, mannitol, and silicic acid; (b) binders, as for example, carboxymethylcellulose, alignates, gelatin, polyvinylpyrrolidone, sucrose and acacia; (c) humectants, as for example, glycerol; (d) disintegrating agents, as for example, agar-agar, calcium carbonate, potato or tapioca starch, alginic acid, certain complex silicates and sodium carbonate; (e) solution retarders, as for example paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethylene glycols, and the like. lid dosage forms such as tablets, dragees, capsules, pills, and granules can be prepared with coatings and shells, such as enteric coatings and others well-known in the art. They may contain opacifying agents and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan or mixtures of these substances, and the like. Besides such inert diluents, the composition can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are preferably suppositories which can be prepared by mixing the compounds of the present invention with suitable nonirritating excipients or carriers such as cocoa butter, polyethylene glycol or a suppository wax, which are solid at ordinary temperatures but liquid at body temperature and therefore, melt in the rectum or vaginal cavity and release the active component.

Dosage forms for topical administration of a compound of this invention include ointments, powders, sprays, and inhalants. The active component is admixed under sterile conditions with a physiologically acceptable carrier and any preservative, buffers, or propellants as may be required. Ophthalmic formulations, eye ointments, powders, and solutions are also contemplated as being within the scope of this invention.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

Animal Models

Conditioned Avoidance Behavior in Rats

The conditioned avoidance model is a standard behavioural test predictive of antipsychotic activity. One of the major pharmacological properties of currently employed clinical antipsychotic drugs in animals is their ability to block conditioned avoidance responding. See e.g., Cook, L. and Davidson, A. B.: Behavioral pharmacology: Animal models involving aversive control of behavior. In Psychopharmacology, A Generation of Progress, ed by M. A. Lipton, A. Dimascio and K. Killam, pp. 563-567, Raven Press, New York, 1978; Davidson, A. B. and Weidley, E. Differential Effects of Neuroleptic and other Psychotropic Agents on Acquisition of Avoidance in Rats, 18 Life Sci. 1279-1284 (1976), incorporated by reference herein in their entireties. There is a high correlation between their activity and potency on a conditioned avoidance test and their clinical efficacy and potencies as antipsychotic drugs. See e.g., Creese, I., Burt, D. R. and Snyder, S. H.: Dopamine receptor binding predicts clinical and pharmacological properties of antischizophrenic drugs. Science (Washington D.C.) 192:481-483,1976, incorporated by reference herein in its entirety.

In a conditioned avoidance test, animals learn to respond during a conditioned stimulus in order to avoid mild shock presentation. A response during the conditioned stimulus is termed an avoidance respone, a response during shock is termed an escape response; a response failure is when the animal fails to respond during either the conditioned stimlus or the shock presentation and is indicative of motor impairment. Animals rapidly learn to avoid 99% of the time. Antipsychotic drugs decrease the percentage of avoidance responses without interfering with the ability of the animal to respond since the animals do emit escape responses. The percentage of response failures is considered a measure of motor impairment.

Rats are required to press a response lever in an experimental chamber in order to avoid or escape foot-shock. Each experimental session consists of 50 trials. During each trial, the chamber is illuminated and a tone presented for a maximum of 10 sec. A response during the tone immediately terminates the tone and the houselight, ending the trial. In the absence of a response during the tone alone, tone+foot-shock (2.0 mA) is presented for a maximum of 10 sec. A response during shock presentation immediately terminates the shock, the tone and the houselight, ending the trial.

For drug screening, an appropriate dose, e.g., 3.0 mg/kg, is administered in an appropriate manner, e.g., i.p. or s.c., for an appropriate time, 30 min, before the start of the experimental session. The treated group may receive only a single dose of the DAO or DDO antagonist or alternatively, may be treated daily (e.g., sid, bid, or tid) for at least 1 day, 3 days, 1 week, 2 weeks, 1 month, or 2 months, prior to the experiment. A drug is considered active if it reduces the % avoidance responding to at least 50% without producing greater than 50% response failures. For active drugs, a dose-response curve is subsequently determined.

Gerbil Foot-Tapping

Male or female Mongolian gerbils (35-70 g) are anaesthetised by inhalation of an isoflurane/oxygen mixture to permit exposure of the jugular vein in order to permit administration of test or control compounds or vehicle in an injection volume of 5 ml/kg i.v. Alternatively, test compounds may be administered orally or by subcutaneous or intraperitoneal routes. The treated group may receive only a signle dose of the test compound or may be treated daily (e.g., sid, bid, or tid) for at least 1 day, 3 days, 1 week, 2 weeks, 1 month, or 2 months, prior to the assay. A skin incision is then made in the midline of the scalp to expose the skull. An anxiogenic agent (e.g. pentagastrin) and/or a control agent (saline, DAO or DDO antagonist, D-Ser, D-Asp, etc.) is infused directly into the cerebral ventricles (e.g. 3 pmol in 5 .mu.1 i.c.v., depending on test substance) by vertical insertion of a cuffed 27 gauge needle to a depth of 4.5 mm below bregma. The scalp incision is closed and the animal allowed to recover from anaesthesia in a clear perspex observation box (25 cm.times.20 cm.times.20cm). The duration and/or intensity of hind foot tapping is then recorded continuously for approximately 5 minutes. Alternatively, the ability of test compounds to inhibit foot tapping evoked by aversive stimulation, such as foot shock or single housing, may be studied using a similar method of quantification. Preferred antagonists of the present invention are able to inhibit induced foot-tapping in the gerbil.

Ferret Emesis

Individually housed male ferrets (1.0-2.5 kg) are dosed orally by gavage with test or control compounds or vehicle. Ten minutes later they are fed with approximately 100 g of tinned cat food. The treated group may receive only a single dose of the test compound or may be treated daily (e.g., sid, bid, or tid) for at least 1 day, 3 days, 1 week, 2 weeks, 1 month, or 2 months, prior to the experiment. At 60 minutes following oral dosing, cisplatin (10 mg/kg) is given i.v. via a jugular vein catheter inserted under a brief period of halothane anaesthesia. The catheter is then removed, the jugular vein ligated and the skin incision closed. The ferrets recover rapidly from the anaesthetic and are mobile within 10-20 minutes. The animals are observed continuously during recovery from the anaesthetic and for 4 hours following the cisplatin injection, after which time the animals are killed humanely. The numbers of retches and vomits occurring during the 4 hours after cisplatin administration are recorded by trained observers.

Separation-Induced Vocalisation

Male and female guinea-pigs pups are housed in family groups with their mothers and littermates throughout the study. Experiments are commenced after weaning when the pups are 2 weeks old. Before entering an experiment, the pups are screened to ensure that a vigorous vocalisation response is reproducibly elicited following maternal separation. The pups are placed individually in an observation cage (55 cm.times.39 cm.times.19 cm) in a room physically isolated from the home cage for 15 minutes and the duration of vocalisation during this baseline period is recorded. Only animals which vocalise for longer than 5 minutes are employed for drug challenge studies (approximately 50% of available pups may fail to reach this criterion). The treated group may receive only a single dose of the test compound or may be treated daily (e.g., sid, bid, or tid) for at least 1 day, 3 days, 1 week, 2 weeks, 1 month, or 2 months, prior to the experiment. On test days each pup receives an oral dose or an s.c. or i.p. injection of test compound or vehicle and is then immediately returned to the home cage with its mother and siblings for 30 to 60 minutes (or for up to 4 hours following an oral dose, dependent upon the oral pharmacokinetics of the test compound) before social isolation for 15 minutes as described above. The duration of vocalisation on drug treatment days is expressed as a percentage of the pre-treatment baseline value for each animal. The same subjects are retested once weekly for up to 6 weeks. Between 6 and 8 animals receive each test compound at each dose tested. Preferred antagonists of the present invention are effective in the attenuation of separation-induced vocalisations by guinea-pig pups as hereinafter defined.

Behavioral Activity Assessment of Mice and Rats in the Omnitech Digiscan Animal Activity Monitors

The purpose of this test is to evaluate compounds for antipsychotic-like central nervous system (CNS) effects and a variety of other behavioral effects generally associated with CNS activity. This test has the capacity to determine drug effects on many aspects of locomotor activity in rodents, including horizontal activity (beam breaks), total distance traveled (in cm), number of movements, movement time (in sec), rest time (in sec), vertical activity (beam breaks), number of vertical movements, vertical time (in sec), stereotypy counts, number of stereotypic episodes, stereotypy time (in sec), margin and center time (in sec), clockwise and counterclockwise revolutions, and time (in sec) spent in each corner of the activity monitor. Generally, however, drug effects on behavior are assessed using total distance traveled (in cm) as the most accurate measure of locomotor activity.

Male CD-1 albino mice weighing 20 to 40 g (Charles River Laboratories) or male Sprague-Dawley rats weighing 150 to 300 g (Harlan Laboratories) are used for these studies. The treated group may receive only a single dose of the test compound before the experiment or may be treated daily (e.g., sid, bid, or tid) for at least 1 day, 3 days, 1 week, 2 weeks, 1 month, or 2 months, prior to the experiment.

The Omnitech Digiscan animal activity monitor consists of a 16″.times.16″.times.12″ plexiglas cubicle enclosed inside 2 sets of 16 infrared photobeam sensors spaced 1 inch apart on all four sides of the bottom of the cubicle. An additional set of photobeam sensors are placed directly above the lower photobeam sensors, which measure vertical activity. Interruption of any beam should generate a flash of the LED indicator located in the center of the monitor mainframe. A diagnostic test of each of the 24 monitors is generally performed prior to the start of an experiment, in which all the photobeams are checked for any interruption. Each activity monitor can be divided into four 8″ square quadrants using a plexiglas insert that fits inside the plexiglas cubicle, of which 2 quadrants (front left and rear right) can be used for activity testing. Generally, this divided arrangement is utilized for mouse activity studies (2 mice per divided monitor) as opposed to rat studies (1 rat per undivided monitor). Up to 999 data samples can be taken for up to 999 minutes duration. Generally, 6 data samples of 10-minute duration each are collected for mice (1-hour test), or 6 samples of 5-minute duration for rats (30-minute test).

Once the animal is placed in the activity chamber, the chambers are individually activated to begin collecting data. Activity levels are generally monitored with the overhead lights turned off, as the dark-stimulation tends to produce less variation in the data. The following types of data (with brief definitions) are collected during each experiment:

Variable 1: Horizontal activity—total number of beam interruptions that occurred in the horizontal sensor.

Variable 2: Total distance (in cm) traveled—a more accurate indicator of ambulatory activity as it takes into account any diagonal movement.

Variable 3: Number of movements—number of discrete movements separated by at least 1 second.

Variable 4: Movement time (sec)—amount of time in ambulation.

Variable 5: Rest time (sec)—difference between sample time and time spent moving.

Variable 6: Vertical activity—total number of beam interruptions that occurred in the vertical sensor as the animal rears up.

Variable 7: Number of vertical movements—each time the animal rears up and interrupts the vertical sensor (separated by at least 1 second).

Variables 8, 9, 10, and 11: Time spent in comers (left and right front, left and right rear)—time spent by the animal in close proximity to two adjoining walls of the cage.

Variable 12: Vertical time (sec)—time spent interrupting the vertical beams during rearing.

Variable 13: Stereotypy counts—number of beam breaks that occur during a period of repeated interruption (stereotypy) of the same beam (or set of beams).

Variable 14: Number of stereotypy—number of times the monitor observes stereotypic behavior, separated by at least 1 second.

Variable 15: Stereotypy time (sec)—total amount of time that stereotypic behavior is exhibited.

Variable 16: Clockwise revolutions—number of times the animal circles with at least a 2″ diameter (will not pick up tighter rotating movements).

Variable 17: Anticlockwise revolutions—number of times the animal circles with at least a 2″ diameter.

Variable 18: Margin time (sec)—time spent by the animal in close proximity (within 1 cm) to the walls of the plexiglas cage.

Variable 19: Center time (sec)—time spent by the animal away from the walls of the cage.

Data can be expressed as either actual counts, time (in sec), centimeters traveled, or percent inhibition of activity relative to vehicle-treated control animals tested concurrently. Significant changes in activity (i.e., cm traveled), relative to controls, are determined by t-test or analysis of variance and Newman-Keul's multiple-range test. Stimulation of activity levels is indicated by negative values. The dose which could be expected to decrease activity levels by 50% (ED.sub.50) and the 95% confidence limits (CL) around that value are estimated by regression analysis using at least three data points which fall on the linear portion of the dose-effect curve.

Blockade of Amphetamine-Stimulated Locomotion in Rats

The blockade of amphetamine-stimulated locomotion procedure is a modification of the Locomotor Activity Protocol in the Omnitech Digiscan Activity Monitors described above. The blockade of amphetamine-stimulated locomotion procedure uses the central nervous system stimulant d-amphetamine to assess antipsychotic activity of dopaminergic agents.

Male Sprague-Dawley rats (Harlan Labs) are used for these studies. The treated group may receive only a single dose of the test compound before the experiment or may be treated daily (e.g., sid, bid, or tid) for at least 1 day, 3 days, 1 week, 2 weeks, 1 month, or 2 months, prior to the experiment. For the IP studies, amphetamine is given 20 minutes prior to the drug, after which a 30 minute locomotor activity test is conducted. For the oral study, drug is dosed 30 minutes prior to the test, while amphetamine is given 15 minutes prior to the test, which allows time for oral absorption. Locomotor activity (centimeters travelled per 30 minute test) is measured in 16″.times.16″ open chambers. Amphetamine generally produces a 2- to 3-fold increase in locomotion over saline controls. Drug effects are reported as percent reversal of amphetamine-stimulated locomotion. Significant changes in amphetamine-stimulated locomotion, relative to amphetamine treated controls, are determined by t-test. The dose which would reverse amphetamine-stimulated locomotion by 50% (ED.sub.50) and the 95% confidence limits are estimated by regression analysis.

Protocol for the Prepulse Inhibition of Acoustic Startle Model in Rats

Prepulse inhibition (PPI) of acoustic startle is a form of sensorimotor gating which occurs when a weak stimulus precedes a startling stimulus, resulting in diminution of the startle response amplitude. Schizophrenic patients exhibit reduced prepulse inhibition of acoustic startle compared to control subjects, consistent with a loss of sensorimotor gating. Thus, an animal model utilizing this phenomenon is quite useful in the study of known and potential antipsychotic agents. In rats, for example, PPI can be blocked with direct dopamine agonists (DA) such as apomorphine, or the indirect DA agonist amphetamine, and this effect can be antagonized with dopamine antagonists such as haloperidol.

Male Sprague-Dawley rats from Harlan Labs (180-280 g) are housed in groups of five rats per cage and maintained on a 12-hour light/dark cycle with free access to food pellets and water. The treated group may receive only a single dose of the test compound before the experiment or may be treated daily (e.g., sid, bid, or tid) for at least 1 day, 3 days, 1 week, 2 weeks, 1 month, or 2 months, prior to the experiment.

Startle chambers (SR-LAB, San Diego Instruments) consisting of a Plexiglas cylinder resting on a Plexiglas frame within a ventilated sound-attenuating enclosure are used. Acoustic stimuli are presented via a loudspeaker mounted above the rat. A piezoelectric device is mounted below the Plexiglas frame, which detects and transduces the motion occurring inside the cylinder during the 100 msec after the onset of the startling stimulus. The average responses during the 100 msec record window (100.times.1 msec readings) are recorded by microcomputer and interface assembly (San Diego Instruments). Each of the chambers are calibrated to one another to ensure consistent levels of loudspeaker performance over a wide range of decibel (dB) levels (67 to 125 dB). Sound levels are assessed with a dB meter (e.g., Radio Shack). Each stabilimeter (which houses the piezoelectric device) is adjusted to produce equal response sensitivity to a constantly vibrating calibrator.

Animals treated with the test compound may receive only a single dose of the test compound before the experiment or may be treated daily (e.g., sid, bid, or tid) for at least 1 day, 3 days, 1 week, 2 weeks, 1 month, or 2 months, prior to the experiment. Prior to the experiment, each animal is pretreated with saline, test, or control compounds (e.g., apomorphine, haloperidol, clozapine, etc.).

Each test session begins with a 5-minute test acclimation period of 70 dB white noise. The test session lasts a total of 30 minutes; several sequential tests are done to obtain an adequate number of rats per treatment group. The first and last trials are 120 dB pulse-alone trials presented 7 to 23 seconds apart, during which time the rats habituate rapidly to the noise bursts. These data are not included in the PPI calculation. The middle trials consists of 120 dB pulse-alone trials and trials of each of the following five trial types in pseudorandom order: (1) no stim, (2) 72 dB prepulse 100 msec prior to 120 dB startle, (3) 74 dB prepulse 100 msec prior to 120 dB startle, (4) 78 dB prepulse 100 msec prior to 120 dB startle, and (5) 86 dB prepulse 100 msec prior to 120 dB startle. The prepulses (2, 4, 8, and 16 dB over 70 dB background noise) are of 20-msec duration, while the startle stimuli were 40-msec duration. When the prepulse is paired with the 120 dB pulse, no obvious acoustic difference can be detected by the human ear as compared to the 120 dB pulse alone. Prepulse inhibition of the acoustic startle reflex is expressed as the percent inhibition of the 120 dB startle amplitude produced when a 2 to 16 dB (over background) prepulse precedes the startling stimulus.

Inhibition of Apomorphine-Induced Climbing Behaviour

In Animal Pharmacology Studies, the antipsychotic activity of the test compounds can be tested by the inhibition of apomorphine-induced climbing behaviour (P.Protais et al: “Psychopharmacology”, 50, 1-6, 1976). Male Swiss mice weighing 22-24 g are used. Animals treated with the test compound may receive only a single dose of the test compound before the experiment or may be treated daily (e.g., sid, bid, or tid) for at least 1 day, 3 days, 1 week, 2 weeks, 1 month, or 2 months, prior to the experiment. Animals are administered orally with test drug or 0.25% agar at time 0. After 60 minutes, apomorphine is subcutaneously injected at a dose of 1 mg/kg, and after further 70 minutes the animal's behaviour is assessed. Two additional assessments are performed at 10 min intervals. For assessment, each animal is placed on the bottom of a small upright box (11.times.7.5.times.4.5 cm). The walls of the box are made of translucent methacrylate except one of the lateral surfaces (7.5 cm wide) which is a 3 mm wire mesh. The position of the animal is scored for 2 minutes according to the following criteria: 0=four paws on the floor; 1=three paws on the floor; 2=two paws on the floor; 3=one paw on the floor; and 4=four paws holding the wire mesh. If an animal keeps several positions within the 2 min observation, the seconds elapsed in each position will be recorded. Finally, mean scoring is calculated. Under these experimental conditions, the effective dose 50% (ED.sub.50) values are calculated.

Inhibition Of DOI-Induced Head Twitches and Scratches

The antipsychotic activity of the test compounds can also be tested by the inhibition test of 1-(2,5-dimethoxy-4-iodophenyl)-2-aminopropane (DOI)-induced head twitches and scratches (M. Oka et al: “J. Pharm. Exp. Ther.”, 264(1), 158-165, 1993). Male N.M.R.I. mice weighing 22-26 g are used. After the animals are weighed, they are individually placed in transparent cages two hours prior to experiment. Animals treated with the test compound may receive only a single dose of the test compound before the experiment or may be treated daily (e.g., sid, bid, or tid) for at least 1 day, 3 days, 1 week, 2 weeks, 1 month, or 2 months, prior to the experiment. Test compound is given p.o. at time 0. At time 60 min DOI at the dose of 3 mg/kg i.p. dissolved in saline is administered. The number of head twitches and scratches were assessed as well as the presence or absence of escape attempts. The effective dose 50% (ED.sub.50) values obtained under the above experimental conditions are calculated.

Human Clinical Trials

The activity of a DAO or DDO antagonist for treating or alleviating schizophrenia, bipolar disorder, or another CNS disorder of the present inventon can be demonstrated by human clinical trials. For example, a study can be designed as a double-blind, parallel, placebo-controlled multicenter trial. Subjects are randomized into four groups, placebo and three increasing dosages tid of test compound, e.g., 25, 50, and 75 mg. The dosages are administered in a manner disclosed herein or practiced by the skilled practitioner, e.g., orally with food. Subjects are observed at four visits to provide baseline measurements. Further visits, e.g., 5-33, are served as the treatment phase for the study.

During the visits, subjects are observed for signs of psychotic behavior or bipolar behavior such as agitation, mood swings, tremor, delirium, social withdrawal, and concentration abilities. Treatment groups are compared with respect to the number and percent of subjects who ever had the symptom during the double-blind portion of the study (visits 5 through 33), at a severity that was worse than during the baseline visits (1 through 4).

DAO, DDO, and Biallelic Markers thereof in Methods of Genetic Diagnostics

The DAO and DDO genomic and cDNA sequences, and the biallelic markers of the present invention can also be used to develop diagnostics tests capable of identifying individuals who express a detectable trait as the result of a specific genotype or individuals whose genotype places them at risk of developing a detectable trait at a subsequent time. The trait analyzed using the present diagnostics may be used to diagnose any detectable trait, including predisposition to schizophrenia or bipolar disorder, age of onset of detectable symptoms, a beneficial response to or side effects related to treatment against schizophrenia or bipolar disorder. Such a diagnosis can be useful in the monitoring, prognosis and/or prophylactic or curative therapy for schizophrenia or bipolar disorder.

The diagnostic techniques of the present invention may employ a variety of methodologies to determine whether a test subject has a genotype associated with an increased risk of developing a detectable trait or whether the individual suffers from a detectable trait as a result of a particular mutation, including methods which enable the analysis of individual chromosomes for haplotyping, such as family studies, single sperm DNA analysis or somatic hybrids.

The diagnostic techniques concern the detection of specific alleles present within the human DAO or DDO genes, preferably within a DAO or DDO exon or coding sequence. More particularly, the invention concerns the detection of a nucleic acid comprising at least one of the nucleotide sequences of SEQ ID Nos. 1 to 6 or a fragment thereof or a complementary sequence thereto including the polymorphic base.

These methods involve obtaining a nucleic acid sample from the individual and, determining, whether the nucleic acid sample contains at least one allele or at least one biallelic marker haplotype, indicative of a risk of developing the trait or indicative that the individual expresses the trait as a result of possessing a particular the human DAO or DDO-related polymorphism or mutation (trait-causing allele).

Preferably, in such diagnostic methods, a nucleic acid sample is obtained from the individual and this sample is genotyped using methods well known in the art, or as described for example in PCT/IB00/00435 incorporated herein by reference. The diagnostics may be based on a single biallelic marker or a on group of biallelic markers.

In each of these methods, a nucleic acid sample is obtained from the test subject and the biallelic marker pattern of one or more of a biallelic marker of the invention is determined.

In one embodiment, a PCR amplification is conducted on the nucleic acid sample to amplify regions in which polymorphisms associated with a detectable phenotype have been identified. The amplification products are sequenced to determine whether the individual possesses one or more human DAO or DDO polymorphisms associated with a detectable phenotype. Alternatively, the nucleic acid sample is subjected to microsequencing reactions to determine whether the individual possesses one or more DAO or DDO-related polymorphisms associated with a detectable phenotype resulting from a mutation or a polymorphism in the DAO or DDO genomic sequence. In another embodiment, the nucleic acid sample is contacted with one or more allele specific oligonucleotide probes which, specifically hybridize to one or more human chromosome DAO or DDO-related alleles associated with a detectable phenotype. In another embodiment, the nucleic acid sample is contacted with a second oligonucleotide capable of producing an amplification product when used with the allele specific oligonucleotide in an amplification reaction. The presence of an amplification product in the amplification reaction indicates that the individual possesses one or more DAO or DDO-related alleles associated with a detectable phenotype. In a preferred embodiment, the detectable trait is schizophrenia or bipolar disorder. Diagnostic kits comprise any of the polynucleotides of the present invention.

These diagnostic methods are extremely valuable as they can, in certain circumstances, be used to initiate preventive treatments or to allow an individual carrying a significant haplotype to foresee warning signs such as minor symptoms.

Diagnostics, which analyze and predict response to a drug or side effects to a drug, may be used to determine whether an individual should be treated with a particular drug. For example, if the diagnostic indicates a likelihood that an individual will respond positively to treatment with a particular drug, the drug may be administered to the individual. Conversely, if the diagnostic indicates that an individual is likely to respond negatively to treatment with a particular drug, an alternative course of treatment may be prescribed. A negative response may be defined as either the absence of an efficacious response or the presence of toxic side effects.

Clinical drug trials represent another application for the markers of the present invention. One or more markers indicative of response to an agent acting against schizophrenia or to side effects to an agent acting against schizophrenia may be identified using the methods described above. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems.

DAO and DDO in the Prevention and Treatment of Disease

In large part because of the risk of suicide, the detection of susceptibility to schizophrenia, bipolar disorder as well as other psychiatric disease in individuals is very important. Consequently, the invention concerns a method for the treatment of schizophrenia or bipolar disorder, or a related disorder comprising the following steps:

selecting an individual whose DNA comprises alleles of a DAO or DDO-related biallelic marker or of a group of DAO or DDO-related markers associated with schizophrenia or bipolar disorder;

following up said individual for the appearance (and optionally the development) of the symptoms related to schizophrenia or bipolar disorder; and

administering a treatment acting against schizophrenia or bipolar disorder or against symptoms thereof to said individual at an appropriate stage of the disease.

Another embodiment of the present invention comprises a method for the treatment of schizophrenia or bipolar disorder comprising the following steps:

selecting an individual whose DNA comprises alleles of a DAO or DDO-related biallelic marker or of a group of DAO or DDO-related markers associated with schizophrenia or bipolar disorder;

administering a preventive treatment of schizophrenia or bipolar disorder to said individual.

In a further embodiment, the present invention concerns a method for the treatment of schizophrenia or bipolar disorder comprising the following steps:

selecting an individual whose DNA comprises alleles of a DAO or DDO-related biallelic marker or of a group of DAO or DDO-related markers associated with schizophrenia or bipolar disorder;

administering a preventive treatment of schizophrenia or bipolar disorder to said individual;

following up said individual for the appearance and the development of schizophrenia or bipolar disorder symptoms; and optionally

administering a treatment acting against schizophrenia or bipolar disorder or against symptoms thereof to said individual at the appropriate stage of the disease.

For use in the determination of the course of treatment of an individual suffering from disease, the present invention also concerns a method for the treatment of schizophrenia or bipolar disorder comprising the following steps:

selecting an individual suffering from schizophrenia or bipolar disorder whose DNA comprises alleles of a DAO or DDO-related biallelic marker or of a group of DAO or DDO-related markers, associated with the gravity of schizophrenia or bipolar disorder or of the symptoms thereof; and

administering a treatment acting against schizophrenia or bipolar disorder or symptoms thereof to said individual.

The invention also concerns a method for the treatment of schizophrenia or bipolar disorder in a selected population of individuals. The method comprises:

selecting an individual suffering from schizophrenia or bipolar disorder and whose DNA comprises alleles of a DAO or DDO-related biallelic marker or of a group of DAO or DDO-related markers associated with a positive response to treatment with an effective amount of a medicament acting against schizophrenia or bipolar disorder or symptoms thereof,

and/or whose DNA does not comprise alleles of a DAO or DDO-related biallelic marker or of a group of DAO or DDO-related markers associated with a negative response to treatment with said medicament; and

administering at suitable intervals an effective amount of said medicament to said selected individual.

In the context of the present invention, a “positive response” to a medicament can be defined as comprising a reduction of the symptoms related to the disease. In the context of the present invention, a “negative response” to a medicament can be defined as comprising either a lack of positive response to the medicament which does not lead to a symptom reduction or which leads to a side-effect observed following administration of the medicament.

The invention also relates to a method of determining whether a subject is likely to respond positively to treatment with a medicament. The method comprises identifying a first population of individuals who respond positively to said medicament and a second population of individuals who respond negatively to said medicament. One or more biallelic markers is identified in the first population which is associated with a positive response to said medicament or one or more biallelic markers is identified in the second population which is associated with a negative response to said medicament. The biallelic markers may be identified using the techniques described herein.

A DNA sample is then obtained from the subject to be tested. The DNA sample is analyzed to determine whether it comprises alleles of one or more biallelic markers associated with a positive response to treatment with the medicament and/or alleles of one or more biallelic markers associated with a negative response to treatment with the medicament.

In some embodiments, the medicament may be administered to the subject in a clinical trial if the DNA sample contains alleles of one or more biallelic markers associated with a positive response to treatment with the medicament and/or if the DNA sample lacks alleles of one or more biallelic markers associated with a negative response to treatment with the medicament. In preferred embodiments, the medicament is a drug acting against schizophrenia or bipolar disorder.

Using the method of the present invention, the evaluation of drug efficacy may be conducted in a population of individuals likely to respond favorably to the medicament.

Another aspect of the invention is a method of using a medicament comprising obtaining a DNA sample from a subject, determining whether the DNA sample contains alleles of one or more biallelic markers associated with a positive response to the medicament and/or whether the DNA sample contains alleles of one or more biallelic markers associated with a negative response to the medicament, and administering the medicament to the subject if the DNA sample contains alleles of one or more biallelic markers associated with a positive response to the medicament and/or if the DNA sample lacks alleles of one or more biallelic markers associated with a negative response to the medicament.

The invention also concerns a method for the clinical testing of a medicament, preferably a medicament acting against schizophrenia or or bipolar disorder or symptoms thereof. The method comprises the following steps:

administering a medicament, preferably a medicament susceptible of acting against schizophrenia or or bipolar disorder or symptoms thereof to a heterogeneous population of individuals,

identifying a first population of individuals who respond positively to said medicament and a second population of individuals who respond negatively to said medicament,

identifying biallelic markers in said first population which are associated with a positive response to said medicament,

selecting individuals whose DNA comprises biallelic markers associated with a positive response to said medicament, and

administering said medicament to said individuals.

In any of the methods for the prevention, diagnosis and treatment of schizophrenia and bipolar disorder, including methods of using a medicament, clinical testing of a medicament, determining whether a subject is likely to respond positively to treatment with a medicament.

Such methods are deemed to be extremely useful to increase the benefit/risk ratio resulting from the administration of medicaments which may cause undesirable side effects and/or be inefficacious to a portion of the patient population to which it is normally administered.

Once an individual has been diagnosed as suffering from schizophrenia or bipolar disorder, selection tests are carried out to determine whether the DNA of this individual comprises alleles of a biallelic marker or of a group of biallelic markers associated with a positive response to treatment or with a negative response to treatment which may include either side effects or unresponsiveness.

The selection of the patient to be treated using the method of the present invention can be carried out through the detection methods described above. The individuals which are to be selected are preferably those whose DNA does not comprise alleles of a biallelic marker or of a group of biallelic markers associated with a negative response to treatment. The knowledge of an individual's genetic predisposition to unresponsiveness or side effects to particular medicaments allows the clinician to direct treatment toward appropriate drugs against schizophrenia or bipolar disorder or symptoms thereof.

Once the patient's genetic predispositions have been determined, the clinician can select appropriate treatment for which negative response, particularly side effects, has not been reported or has been reported only marginally for the patient.

The biallelic markers of the invention have demonstrated an association with schizophrenia and bipolar disorders. However, the present invention also comprises any of the prevention, diagnostic, prognosis and treatment methods described herein using the biallelic markers of the invention in methods of preventing, diagnosing, managing and treating related disorders, particularly related CNS disorders.

### EXAMPLES

Construction of the Plasmids for the Protein Expression in Bacteria and in Yeast

Expression of the recombinant proteins without tag was carried out with pET11a vector (Stratagen). The coding sequences with appropriated sites (Nde1 in 5′ and HindIII in 3′) were obtained by PCR (TaqPlusPrecision System, Stratagen) with the primers corresponding to the ORF limits.

The generated PCR products were purified (Qiaquick PCR, Qiagen), digested with Nde1and HindIII, gel purified (Microspin, PolyLabo), and ligated into a vector open with the same enzymes. The constructs were transfected into the DH10B bacterial host (Gibco BRL), plasmid DNAs were extracted and sequenced to select proper coding sequences.

The plasmids for expression of human DAAO and g34872 in yeast were constructed with pESC-LEU shuttle vector (Stratagen).

Expression and Purification of the Recombinant g34872 Protein without Tag

The plasmids were then transfected into the BL21(DE3) CodonPlus RIL bacterial host (Stratagen), the bacteria were allowed to grow in 0.8 liter of LB media until an A600 of 0.7 was achieved. Expression of fusion proteins was induced by the addition of 1 mM isopropyl-1-thio-D-galactopyranoside and further cultured for 3 h. Bacterial pellets were prepared and immediately frizzed (−80° C.), then thawed in the water bath at 30° C.; AEBSF was added at 2mM. Bacterial cells were suspended in 25 ml of BugBuster extraction agent (Novagen) supplemented with protease inhibitor mixture (SetIII, Calbiochem) and with 10 mM EGTA. The suspension was incubated 30 min at room temperature, then benzonase was added (Novagen) and incubation was continued for 15 min. The lysate was centrifuged at 10,000×g at 4° C. for 30 min. Bacterial proteins were fractionated from the supernatant by salt precipitation. The protein pellet corresponding to 35-55% of ammonium sulfate saturation was dissolved in 2 ml of 50 mM TrisHCl buffer pH8/50 mM NaCl with 10 mM DTT, the solution was clarified by centrifugation and applied on Ultragel AcA44 (Pharmacia) column (1.6×65 cm) equilibrated with 20 mM TrisCl buffer pH8/50 mM NaCl buffer. Eluted proteins were analyzed by electrophoresis, the fractions containing MN2R protein were pooled and concentrated by ultrafiltration (10K cut, Biomax-15, Sigma). The proteins were then applied on DEAE-Macroprep (Bio-Rad) column (1×2 cm) equilibrated with 20 mM TrisCl pH8 and eluted with linear salt gradient (from 0 to 1M NaCl, 20 column volumes). The fractions containing MN2R protein were pooled, concentrated by ultrafiltration (10K cut) and applied on Superdex 75 (Pharmacia) column (1×27 cm) equilibrated with 20 mM TrisCl pH8 buffer. The fractions from single major pique were pooled, concentrated to 5 mg /ml and saved at 4° C. Yield of the purified electrophoretically homogenous protein was typically 5 mg per liter of bacterial culture.

Denaturing electrophoresis of the proteins in 10% NuPage custom gels was done according the manufacturer recommendations (NuPage by Novagen), MES/SDS running buffer was used. Molecular weight markers See-Blue were from Invitrogen. Proteins were visualized after staining with Coomassie Brilliant Blue G colloidal solution (Sigma).

Purification of the Natural DAAO from Pig Kidney

The crude preparation of pig kidney DAAO was purchased from Sigma. Proteins were dissolved in 50 mM TrisCl pH8 (1 g in 10 ml); the solution was clarified by centrifugation and applied on Sephadex G-50 medium column (2.6×40 cm) equilibrated with 10 mM TrisCl pH8/100 mM NaCl. The desalted proteins were then concentrated 3 fold by ultrafiltration (30K cut, Biomax-15, Sigma) equilibrated with 10 mM TrisCl pH8/100 mM NaCl/10 mM DTT/10 mM ATP and applied on DEAE-Sepharose column (1.6×7 cm) in the same buffer without ATP. The column was washed with two column volumes of 10 mM TrisCl pH8/100 mM NaCl, followed by one volume of 10 mM TrisCl pH8/125 mM NaCl and then proteins were eluted with 10 mM TrisCl pH8/150 mM NaCl buffer. The fractions were assayed for DAAO enzyme activity, pooled and concentrated by ultrafiltration. The proteins were then applied on Ultragel AcA44 column (1.6×65 cm) equilibrated with 10 mM TrisCl pH8/100 mM NaCl/1 mM DTT and eluted with the same buffer. The fractions containing electrophoretically pure DAAO were concentrated by ultrafiltration and kept at 4° C.

Expression and Purification of the Recombinant Human DAAO

The plasmid was transfected into the BL21(DE3) CodonPlus RIL bacterial host (Stratagen), the bacteria were allowed to grow in 3 liters of LB media until an A600 of 0.7 was achieved. Expression of fusion proteins was induced by the addition of 1 mM isopropyl-1-thio-beta-D-galactopyranoside and further cultured for 5 h. Bacterial pellets were extracted with BugBuster extraction agent (Novagen) in presence of 2 mM AEBSF, benzonase was routinely used. The lysate was adjusted to pH8 with 50 mM TrisCl and centrifuged at 10,000×g at 4° C. for 20 min. The proteins were precipitated from the extract with ammonium sulfate (from 30 to 50% saturation) , collected by centrifugation at 10,000×g at 4° C. for 60 min and dissolved in 50 mM TrisCl pH8 (10 ml); the solution was clarified by centrifugation and applied on Sephadex G-75 column (2.6×40 cm) equilibrated with 10 mM TrisCl pH8/100 mM NaCl. The following steps of the purification were almost identical to those described for pig kidney DAAO. The only exception was the elution of the protein from DEAE-resin: it was done with 10 mM TrisCl pH8/300 mM NaCl buffer. The yield of the purified electrophoretically homogenous DAAO protein was 0.7 mg per liter of bacterial culture.

Expression and Extraction of g34872 and Human DAAO Proteins in Yeast S. cerevisiae

Yeast S.cerevisiae YPH499 and FY1679-18B strains were grown on YPD rich medium. The plasmids were transfected in yeast cells by standard lithium acetate method; the transformants were selected on YNG synthetic medium, grown at 30° C. in 1 liter of synthetic medium lacking leucine with 2% raffinose as a carbon source up to culture density 1 u A600/ml . The cells were collected by centrifugation at 20° C., the medium was replaced by YNGal (with 2% D-galactose) and the incubation were continued for 20 h. The cells were pelleted, washed with ice cold water, resuspended in 20 mM TrisCl buffer pH8/2 mM AEBSF and vortexed 8 times for 1 min with a glass beads (Sigma) to extract the proteins. The lysate was centrifuged at 10,000×g at 4° C. for 30 min, the supernatant (S1) was collected and kept at 4° C. The pellet was resuspended in 20 mM TrisCl buffer pH8/2 mg/ml saponine/0.3% sarkosyl and vortexed 3 times for 1 min. The pellet extract was clarified by centrifugation (S2) and immediately frozen at −20° C. The protein concentration was detected by Bradford reagent (Bio-Rad), the expression was confirmed by Western blot procedure with rabbit anti-g34872-his6 serum (dilution 1/5000) and by DAAO enzyme activity detection with D-serine a substrate.

DAAO Enzyme Activity Detection

The assay mixture was typically composed of D-serine (Aldrich) 200 mM, FAD (Sigma) at 0.1 mM, sodium phosphate buffer pH8 at 75 mM, HR-peroxidase (Sigma) 1U/ml. The mixture was air-saturated just before use. o-Dianizidine (Sigma) was added in the mixture. In the typical assay 5 μl of the enzyme (DAAO and mixes) was added to 25 μl of the assay mixture, the incubation was stopped with 50 μl of 20% H2SO4. The activity was observed as absorbance of the peroxidase-oxidized o-Dianizidine at 540 nm. The reactions containing high protein concentrations were centrifuged 15 min at 14000 rpm before absorbance measuring.

The control experiences were done to establish that g34872 protein do not influence peroxidase enzyme activity. The assay of peroxidase was done in the conditions identical to those for DAAO assay; hydrogen peroxide (Gibco) was used as a substrate, and no effect of g34872 on HRP activity was confirmed.

### Example 1

Yeast cells were transformed with the plasmids constructed in pESC-Leu expression vector. One plasmid can express hDAAO, second one—C-terminal tagged g34872, third—is the vector without insertion (control). After the induction of the expression (2% galactose in the medium) these cells were incubated for 24 h, than the extracts were maid and combined as follow: different volumes of DAAO extract were mixed with either g34872-cHis6 or with the vector extracts. The same volumes of DAAO extracts were also mixed with BSA (external control). After 30 min of the pre-incubation the combined extracts were used for DAAO activity measuring. All yeast extracts and BSA solution had the same total protein concentration. DAAO activity was determined with D-serine at 37° C. See FIG. 1 for demonstration that g34872 activates DAO.

### Example 2

Purified recombinant human DAAO was added in the E.coli extract containing expressed g34872 and in BSA solution. The concentrations of DAAO were 0.5 and 0.3 mg/ml. Total protein concentrations of the bacterial extract and of BSA were 12 mg/ml. After 30 min pre-incubation the mixes were used for DAAO activity measuring at 37° C. D-serine was used as a substrate. (FIG. 2)

### Example 3

In vitro Activation of Purified DAAO in the Presence of g34872 Protein : the Effect of Activation Depends on g34872 Concentration

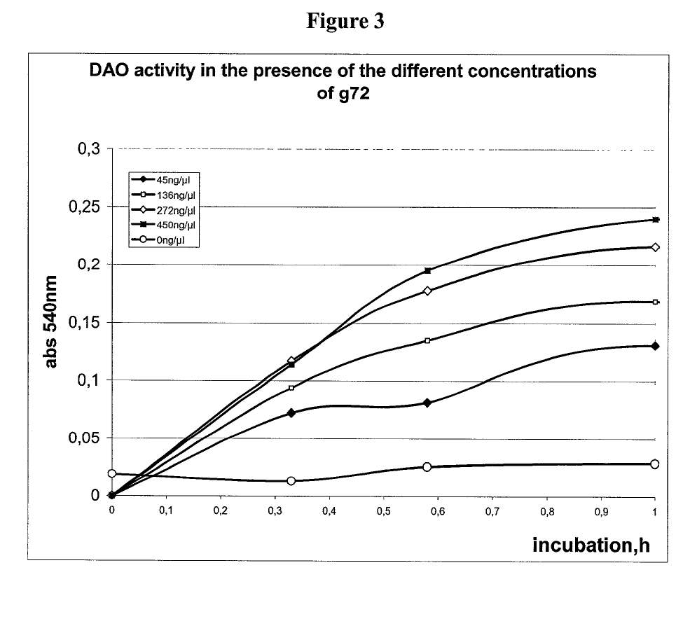

Purified DAAO and g34872 were mixed and incubated 50 min before activity essay, T°amb. Total protein concentration was the same in all the mixes. D-Serine was used as the substrate for DAAO, the pH of the reaction was 8,0. Proteins used: purified porcine DAAO, concentrations in the mixes were always 50 ng/μl purified recombinant g34872 concentrations in the mixes were from 0 to 450 ng/μl bovine serum albumin (BSA) concentrations in the mixes were from 0 to 450 ng/μl. The range of the concentrations of g34872 protein can be considered as physiological as corresponding to the data found for lumenal Golgi proteins. See FIG. 3.

### Example 4

Estimation of the Limits of g34872 Concentrations Necessary for DAAO Activation in vitro

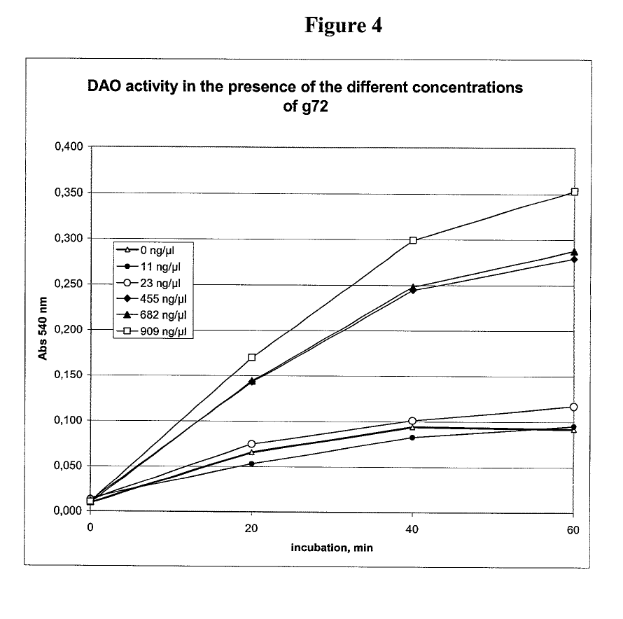

Pig kidney DAAO was mixed with g34872 in PBS and incubated 50 min at 20° C. DAAO concentration was 50 ng/μl in all the mixes. The enzymatic activity of DAAO was measured at 20° C with 200 mM D-serine, pH was 8.0 Pig kidney DAAO was mixed with g34872 in PBS and incubated 1 h at 20° C. DAAO concentration was 50 ng/μl in all the mixes. The enzymatic activity of DAAO was measured at 20° C. with 200 mM D-serine, pH was 8.0. See FIG. 4.

### Example 5

DAAO Kinetics in the Presence of g34872 Protein: g34872 is an Allosteric Activator of DAAO

Pig kidney DAAO was mixed with g34872 in PBS and incubated 30 min at 20° C. DAAO concentration was 200 ng/μl and g34872 concentration was 2 μg/μl in the protein mixture. The control mixture (without g34872) was composed of 200 ng/μl DAAO and 2 μg/μl BSA. The enzymatic activity of DAAO was measured at 20° C. with D-serine, the substrate concentration used were from 0 to 100 mM, other corposants of the mixture and pH were standard.

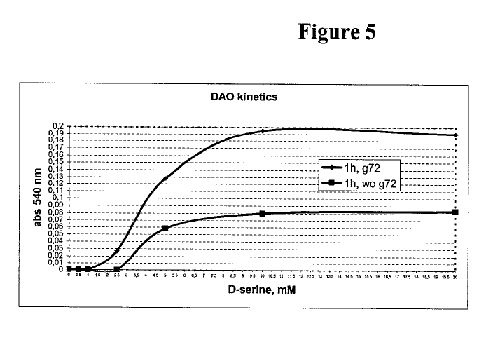

Vmax observed for g34872&DAAO mix corresponds to Km=4 mM, Vmax observed for DAAO&BSA mix corresponds to Km=4 mM. This result shows no change in DAAO affinity for its substrate (D-serine) and suggests that g34872 interacts with DAAO in the site other than the active site of the enzyme. See FIG. 5.

### Example 6

Biallelic Markers of the Invention

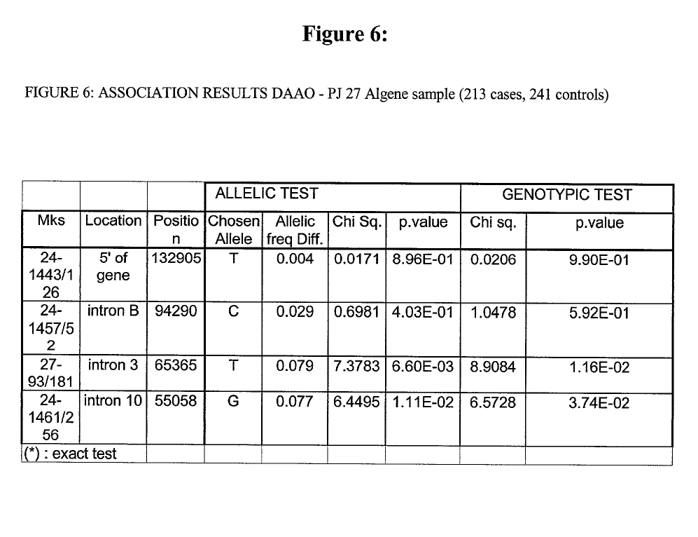

Validated polymorphisms (occurring at a frequency of >5% in the general population) have been discovered in the DAO gene (SEQ ID NO: 1). These polymorphisms, also referred to as Biallelic markers, are represented by SEQ ID NOs: 23-26 and by numbers 24/1443-126, 24/1457-52, 27/93-181, and 24/1461-256, respectively, wherein the polymorphic base is located at position 24. Polynucleotides comprising amplicons and microsequencing primers for detecting each DAO biallelic marker of the invention are described in SEQ ID NO: 1. As shown in FIG. 6, Marker 27-93/181(SEQ ID NO: 25) and 24-1461/256 (SEQ ID NO: 26) have been determined to be significantly associated with schizophrenia p=0.0066 and 0.0111, respectively. Markers of the invention can be further used to determine if an individual is at risk for schizophrenia, as demonstrated in FIG. 6, as well as other related CNS disorders, preferably depression and bipolar disorder.

### Example 7

Syntheses of Compounds or Compositions of the Invention.

Compound Preparation:

The DAO and DDO antagonist compositions and compounds of the invention can be prepared by a variety of methods which are well known to one of skill in the art. General schemes include but are not limited to those described infra.

Preparation of Compounds of Formula I, Ia, Ib

A vast number of the compounds of Formulae I, Ia, and Ib are commercially available or readily synthesized via common methods known to the skilled artisan from commercially available compounds. Specifically, substituents can be introduced into aromatic rings such as phenyl, naphthyl or substituted naphthyl or phenyl by way of electrophilic substitution reactions such as Friedel Crafts alkylations, acylations, and nitration in concentrated nitric acid. Transforming aromatic groups into organometallic salts such as Grignard reagents or introduction of substituents via aryl diazonium compounds are also common methods of aromatic ring modification. Example of these manipulations and other relevant transformations are discussed in standard texts such as March, Advanced Organic Chemistry(Wiley), Carey and Sundberg, Advanced Organic Chemistry(Vol 2.) and Keeting, Heterocyclic Chemistry (all 17 volumes).

Preparation of Compounds of Formula II.

Compounds of Formula II are commercially available or readily synthesized by the skilled artisan utilizing known synthetic techniques.

Preparation of Compounds of Formula IV Substituted at Position Z

For the manipulation of R₁ it is understood that the skilled artisan may choose to prepare R₁ before, after or concurrent with the preparation of the heterocyclic ring. For compounds in which A is nitrogen, a preferred method of making the compounds is.

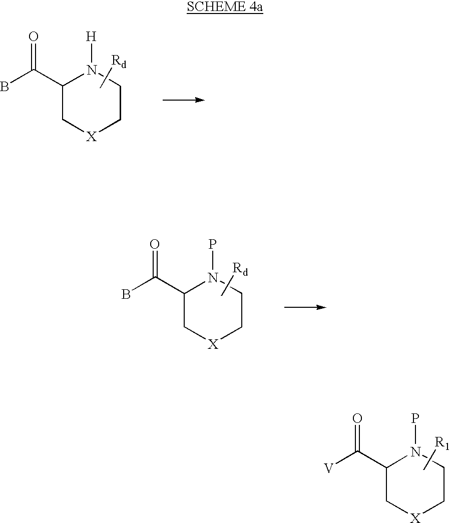

Where R_{d} is a derivatizable group or can be manipulated or substituted, such compounds are known and can be prepared by known methods. (P) is a protecting group such as aryl and (B) is a suitable blocking group. For clarity, groups at position (Y) of formula IV are not shown.

For preparation and elaboration of the heterocyclic ring it is understood that the skilled artisan may choose to prepare R₁ before, after or concurrent with the preparation of the heterocylic ring. For clarity, the substituents at Z and Y are not shown. For compounds in which X is nitrogen , a preferred method of manipulating R₂ is shown. In the schemes below, L is any acceptable leaving group, and B is a blocking group as above. Boc is an example of a preferred, and art recognized blocking group. The skilled artisan will recognize that the choice of blocking group is within the skill of the artisan working in organic chemistry.

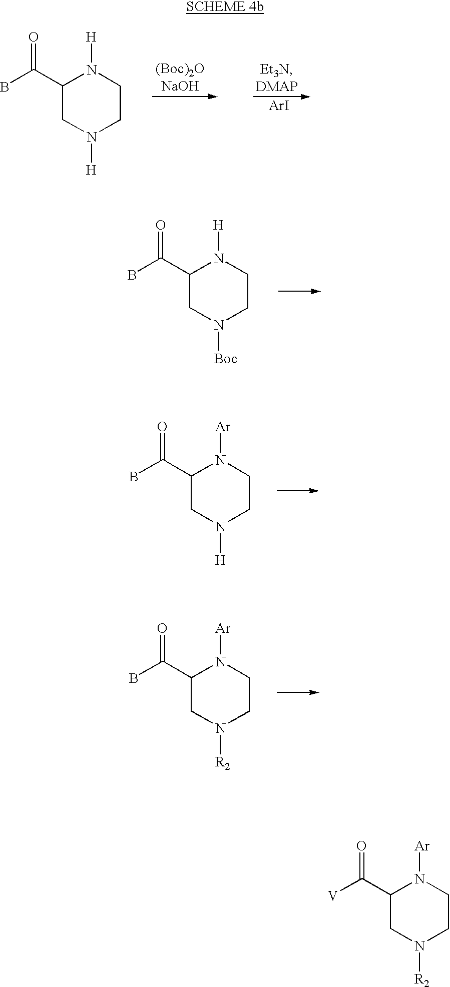

For compounds containing a sulfur in the heteroyclic ring the preferred methods of ring formation are shown. For the preparation and elaboration of the heterocyclic ring it is understood that the skilled artisan may choose to prepare R₁ before, after or concurrent with the preparation of the heterocyclic ring. For clarity groups at position Z and Y are not shown.

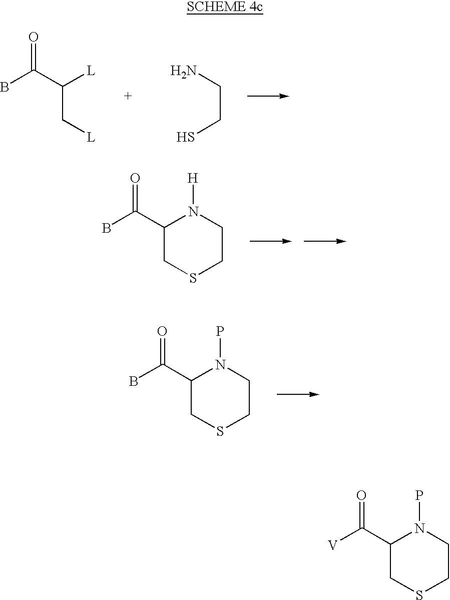

Where X is sulfur, further elaboration of the heterocyclic ring can be accomplished after the ring has been formed. For example, oxidation of the ring sulfur atom using known methods can provide the corresponding sulfoxides and sulfones as shown.

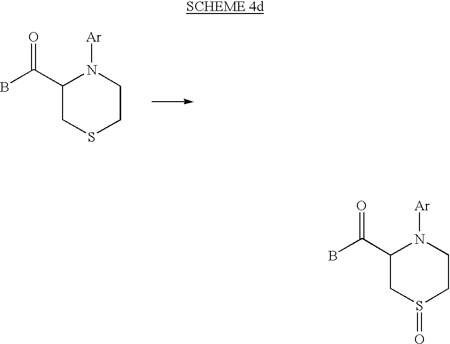

For compounds containing an oxygen in the heterocylic ring, the preferred methods of ring formation are shown. A bifunctional moiety, for example a halo hydroxy species is reacted with an aziridine below. The halo moiety serves as a leaving group, useful in ring closure reactions. Upon formation of the ring, elaboration of the invention proceeds as described above.

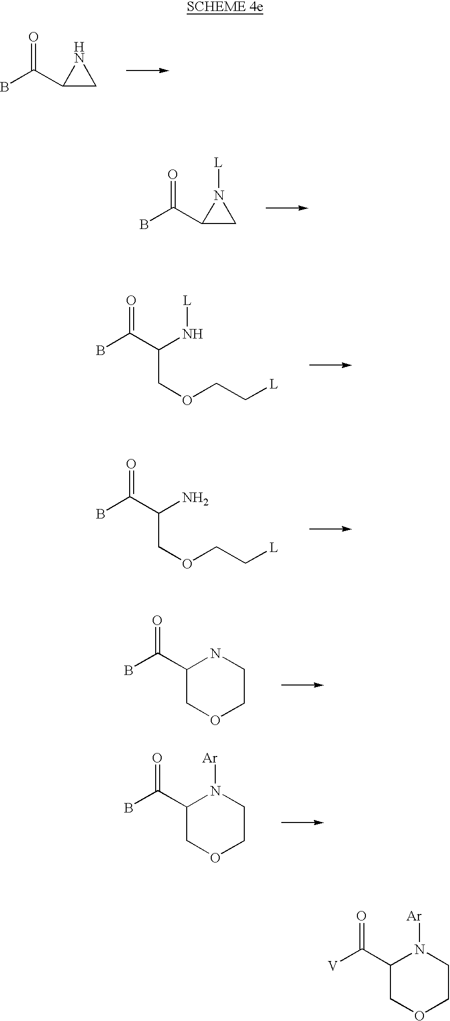

Another acceptable strategy for making the heterocyclic ring of the invention, having E as sulfur, nitrogen or oxygen includes the following scheme. This is a preferred route by which to also prepare compounds in which A is nitrogen and A-B is unsaturated.

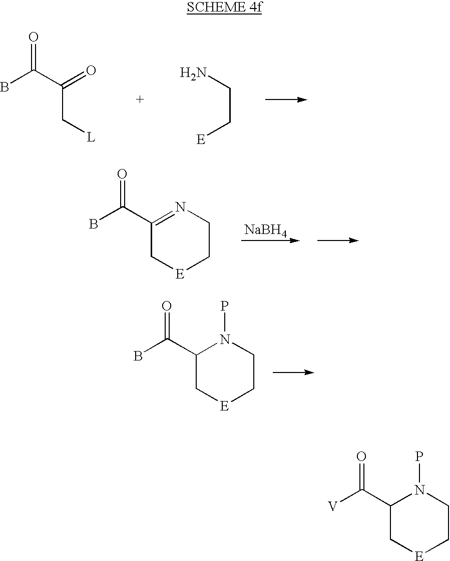

Preparation of other Preferred Compounds of Formula IV and Formula III

Of course the skilled artisan will recognize that scheme I can be applied to a substituent at (Y) for all of the described groups. Where Z is a ketal or thioketal the compounds of the invention may be prepared from a compound having a carbonyl in the ring. Such compounds are prepared by known method, and many of such compounds are known or commercially available. Thus the skilled artisan will appreciate that a hydroxy, amino, imino, alkoxy or other group may be manipulated into a carbonyl compound.

The skilled artisan will also recognize that the above synthetic routes for compounds of formula IV can be applied to compounds of formula III in which the ring size is seven and eight members in size. Symbols B, L, P and V are defined as described above. The following example is exemplary but not limiting.

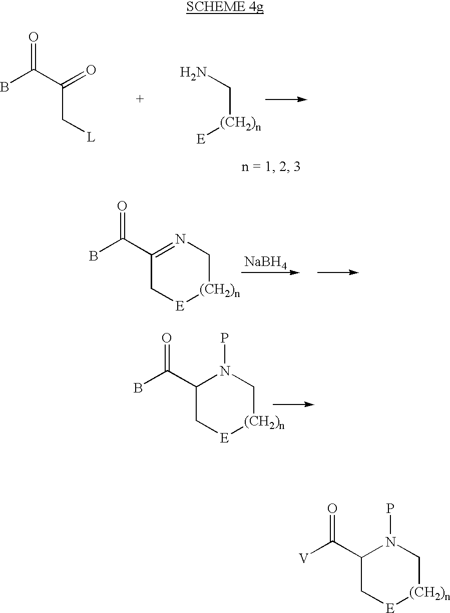

Synthesis of Compounds of Formula Va

Compounds of Formula Va can be synthesized by a variety of methods. The best known route, which can be used for different alpha amino acids is the Strecker synthesis route. In that method a suitable aldehyde is treated with ammonia and HCN, so that an alpha-amino nitrile is formed, which is subsequently subject to a hydrolysis reaction.

Another acceptable strategy for the synthesis of compounds of formula Va is through the following scheme:

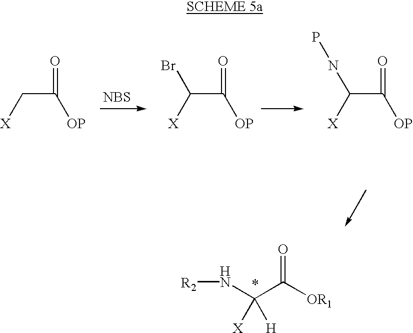

in which P is a protecting group such as tertiary butyl which may be the same as R₁. X is a group as described above. The protected compound is brominated using a halogenating reagent such as PBr₃, NBS or CBr₄ followed by halogen displacement using NH₃ or protected amine derivatives such as potassium phthalimide. Incorporation of R₁ and R₂ can be readily accomplished by the skilled artisan.

Synthesis of Compounds of Formula Vb in which X and Y Comprise a Cyclopropane Ring

Among the various routes for the construction of α-amino acids, 1,3-dipolar cycloaddition of diazoalkanes with α,β-dehydroamino acid derivatives has been widely utilized. Hence the scheme below demonstrates that R₃ substituents of dehydroamino acids which are preferably alkyl or Ar¹ can be protected as the imino esters, where Ar¹ is as defined above. The skilled artisan will recognize that such compounds can be reacted with diazo substituted compounds which are preferably alkyl or Ar¹ to produce the resulting protected cycolpropane derivative. Reaction of such compounds with basic alcoholic solutions such as sodium methoxide followed by acid hydrolysis can provide the corresponding R₃, R₄ substituted cyclopropane amino acids. Further derivativization of R₁ and R₂ can be readily accomplished by known methods.

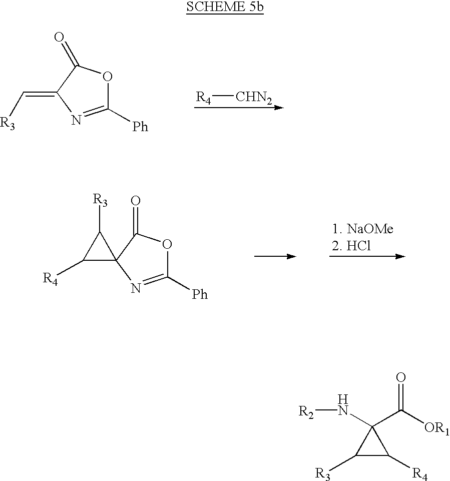

Synthesis of Compounds of Formula Vb in Which X and Y Comprise Rings of 5-8 Members

Substituted carbocyclic or heteroatom containing rings of preferably 5, 6, 7, 8 members can be transformed into amino acid derivatives consistent with the compounds represented by formula Vb. One of several well established routes is the conversion of a cyclic ketone containing compound to the corresponding amino acid derivative. Such cyclic keto compounds are abundant in the literature and are readily synthesized by the skilled artisan. The starting compound may be protected or unprotected. Trimethylsilyl cyanide addition to an imine derivative of the starting ketone provides cyano addition products. Hydrolysis and reductive cleavage of the protected amine generates the amino acid. Further derivativization of R₁ and R₂ can be readily accomplished by known methods.

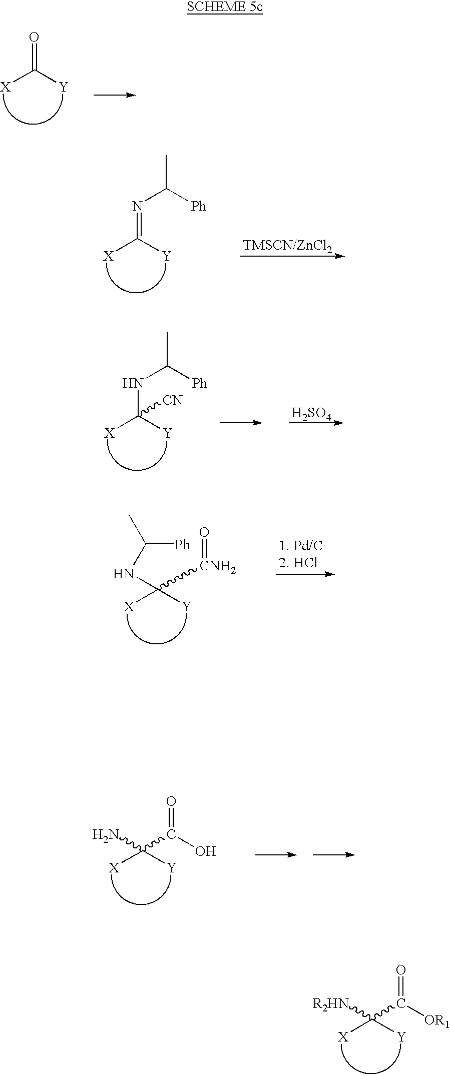

Synthesis of Compounds of Formula Vc

Compounds of formula Vc can be synthesized from sulfenimine derivatives of compounds substituted with R₃ where R₃ is preferably alkyl or aryl. There are several routes to the preparation of substituted sulfenimines that can be readily synthesized by the skilled artisan.

Addition of R₄ in the form of an organometallic reagent such as alkyl magnesium bromide followed by treatment with trifluoroacetic acid provides the corresponding disubstituted amino acid which can be further derivatized at R₂ and R₂ by known methods.

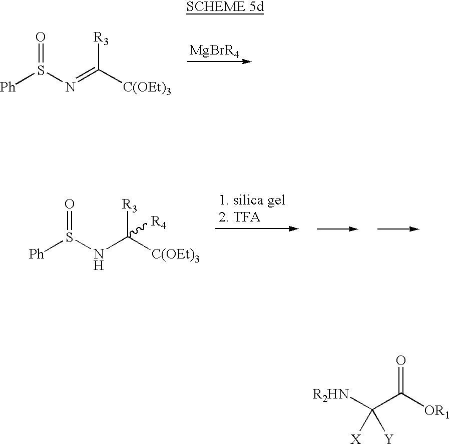

Synthesis of Compounds of Formula VI

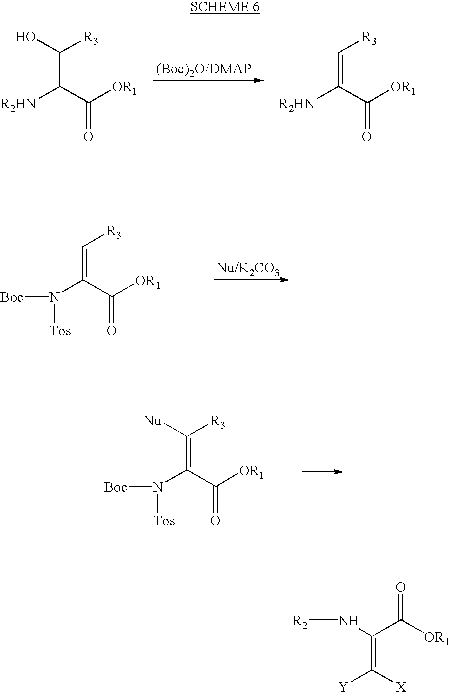

Mono or disubstituted dehydroamino derivatives can be synthesized from a substituted amino alcohol. Such amino alcohols are readily synthesized by one skilled in the art by methods similar to the procedures described earlier. Dehydration of the monosubstituted amino alcohol by (Boc)₂O/DMAP provides the dehydroamino derivative. Addition of nucleophiles (Nu) in the presence of base generates the disubstituted dehydroamino derivative

These steps may be varied to increase yield of desired product. The skilled artisan will also recognize the judicious choice of reactants, solvents and temperatures is an important component in successful synthesis. While the determination of optimal conditions, etc. is routine, it will be understood that to make a variety of compounds can be generated in a similar fashion, using the guidance of the schemes above.

It is recognized that the skilled artisan in the art of organic chemistry can readily carry out standard manipulations of organic compounds without further direction; that is, it is well within the scope and practice of the skilled artisan to carry out such manipulations. These include but are not limited to, reduction of carbonyl compounds to their corresponding alcohols, oxidations of hydroxyls and the like, acylations, aromatic substitutions, both electrophilic and nucleophilic, etherfications, esterfications and saponifications and the like. Example of these manipulations are discussed in standard texts such as March, Advanced Organic Chemistry(Wiley), Carey and Sundberg, Advanced Organic Chemistry(Vovl 2.) and Keeting, Heterocyclic Chemistry (all 17 volumes).

The skilled artisan will readily appreciate that certain reactions are best carried out when other functionality is masked or protected in the molecule, thus avoiding any undesirable side reactions and/or increasing the yield of the reactions. These reactions are found in the literature and are also well within the scope of the artisan. Examples of many of these manipulations can be found in T. Greene, Protecting Groups in Organic Synthesis. Of course, amino acids used as starting materials with reactive side chains are preferably blocked to prevent undesired side reactions.

TABLE I

| NAME | STRUCTURE |
|---|---|
| 2-aminobenzoate | 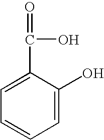 |
| 2-hydroxybutryrate | 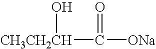 |
| 2-hydroxybutryrate | 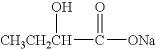 |
| 3-aminobenzoate | 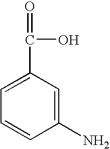 |
| 3-hydroxybutryrate | 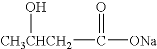 |
| 4-hydroxphenylpryuvate | 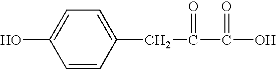 |
| Acetone dicarboxylate | 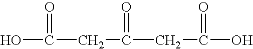 |
| Aminoguanidine bicarbonate | 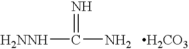 |
| Aminoguanidine HCL | 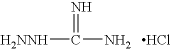 |
| Aminoguanidine hemisulfate | 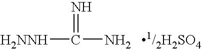 |
| Aminoguanidine nitrate | 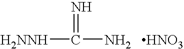 |
| Aminoguanidine sulfate | 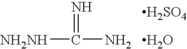 |
| Benzoic acid copper salt | 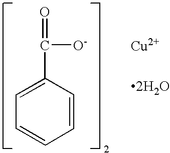 |
| Benzoic acid | 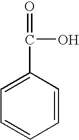 |
| Benzoylformic acid | 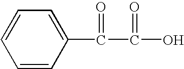 |
| cysteamine | H₂NCH₂CH₂SH |
| dansylchloride | 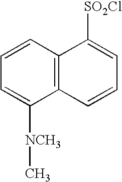 |
| dansylfluoride | 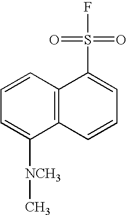 |
| dansylglycine | 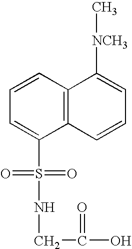 |
| diaminetetrazole | 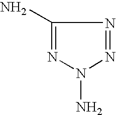 |
| dinitrophenylglycine | 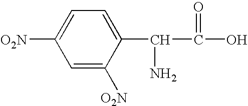 |
| d-leucine | 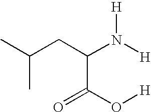 |
| DL-tartarate | 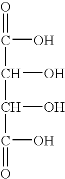 |
| d-malicacid | 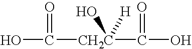 |
| D-tartarate | 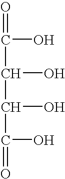 |
| Ethyl-2-picolinate | 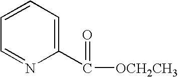 |
| ehtylanthranilate | 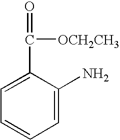 |
| ethylbromopyruvate | 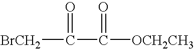 |
| ethylmethylnicotinate | 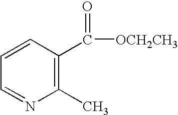 |
| HydroxylamineHCL | H₂NOH.HCl |
| Indole-3-acetaldehyde sodium bisulfite | 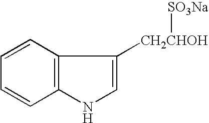 |
| Indole-3-acetamide | 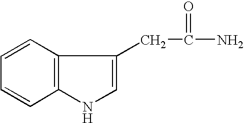 |
| Indole-3-acetate | 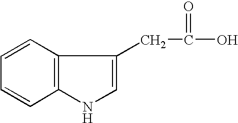 |
| Indole-3-aceticmethylester | 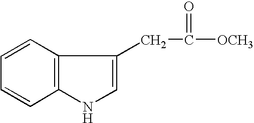 |
| Indole-3-acetone | 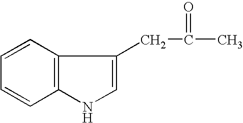 |
| Indole-3-acetylalanine | 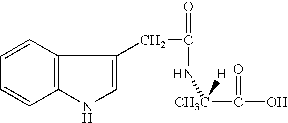 |
| Indole-3-acetyl ASP | 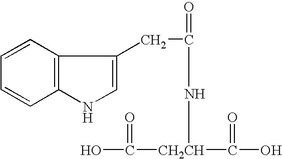 |
| Indole-3-carboxylate | 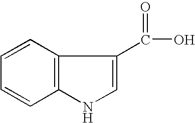 |
| Indole-3-propionate | 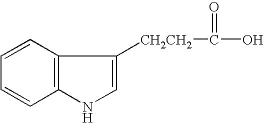 |
| Indole-3-pyruvate | 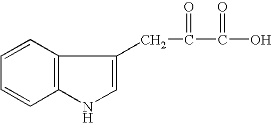 |
| Kiojic acid | 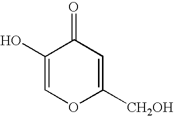 |
| L-leucine | 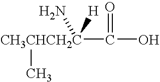 |
| L-leumethylester HCL | 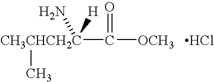 |
| L-malic acid | 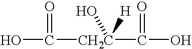 |
| Magnesium benzoate | 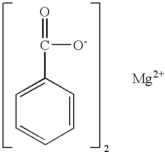 |
| methimazole | 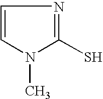 |
| Methyl-4-nitrobenzenesulfonate | 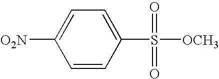 |
| methylanthranilate | 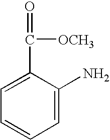 |
| methylbenzlthiocyanate | 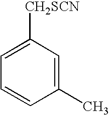 |
| methylmethylnicotinate | 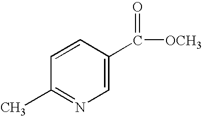 |
| methylpyruvate | 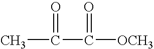 |
| methylsalicylate | 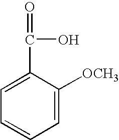 |
| methyluracil | 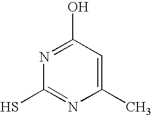 |
| MGAG diHCL | 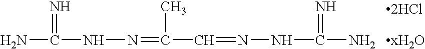 |
| N-acetyl-D-leucine | 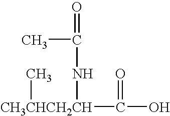 |
| N-acetyl-D-L-propargylglycine | 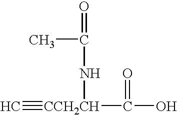 |
| Nitroethane | CH₃CH₂NO₂ |
| p-aminobenzoate | 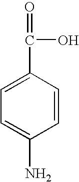 |
| Phenylglycoxal monohydrate | 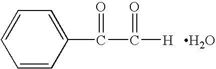 |
| phenylpyruvate | 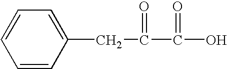 |
| Potassium tartrate | 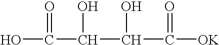 |
| progesterone | 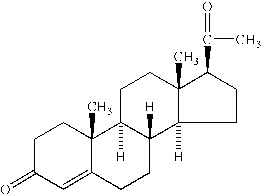 |
| proparglyglycine | 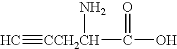 |
| pyruvate | 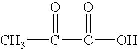 |
| RPP | 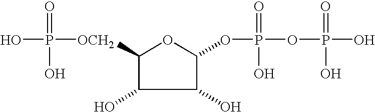 |
| salicylate | 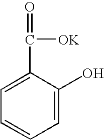 |
| Sodium benzoate | 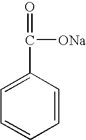 |
| Sodium alpha-ketoisovalerate | 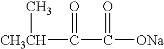 |
| Sodium phenylpyruvate | 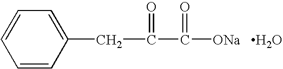 |
| Sodium pyruvate | 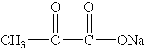 |
| Sodium salicylate | 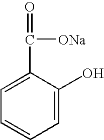 |
| Sodium sulfathiazole | 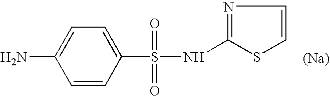 |
| Sulfathiazole | 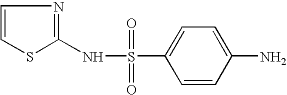 |
| thiosemicarbazide | 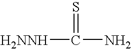 |
| thiourea | 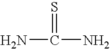 |
| Trigonelline | 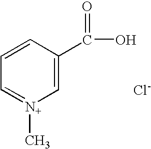 |
| Unsubstituted tetrazole | 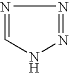 |

What is claimed:

1. A method of assessing a candidate molecule for the treatment of a CNS disorder, said method comprising:
a) providing a test DAO-inhibitor or DDO-inhibitor compound; and
b) administering said compound to an animal model of schizophrenia or bipolar disorder,
wherein a determination that said compound ameliorates a characteristic representative of a CNS disorder in said animal model indicates that said compound is a candidate molecule for the treatment of a CNS disorder; and alternatively one or more of the following:
i.) wherein said compound selectively bind to said polypeptide;
ii.) wherein said compound selectively inhibits the activity o f said polypeptide;
iii.) wherein said compound is capable of inhibiting the oxidation or degradation of a D-amino acid selected from the group consisting of D-Met, D-Pro, D-Phe, D-Tyr, D-Ile, D-Leu, D-Ala, D-Val, D-Ser, D-Arg, D-His, D-norleucine, D-Trp, D-Ornithine, cis-4-hydroxy-D-proline, D-Thr, D-Trp-methyl ester, N-acetyl-D-Ala, D-Lys, D-Asp, D-Glu, D-Asn, D-Gln, D-Asp-dimethyl-ester and N-methyl-D-Asp; and further alternatively wherein the compound of claim iii is capable of inhibiting the oxidation or degradation of D-serine.

2. The method of claim 1, wherein said test compound is selected from the group consisting of:
(1) a compound represented by the structure comprising: 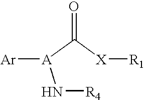
or a pharmaceutically acceptable salt thereof, wherein:
a) A is alkyl such as methyl, ethyl, propyl or butyl; branched chain alkyl such as isobutyl, isopropyl, isopentyl or cycloalkyl such as cyclopropyl, cyclopentyl or cyclohexyl. Such groups may themselves be substitued with C₁-C₆ alkyl, halo, hydroxyl or amino;
b) X is O or N;
c) Ar is an aromatic mono-, bi- or tricyclic fused heterocyclic ring, wherein the ring is either unsubstituted or substituted in one to five position(s) with hydrogen, halogen, hydroxyl, —CN, COR₂, —CONR₂R₃, —S(O)ₙR₂,—OPO(OR₂)OR₃, —PO(OR₃)R₃, OC(O)NR₂R₃, —COOR₂, —CONR₂R₃, —SO₃H, —NR₂R₃,—NR₂COR₃, —NR₃COOR₃, —SO₂NR₂R₃, —N(R₂)SO₂R₃, —NR₂CONR₂R₂, ——SO₂NHCOR₂, —CONHSO₂R₂, —SO₂NHCN, —OR₁, C₁-C₆ straight or branched chain alkyl or alkenyl, or C₁-C₆ branched or straight chain alkyl or alkenyl which is substituted with one or more, halogen, hydroxyl, amino, carboxy, carboxamide, nitrile, nitro, alkoxy, trifluoromethyl, sulfur, sulfonate, phosphonate, phosphate, Ar¹, N₃ or a combination thereof and wherein the heterocyclic ring contains 1-6 heteroatom(s) selected from the group consisting of O, N, S, and a combination thereof;
d) R₄ is H, alkyl, Ar¹, O, substituted alkyl;
e) R¹ is (C₁-C₆)alkyl, Ar¹, (C₁-C₄)alkoxycarbonylmethyl, substituted alkyl;
f) R₂ and R₃ are each, independently, hydrogen, C₁-C₆ straight or branched chain alkyl or alkenyl, or C₁-C₆ branched or straight chain alkyl or alkenyl which is substituted with one or more, halogen, hydroxyl, amino, carboxy, carboxamide, nitrile, nitro, alkoxy, trifluoromethyl, sulfur, sulfonate, phosphonate, phosphate, Ar¹, or N₃; and
g) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 3-7 members; and wherein the heterocyclic ring contains 1-6 heteroatom(s) selected from the group consisting of O, N, S, and a combination thereof;
(2) a compound represented by the structure comprising: 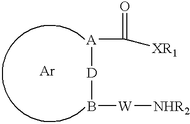
wherein:
a) A and B consist of C or N and D may contain 0-2 members consisting of C or N;
b) W is C₁-C₄ alkyl such as (CH₂)ₙ, branched chain alkyl;
c) n is 0-4. Further, when n=0 it is assumed that —NHR₂ is covalently bound to B;
d) X is O or N;
e) R₂ is H, alkyl, Ar¹, or O substituted alkyl;
f) R¹ is (C₁-C₆)alkyl Ar¹, (C₁-C₄)alkoxycarbonylmethyl, or substituted alkyl;
g) Ar is an aromatic mono-, bi- or tricyclic fused heterocyclic ring, wherein the ring is either unsubstituted or substituted in one to six position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, C₃-C₆ cycloalkyl or a combination thereof; wherein the individual ring sizes are 5-6 members; and wherein the heterocyclic ring contains 1-6 heteroatom(s) selected from the group consisting of O, N, S, and a combination thereof; and
h) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 3-7 members; and wherein the heterocyclic ring contains 1-6 heteroatom(s) selected from the group consisting of O, N, S, and a combination thereof; and
(3) a compound represented by the structure comprising: 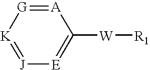
wherein:
a) A, G, K, J, E are members of a six membered carbo or heterocyclic aromatic ring, wherein the heterocyclic ring contains 1-6 heteroatom(s) selected from the group consisting of C, N and a combination thereof;
b) A, G, K, J, E may each independently be unsubstituted or substituted with hydrogen, halogen, hydroxyl, —CN, COR₂, —CONR₂R₃, —S(O)ₙR₂, —OPO(OR₂)OR₃, —PO(OR₃)R₃, —OC(O)NR₂R₃, —COOR₂, —CONR₂R₃, —SO₃H, —NR₂R₃, —NR₂COR₃, —NR₃COOR₃, —SO₂NR₂R₃, —N(R₂)SO₂R₃, —NR₂CONR₂R₂, —SO₂NHCOR₂, —CONHSO₂R₂, —SO₂NHCN, —OR₁, C₁-C₆ straight or branched chain alkyl or alkenyl, or C₁-C₆ branched or straight chain alkyl or alkenyl which is substituted with one or more halogen, hydroxyl, amino, carboxy, carboxamide, nitrile, nitro, alkoxy, trifluoromethyl, sulfur, sulfonate, phosphonate, phosphate, Ar¹, or N₃;
c) R₁ is CN, COR₂, —CONR₂R₃, —S(O)ₙR₂,—OPO(OR₂)OR₃, —PO(OR₃)R₃, —OC(O)NR₂R₃, —COOR₂, —CONR₂R₃, —SO₃H, —NR₂R₃, —NR₂COR₃, —NR₃ COOR₃, —SO₂NR₂R₃, —N(R₂)SO₂R₃, —NR₂CONR₂R₂, —SO₂NHCOR₂, —CONHSO₂R₂, —SO₂NHCN, SCN, COCO₂H, C₁-C₆ straight or branched chain alkyl or alkenyl, or C₁-C₆ branched or straight chain alkyl or alkenyl which is substituted with one or more halogen, hydroxyl, amino, carboxy, carboxamide, nitrile, nitro, alkoxy, trifluoromethyl, sulfur, sulfonate, phosphonate, phosphate, Ar¹, or N₃;
d) W is N, (CH₂)ₓ, or —NCH₂;
e) x=0-4;
f) n=0-2;
g) R₂ and R₃ are each, independently, hydrogen, C₁-C₆ straight or branched chain alkyl or alkenyl, or C₁-C₆ branched or straight chain alkyl or alkenyl which is substituted with one or more halogen, hydroxyl, amino, carboxy, carboxamide, nitrile, nitro, alkoxy, trifluoromethyl, sulfur, sulfonate, phosphonate, phosphate, Ar¹, or N₃; and
h) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 5-6 members; and wherein the heterocyclic ring contains 1-6 heteroatom(s) selected from the group consisting of O, N, S, and a combination thereof.

3. The method of claim 1, wherein said test compound is selected from the group consisting of
(1) a compound represented by the structure comprising: 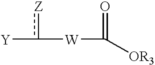
wherein:
a) W=(CH₂)ₙ
b) n=0-5;
c) Z is O or hydroxyl;
d) Y═H, Ar¹, R₄(CH₂)ₓ, R₁S(CH₂)ₓ—, R₁SO(CH₂)ₓ—, R₁SO₃(CH₂)ₓ—, HNR₁SO₂(CH₂)ₓ—, R₁R₂N(CH₂)ₓ, R₁O(CH₂)—, CF₃, or OH;
e) x=0-6;
f) R₁, R₂ and R₃ are each independently hydrogen, C₁-C₆ straight or branched chain alkyl or C₁-C₆ branched or straight chain alkyl substituted with one or more halogen, hydroxyl, amino, carboxy, carboxamide, nitrile, nitro, alkoxy, trifluoromethyl, sulfur, sulfonate, phosphonate, phosphate, or Ar¹;
g) R₄ is halogen, CN, N₃, C₁-C₆ straight or branched chain alkyl or C₁-C₆ branched or straight chain alkyl substituted with one or more halogen, hydroxyl, nitro, alkoxy, trifluoromethyl, sulfonate, phosphonate, phosphate, Ar¹, —COR₁, —COOR₁, —CONR₁R₂, CN,—NR₁, —NR₁R₂, —SR₁, —SO₂NHCN, or N₃; and
h) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 5-6 members; and wherein the heterocyclic ring contains 1-6 heteroatom(s) selected from the group consisting of O, N, S, and a combination thereof; and
(2) a compound represented by the structure comprising: 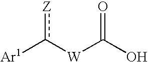
wherein:
a) Y is Ar¹;
b) Z is a carbonyl or hydroxyl;
c) W is (CH₂)ₙ wherein (n=0,1, or 2) and R₃═H; and
d) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 5-6 members; and wherein the heterocyclic ring contains 1-6 heteroatom(s) selected from the group consisting of O, N, S, and a combination thereof.

4. The method of claim 1, wherein said test compound is represented by the structure comprising: 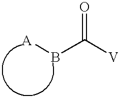
wherein:
a) A and B taken together, form a 5-8 membered saturated or partially unsaturated heterocyclic ring containing at least one additional O, S, SO, SO₂, NH, or NR¹ heteroatom in any chemically stable oxidation state;
b) V is O, OR₁, NR₂, NR₁R₂, CHR₁R₂, CH₂R₃, CHR₃R₄, or CH₂N₃;
c) R₁ and R₂ are independently hydrogen, C₁-C₆ straight or branched chain alkyl or C₁-C₆ branched or straight chain alkyl substituted with one or more halogen, hydroxyl, amino, carboxy, carboxamide, nitro, alkoxy, trifluoromethyl, sulfur, sulfonate, phosphonate, or Ar¹;
d) R₃ and R₄ are either halogen, C₁-C₆ straight or branched chain alkyl or C₁-C₆ branched or straight chain alkyl substituted with one or more hydroxyl, amino, carboxy, carboxamide, nitro, alkoxy, trifluoromethyl, sulfur, sulfonate, phosphonate, Ar¹, —OC(O)R₁, —COOR₁, —CONR₁R₂, CN, NR,, NR₁R₂, SR₁, SO₂NHCN, or N₃; and
e) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 5-6 members; and wherein the heterocyclic ring contains 1-6 heteroatom(s) selected from the group consisting of O, N, S, and a combination thereof.

5. The method of claim 4, wherein said compound is cystathionine ketimine or cyclothionine

6. The method of claim 1, wherein said test compound is represented by the structure comprising: 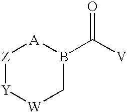
wherein:
a) W—Y-Z-A-B comprise a six membered saturated or partially saturated carbocyclic or heterocylic ring, wherein the heterocyclic ring contains heteroatom(s) selected from the group consisting of O, N, S, and any combination thereof;
b) B is either C, CH or N;
c) A, W,Y, Z are each independently CH₂, CHR₃, CR₃R₄, O, S, SO, SO₂, NH, NR₁, NR₁R₂, or C═O;
d) V is O, OR₁, NR₂, NR₁R₂, CHR₁R₂, CH₂R₃, CHR₃R₃, or CH₂N₃;
e) R₁ and R₂ are independently hydrogen, C₁-C₆ straight or branched chain alkyl or C₁-C₆ branched or straight chain alkyl substituted with one or more, halogen, hydroxyl, amino, carboxy, carboxamide, nitrile, nitro, alkoxy, trifluoromethyl, sulfur, sulfonate, phosphonate, phosphate, or Ar¹;
f) R₃ and R₄ are each independently halogen, —OC(O)R₁, —COOR₁, —CONR₁R₂, CN, —NR₁, —NR₁R₂, —SR₁, —SO₂NHCN, N₃, C₁-C₆ straight or branched chain alkyl or C₁-C₆ branched or straight chain alkyl substituted with one or more halogen, hydroxyl, nitro, alkoxy, trifluoromethyl, sulfonate, phosphonate, Ar¹, —OC(O)R₁, —COOR₁, —CONR₁R₂, CN, —NR₁, —NR₁R₂, —SR₁, —SO₂NHCN, or N₃; and
g) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 5-6 members; and wherein the heterocyclic ring contains 1-6 heteroatom(s) selected from the group consisting of O, N, S, and any combination thereof.

7. The method of claim 6, wherein said compound is selected from the group consisting of: Aminoethylcysteine-ketimine (2H-1,4-thiazine-5,6-dihydro-3-carboxylic acid), Thiomorpholine-2-carboxylic acid, Lanthionine ketimine, and 1,4-Thiomorpholine-3,5-dicarboxylic acid.

8. The method of claim 1, wherein said test compound is selected from the group consisting of
(1) a compound represented by the structure comprising: 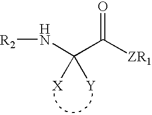
wherein:
a) Z is O or NH;
b) R¹ is (C₁-C₆)alkyl, Ar¹, or (C₁-C₄)alkoxycarbonylmethyl;
c) X, Y, independently of one another, are H, Ar¹, (C₁-C₆)alkyl (which can be interrupted or substituted by heteroatoms, such as N, P, O, S or Si, it being possible for the heteroatoms themselves to be substituted by (C₁-C₃)alkyl once or several times), (C₂-C₆)alkenyl, (C₁-C₆) haloalkyl,or halogen. When X and Y are each carbon they may be covalently joined to form a saturated or partially unsaturated carbocyclic compound of 3-8 members consisting independently of C, N, O, and S, further wherein ring members may themselves be unsubstituted or substituted with halo, hydroxyl, carboxy, nitro, trifluoromethyl, C₁-C₆ straight or branched chain alkyl or alkenyl, C₁-C₄ alkoxy, C₁-C₄ alkenyloxy, phenoxy, benzyloxy, amino, substituted alkyl, Ar¹, or a combination thereof;
d) R₂ is H, alkyl, Ar¹, or O substituted alkyl; and
e) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 3-7 members; and wherein the heterocyclic ring contains 1-6 heteroatom(s) selected from the group consisting of O, N, S, and any combination thereof;
(2) a compound represented by the structure comprising: 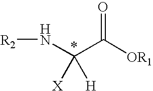
wherein:
a) *=asymmetric center and
b) R¹=(C₁-C₆)alkyl, Ar¹, (C₁-C₄)alkoxycarbonylmethyl and
c) X is H, (C₁-C₆)alkyl (which can be interrupted or substituted by heteroatoms, such as N, P, O, S or Si, it being possible for the heteroatoms themselves to be substituted by (C₁-C₃)alkyl once or several times), (C₂-C₆)alkenyl, (C₁-C₆) haloalkyl, halogen, or Ar¹;
d) R₂ is H, alkyl, Ar¹, or O substituted alkyl;
e) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 3-7 members; and wherein the heterocyclic ring contains 1-6 heteroatom(s) selected from the group consisting of O, N, S, and any combination thereof;
(3) a compound represented by the structure comprising: 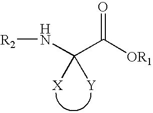
wherein:
a) X and Y are each carbon;
b) X and Y are connected by a saturated or partially saturated ring of 3-8 carbons and such a ring may itself be substituted in one to five position(s) with halo, hydroxyl, carboxy, amino, nitro, cyano, trifluoromethyl, C₁-C₆ straight or branched chain alkyl or alkenyl, C₁-C₄ alkoxy, C₁-C₄ alkenyloxy, or substituted alkyl groups;
c) R¹ is (C₁-C₆)alkyl, Ar¹, or (C₁-C₄)alkoxycarbonylmethyl;
d) R₂ is H, alkyl, Ar¹, or O substituted alkyl; and
e) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 3-7 members; and wherein the heterocyclic ring contains 1-6 heteroatom(s) selected from the group consisting of O, N, S, and any combination thereof; and
(4) a compound represented by the structure comprising: 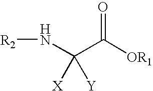
wherein:
a) X, Y, independently of one another, are H, Ar¹, (C₁-C₆)alkyl (which can be interrupted or substituted by heteroatoms, such as N, P, O, S or Si, it being possible for the heteroatoms themselves to be substituted by (C₁-C₃)alkyl once or several times), (C₂-C₆)alkenyl, (C₁-C₆) haloalkyl, or halogen such as naphthyl or phenyl;
b) R₂ is H, alkyl, Ar¹, or O substituted alkyl; and
c) Ar¹ is a mono-, bi- or tricyclic, carbo- or heterocyclic ring, wherein the ring is either unsubstituted or substituted in one to three position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, amino, or a combination thereof; wherein the individual ring sizes are 3-7 members; and wherein the heterocyclic ring contains 1-6 heteroatom(s) selected from the group consisting of O, N, S, and any combination thereof.

9. The method of claim 1, wherein said test compound is represented by the structure comprising: 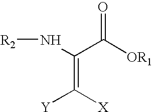
wherein:
a) R¹ is (C₁-C₆)alkyl, Ar¹, or (C₁-C₄)alkoxycarbonylmethyl;
b) R₂ is H, alkyl, Ar¹, or O substituted alkyl;
c) Y is H, Ar¹, (C₁-C₆)alkyl (which can be interrupted or substituted by heteroatoms, such as N, P, O, S or Si, it being possible for the heteroatoms themselves to be substituted by (C₁-C₃)alkyl once or several times), (C₂-C₆)alkenyl, (C₁-C₆) haloalkyl, or halogen; and
d) X is alkyl or phenyl.

10. A method of diagnosing, detecting a predisposition to or susceptibility to schizophrenia, depression or bipolar disorder in a subject, comprising
(a) obtaining a nucleic acid sample from said subject; and
(b) determining the identity of a nucleotide at a DAO-related polymorphism, or the complement thereof in said biological sample.

11. A isolated or purified nucleic acid encoding a DAO polypeptide or DAO polypeptide selected from the group consisting of:
(i) a nucleic acid molecule encoding a polypeptide comprising an amino acid sequence selected from the group of sequences consisting of SEQ ID NOS 8 to 10; and
(ii) a nucleic acid molecule comprising a nucleic acid sequence selected from the group of sequences consisting of SEQ ID NOS 1 to 6, or a sequence complementary thereto;
(iii) a purified or isolated DAO polypeptide comprising an amino acid sequence selected from the group of sequences consisting of SEQ ID NOS 8 to 10.
(iv) a polypeptide encoded by a nucleic acid molecule comprising a nucleic acid sequence selected from the group of sequences consisting of SEQ ID NOS 1 to 6, or a sequence complementary thereto.

12. The method of claim 1, wherein said test compound (i) binds to a DAO or DDO polypeptide, or (ii) inhibits the activity of a DAO or DDO polypeptide.

13. A method of identifying a candidate molecule for the treatment of a CNS disorder, said method comprising:
(a) contacting a DAO or DDO polypeptide or a biologically active fragment thereof with a test compound;
(b) determining whether said compound (i) binds to said polypeptide, or (ii) inhibits the activity of said polypeptide; and
(c) if said compound binds to said polypeptide or inhibits said polypeptide, administering said compound to an animal model of schizophrenia, depression or bipolar disorder,
wherein a determination that said compound ameliorates a characteristic representative of CNS disorder in said animal model indicates that said compound is a candidate molecule for the treatment of a CNS disorder.
